(19) United States
(12) Patent Application Publication
(10) Pub. No.: US 2009/0324617 A1
(43) Pub. Date: Dec. 31, 2009

(54) Cancer specific glycans and use thereof

(75) Inventors: Tero Satomaa, Helsinki (FI); Jari Natunen, Vantaa (FI); Annamari Heiskanen, Helsinki (FI); Anne Olonen, Lahti (FI); Noora Salovuori, Helsinki (FI); Juhani Saarinen, Helsinki (FI); Jari Helin, Vantaa (FI)

Correspondence Address: BIRCH STEWART KOLASCH & BIRCH, PO BOX 747, FALLS CHURCH, VA, 22040-0747, US

(73) Assignee: Glykos Finland Ltd., Helsinki (FI)

(21) Appl. No.: 12/320,297

(22) Filed: Jan. 22, 2009

Related U.S. Application Data
(60) Provisional application No. 61/006,641, filed on Jan. 24, 2008.

(51) Int. Cl. A61K 39/395; C12Q 1/02; G01N 33/53; C07H 15/00; A61K 39/00; A61P 35/00

(52) U.S. Cl. 424/174.1; 435/29; 435/7.1; 536/123.1; 424/277.1

(57) ABSTRACT

The present invention describes glycans, which are specifically expressed by certain cancer cells, tumours and other malignant tissues. The present invention describes methods to detect cancer specific glycans as well as methods for the production of reagents binding to said glycans. The invention is also directed to the use of said glycans and reagents binding to them for the diagnostics of cancer and malignancies. Furthermore, the invention is directed to the use of said glycans and reagents binding to them for the treatment of cancer and malignancies. Moreover, the present invention comprises efficient methods to differentiate between malignant and benign tumors by analyzing glycan structures.

### RELATED APPLICATIONS

This Nonprovisional application claims priority under 35 U.S.C. § 119(e) on U.S. Provisional Application No. 61/006,641 filed on Jan. 24, 2008, the entire contents of which is hereby incorporated by reference into the present application.

### FIELD OF THE INVENTION

The present invention relates to glycans, which are specifically expressed by certain cancer cells, tumours and other malignant tissues. The present invention describes methods to detect cancer specific glycans as well as methods for the production of reagents binding to said glycans. The invention is also directed to the use of said glycans and reagents binding to them for the diagnostics of cancer and malignancies. Furthermore, the invention is directed to the use of said glycans and reagents binding to them for the treatment of cancer and malignancies.

### SUMMARY OF THE INVENTION

It is generally acknowledged that cancerous transformation of human tissues is associated with changes in the complex carbohydrate structures, glycans, which are elementary components of the glycoproteins, glycolipids, and proteoglycans that cover all human cell surfaces. Several individual glycan molecular structures have been identified as cancer-associated glycans (Dube & Bertozzi, 2005). These glycan structures have also been pursued as molecular drug targets for treatment of malignant breast cancer (Holmberg & Sandmeier, 2001) and melanoma (Fernandez, 2003). However, the whole spectrum of cancer-associated glycan changes has remained unknown because of lack of suitable analysis technology. The present invention is directed to such novel analytical methods and the application of the methods to analyses of tissue samples from human patients. The methods are applicable to clinical cancer diagnostics. The novel cancer-associated molecules discovered by the inventors are targets for cancer therapy and diagnostics.

The present invention reveals novel methods for producing and analyzing novel carbohydrate compositions, glycomes, from tissues. A preferred use of the present invention is analysis of cancer-associated glycan structures and glycan profiles. The invention further represents methods for analysis of the glycomes, especially mass spectrometric methods, and diagnostic methods thereof to detect cancer. As demonstrated in the Examples of the present invention, the inventors found novel methods to efficiently discriminate between cancerous and healthy tissue samples as well as malignant and benign tumors by mass spectrometric glycome analysis of tissue materials extracted from human patients.

The present invention reveals novel glycosylation features and glycan molecular groups generally associated with major human cancer types. The present invention is especially directed to novel glycosylation features of lung cancer, especially non-small cell lung adenocarcinoma, breast cancer, especially ductale and lobulare breast adenocarcinoma, colon carcinoma, especially colon carcinoma adenomatosum, kidney cancer, especially kidney carcinoma and hypemephroma, ovarian cancer, especially ovarian cystadenocarcinoma, gastric cancer, liver cancer, pancreas cancer, and larynx cancer. The present invention is further directed to novel glycosylation features of benign and malignant human tumors and the discrimination between benign and malignant growth, especially ovarian cystadenoma and ovarian cystadenocarcinoma, and colon adenoma and colon carcinoma adenomatosum. In another embodiment, the present invention is further directed to novel cancer type specific glycosylation features and their use for detection of specific cancer types from tissue materials.

The tissue substrate materials can be total tissue samples and fractionated tissue parts, such as serums, secretions and isolated differentiated cells from the tissues, or artificial models of tissues such as cultivated cell lines. In a preferred embodiment the invention is directed to special methods for the analysis of the surfaces of tissues. The invention is further directed to the compositions and compositions produced by the methods according to the invention, and cancer treatment methods derived thereof.

The invention represents effective methods for purification of glycan fractions from tissues, preferably from animal tissues, and more preferably from human and mammalian tissues, especially in very low scale. The prior art has shown analysis of separate glycome components from tissues, but not total glycomes. It is further realized that the methods according to the invention are useful for analysis of glycans from isolated proteins or peptides. The invention represents effective methods for the practical analysis of glycans from isolated proteins especially from very small amounts of samples.

The invention is further directed to novel quantitative analysis methods for glycomes. Typical glycomes comprise of subgroups of glycans, including N-glycans, O-glycans, glycolipid glycans, and neutral and acidic subglycomes. The glycome analysis produces large amounts of data. The invention reveals methods for the analysis of such data quantitatively and comparison of the data between different samples. The invention is especially directed to quantitative two-dimensional representation of the data and generation of reference data from different clinical states of tissue materials to detect disease-associated changes in glycosylation. The invention is further directed to simultaneous analysis of multiple cancer-associated glycan changes to detect cancer or clinical state of cancer.

The preferred analysis method includes:
1) Preparing a tissue sample containing glycans for the analysis
2) Releasing total glycans or total glycan groups from a tissue sample, or extracting free glycans from a tissue sample
3) Optionally modifying glycans
4) Purification of the glycan fraction/fractions from biological material of the sample
5) Optionally modifying glycans
6) Analysis of the composition of the released glycans preferably by mass spectrometry
7a) Optionally presenting the data about released glycans quantitatively and
7b) Comparing the quantitative data set with another data set from another tissue sample or
8) Comparing data about the released glycans quantitatively or qualitatively with data produced from another tissue sample.

The invention is further directed to structural analysis of glycan mixtures present in tissue samples and using the cancer-associated glycan structures in cancer therapy. Preferred forms of cancer therapy according to the present invention include glycan specific antibodies and cancer vaccines for passive or active immunotherapy against the cancer-associated glycan molecules, respectively.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1. Example of α-mannosidase digestion schemes of, A. healthy lung and lung cancer tumor sample pairs from 7 patients with non-small cell lung adenocarcinoma, and B. breast cancer tumor samples from 7 patients with ductale breast adenocarcinoma. The m/z figures in the x-axis correspond to the approximate m/z values of [M+Na]⁺ adduct ions. The figures in the y-axis correspond to relative glycan signal intensities. The difference between glycan intensities before and after α-mannosidase digestion is equal to the amount of non-reducing terminal α-mannose containing structures.

FIG. 2. Structural features of Structure group 1 glycans, namely A. non-fucosylated glycans Manα₀₋₃Manβ4GlcNAcβ4GlcNAc(β-N-Asn), and B. fucosylated glycans Manα₀₋₄Manβ4GlcNAcβ4(Fucα6)GlcNAc(β-N-Asn).

FIG. 3. Example of mass spectrometric analysis of glycans expressed in tumor and healthy control tissues of a non-small cell lung adenocarcinoma patient. A. Neutral glycan mass spectrum from the lung cancer tumor, B. neutral glycan mass spectrum from healthy lung tissue from the same patient, and C. illustration of the differences in spectra A. and B, with regard to the present structure group glycans. The m/z figures in the x-axis correspond to the approximate m/z values of [M+Na]⁺ adduct ions. The figures in the y-axis correspond to relative glycan signal intensities.

FIG. 4. Graph presenting the average relative glycan signal intensities in breast cancer tumor-healthy breast tissue sample pairs from 9 patients with ductale breast adenocarcinoma. The m/z figures in the x-axis correspond to the approximate m/z values of [M+Na]⁺ adduct ions. The figures in the y-axis correspond to relative glycan signal intensities.

FIG. 5. Graph presenting the average relative glycan signal intensities in breast cancer tumor-healthy breast tissue sample pairs from 7 patients with lobulare breast adenocarcinoma. The m/z figures in the x-axis correspond to the approximate m/z values of [M+Na]⁺ adduct ions. The figures in the y-axis correspond to relative glycan signal intensities.

FIG. 6. Example of α-mannosidase digestion schemes of healthy lung and lung cancer tumor sample pairs from 7 patients with non-small cell lung adenocarcinoma. The m/z figures in the x-axis correspond to the approximate m/z values of [M+Na]⁺ adduct ions. The figures in the y-axis correspond to relative glycan signal intensities. The glycan signal intensities that remain after α-mannosidase digestion are indicative of the presence of α-mannosidase resistant structures.

FIG. 7. Structural features of Structure group 2 glycans, including a) Core 2 type O-glycan structures, and b) extended Core 1 type O-glycan structures.

FIG. 8. Example of mass spectrometric analysis of glycans expressed in tumor and healthy control tissues of a lung cancer patient. A. Neutral glycan sample from non-small cell lung adenocarcinoma. B. Neutral glycan sample from healthy lung from the same patient. The m/z figures in the x-axis correspond to the approximate m/z values of [M+Na]⁺ adduct ions. The figures in the y-axis correspond to relative glycan signal intensities.

FIG. 9. Graph presenting the average glycan signal intensities in breast cancer tumor-healthy breast tissue sample pairs from 9 patients with ductale breast adenocarcinoma. The m/z figures in the x-axis correspond to the approximate m/z values of [M+Na]⁺ adduct ions. The figures in the y-axis correspond to relative glycan signal intensities.

FIG. 10. Graph presenting the average glycan signal intensities in breast cancer tumor-healthy breast tissue sample pairs from 7 patients with lobulare breast adenocarcinoma. The m/z figures in the x-axis correspond to the approximate m/z values of [M+Na]⁺ adduct ions. The figures in the y-axis correspond to relative glycan signal intensities.

FIG. 11. The structure of the O-glycan fragment Neu5Acα3Galβ4GlcNAcβ6(2-acetamido-3-amino-2,3-dideoxy)hexose, and experiments done to characterize the structure.

FIG. 12. MALDI-TOF mass spectrometric fragmentation analysis (PSD) of: A. deuteroacetylated m/z 899 [M+Na]⁺ glycan fragment at m/z 944, and B. deuteroacetylated m/z 608 [M+Na]⁺ glycan fragment at m/z 653. The m/z figures in the x-axis correspond to the approximate m/z values of either [M+Na]⁺ or [M+H]⁺ adduct ions. The figures in the y-axis correspond to relative glycan signal intensities. The nomenclature of the fragment ions is after Domon and Costello (1988).

FIG. 13. Example MALDI-TOF mass spectra from tumor tissue samples of a non-small cell lung adenocarcinoma patient. A. Positive-ion mode mass spectrum of the neutral glycan fraction, B. negative-ion mode mass spectrum of the neutral glycan fraction, and C. negative-ion mode mass spectrum of the sialylated glycan fraction. The m/z figures in the x-axis correspond to the approximate m/z values of either [M+Na]⁺ adduct ions (A.) or [M−H]⁻ ions (B. and C.). The figures in the y-axis correspond to relative glycan signal intensities.

FIG. 14. Example of MALDI-TOF mass spectrometric analysis of glycans expressed in tumor and healthy control tissues of patients with either malignant ovarian cystadenocarcinoma (A. and C.) or benign ovarian cystadenoma (B. and D.). A. and B. Negative-ion mode mass spectra of the sialylated glycan fractions. C. and D. Positive-ion mode mass spectra of the neutral glycan fractions. The m/z figures in the x-axis correspond to the approximate m/z values of either [M−H]⁻ ions (A. and B.) or [M+Na]⁺ adduct ions (C. and D.). The figures in the y-axis correspond to relative glycan signal intensities.

FIG. 15. Example of mass spectrometric analysis of glycans expressed in tumor and healthy control tissues of 9 ductale breast cancer patients. The m/z figure in the x-axis corresponds to the [M+Na]⁺ adduct ion with an approximate m/z value of 899. The figures in the y-axis correspond to relative glycan signal intensities in 1/1000.

FIG. 16. Example of mass spectrometric analysis of glycans expressed in tumor and healthy control tissues of 7 lobulare breast cancer patients. The m/z figure in the x-axis corresponds to the [M+Na]⁺ adduct ion with an approximate m/z value of 899. The figures in the y-axis correspond to relative glycan signal intensities in 1/1000.

FIG. 17. Neutral protein-linked glycan profiles from non-small cell lung adenocarcinoma patients. x-axis: approximate mass-to-charge ratio (m/z) of the glycan signals, corresponding to mass of [M+Na]⁺ ions; y-axis: relative abundance of the glycan within the neutral glycan fraction. Light columns: healthy lung; Dark columns: non-small cell lung adenocarcinoma.

FIG. 18. MALDI-TOF mass spectrometric analysis of sialylated high-molecular weight protein-linked glycans from A. normal ovary tissue, B. benign ovarian cystadenoma tumor, and C. malignant ovarian cystadenocarcinoma tumor. x-axis: mass-to-charge ratio (m/z) of the detected signals at 1500-4000 Da, corresponding to mass of [M−H]⁻ ions; y-axis: relative signal intensity, 0-100%; N: [M−2H+Na]⁻ ion at +22 Da; K: [M−2H+ K]⁻ ion at +38 Da; asterisk: H₂O elimination product at −18 Da; numbering of glycan signals refers to Table 8.

FIG. 19. Neutral protein-linked glycan profiles from A. normal ovary tissue (1 patient), B. benign ovarian cystadenoma tumor (average of 5 patients), and C. malignant ovarian cystadenocarcinoma tumor (average of 4 patients). x-axis: approximate mass-to-charge ratio (m/z) of the glycan signals, corresponding to mass of [M+Na]⁺ ions; y-axis: relative abundance of the glycan within the neutral glycan fraction. Column code from left to right, dark columns: normal ovary; blank columns: benign tumor; light columns: malignant tumor.

FIG. 20. MALDI-TOF post-source decay (PSD) mass spectrometric fragmentation analysis of two isomeric glycan structures (I and II) present in a major sialylated glycan signal of ovarian tumors and normal ovarian tissue. The parent ion corresponds to the sodium adduct ion of permethylated NeuAc₁Hex₄HexNAc₅dHex₁. The fragment ions appear as sodium adduct ions, except for m/z 376.5 that is apparently protonated, and they are abbreviated as described by Domon and Costello (1988). The evidence suggests that structure I carries a non-reducing terminal HexNAc-HexNAc sequence whereas structure II carries a non-reducing terminal NeuAc-HexNAc-HexNAc sequence.

FIG. 21. Discrimination analysis of neutral protein-linked glycans analysis results of malignant and benign tumors and healthy tissue samples from the same tissues of A. breast, B. ovary, and C. colon. The discrimination is based on relative abundances of three neutral protein-linked glycans identified in principal component analysis and an experimental discrimination formula derived from a randomly picked training group of breast cancer patients, as described in the text. The scores resulting from individual samples are plotted on the y-axis.

FIG. 22. Example of glycan signal analysis of MALDI-TOF mass spectrometric data. A. Mass spectrometric raw data showing a window of neutral N-glycan mass spectrum in positive ion mode, B. Glycan profile generated from the data in A.

FIG. 23. Example of glycan signal analysis of MALDI-TOF mass spectrometric data. A. Mass spectrometric raw data showing a window of sialylated N-glycan mass spectrum in negative ion mode, B. Glycan profile generated from the data in A.

FIG. 24. Neutral protein-linked glycans of normal lung tissue (light columns) and lung cancer tumor (dark columns).

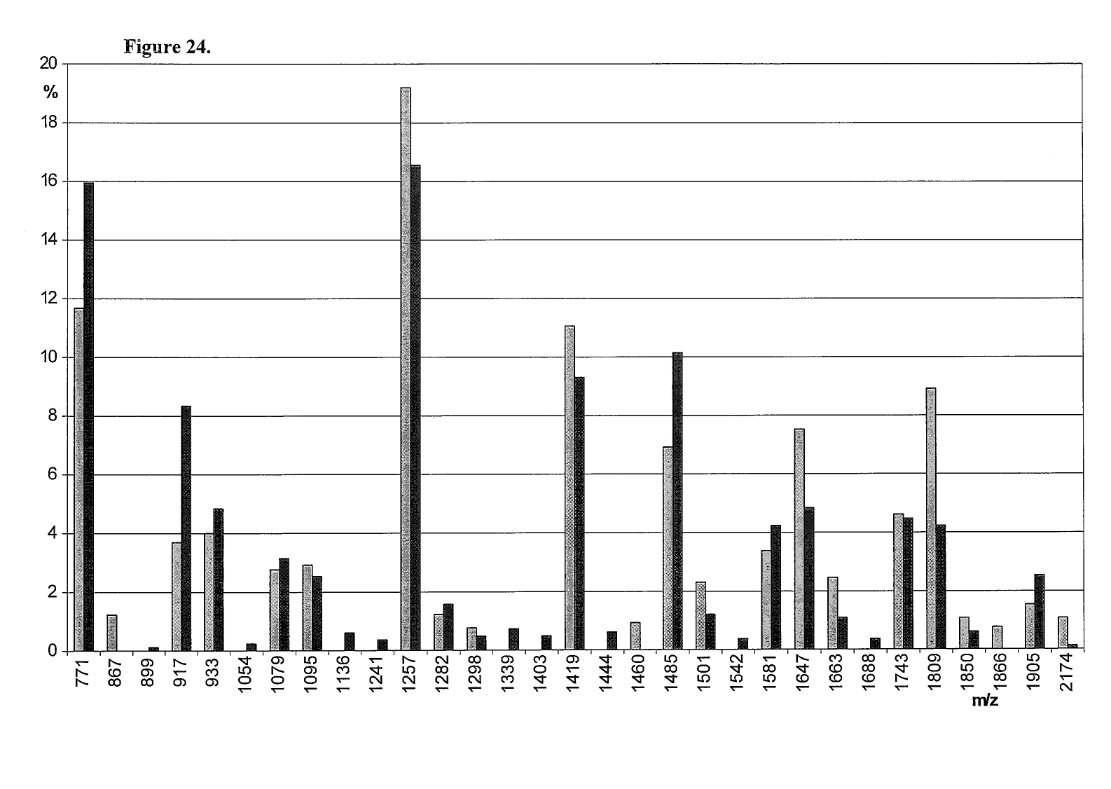

FIG. 25. Neutral and acidic N-glycan profiles of lysosomal protein sample.

FIG. 26. Reference neutral N-glycan structures for NMR analysis (A-C) in Table 11.

FIG. 27. Reference neutral N-glycan structures for NMR analysis (D-G) in Table 12.

FIG. 28. Reference acidic N-glycan structures for NMR analysis (A-E) in Table 13.

FIG. 29. Neutral protein-linked glycans of normal breast tissue (light columns) and ductale type breast carcinoma tumor (dark columns) from the same patient.

FIG. 30. Neutral protein-linked glycans of normal lymph node tissue (light columns) and ductale type breast carcinoma lymph node metastasis (dark columns) from the same patient.

FIG. 31. Acidic protein-linked glycans of normal breast tissue (light columns) and ductale type breast carcinoma tumor (dark columns) from the same patient.

FIG. 32. Acidic protein-linked glycans of normal lymph node tissue (light columns) and ductale type breast carcinoma lymph node metastasis (dark columns) from the same patient.

FIG. 33. Periodate oxidation analysis of cancer derived neutral N-glycans.

FIG. 34. Periodate oxidation analysis of cancer derived low-mannose type N-glycan.

### DETAILED DESCRIPTION OF THE INVENTION

### Analysis of Cancer Glycomes

The present invention revealed that quantitative analysis of human glycomes is useful for analysis of human cancers. The glycome analysis revealed profiles of glycan expression, which can be used for the analysis of glycomes even without knowledge of exact structures of glycans. The present invention is directed in a preferred embodiment quantitative mass spectrometric profiling of human cancers according to the invention and analysis of alterations in cancer in comparison with normal corresponding normal tissues. The analysis can be performed based on signals corresponding to glycan structures, these signals were translated to likely monosaccharide compositions and further analysed to reveal structures and correlations between the signals. The invention is especially directed to analysis of N-glycan and/or O-glycan glycomes derived from cancer proteins. The glycans are analysed as neutral and/or acidic signals and glycan mixtures, multiple analysis methods are preferred to obtain maximal amount of data. The invention is also directed to methods for analysis of mixture of N-glycans and O-glycans released together.

### O-Glycan, Mannose-N-Glycans and Terminal HexNAc-Glycans as Cancer Targets

The invention is directed to a method of evaluating the malignancy of a patient sample comprising the step of detecting the presence or amount of cancer related glycan structures in the sample when these comprise specific O-glycan and/or terminal Man-N-glycan and/or terminal HexNAc glycans including terminal GlcNAc-glycans and lacdiNAc-glycans according to the invention. in a preferred embodiment at least the O-glycan is analyzed, in a second preferred embodiment at least the N-glycans is analyzed and in third preferred embodiment the terminal HexNAc-structure is analyzed. In yet another preferred embodiment the 2 of the glycans structures are analyzed including i) O-glycan and terminal Man N-glycan ii) O-glycans and terminal HexNAc structure iii) terminal Man N-glycan and terminal HexNAc structure.

The invention revealed two major methods for recognition of the structures. The is preferably performed by
i) recognition of a terminal glycan epitope of the glycan by a specific binder reagent preferably by a monoclonal antibody and/or
ii) by recognition of a characteristic monosaccharide composition corresponding to the glycan structures by chemical methods, preferably by mass spectrometry and optionally further with specific binder molecule.

It is realized that the mass spectrometric methods recognizing glycan compositions are effective screening methods and in a preferred embodiment the mass spectrometric method is confirmed by specific binder recognition such as by monoclonal antibody.

### Use of Binder Molecules

The invention is especially directed the recognition of the glycans, wherein the glycan is recognized by a binder molecule specific for a terminal non-reducing end structure of the glycan comprising at least two monosaccharide residues and glycosidic linkage between them. More preferably at least two monosaccharide residues and two linkages are recognized

The invention is directed to the use of the binder molecules for analysis or diagnosis of cancer and/or for treatment of cancer.

The binder is preferably antibody, more preferably monoclonal antibody or fragment or derivative thereof. Other preferred binders includes glycosyltransferring enzymes such as glycosidases or glycosyltransferases, β-galactosyltransferase is especially preferred for the recognition of terminal GlcNAc structures.

The invention is especially directed to glycan marks, wherein glycan is expressed in binder accessible form in cancer or on human cancer. For therapy or the in vivo targeting the binder accessible form is a cell surface form or form accessible from necrotic cancer cells in vivo in the cancer tissue. In a preferred embodiment the invention is directed antigens present on the surface of living cancer cells. In the embodiment of targeting necrotic cancer cells the invention is directed to use of radiolabelled or other longer range destructive conjugates of the binder molecules, therapies directing to markers present in or on necrotic cancer cells are known in the art. The invention is further directed to in vitro diagnostic methods recognizing the glycans present in cell bound form on cell surfaces or in another embodiment intracellularly. It is realized that in vitro diagnostic can be also targeted to intracellular markers. It is realized that the glycosylations of secreted protein in background differ from cell associated glycosylations.

The present invention is especially directed to human cancers and specific human cancer subindications according to the invention. It is realized that background from other species is not relevant.

In a separate embodiment the invention is directed to secreted cancer antigens and their analysis from body fluids such as serum, urine, spinal cord solutions, saliva, bronchial secretions, breast secretions, or mucins secreted to feces.

In a preferred embodiment the glycan is expressed in cancer metastasis and the binder is preferably antibody, more preferably monoclonal antibody or fragment or derivative thereof.

### Preferred N-Acetyllactosamine Comprising O-Glycans

The preferred O-glycans comprise N-acetyllactosamine epitope GalβGlcNAc including type 1 Galβ3GlcNAc and type II Galβ4GlcNAc epitopes. In a preferred embodiment the O-glycan comprising N-acetyllactosamine Galβ4GlcNAc β6 or β3-linked to O-glycan core structure, with the proviso that when the structures is core 2 sialyl-Lewis x the cancer is not primary colon cancer, or when the structure is Galβ4GlcNAcβ6(Galβ3)GalNAcα or Galβ4GlcNAcβ6GalNAcα, then the cancer is not primary ovarian cancer?? and optionally

The invention reveled major type of core II O-glycans present in human cancers and tumors The preferred O-glycans includes sialylated or neutral O-glycan structures according to the Formula O1:

[SAαX₁]ₙ₄{Galβ4[(Fucα3)]ₙ₁GlcNAcβX₂}ₘGalβ4[(Fucα3)]ₙ₂GlcNAcβ6([SAαX₃]ₙ₃Galβ3) GalNAc[αSer/Thr]ₚ

wherein n1, n2, n3 and n4 and p are either independently 0 or 1 and m is 0, 1 or 2;

### X₁, X₂, and X₃ are 3 or 6;

SA is sialic acid (preferably Neu5Ac) or in a separate embodiment alternatively SAαX is A1 as defined in Formula BL
{ } and [ ] indicate determinant either being present or absent depending on the value of m, n1, n2, and n3; and
( ) indicates a branch in the structure.
Woptionally with the provisio that when n4, n2 are 1 and m is 0, structure being NeuNAcα3Galβ4(Fucα3)GlcNAcβ6(Galβ3)GalNAcαSer/Thr, then the cancer is not primary colon cancer and optionally with the provisio that when m, n4, n3, n2 and p are 0, structure being Galβ4GlcNAcβ6(Galβ3)GalNAcα or Galβ4GlcNAcβ6GalNAcα, then the cancer is not primary ovarian cancer?

### Preferred O-Glycan Structures and Epitopes

The invention is preferably directed to O-glycans including

a) neutral O-glycan oligosaccharide sequence has a structure of Formula:

Galβ4[(Fucα3)]ₙGlcNAcβX[(]ₘGalβ3[)]ₘGalNAc[αSer/Thr]ₚ

wherein p, n and m are either independently 0 or 1, [ ] indicates determinant either being present or absent depending on the value of m and n, ( ) indicates a branch in the structure. X is 3, when m is 0; and X is 6 when m is 1.
b) sialylated O-glycan has a structure of Formula:

SAαX₁{Galβ4[(Fucα3)]ₙ₁GlcNAcβX₂}ₘGalβ4[(Fucα3)]ₙ₂GlcNAcβ6([SAαX₃]ₙ₃Galβ3) GalNAc[αSer/Thr]ₚ

wherein n1, n2, n3 and p are either independently 0 or 1 and m is 0, 1 or 2;

### X₁, X₂, and X₃ are 3 or 6;

{ } and [ ] indicate determinant either being present or absent depending on the value of m, n1 and n2; and
( ) indicates a branch in the structure,
Optionally with the provisio that when n2 is 1 and m is 0 then the cancer is not primary colon cancer
and X₂, and X₃ are preferably 3; X₁ is preferably 3, when n1 is 1; in preferred embodiment all X₁, X₂, and X₃ are 3.

The preferred shorter terminal core II comprising GalβGlcNAc has a structure selected from the group: Galβ4(Fucα3)GlcNAcβ6(Galβ3)GalNAc, Galβ4GlcNAcβ6(Galβ3)GalNAc, Galβ4(Fucα3)GlcNAcβ6GalNAc, and Galβ4GlcNAcβ6GalNAc and
Galβ4(Fucα3)GlcNAcβ6(Galβ3)GalNAcαSer/Thr,
Galβ4GlcNAcβ6(Galβ3)GalNAcαSer/Thr, Galβ4(Fucα3)GlcNAcβ6GalNAcαSer/Thr, and Galβ4GlcNAcβ6GalNAcαSer/Thr.
   and
NeuNAcα3Galβ4(Fucα3)GlcNAcβ6(Galβ3)GalNAc,
NeuNAcα3/6Galβ4GlcNAcβ6(Galβ3)GalNAc, NeuNAcα3Galβ4(Fucα3)GlcNAcβ6GalNAc, and NeuNAcα3/6Galβ4GlcNAcβ6GalNAc and
NeuNAcα3Galβ4(Fucα3)GlcNAcβ6(Galβ3)GalNAcαSer/Thr,
NeuNAcα3/6Galβ4GlcNAcβ6(Galβ3)GalNAcαSer/Thr,
NeuNAcα3Galβ4(Fucα3)GlcNAcβ6GalNAcαSer/Thr, and
NeuNAcα3/6Galβ4GlcNAcβ6GalNAcαSer/Thr.

### The Preferred Terminal Epitopes of O-Glycans:

The invention revealed useful terminal epitopes to be recognized on O-glycans by the specific binder molecules for the analysis, therapy and targeting according to the invention. It is relized that varying levels of specificity of the binder is needed depending on background (healthy) glycosylation present in a normal tissue(s), which may interfere the process.

### Preferred General Terminal Epitope

The invention is directed to binder of the cancer O-glycans wherein the binder recognizes at least terminal epitope according to the formula TE1

[SAαX₁]ₙ₄Galβ4[(Fucα3)]ₙ₂GlcNAc{β3Galβ[(Fucα3)]_{n1[}4GlcNAc]ₙ₅}m{β6[GalNAc]ₙ₆}ₙ₇

wherein n1, n2, n4, n5, n6 and n7 and p are either independently 0 or 1 and m is 0, 1 or 2 (repeating structure);

### X₁, X₂, and X₃ are 3 or 6;

SA is sialic acid (preferably Neu5Ac) or in a separate embodiment alternatively SAαX is A1 as defined in Formula BL
{ } and [ ] indicate determinant either being present or absent depending on the value of m,
n1, n2, n4, n5, n6 and n7; and
( ) indicates a branch in the structure.
Optionally with the provisio that when n4, n2 are 1 and m is 0, and X is 3, then the cancer is not primary colon cancer.

The invention is directed to binder of the cancer O-glycans, wherein the terminal epitope recognized includes at least structure according to the formula TE2 [SAαX₁]ₙ₄Galβ4[(Fucα3)]ₙ₂GlcNAc[β]ₙ₈, wherein n8 is either 1 or 0 and other variables are as in Formula TE1. This is most general binder type, which is preferably optimized not to recognize GlcNAcβMan-structures such as GlcNAcβ2/4/6Man and/or (preferably and) glycolipid or N-glycan Lactosamine structures.

The invention is directed to binder of the cancer O-glycans, wherein the terminal epitope recognized includes at least structure according to the formula TE3 [SAαX₁]ₙ₄Galβ4[(Fucα3)]ₙ₂GlcNAcβ6[GalNAc]ₙ₆, wherein variables are as in Formula TE1. This group includes preferred recognition of the specific β6-linkage and optionally at least part of GalNAc. This is preferably optimized not to recognize GlcNAcβMan-structures such as GlcNAcβ6Man and/or (preferably and) glycolipid or N-glycan Lactosamine structures.

The preferred terminal target epitopes according to TE3 includes terminal epitopes selected from the group
Galβ4GlcNAcβ6, Galβ4GlcNAcβ6GalNAc,
Galβ4(Fucα3)GlcNAcβ6, Galβ4(Fucα3)GlcNAcβ6GalNAc, and
NeuNAcα3Galβ4GlcNAcβ6, NeuNAcα3Galβ4GlcNAcβ6GalNAc,
NeuNAcα3Galβ4(Fucα3)GlcNAcβ6, NeuNAcα3Galβ4(Fucα3)GlcNAcβ6GalNAc

The invention is further directed binders and their use, wherein the terminal epitope recognized includes at least structure according to the formula TE4 [SAαX₁]ₙ₄Galβ4[(Fucα3)]ₙ₂GlcNAcβ6{([SAαX₃]ₙ₃Galβ3)}ₙ₉[GalNAc]ₙ₆, wherein variables n1 and n9 are 0 or 1, independently, and X₃ is 3 or 6, preferably 3, and other variables are as in Formula TE1, with the provisio that at least either n6 or n9 is 1.

This binder types includes recognition of O-glycan by recognition of GalNAc-residue and/or the other branch present in the structures.

The invention is further directed binders and their use, wherein the terminal epitope recognized includes at least structure according to the formula TE5 [SAαX₁]ₙ₄Galβ4[(Fucα3)]ₙ₂GlcNAc{β3Galβ[(Fucα3)]_{n1[}4GlcNAc]ₙ₅}ₘ, wherein variables are as in Formula TE1.

This binder type includes preferred poly-N-acetyllactosamine type sequences revealed on O-glycans by the invention. It is realized that the O-glycans, especially the sialylated O-glycans are preferred and the poly_Nacetyllactosamines form characteristic epitopes on cell surfaces. The polylactosamine epitopes are in an embodiment recognized without the context of O-glycans.

The invention is further directed binders and their use, wherein the binder recognizes terminal epitope selected from the group
GalβGlcNAc, Galβ4GlcNAcβ, Galβ4GlcNAcβ6, Galβ4GlcNAcβ6GalNAc,
Galβ4GlcNAcβ6(Galβ3)GalNAc, Galβ4GlcNAcβ6GalNAcα,
Galβ4GlcNAcβ6(Galβ3)GalNAcα,
Galβ4(Fucα3)GlcNAc, Galβ4(Fucα3)GlcNAcβ3, Galβ4(Fucα3)GlcNAcβ6,
Galβ4(Fucα3)GlcNAcβ6GalNAc, Galβ4(Fucα3)GlcNAcβ6(Galβ3)GalNAc,
Galβ4(Fucα3)GlcNAcβ6GalNAcα, and Galβ4(Fucα3)GlcNAcβ6(Galβ3)GalNAcα, and
NeuNAcα3GalβGlcNAc, NeuNAcα3Galβ4GlcNAcβ, NeuNAcα3Galβ4GlcNAcβ6,
NeuNAcα3Galβ4GlcNAcβ6GalNAc, NeuNAcα3Galβ4GlcNAcβ6(Galβ3)GalNAc,
NeuNAcα3Galβ4GlcNAcβ6GalNAcα, NeuNAcα3Galβ4GlcNAcβ6(Galβ3)GalNAcα,
NeuNAcα3Galβ4(Fucα3)GlcNAc, NeuNAcα3Galβ4(Fucα3)GlcNAcβ,
NeuNAcα3Galβ4(Fucα3)GlcNAcβ6, NeuNAcα3Galβ4(Fucα3)GlcNAcβ6GalNAc,
NeuNAcα3Galβ4(Fucα3)GlcNAcβ6(Galβ3)GalNAc,
NeuNAcα3Galβ4(Fucα3)GlcNAcβ6GalNAcα, and
NeuNAcα3Galβ4(Fucα3)GlcNAcβ6(Galβ3)GalNAcα

### Terminal Mannose N-Glycan

Preferred terminal mannose N-glycan contains as non-reducing terminal monosaccharide residue or residues at least one Manα/β-residue(s) and optionally a non-reducing end branching Fucα6-residue and the core structure of the native glycan comprises Manβ4GlcNAcβ4GlcNAc,

The preferred N-glycan oligosaccharide sequence have a structures according to the Formula:

[Manα2]ₙ₁[Manα3]ₙ₂[Manα2]ₙ₃[Manα6]ₙ₄[Manα6]ₙ₅[Manα2]ₙ₆[Manα2]ₙ₇[Manα3]ₙ₈)Manβ4GlcNAcβ4[(Fucα6)]ₘGlcNAc[β-N-Asn]ₚ

wherein p, n1, n2, n3, n4, n5, n6, n7, n8, and m are independently either 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; the sum of n1, n2, n3, n4, n5, n6, n7, and n8 is less than or equal to (m+3); and preferably n1, n3, n6, and n7 are 0 when m is 0; [ ] indicates determinant either being present or absent depending on the value of n1, n2, n3, n4, n5, n6, n7, n8, and m; and
( ) indicates a branch in the structure.

The preferred terminal Man glycan to be recognized has preferably at least one structure selected from the group:
Manβ, Manβ4GlcNAc, Manβ4GlcNAcα, Manβ4GlcNAcβ4GlcNAc,
Manβ4GlcNAcβ4(Fucα6)GlcNAc, Manβ4GlcNAcβ4GlcNAcβ,
Manβ4GlcNAcβ4(Fucα6)₀ₒᵣ₁GlcNAcβ, Manβ4GlcNAcβ4GlcNAcβAsn, and
Manβ4GlcNAcβ4(Fucα6)GlcNAcβAsn;
Manα3Man, Manα6Man, Manα3Manβ, Manα6Manβ, Manα3Manα and Manα6Manα,
Manα3 (Manα6)Man, Manα3 (Manα6)Manβ, and Manα3 (Manα6)Manα.

The invention is further directed binders and their use, wherein the binder recognizes at least terminal Manα6/Manα3/Manβ epitope according to the formula TEM1:

[Manα6]ₙ₁([Manα3]ₙ₂)Man[α]ₘ₂[β]_{n3[}4GlcNAcβ]ₙ₄[(Fucα6)]_{m[}4GlcNAc]ₙ₅[β-N-Asn]ₚ

wherein p, n1, n2, n3, n4, n5, m and m2 are independently either 0 or 1; ( ) indicates a branch in the structure, and with provisio that when m2 is 1, then n3, n4, n5, m and p are 0

The invention is farther directed binders and their use, wherein the binder recognizes at least terminal Manα6/Manα3-epitope according to the Formula TEM2:

[Manα6]ₙ₁([Manα3]ₙ₂)Man

wherein the variables are as in formula TEM1.

The invention is further directed binders and their use, wherein the binder recognizes at least terminal Manα6/Manα3-Manα epitope according to the formula TEM3:

[Manα6]ₙ₁([Manα3]ₙ₂)Manα

wherein the variables are as in formula TEM1.

The invention is further directed binders and their use, wherein the binder recognizes at least terminal Manα6/Manα3-Manβ epitope according to the formula TEM4:

[Manα6]ₙ₁([Manα3]ₙ₂)Manβ

wherein the variables are as in formula TEM1.

The invention is further directed binders and their use, wherein the binder recognizes at least terminal Manα6/Manα3-Manβepitope according to the formula TEM5:

[Manα6]ₙ₁([Manα3]ₙ₂)Manβ4GlcNAcβ[(Fucα6)]_{m[}4GlcNAc]ₙ₅

wherein the variables are as in formula TEM1.

The invention is further directed binders and their use according to TEM5, wherein either m and/or n5 is 1.

This group is preferred for the recognition of the reducing end fucose and/or GlcNAc residues.

The invention is further directed binders and their use according to TEM5, wherein m and n5 is 1 and at least fucose residue is effectively recognized.

This group is preferred for the recognition of the reducing end fucose and GlcNAc residues.

The invention is especially directed to recognition of core fucosylated unusual glycans. The core fucosylation is also preferred with novel terminal HexNAc(GlcNAc) structures according to the invention, such as Lec14 and or Lec18 like structures.

The invention is further directed binders and their use according to TEM5, wherein either n1 and/or n2 is 1.

This group is preferred for the recognition of at least one non-reducing end Manα-residue or both of these: Manα6Manβ, Manα3Manβ, Manα6(Manα3)Manβ, Manα6Manβ4GlcNAc, Manα3Manβ4GlcNAc, Manα6(Manα3)Manβ4GlcNAc, Manα6Manβ4GlcNAcβ, Manα3Manβ4GlcNAcβ, Manα6(Manα3)Manβ4GlcNAcβ. More preferably specific branched epitope is recognized, with increasing preference with longer epitope Manα6(Manα3)Manβ, more preferably Manα6(Manα3)Manβ4GlcNAc, and most preferably Manα6(Manα3)Manβ4GlcNAcβ.

The invention is preferably directed to the recognition of the specific glycan epitopes by specific monoclonal antibodies or by lectins or enzymes, most preferably by monoclonal antibodies.

The invention is further directed binders and their use according to TEM5, wherein n1 and n2 is 0.

In this embodiment the invention

### Mass Spectrometric Profiling of the Glycan Compositions

The invention is further directed to recognition of the preferred structures from human cancers, wherein the marker glycan structures are recognized based on alteration of signals corresponding to specific monosaccharide compositions, when the alteration is measured in comparison to normal tissue. The method preferably includes verification of the process by analyzing the specific structures of the cancer type of the same patient or a large group of patients with the same cancer type by methods such as glycosidase digestion, fragmentation mass spectrometry, or NMR. In a preferred profiling method both O-glycans and N-glycans are measures.

In a preferred profiling process at least one internal standard molecule preferably a glycan molecule is added to glycans to be analyzed. The glycan molecule is in a preferred embodiment a chemical derivative of a normal N-glycan or O-glycan or human milk oligosaccharide, the preferred derivatization occurs at limited amount of positions leaving substantial similarity to native glycans, preferred modification position is the reducing end of the glycans, and a preferred example of modification is reductive amination with a hydroxyl-alkyl-amine molecule, when the length of the allyl is selected to avoid overlap with regular glycans in the profiles. In a preferred embodiment the modification is modification by stable isotopes such as C13 or H2(deuterium) as well known in the art. Preferably the internal standard has molecular weight different from the molecular weights of the cancer glycans. More preferably the internal standard comprises specific amount of standard molecule, which is in range of the individual signals to be detected from the sample. More preferably at least two internal standards of different molecular weights are used, even more preferably the at least two internal standards comprise different amounts of the marker molecule allowing detection of efficacy of the measurement, the two standards with different amounts are preferably have similar structures (e.g. different reducing end derivatization) molecular weights close to each other, preferably within 200 Da, more preferably within 100 Da and most preferably within about 80 Da or especially for neutral glycans about 60 Da, but difference larger than the isotope patterns/ion forms do not over lap, preferably over 20 Da for neutral glycans and for acidic glycans over about 40 Da more preferably over 50 Da. The invention is further directed to use of internal standard molecules, or pairs of molecules with differing amounts, so that at least one molecule or molecule pair is at lower mass range, and one molecule or pair at higher mass range, or even more preferably additionally one molecule or pair of molecules in the middle of the mass range.

The invention is directed to a method of evaluating the malignancy of a patient sample comprising the step of detecting the presence or amount of a cancer related oligosaccharide sequence in the sample, said oligosaccharide sequence comprising any one of the structures from the following groups:

a) low mannose N-glycans with monosaccharide compositions Hex₁₋₄HexNAc₂ or Hex₁₋₅HexNAc₂dHex₁, containing one or more terminal Man residues;
b) neutral O-glycans with monosaccharide compositions Hex₂HexNAc₂dHex₀₋₁, containing one or more terminal Galβ4 residues; and
c) sialylated O-glycans with monosaccharide compositions SA₁₋₂HexₘHexNAcₘdHexₙ, wherein 2≦m≦4 and n<m, containing one or more terminal SAα3 residues, wherein preferably the method involve mass spectrometric profiling of a cancer sample and determining monosaccharide compositions of both O-linked and/or N-linked glycans

### Metastasis Specific Structures and Preferred Uses

The invention is especially directed to the recognition of metastasis specific structures by the methods according to the invention. The invention is especially directed to treatment or prevention of metastasis by therapies such as antibody directed/using therapies against metastatic cells. The invention is further directed to recognition and identification the origin tissues of metastasis based on the glycan structures, such as blood groups structures described in the invention.

The invention is especially directed to uses of N-acetyllactosamine recognizing antibodies with specificities for fucosylated, sialylated and/or sulfated (sulfoester) N-acetyllactosamines for the recognition of metastasis.

The inventors found that various glycan classes are expressed differentially in metastases as described in Example 24 and other Examples of the present invention and the present invention is specifically directed to detecting and using corresponding glycan structures according to the uses of the present invention. The invention is especially directed to non-fucosylated and fucosylated hybrid-type or monoantennary N-glycans, complex-type N-glycans, more specifically non-fucosylated, fucosylated, multifucosylated, terminal HexNAc-type complex-type N-glycans, and mono- and multisialylated N-glycans; neutral O-glycans according to the present invention; soluble glycans according to the present invention; sialylated O-glycans according to the present invention including Core1 type, and larger O-glycans including Core2 type, mono- and multisialylated, fucosylated, and sulfated and/or phosphorylated O-glycans; as detailed for glycan signals corresponding to these glycan groups as listed in Table 15 and as described in the present invention. Specifically, these glycan groups contain terminal structures groups GalβGlcNAc, GalβGalNAc, HexNAcβincluding GlcNAcβ, and sialylated and/or fucosylated and/or sulphated and/or phosphorylated variants thereof as described in the present invention, which are differentially expressed in specifically varying amounts with regard to malignancies and metastases as described in the Examples. The present examples give guidelines to detecting and targeting such glycan structures according to the invention.

The present invention is further especially directed to using such glycan structures in detecting or manipulation of metastases, preferably lymph node metastases and/or liver metastases as described in the present invention.

### Novel Preferred Blood Group Cancer Target Structures

The invention is directed to the recognition of novel cancer associated blood group glycan epitopes detected by mass spectrometry and or by specific binders such as antibodies including blood group specific antibodies used in hematology, the preferred antibodies have specificity for terminal epitopes Fucα2Galβ1-(H-antigens), Galα3 (Fucα2)Galβ1-(B-antigens) and GalNAcα3(Fucα2)Galβ1-(A-antigens) and the invention is directed to optimization of the antibodies based on the carrier structures preferably antibodies recognizing the structures on O-glycan cores such as Galβ3GalNAc, wherein the Galβis preferably the Galβof the A/B/H-antigens above or on one or two Galβof core 2 GalβGlcNAcβ6(Galβ3)GalNAc. The invention is especially directed to recognition of the structures in context of metastasis and indications as described below and in examples.

The inventors found that specific ABO blood group type O-glycan structures serve as useful markers of epithelial cancer derived metastases, such as described in Example 23. The present invention is especially directed to such metastasis associated epitopes, detecting such epitopes, and using them according to the present invention. The invention is further directed to ABO blood group specific epitopes depending on the subject's ABO blood group expression, as detailed below in sections 1), 2), and 3).

1) In all patients with A/B/O blood group, the invention is directed to glycans with the structures HexₓHexNAc_{y}dHex_{z}, wherein preferably x=y; preferably Fucα_{z}[(GalβGlcNAcβ)ₓ₋₁Galβ3GalNAc], wherein preferably x≧2, more preferably 3≧x≧2, most preferably x=2; and preferably z≧1, more preferably 2≧Z≧1; in a preferred embodiment including signal at m/z 1063 corresponding to glycan Hex₂HexNAc₂dHex₂, preferably with the structure Fucα?[(GalβGlcNAcβ)Galβ3GalNAc], more preferably including non-reducing terminal epitope Fucα2Galβ.

2) Specifically in patients with A blood group, the invention is directed to glycans with the structures HexₓHexNAc_{y}dHex_{z}, wherein preferably x+1=y; preferably Fucα_{z}(GalNAcα3)[(GalβGlcNAcβ)ₓ₋₁Galβ3GalNAc], wherein preferably x≧2, more preferably 3≧x≧2, most preferably x=2; and preferably z≧1, more preferably 2≧z≧1; in a preferred embodiment including signal at m/z 1120 corresponding to glycan Hex₂HexNAc₃dHex₁, preferably with the structure Fucα2(GalNAcα3)[(GalβGlcNAcβ)Galβ3GalNAc]; and in a further preferred embodiment including signal at m/z 1266 corresponding to glycan Hex₂HexNAc₃dHex₂, preferably with the structure Fucα2(GalNAcα3)[(GalβGlcNAcβ)Galβ3GalNAc], more preferably including non-reducing terminal epitope Fucα2(GalNAcα3)Galβ.

3) Specifically in patients with B blood group, the invention is directed to glycans with the structures HexₓHexNAc_{y}dHex_{z}, wherein preferably y+1=x; preferably Fucα_{z}(Galα3)[(GalβGlcNAcβ)_{x, Galβ}3GalNAc], wherein preferably y≧2, more preferably 3≧y≧2, most preferably y=2; and preferably z1, more preferably 2≧z≧1; in a preferred embodiment including signal at m/z 714 corresponding to glycan Hex₂HexNAc₁dHex₁, preferably with the structure Fucα2(Galα3)Galβ3GalNAc; and in a further preferred embodiment including signal at m/z 1225 corresponding to glycan Hex₃HexNAc₂dHex₂, preferably with the structure Fucα2(Galα3)[(GalαGlcNAcβ)Galβ3GalNAc], more preferably including non-reducing terminal epitope Fucα2(Galα3)Galβ.

### Preferred Elongated Blood Group O-Glycans

The specific blood group O-glycans are especially preferred for the gastrointestinal cancers, preferably stomach cancer; or female reproductive cancer such as ovarian cancer or cervical cancers or breast cancer, in preferred embodiment for breast cancer.

The inventors assigned the blood group O-glycans, based on the monosaccharide compositions and known biosynthetic preferences.

It is realized that epithelial tissues of GI tract or reproductive tissues and in separate embodiment like ovarian/cervical tissues and other similar tissues express type I Galβ3GlcNAc structures and these are preferred blood group epitopes in the cancer samples, the type II structures are preferred on core II due to biosynthetic preferences of Galb3-transferases. It is realized that a few effective terminal epitopes for recognition by specific blood group, A, B, H-, Lewis y and Lewis b-antigens can be obtained from the analysis. The invention is directed to use of regular blood group/lewis antibodies and highly specific variants recognizing more core epitope for recognition of cancer according to the invention.

### Core I O-glycans

The invention is in a preferred embodiment

Directed to core 1 O-glycans according to the Formula BL core 1

[Gal(NAc)ₙα3]ᵣ(Fucα2)Galβ3GalNAc[αSer/Thr]ₚ

wherein n, p, and r are 0 or 1 independently
preferred structures includes A,B and 0 (H)-antigens
GalNAcα3(Fucα2)Galβ3GalNAc[αSer/Thr]ₚ
Galα3(Fucα2)Galβ3GalNAc[αSer/Thr]ₚ
Fucα2Galβ3GalNAc[αSer/Thr]ₚ

Elongated core 1 epitopes. The invention revealed structures with multiple fucose indicating branching and core II structures, in a separate embodiment the invention is further directed to elongated core I structures, it is realized that the terminal elongated epitopes are same or similar to elongated core II terminal epitopes

According to the Formula BL core 1 elongated:

A1{Galβ4/3[(Fucα4/3)]ₙ₁GlcNAcβ3}ₘGalβ4[(Fucα3)]ₙ₂GlcNAcβ3Galβ3GalNAc[αSer/Thr]ₚ

wherein A1 and A2 are independently [Gal(NAc)ₙα3]ᵣ(Fucα2)
n, n1, n2, n3, n4, p and r are either independently 0 or 1 and m is 0, 1 or 2;
{ } and [ ] indicate determinant either being present or absent depending on the value of m, n1 and n2; and
( ) indicates a branch in the structure, with the provisio that preferably n is either 0 or 1 for both A1 and A2.

### Recognition of Terminal Epitopes of the Preferred Glycans Structures

The invention is in a preferred embodiment directed to the terminal epitopes recognized by specific binders (binding molecules preferably antibodies), the invention is further directed to antibodies recognizing the terminal core I epitopes as a branches of core II O-glycans and other branches of core II and I structures and elongated variants thereof. Numerous antibodies for the blood group antigens are known and can be selected against glycan libraries recognizing the structures. The preferred epitopes include preferably the reducing end α-anomer and optionally preferably further part of Ser/Ther residue. The more elongated epitopes with longer glycan sequences are more preferred due to higher specificity. The invention is especially directed to recognition of non-reducing end terminal structure derived from the formulas according to the invention, the non-reducing end terminals structures are preferably not modified by any other structures, except from the reducing end.

### Elongated Core II and Core II Structures

The invention revealed eloganted bollogroup antigens

According to the Formula BL:

A1{Galβ4/3[(Fucα4/3)]ₙ₁GlcNAcβ3}ₘGalβ4[(Fucα3)]ₙ₂GlcNAcβ6([A2]ₙ₃Galβ3)ₙ₄GalNAc[αSer/Thr]ₚ

wherein A1 and A2 are independently [Gal(NAc)ₙα3]ᵣ(Fucα2)
n, n1, n2, n3, n4, p and r are either independently 0 or 1 and m is 0, 1 or 2;
{ } and [ ] indicate determinant either being present or absent depending on the value of m, n1 and n2; and
( ) indicates a branch in the structure, with the provisio that preferably n is either 0 or 1 for both A1 and A2.

The preferred shorter terminal core II comprising GalβGlcNAc has a structure A1Galβ4[(Fucα3)]ₙ₂GlcNAcβ6([A2]ₙ₃Galβ3)ₙ₄GalNAc[(Ser/Thr]ₚ

And the variables are as described for BL
selected from the group:
[Gal(NAc)ₙα3]ᵣ(Fucα2)Galβ3)GalNAc,
[Gal(NAc)ₙα3]ᵣ(Fucα2)Galβ4GlcNAcβ6([Gal(NAc)ₙα3]ᵣ(Fucα2)Galβ3)GalNAc,
[Gal(NAc)ₙα3]ᵣ(Fucα2)Galβ4(Fucα3)GlcNAcβ6GalNAc, and
[Gal(NAc)ₙα3]ᵣ(Fucα2)Galβ4GlcNAcβ6GalNAc and
GalNAcα3(Fucα2)Galβ4(Fucα3)GlcNAcβ6(Fucα2Galβ3)GalNAc,
(Fucα2)Galβ4(Fucα3)GlcNAcβ6(GalNAcα3 (Fucα2)Galβ3)GalNAc
GalNAcα3 (Fucα2)Galβ4GlcNAcβ6(Fucα2Galβ3)GalNAc,
(Fucα2)Galβ4GlcNAcβ6(GalNAcα3 (Fucα2)Galβ3)GalNAc
GalNAcα3(Fucα2)Galβ4(Fucα3)GlcNAcβ6(Galβ3)GalNAc,
Galβ4(Fucα3)GlcNAcβ6(GalNAcα3(Fucα2)Galβ3)GalNAc
GalNAcα3 (Fucα2)Galβ4GlcNAcα6(Galβ3)GalNAc,
Galβ4GlcNAcβ6(GalNAcα3 (Fucα2)Galβ3)GalNAc and
Galα3 (Fucα2)Galβ4(Fucα3)GlcNAcβ6(Fucα2Galβ3)GalNAc,
(Fucα2)Galβ4(Fucα3)GlcNAcβ6(Galα3 (Fucα2)Galβ3)GalNAc
Galα3 (Fucα2)Galβ4GlcNAcβ6(Fucα2Galβ3)GalNAc,
(Fucα2)Galβ4GlcNAcβ6(Galα3 (Fucα2)Galβ3)GalNAc
Galα3 (Fucα2)Galβ4(Fucα3)GlcNAcβ6(Galβ3)GalNAc,
Galβ4(Fucα3)GlcNAcα6(Galα3 (Fucα2)Galβ3)GalNAc
Galα3 (Fucα2)Galβ4GlcNAcβ6(Galβ3)GalNAc,
Galβ4GlcNAcβ6(Galα3 (Fucα2)Galβ3)GalNAc

### And

Fucα2Galβ4(Fucα3)GlcNAcβ6(Fucα2Galβ3)GalNAc,
Fucα2Galβ4(Fucα3)GlcNAcβ6(Galβ3)GalNAc
Galβ4(Fucα3)GlcNAcβ6(Fucα2Galβ3)GalNAc
Fucα2Galβ4GlcNAcβ6(Fucα2Galβ3)GalNAc,
(Fucα2)Galβ4GlcNAcβ6(Galβ3)GalNAc
Galβ4GlcNAcβ6(Fucα2Galβ3)GalNAc

Preferred terminal epitopes to be recognized includes type II antigen structures

A1Galβ4[(Fucα3)]ₙ₂GlcNAcβ[6]ₙ₄[GalNAc]ₙ₃[αSer/Thr]ₚ,

Wherein variables are as in BL
[Gal(NAc)ₙα3]ᵣ(Fucα2)Galβ4GlcNAcβ6
[Gal(NAc)ₙα3]ᵣ(Fucα2)Galβ4(Fucα3)GlcNAcβ6GalNAc
[Gal(NAc)ₙα3]ᵣ(Fucα2)Galβ4(Fucα3)GlcNAcβ6GalNAc, and
[Gal(NAc)ₙα3]ᵣ(Fucα2)Galβ34GlcNAcβ6GalNAc

Preferred terminal epitopes to be recognized includes elongated core II antigen structures

A1{Galβ4/3[(Fucα4/3)]ₙ₁GlcNAcβ3}ₘ{Gal[β4[(Fucα3)]ₙ₂GlcNAcβ6]ₙ₄}ₙ₆

wherein variables are as in BL and n6 is 0 or 1

Preferably type N-acetyllactosamine epitopes
[Gal(NAc)ₙα3]ᵣ(Fucα2)Galβ4(Fucα3)GlcNAcβ3Gal
GalNAcα3(Fucα2)Galβ4(Fucα3)GlcNAcβ3Gal
Galα3(Fucα2)Galβ4(Fucα3)GlcNAcβ3Gal
Fucα2Galβ4(Fucα3)GlcNAcβ3Gal
[Gal(NAc)ₙα3]ᵣ(Fucα2)Galβ4GlcNAcβ3Gal
GalNAcα3 (Fucα2)Galβ4GlcNAcβ3Gal
Galα3 (Fucα2)Galβ4GlcNAcβ3Gal
Fucα2Galβ4GlcNAcβ3Gal

### Or

Preferably type I N-acetyllactosamine epitopes
[Gal(NAc)ₙα3]ᵣ(Fucα2)Galβ3(Fucα4)GlcNAcβ3Gal
GalNAcα3 (Fucα2)Galβ3 (Fucα4)GlcNAcβ3Gal
Galα3 (Fucα2)Galβ3 (Fucα4)GlcNAcβ3Gal
Fucα2Galβ3(Fucα4)GlcNAcβ3Gal (elongated Lewis b)
[Gal(NAc)ₙα3]ᵣ(Fucα2)Galβ3GlcNAcβ3Gal
GalNAcα3 (Fucα2)Galβ3GlcNAcβ3Gal
Galα3 (Fucα2)Galβ3GlcNAcβ3Gal
Fucα2Galβ3GlcNAcβ3Gal

### Uses of the Binders for Isolation of Cellular Components and Mixtures Thereof.

The invention revealed novel binding reagents are in a preferred embodiment used for isolation of cellular components from cancer cells/tissues comprising the novel target/marker structures. The isolated cellular are preferably free glycans or glycans conjugated to proteins or lipids or fragment thereof.

The invention is especially directed to isolation of the cellular components comprising the structures when the structures comprises one or several types glycan materials selected
a) Free glycans released from the cancer cell materials and/or
b) Glycan conjugate material such as
   1) glycosamino acid materials including
      b1a) glycoproteins
      b1b) glycopeptides including glyco-oligopeptides and glycopolypeptides and/or
   b2) lipid linked materials comprising the preferred carbohydrate structures revealed by the invention.

### General Method for Isolation Cellular Components Comprising the Target Structures

The isolation of cellular components according to the invention means production of a molecular faction comprising increased amount of the glycans comprising the target structures according to the invention in method comprising the step of binding of the binder molecule according to the invention to the target structures.

The preferred method to isolate cellular component includes following steps
1) Providing a cancer cell sample.
2) Contacting the binder molecule according to the invention to the target structures.
3) Isolating the complex of the binder and target structure at least part of cellular materials.

It is realized that the components are in general enriched in specific fractions of cellular structures such as cellular membrane fractions including plasma membrane and organelle fractions and soluble glycan comprising fractions such as soluble protein, lipid or free glycans fractions. It is realized that the binder can be used to total cellular fractions.

In a preferred embodiment the target structures are enriched within a fraction of cellular proteins such as cell surface proteins releasable by protease or detergent soluble membrane proteins.

More preferably the invention is directed to purification of the target structure fraction in the isolation step. The purification is in a preferred mode of invention is at least partial purification. Preferably the target glycan containing material is purified at least two fold, preferably among the components of cell fraction wherein it is expressed. More preferred purification levels includes 5-fold and 10 fold purification, more preferably 100, and even more preferably 1000-fold purification. Preferably the purified fraction comprises at least 10% of the target glycan comprising molecules, even more preferably at least 30%, even more preferably at least 50%, even more preferably at least 70% pure and most preferably at least 90% pure. Preferably the % value is mole percent in comparison to other non-target glycan comprising glycoconjugate molecules, more preferably the material is essentially devoid of other major organic contaminating molecules.

### Preferred Purified Target Glycan Compositions and Target Glycan-Binder Complexes

The invention is also directed to isolated or purified target glycan-binder complexes and isolated target glycan molecule compositions.

Preferably the purified target glycan-binder complex compositions comprises at least 10% of the target glycan comprising molecules in complex with binder, even more preferably at least 30%, even more preferably at least 50%, even more preferably at least 70% pure and most preferably at least 90% pure target glycan comprising molecules in complex with binder.

Preferably the purified target glycan composition comprises at least 10% of the target glycan comprising molecules, even more preferably at least 30%, even more preferably at least 50%, even more preferably at least 70% pure and most preferably at least 90% pure target glycan comprising molecules.

Preferred N-Glycan and/or O-Glycan Glycomes and Characteristic Glycan Groups Therein

The invention revealed glycan groups, which are characteristically changed in cancer in protein derived N-glycans and O-glycans. Among N-glycans preferred N-glycans are glycans with non-reducing end terminal Man-residues (terminal Man-glycans), and complex type N-glycans with specific structural characteristics such as specific HexNAc-structures or fucosylstructures.

Preferred O-glycans characteristic for cancer are referred as LacNAc O-glycans, which comprise at least one N-acetyllactosamine unit with possible sialic acid and or fucosyl-modifications, and which is linked a specific O-glycan core type Galβ3GalNAc forming characteristic monosaccharide compositions in O-glycan mass spectra.

### Glycome Analysis by Quantitative Analysis of Glycan Groups in a Glycome

The invention is especially directed to analysis of glycomes as groups of related structures such as low-Man, high-Man, hybrid type, fucosylated, complex N-glycans and subgroups thereof etc. as these biosynthetically related groups characterize cancer tissues and cells. The invention is especially directed to methods of calculation %-part of a specific glycan groups and comparing the % values, e.g. in form of Table. It is notable that the glycan groups may occasionally comprise unusual/uncharacteristic glycans, which do not exactly correspond the title of group, but presence of such material would likely increase the characteristics of the analysis in comparisons between tissue materials. The glycan score analysis according to the invention revealed especial usefulness of the analysis by glycan groups.

### Preferred Terminal Man-Glycans

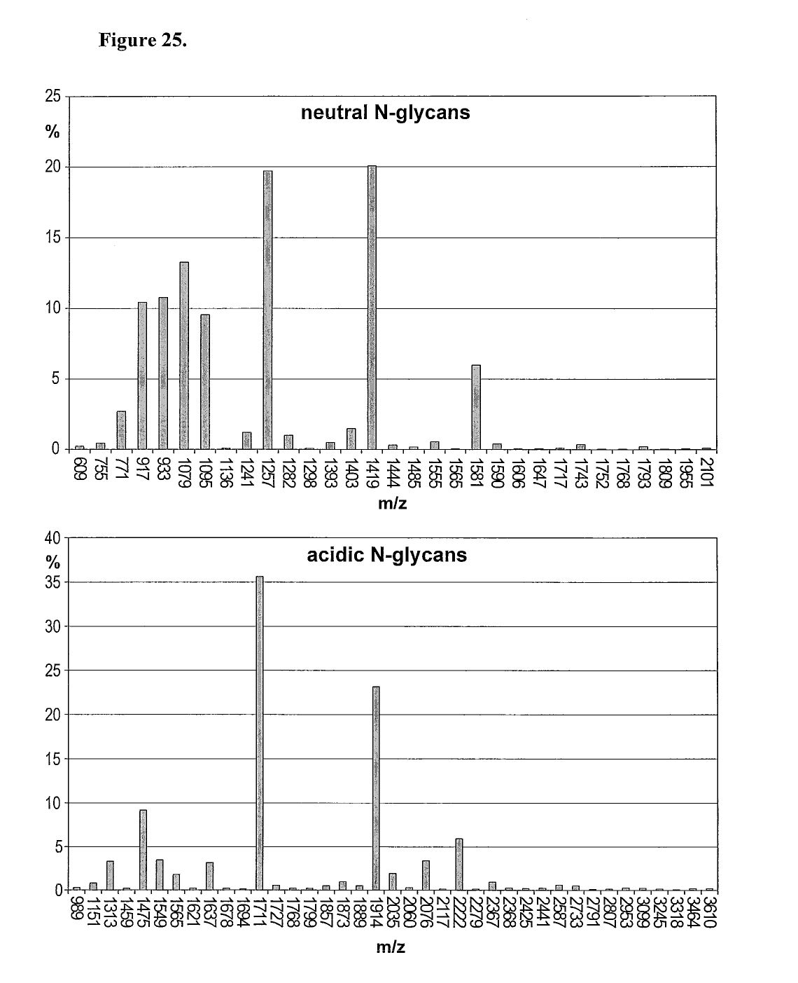

Preferred terminal Man-glycans includes low-Man and high-Man glycans and acidic derivatives thereof, especially phosphorylated derivative. The invention revealed that there is alterations in high-Man type glycans, but that there is especially characteristic alterations in low-Man and acidic glycans. The invention revealed that the low-Man glycans share much similarity with glycans produced by lysosomal enzyme for lysosomal proteins, FIG. 25. The invention thus revealed that the structures altering reflects major changes in balance of cellular organelles in cancers, which is reasonable when considering morphological and other alterations in cancer. The change in high-Man glycans is also reasonable when considering the change of balance between the organelles as these glycans are related to endoplasmic reticulum (ER) and very early Golgi-structures, and the changes in specific types of complex glycans synthesized in Golgi may also in part reflect the intracellular changes in cancer. Furthermore the invention revealed additional novel soluble N-glycan type glycome, comprising terminal-Man structures and giving additional characteristics for cancer glycomes, when included in the glycome fraction.

### Analysis by Specific Binding Molecules

It is further realized that key alterations in glycomes can be also analysed by other methods such as specific binding reagents after altering structure has been determined. The invention is directed to analysis of altering structures when the amount of the structure increases or decreases in a specific cancer. The invention is most preferably directed to use of a binding reagent with regard to a structure, which increases in cancer.

The analysis by binding method molecule may be preferred as a fast test, though the current mass spectrometric screening method is also quite fast and cost effective, only draw being requirement of mass spectrometer, which includes some capital investment for the method. The analysis by the specific binder may be also performed directly from the tissue and better information of tissue and/or cellular localization of the materials can be obtained. It also realized that combinations of at least two binding molecules recognizing different structures would be especially useful for analysis of cancer and multiple selected specific binding molecules would approach the effectively of the mass spectrometric screening methods.

### Preferred Structures in Glycomes to be Analyzed by the Binding Molecules and Target Epitopes Therein

The invention reveled characteristic structures among the N- and O-glycan glycomes. The invention is especially directed to these structures as targets for recognition by specific binding molecules. The invention is furthermore directed to the screening methods. The preferred structures to be recognized among the glycan groups according to the invention includes preferred terminal groups preferably recognized on the preferred glycan core structures according to the invention.

### Preferred Terminal Man-Glycans

Preferred terminal Man-glycans includes low-Man and high-Man glycans and acidic derivatives thereof, especially phosphorylated derivative. The preferred terminal Man glycans comprise non-reducing end Man-residue(s), which Manα- or Manβ-residue, preferably being either one or more Manα-residues or a single Manβ3-residue. Preferably the glycan comprise the Man residue(s) and optionally an additional Fuc-residue (preferably Fucα, more preferably Fucα6-branching residue at the reducing end GlcNAc residue) as the only non-reducing end terminal monosaccharide types.

### Preferred Recognition of Terminal Manβ-Residue Comprising Low-Man Glycans

When the terminal Man residue is Manβ-residue, the structure is the minimal low mannose glycan

Manβ4GlcNAcβ4(Fucα6)₀ₒᵣ₁GlcNAcβAsn. The preferred minimal epitopes to be recognized includes Manβ and terminal disaccharides and tri/tetrasaccharides either including reducing end anomeric structure or not and/or reducing end amino acid residue and/or part of the peptide chain of the potential carrier protein (marked in following by Asn) Manβ,

### Manβ4GlcNAc, Manβ4GlcNAcβ, Manβ4GlcNAcβ4GlcNAc,

### Manβ4GlcNAcβ4(Fucα6)GlcNAc, Manβ4GlcNAcβ4GlcNAcβ,

Manβ4GlcNAcβ4(Fucα6)₀ₒᵣ₁GlcNAcβ, Manβ4GlcNAcβ4GlcNAcβAsn, and Manβ4GlcNAcβ4(Fucα6)GlcNAcβAsn.

The specificity of the binding reagent such as binding protein should such that the binding side covers the terminal Manβ-so that the binding molecule does not cross-react with elongated N-glycans, or less preferably cross reacts only with other preferred low-Man structures according to the invention such as the core structures elongated only by single Manα3/6-residue. The invention is directed to betα-mannosidase enzymes and corresponding engineered lectins used for sequencing N-glycans as examples specific binding reagents. The preferred minimum epitopes includes Manβ, Manβ4GlcNAc, Manβ4GlcNAcβin a preferred embodiment so that the branching fucosyl residue does not affect the binding, or when fucose specific recognition is needed, the binding molecule is selected so that the residue affects binding in desired manner. It is realized that smaller binding epitopes are generally enough for specific recognition as similar structures are rare/non-existing in animal/human materials.

### Preferred Recognition of Terminal Manα-Residue Comprising Low-Man Glycans

The preferred Manα-residue comprising target low-Man glycans includes both isomers of dimannosyl structures, preferably ones comprising Manα6Man, which was analysed to have higher predominance at least in part of cancers, the trimannosyl core structure low-Man glycans, tetra-Mannosylisomers and the pentamannosyl Low-Man glycans according to the invention. The dimannosyl, trimannosyl- and pentamannosyl structures are preferred structures, and the branched trimannosyl and pentamannosyl structures are especially preferred due to prevalence observed and larger homogeneity (less isomers in target). The pentamannosyl structure is especially preferred as a major control step glycan toward low-Man glycans, and the trimannosyl-glycans is separately a preferred control step glycan with different branched accessibility for mannosidases and for synthesis of lower size low mannoses.

The terminal epitopes of antibodies against low-Man structures should recognize linear and/or branched Manα3/6Man-epitopes. The minimal epitopes includes non-reducing end terminal disaccharide and trisaccharide epitopes of the low-Man N-glycans.

Preferred minimal disaccharide epitopes for recognition of Low-Man glycan includes the disaccharides without next anomeric linkage Manα3Man, Manα6Man, and more specific epitopes disaccharides with next anomeric linkage depending on the level of the Manα3Manβ, Manα6Manβ, Manα3Manα and Manα6Manα.

Preferred branched trisaccharides includes Manα3(Manα6)Man, Manα3(Manα6)Manβ, and Manα3(Manα6)Manα. It is furthermore realized that the binding specificity may included further structures in the core of the N-glycans, but the core structures and especially possible branching Fucα-residue does not interfere with the binding. In a specific embodiment the terminal epitope is designed to be recognized by Fuc-sensitive manner.

The specificity of the binding reagent such as binding protein should such that the binding side covers the terminal Manα3/6-structures so that the binding molecule does not cross-react with elongated complex type N-glycans (GlcNAc-modifications on Manα3/6). The invention is directed to such Manα3/6-specific N-glycan core specific antibodies and lectins. The invention is further directed to development of Manα3/6-specific mannosidases to corresponding engineered lectins as examples specific binding reagents.

### Examples of useful antibody types for the recognition of low-Man glycans in cancer include

1) antibodies with specificity similar to antibody IgM mAb 100-4G11-A (van Remoorte A. et al. Glycobiology (2003) vol. 13, 217-225) associated with S. mansoni infection in mouse, which antibody has specificity to branched Manα3(Manα6)Manβ-epitope, but not to pentamannose epitope of RNAse B Manα3[Manα3 (Manα6)Manα6]Manβ-. The antibody is produced naturally in mouse without major autoimmunune complications, and it reacts with only few normal mammalian proteins indicating that the antibody is not harmful even for in vivo applications in mammals.
2) Antibodies with specificity similar to L3 and L4 antibodies (Schmitz B et al. et al. Glycobiology (1993) vol. 3, 609-17) with highest binding for Manα3[Manα3(Manα6)Manα6]Manβ- and lower activity for high-Man glycans.
3) Antibodies with specificity similar to OMR6 and OMR5 described by Ozawa et al. in 1997 (Arch. Biochem. Biophys. 342, 48-57), which bind to Man2 low-mannose type N-glycans, including Manα6Manβ4GlcNAc non-reducing epitope and/or Manα6Manβ4GlcNAcβ4(Fucα6)GlcNAc non-reducing epitope (OMR6), and Manα3Manβ4GlcNAc non-reducing epitope (OMR5). Neither of these antibodies bind to high-mannose type, hybrid-type, or complex-type N-glycans.

### Preferred Antibody Reagents for Recognition of Complex Type N-Glycans

The invention is directed to recognition of specific complex type N-glycans with terminal GlcNAcβ2Manα3/6-structures by antibodies binding to the terminal structures, preferably by antibodies similar to and produced by similar method as described for non-reducing terminal GlcNAcβ2Man specific antibody OMB4 in Ozawa H. et al. (1997) Arch. Biochem. Biophys. 342, 48-57. The present inventors has been revealed terminal GlcNAc-oligosaccharide sequence recognizing natural human antibodies (PCT/FI2003/000615). Such antibodies can be selected from by page display technologies and produced by recombinant antibody technologies. The invention is further directed to the use of such antibodies in context of cancer glycomes preferably with one or more other antibodies according to the invention.

The present inventors has been revealed terminal [NeuNAcα]₀ₒᵣ₁GalNAcβ4GlcNAc-oligosaccharide sequence recognizing natural human antibodies (e.g. U.S. application Ser. No. 10/486,714). Such antibodies can be selected from by page display technologies and produced by recombinant antibody technologies. The invention is further directed to the use of such antibodies in context of cancer glycomes preferably with one or more other antibodies according to the invention.

### LacNAc O-glycans

Preferred O-glycans characteristic for cancer are referred as LacNAc O-glycans, which includes preferred Groups 2 and 3, and which comprise at least one N-acetyllactosamine unit with possible sialic acid (group 3 or c in discussion) and or fucosyl-modifications, and which is linked a specific O-glycan core type Galβ3GalNAc forming characteristic monosaccharide compositions in O-glycan mass spectra.

The preferred structures among the LacNAc O-glycans includes core II structure-type O-glycan structures comprising core oligosaccharide sequence LacNAcβ6GalNAc, more preferably the branched structure LacNAcβ6(Galβ3)GalNAc, even more preferably LacNAcβ6(Galβ3)_{0 or 1}GalNAcα, which is in a preferred embodiment recognizable in a protein linked form from a cancer sample. The preferred LacNAc unit is type Galβ4GlcNAc, though it is realized that the types of LacNAcs may vary between cancer types. Preferred terminal structures to be recognize from the preferred core II O-glycans includes at least the terminal epitope LacNAcβ6GalNAc, with possible modifications, thus including structures according to the Formula:

{NeuNAcαX}ₙ₂Galβ4[(Fucα3)]ₙ₁GlcNAcβ6[(Galβ3)]ₙ₃GalNAc[α]ₙ₄[Ser/Thr]ₙ₅

wherein X is linkage position 3 or 6,
( ) indicates branch in the structure and
n1, n2, n3, n4 and n5 are 0 or 1, independently.

The invention is in a preferred embodiment directed to the recognition of minimal epitopes (when n4 and n5 are 0) with (n3 is 1) or without the branch (n3 is 0), which is the very minimal epitope, but could be also more easily achieved by an antibody or another binding molecule. The invention is especially directed to the binders of the minimal structures when the structure is further complicated by fucose or sialic acid substitutions. It realized that on tissue proteins the non-galactosylated form is a potential degradation form of the actual core 2 and these are can occur together. The actual core 2 structures are preferred as actual major target structures, when the Galβ3-is not recognized by the binding molecule, the specificity of the binder such as an antibody is preferably such as the Gal-structure is not preventing the binding, i.d. the antibody has dual specificity for Gal and non-Gal-structures.

The invention is especially directed to recognition of minimal structures by reagents which do not include additional specificity directing the reagents to glycolipids as known for certain antibodies or preferably the antibody favours the recognition of the epitopes on a protein or part of the peptide sequence such as Ser/Thr-residue is included in the antibody specificity. Preferred terminal sialylated (group 3) structures minimal structures to be recognized includes
NeuNAcα3/6Galβ4(Fucα3)GlcNAcβ6(Galβ3)GalNAc,
NeuNAcα3/6Galβ4GlcNAcβ6(Galβ3)GalNAc,
NeuNAcα3/6Galβ4(Fucα3)GlcNAcβ6GalNAc, and NeuNAcα3/6Galβ4GlcNAcβ6GalNAc and
   the preferred terminal non-sialylated minimal (group 2) structures include
Galβ4(Fucα3)GlcNAcβ6(Galβ3)GalNAc, Galβ4GlcNAcβ6(Galβ3)GalNAc,
Galβ4(Fucα3)GlcNAcβ36GalNAc, and Galβ4GlcNAcβ6GalNAc.

Preferred terminal sialylated (group 3) peptide epitope including structures to be recognized includes NeuNAcα3/6Galβ4(Fucα3)GlcNAcβ6(Galβ3)GalNAcαSer/Thr, NeuNAcα3/6Galβ4GlcNAcβ6(Galβ3)GalNAcαSer/Thr, NeuNAcα3/6Galβ4(Fucα3)GlcNAcβ6GalNAcαSer/Thr, and NeuNAcα3/6Galβ4GlcNAcβ6GalNAcαSer/Thr.

the preferred terminal peptide epitope including structures non-sialylated (group 2) structures include Galβ4(Fucα3)GlcNAcβ6(Galβ3)GalNAcαSer/Thr, Galβ4GlcNAcβ6(Galβ3)GalNAcαSer/Thr, Galβ4(Fucα3)GlcNAcβ36GalNAcαSer/Thr, and Galβ4GlcNAcβ6GalNAcαSer/Thr. 3/6 indicates either of the linkages of the sialic acid and Ser/Thr either of the linkage amino acid residues.

The neutral non-fucosylated structures Galβ4GlcNAcβ6GalNAc and Galβ4GlcNAcβ6(Galβ3)GalNAc are especially preferred in α-anomeric forms and to be specifically recognized in alfa-anomeric form, preferably linked to an Ser/Thr residue and/or being recognizable on protein and/or preferably being recognized on cancer protein. Background describes such structure on a lipid and it is known that similar branched structures do occur on galactosylglobosides (at least in mice) and GalNAcα-substitutable by branching β6-GlcNAc transferases is not known from human glycolipids. As the target structures are by different chemical linkage on different carrier both factors effective affecting immunorecognition, there is clear difference to the very limited and unique background in single cancer type.

Examples of useful antibody types for the recognition of neutral core II O-glycans in cancer include antibodies reported to bind Galβ4GlcNAcβ6(Galβ3)GalNAc (Hep 27-Mouse monoclonal antibody, Sandee D. et al. (2002) J. Biosci. Bioengin. (1993) 266-273, possible related application JP 10084963, TOSOH corp; different antibody JP 6046880 Ryuichi Horie. et al), and Galβ4GlcNAcβ6GalNAc (Chung Y-S et al. EP 0601859, TOSOH corp). The Sandee publication reports reactivity of the antibody with a human cultivated hepatocellular carcinoma cell line (HCC-S102, not actual cancer), fetal liver, possible undisclosed cancers (relevance to human cancers or to human cultivated cell lines cannot be known) and not to adult liver, the article discusses heaptocellular carcinoma HCC based on the cell line result. Galβ4GlcNAcβ6GalNAcαCer (lipid) binding antibody (FI alpha-75) was suggested for cancers of digestive organ, specifically stomach (Chung Y-S et al.), results show 79% reaction with stomach cancers, when faint labelling is counted, 38% reactivity with colon cancers and, 57% for colon cancers in immunohistochemistry. The antibodies were reported to be specific for glycolipids but the present invention reveals that this type of antibodies are also useful for analysis of cancer glycoproteins. The invention furthermore reveals new indication for the antibodies, especially one specific for Galβ4GlcNAcβ6(Galβ3)GalNAc. Though the prior art implied certain indications usefulness for other indications cannot be known and based on the interest of the company producing the antibody it would appear likely that they would have tested all possible cancers but unfortunately failed with revealing the novel present indications, to which phe present invention is especially directed to.

Examples of useful antibody types for the recognition of acidic core II O-glycans in cancer include antibody CHO-131 (Walcheck B. et al. Blood (2002) 99, 4063-69) reported to bind specifically core II sLex glycan NeuNAcα3Galβ4(Fucα3)GlcNAcβ6(Galβ3)GalNAcαSer/Thr-peptide. The antibodies were reported to be specific for certain immune cells but the present invention reveals that this type of antibodies are also useful for analysis of cancer glycoproteins according to the invention.

The present invention furthermore reveals novel indications for core 2-sLex structures in human cancer.

### Additional Analysis of Glycans in Cancer Involving Recognition of Carrier Proteins

The invention is further directed to methods of analysing any of the glycans according to the invention from cancer derived proteins, preferably integral (cell bound/transmembrane) cancer tissue or cell released proteins and assigning the glycan structures with specific carrier protein, preferably by specific purification of the protein, e.g. by affinity methods such as immunoprecipitation or by sequencing, preferably by mass spectrometric sequencing, glycopeptides including sequencing and recognizing peptides and thus proteins linked to the proteins.

### The Target Cancer Tissue

The present invention is directed to analysis of un-normally transformed tissues, when the transformation is benign and/or malignant cancer type transformation referred as cancer (or tumor). It is realized that benign transformation may be a step towards malignant transformation, and thus the benign cancers are also useful to be analysed and differentiated from normal tissue, which may have also non cancerous or non-transformation related alterations such as swelling or trauma related to physical or e.g. infectious trauma, and it is useful and preferred to differentiate with benign and malignant cancers

Preferably the tissue is human tissue or tissue part such as liquid tissue, cell and/or solid polycellular tumors, and in another embodiment preferably a solid human tissue. The solid tissues are preferred for the analysis and/or targeting specific glycan marker structures from the tissues, including intracellulalarily and extracellullarily, preferably cell surface associated, localized markers. In a preferred embodiment the invention is specifically directed to the recognition of cell surface localized and/or mostly cell surface localized marker structures from solid tumor tissues or parts thereof. It is realized that the contacts between cells and this glycomes mediating these are affected by presence of cells as solid tumor or as more individual cells. The preferred individual cell type cancers or tumors include preferably blood derived tumors such as leukemias and lymphomas, while solid tumors are preferably includes solid tumors derived from solid tissues suchs gastrointestinal tract tissues, other internal organs such as liver, kidneys, spleen, lungs, gonads and associated organs including preferably ovary, testicle, and prostate. The invention further reveals markers from individually or multicellularily presented cancer cells in contrast to solid tumors. The preferred cancer cells to be analyzed includes metastatic cells released from tumors/cancer and blood cell derived cancers, such as leukemias and/or lymphomas. Metastasis from solid tissue tumors forms a separately preferred class of cancer samples with specific characteristics.

The invention is furthermore directed to the analysis of secretions from tumors such as ascites and/or cyst fluids, and cancer secreted materials present in general body fluids such as blood (or its derivatives such as serum nor plasma), urine, mucous secretions, amnion fluid, lymphatic fluid or spinal fluid, preferably blood, urine or mucous secretions, most preferably blood.

The cancer tissue materials to be analyzed according to the invention are in the invention also referred as tissue materials or simply as cells, because all tissues comprise cells, however the invention is preferably directed to unicellularily and/or multicellularily expressed cancer cells and/or solid tumors as separate preferred characterisitics. The invention further reveals normal tissue materials to be compared with the cancer materials. The invention is specifically directed to methods according to the invention for revealing status of transformed tissue or suspected cancer sample when expression of specific structure of a signal correlated with it is compared to a expression level estimated to correspond to expression in normal tissue or compared with the expression level in an standard sample from the same tissue, preferably a tissue sample from healthy part of the same tissue from the same patient.

The invention is in a preferred embodiment directed to analysis of the marker structures and/or glycome profiles from both cancer tissue and corresponding normal tissue of the same patient because part of the glycosylations includes individual changes for example related to rare glycosylation related diseases such as congenital disorders in glycosylation (of glycoproteins/carbohydrates) and/or glycan storage diseases. The invention is furthermore directed to method of verifying analysing importance and/or change of a specific structure/structure group or glycan group in glycome in specific cancer and/or a subtype of a cancer optionally with a specific status (e.g. primary cancer, metastase, benign transformation related to a cancer) by methods according to the present invention.

The present invention is directed to a set of glycan structures which are expressed by human cancers. The presence or increased amount of one or more of these glycans in a patient sample indicates cancerous status of the sample as described below in detail. The glycan structures can be divided to three basic groups:

1) neutral low-mannose type N-glycans having terminal Man, preferentially Manα structure; for example Manα₀₋₃Manβ4GlcNAcβ4GlcNAc(β-N-Asn) or Manα₀₋₄Manβ4GlcNAcβ4(Fucα6)GlcNAc(β-N-Asn);
2) neutral O-glycans having terminal Galβ4 structure; for example Galβ4[(Fucα3)]ₙGlcNAcβ6(Galβ3)GalNAc[αSer/Thr]ₚ wherein p and n are either independently 0 or 1; and
3) sialylated Core 2 type O-glycans having terminal SAα3 structure, for example Neu5Acα3 (Galβ4[±Fucα3]GlcNAcβ3)₀₋₂Galβ4[±Fucα3]GlcNAcβ(6→GalNAc) and Neu5Acα3(Galβ3[±Fucα4]GlcNAcβ3)₀₋₂Galβ4[±Fucα3]GlcNAcβ(6GalNAc).
Groups 2 and 3 can be considered together as terminal LacNAc O-glycan group. The group 1 may be further included in a larger group of terminal man-glycans including high-Man glycans, which also represent cancer specific changes, but in a more modest scale.

The invention further revealed structures and structural groups, which presence or alterations of amounts of which are characteristic for cancers such as an additional group of HexNAc comprising complex N-glycans, which can be further divided to two groups and level of which would give more specific information about the cancer. The HexNAc comprising N-glycans can be divided to two major groups:

Group 4) glycans with terminal (NeuAcα)₀₋₁HexNAcβHexNAcβ sequences, preferably terminal LacdiNAc sequences
Group 5) terminal 1-linked GlcNAc glycans
Besides the major groups above the present invention reveal additional groups such as Group 6) a group of complex N-glycans including fucosylated multiple N-acetyllactosamine N-glycans revealed as extensively altered structures e.g. in analysis of Table 18 and
Group 7) including multiple fucosylated (or deoxyhexose, dHex, comprising) structures, e.g. present in samples of pancreatic cancer (Tables 11-13 and corresponding examples) and
Group 8) including phosphorylated and/or sulphated glycans, which are also preferred as separate groups and modifications of terminal Man-glycans.

The invention revealed furthermore useful additional glycan groups such as: control groups and/or glycan groups with usually modest changes, such as regular complex type glycans or structural groups otherwise characteristic such as blood groups structure glycans, which have carry over in the metastasis samples.

The invention revealed that mass spectrometric profiling of glycan group is very effective method for analysing cancer samples as multiple characteristic groups can be analyzed simultaneously.

### More Detailed Analysis of Key Groups

### Group 1—Low Mannose Glycans

Low-mannose N-glycans are smaller and more rare than the common high-mannose N-glycans (Man₅₋₉GlcNAc₂). The low-mannose N-glycans detected in tumor tissues fall into two subgroups: 1) non-fucosylated, with composition ManₙGlcNAc₂, where 1≦n≦4, and 2) core-fucosylated, with composition ManₙGlcNAc₂Fuc₁, where 1≦n≦5. The largest of the detected low-mannose structure structures is Man₅GlcNAc₂Fuc₁ (m/z 1403 for the sodium adduct ion), which due to biosynthetic reasons most likely includes the structure below (in the figure the glycan is free oligosaccharide and β-anomer; in glycoproteins in tissues the glycan is N-glycan and β-anomer):

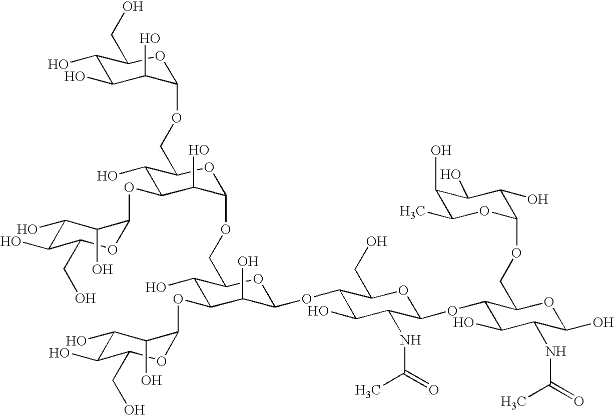

### Preferred General Molecular Structural Features of Group 1Glycans

The low Man glycans described above can be presented in a single Formula:

[Manα2]ₙ₁[Manα3]ₙ₂([Manα2]ₙ₃[Manα6)]ₙ₄)[Manα6]ₙ₅([Manα2]n6[Manα2]ₙ₇[Manα3]ₙ₈)Manβ4GlcNAcβ4[(Fucα6)]ₘGlcNAc[β-N-Asn]ₚ

wherein p, n1, n2, n3, n4, n5, n6, n7, n8, and m are either independently 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; the sum of n1, n2, n3, n4, n5, n6, n7, and n8 is less than or equal to (m+3); and preferably n1, n3, n6, and n7 are 0 when m is 0; [ ] indicates determinant either being present or absent depending on the value of n1, n2, n3, n4, n5, n6, n7, n8, and m; and
( ) indicates a branch in the structure.

Preferred non-fucosylated low-mannose glycans are according to the formula:

[Manα3]ₙ₁[(Manα6)]ₙ₂[Manα4]ₙ₃[(Manα3)]ₙ₄Manβ4GlcNAcβ4GlcNAc[βAsn]ₚ

wherein p, n1, n2, n3, n4 are either independently 0 or 1,
with the proviso that when n3 is 0, also n1 and n2 are 0, and preferably either n1 or n2 is 0,
[ ] indicates determinant either being present or absent depending on the value of n1, n2, n3, n4,
( ) indicates a branch in the structure.

Preferred fucosylated low-mannose glycans are according to the formula:

[Manα3]ₙ₁[(Manα6)]ₙ₂[Manα6]ₙ₃[(Manα3)]ₙ₄Manβ4GlcNAcβ4(Fucα6)GlcNAc[Asn]ₚ

wherein p, n1, n2, n3, n4 are either independently 0 or 1, with the provisio that when n3 is 0, also n1 and n2 are 0, [ ] indicates determinant either being present or absent depending on the value of n1, n2, n3, n4, ( ) indicates a branch in the structure;
and wherein n1, n2, n3, n4 and m are either independently 0 or 1,
with the provisio that when n3 is 0, also n1 and n2 are 0,
[ ] indicates determinant either being present or absent depending on the value of n1, n2, n3, n4 and m,
( ) indicates a branch in the structure.

### Group 2-Neutral O-Glycans

The group 2 represents neutral O-glycans. The major structures represent O-glycans with LacNAc epitope (Galβ4GlcNAc), and fucosylated so-called Lewis x-structure (Galβ4[Fucα3]GlcNAc). The preferred structures include the Core 2 type O-glycans of the figures below. Alternative variants include Galβ4(Fucα3)₀₋₁GlcNAcβ3Galβ3GalNAc Core 1 type O-glycan structures (in the figures the glycans are free oligosaccharides and β-anomers; in glycoproteins in tissues the glycans are O-glycans and α-anomers):

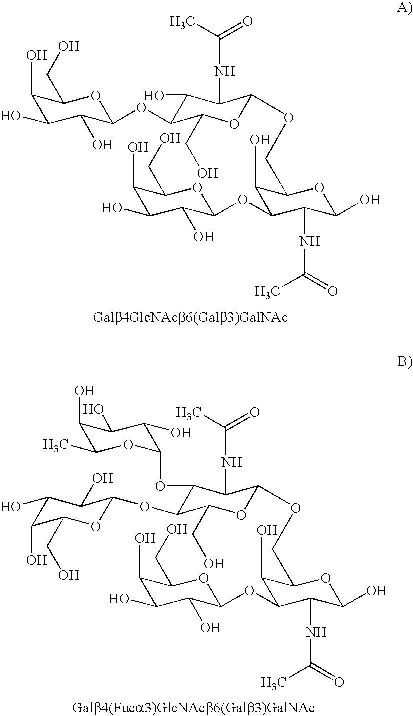

### Preferred Molecular Structural Features of Group 2

Based on the enzymatic digestion data and the release by β-elimination the neutral O-glycans include the following preferred structures

Galβ4[(Fucα3)]ₙGlcNAcβX[(]ₘGalβ3[)]ₘGalNAc[αSer/Thr]ₚ

wherein p, n and m are either independently 0 or 1, [ ] indicates determinant either being present or absent depending on the value of m and n, ( ) indicates a branch in the structure. X is 3, when m is 0; and X is 6 when m is 1.

The most preferred structures are according to the formula (when m is 1)

Galβ4[(Fucα3)]ₙGlcNAcβ6(Galβ3)GalNAc[αSer/Thr]ₚ

wherein
p and n are either independently 0 or 1.

In another embodiment the neutral O-glycans are according to the formula

Galβ4[(Fucα43)]ₙGlcNAcβ3Galβ3GalNAc[αSer/Thr]ₚ

wherein
p and n are either independently 0 or 1.

The Core 2 O-glycan structures are likely produced in Golgi apparatus through Core 2 structure GlcNAcβ6(Galβ3)GalNAcα, the inventors have also analysed larger Core 2 glycans from tissues, as described in the Examples.

### Group 3-Sialylated O-Glycans

The group 3 represents sialylated O-glycans. The major structure is a Core 2/4 type O-glycan with sialyl-LacNAc, NeuNAcα3Galβ4GlcNAcβ6(R-3)GalNAcαSer/Thr. The R substituent at 3-position of GalNAc is preferentially β1,3-linked Gal (Core 2) or β1,3-linked GlcNAc (Core 4).

### Comparison to O-Glycan Biosynthesis

The present structures are different from sialyl-Tn, T and sialyl-T O-glycan structures indicated previously for cancer. Previously sialylation of Core 1 has been considered to prevent Core 2 synthesis in certain cancer models, leading to increased expression of small O-glycan antigens, such as sialyl-Tn. The present invention shows opposite result, the increase of Core 2 structures in tumours, especially in malignant vs. benign tumours.

### Molecular Structures

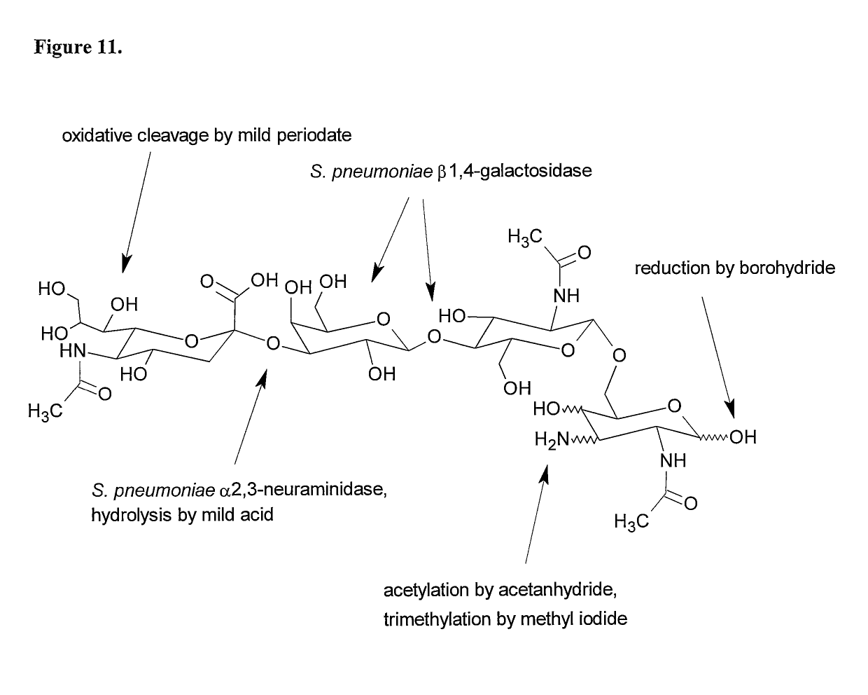

The group 3 structures are a large group of sialylated O-glycans, which have in common the O-glycan core structure GlcNAcβ6(R-3)GalNAc(α-Ser/Thr), where R is a possibly variable structure. In the conditions used for glycan isolation, typical fragment structures are produced from these glycans. The most typical such fragment (at m/z 899 for the sodium adduct ion) is depicted in FIG. 11 together with the experiments done to characterize the structure.

The most preferred structures of group 3 are according to the formula (when m is 1):

NeuNAcαX₁{Galβ4[(Fucα3)]ₙ₁GlcNAcβX₂}Galβ4[(Fucα3)]ₙ₂GlcNAcβ6([NeuNAcαX₃]ₙ₃Galβ3)GalNAc[αSer/Thr]ₚ

wherein n1, n2, n3 and p are either independently 0 or 1, m is 0, 1 or 2, { } and [ ] indicate determinant either being present or absent depending on the value of m, n1 and n2 ( ) indicates a branch in the structure. When m is 2, either or both of the Galβ4[(Fucα3)]ₙ₁GlcNAc-units may be fucosylated
X₁, X₂, and X₃ are 3 or 6, more preferably 3
When m is 2 one X₂ may be 3 and the other one 6 in a branched structure on the next Gal residue.

### Methods for Evaluating the Malignancy of Patient Samples

The present invention is directed to a method of evaluating the malignancy of a patient sample comprising the step of detecting the presence of cancer related oligosaccharide sequences in the sample, said oligosaccharide sequences comprising structures with terminal monosaccharides Manα, Galβ4, and SAα3.

Furthermore, the present invention is directed to a method of evaluating the malignancy of a patient sample comprising the step of detecting the presence or amount of a cancer related oligosaccharide sequence in the sample, said oligosaccharide sequence comprising any one of the structures from the following groups 1-3, more preferably from Groups 1-5 and/or optionally any one of the structures in Groups 5-8 and/or additional groups according to the invention.

The invention is further directed to method including analysis of at least low-mannose (abbreviated low-Man) one structure according of group 1. The invention is further directed to method including analysis of at least one LacNAc O-glycans one structure of groups 2 or 3.

### Combined Use of Different Groups

To increase the effectively of analysis a mixture of low-Man and LacNAc O-glycans or at least one low-Man and one LacNAc O-glycans is analyzed. In a preferred embodiment neutral glycans of groups 1 and 2 are analyzed.

To increase the specificity of the analysis one or two structures of Groups 1-3, preferably as preferred above is analyzed with at least one structure (preferably 1, 2 or 3 structures) selected from the groups 4-5, or more preferably selected from the groups 4-8, preferably so that when at least two structures are selected, these are selected from different groups.

Group 1) low-mannose N-glycans with Formula

[Manα2]ₙ₁[Manα3]ₙ₂([Manα2]ₙ₃[Manα6)]ₙ₄)[Manα6]ₙ₅([Manα2]ₙ₆[Manα2]ₙ₇[Manα3]ₙ₈)Manβ4GlcNAcβ4[(Fucα6)]ₘGlcNAc[3-N-Asn]ₚ

wherein p, n1, n2, n3, n4, n5, n6, n7, n8, and m are either independently 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; the sum of n1, n2, n3, n4, n5, n6, n7, and n8 is less than or equal to (m+3); and preferably n1, n3, n6, and n7 are 0 when m is 0; [ ] indicates determinant either being present or absent depending on the value of n1, n2, n3, n4, n5, n6, n7, n8, and m; and
( ) indicates a branch in the structure.

Preferred structures according to the present invention are described in the Examples, in which association of the structures with cancer was found in major human cancer types.

Group 2) Neutral O-Glycans with Formula

Galβ4[(Fucα3)]ₙGlcNAcβX[(]ₘGalβ3[)]ₘGalNAc[α-O-Ser/Thr]ₚ

wherein p, n and m are either independently 0 or 1, [ ] indicates determinant either being present or absent depending on the value of m and n, ( ) indicates a branch in the structure. X is 3, when m is 0; and X is 6 when m is 1; and

Preferred structures according to the present invention are described in the Examples, in which association of the structures with cancer was found in major human cancer types.

Group 3) Sialylated O-Glycans with Formula

SAαX₁{Galβ4[(Fucα3)]ₙ₁GlcNAcβX₂}ₘGalβ4[(Fucα3)]ₙ₂GlcNAcβ6([SAαX₃]ₙ₃Galβ3) GalNAc[α-O-Ser/Thr]ₚ

wherein n1, n2, n3 and p are either independently 0 or 1; m is 0, 1 or 2; X₁ and X₃ are independently 3 or 6, however in the most preferred embodiment both X₁ and X₃ are 3; X₂ is either 3 or 6, however in the most preferred embodiment X₂ is 3; SA is a sialic acid residue, preferentially Neu5Ac or Neu5Gc, however in the most preferred embodiment SA is Neu5Ac; { } and [ ] indicate determinant either being present or absent depending on the value of m, n1 and n2; ( ) indicates a branch in the structure.

Preferred structures according to the present invention are described in the Examples, in which association of the structures with cancer was found in major human cancer types.

### Cancer Indicating Structures and Combinations of Structures

The inventors found that an increased amount of any one of the abovementioned cancer-related oligosaccharide sequences in said patient sample indicates the cancerous nature of the sample. Furthermore, simultaneous increase of more than one of the abovementioned cancer-related oligosaccharide sequences is highly indicative of cancer, especially when more than one of the abovementioned oligosaccharide sequences, even more preferentially selected from more than one of the abovementioned oligosaccharide sequence groups, is simultaneously expressed in elevated amounts compared to healthy human tissues. The above-mentioned glycan structure groups were found to be cancer-associated in all cancer types studied.

It was found that the increased amount of the abovementioned oligosaccharide sequences was indicative of the malignancy of human tumors, though no such increase was found in benign tumors of the colon and the ovaries. The inventors found that malignant and benign growth in human patients could be distinguished by analyzing protein-linked glycan structures for the expression of the abovementioned oligosaccharide sequences according to the present invention. Furthermore, the analysis specificity was increased by combination with analysis of cancer type specific glycan features, as described below. The present invention is especially directed to cancer diagnostics or analysis of clinical state of cancer by analysing several glycan structures simultaneously according to the invention.

The present findings are considered medically very interesting. The novel methods to detect and diagnose cancer described in the present invention can be used in the clinical setting e.g. to give tools and data for decisions how to treat human patients. It is realized that ability to detect malignant cancer and to differentiate between benign and malignant tumors is of utmost importance in terms of efficient clinical decision-making and selection of the correct therapy.

The inventors further discovered that individual differences occur in normal tissue glycosylation and tumor-associated glycosylation changes, and in some patients the cancer-associated glycan changes are more prominent than in others. The present invention is further directed to using the methods for selecting patients for most effective therapy options according to their individual glycosylation profiles and the glycosylation profiles expressed in the disease, preferentially in the malignant tumor.

### Cancer Type Specific Glycan Groups

The inventors also found that changes in the expression of two additional glycan structure groups were indicative of malignant cancer in tumors originating from specific tissues, in addition to the abovementioned oligosaccharide sequences. These indicative glycan structures can be used to detect cancer or to distinguish malignant and benign growth in human patients, either in combination with the abovementioned three glycan groups, or separately.

Both of these structure groups are characterized by a common feature, the presence of a non-reducing terminal β-linked N-acetylhexosamine residue (HexNAcβ). In glycan profiling analyses where monosaccharide compositions can be assigned to analysed glycans, these glycans are indicated among the resulting glycan signals by the formula:

n(HexNAc)>n(Hex)≧2,

wherein n(component) in the amount of the monosaccharide component in a glycan molecular formula. Oligosaccharide sequences that fulfill the formula can be used to distinguish between normal and cancerous tissue materials, and/or benign and malignant tumors according to the present invention. Preferentially, the presence or amount of these oligosaccharide sequences is determined, and optionally compared with the presence or amount of other types of oligosaccharide sequences in the sample and/or specifically chosen oligosaccharide sequences groups according to the present invention. However, these HexNAcβ structures were found to be different in specific human tissues and tumors originating from them, as described below in more detail and described in the Examples. The present findings and uses thereof as described in the present invention are considered novel and medically significant.
Group 4) Glycans with Terminal (NeuAcα)₀₋₁HexNAcβHexNAcβ sequences

The inventors analyzed protein-linked glycans from patients with ovarian cancer or benign ovarian tumors. The patient samples were from multiple patients with benign ovarian cystadenoma or malignant ovarian cystadenocarcinoma, and a sample from normal ovary. It was found that terminal HexNAc structures were present in all the samples in slightly elevated amounts compared to other human tissues, but specifically in benign ovarian tumors these structures were highly increased and were among the major glycan components, as described in the Examples of the present invention. However, in corresponding malignant tumors, the amounts of terminal HexNAcβstructures were even decreased compared to the normal ovary. These changes were found to be consistent in all the studied samples, indicating that the phenomenon is common in the human ovary and its tumors. Detection of the ovary-specific terminal HexNAcβoligosaccharide sequence was found to effectively distinguish between benign and malignant tumors of the ovary, and contribute to the separation of the normal and malignant ovary samples.

The inventors characterized the ovary-specific HexNAcβstructures in further detail, as described in the Examples of the present invention. The results suggested that the observed glycan structures associated with benign ovarian tumors structures include GalNAcβGlcNAcβ and Neu5AcαGalNAcβGlcNAc non-reducing terminal sequences, or non-sialylated and sialylated di-N-acetyllactosediamine (LacdiNAc), occurring mainly in N-glycans. The inventors have previously characterized LacdiNAc structures in human tumors and described novel methods and reagents for the detection and modification of LacdiNAc structures as well as harnessing immune responses against LacdiNAc structures. However, the differences between normal, benign, and malignant tissue materials and especially ovarian tissue materials of the present invention are novel. It is realized that the present indication represents further uses also for previously described methods and reagents, and the present invention is specifically directed to using LacdiNAc-specific reagents and methods for the detection of cancer, preferentially ovarian cancer and especially to distinguish between glycans or glycoconjugates originating from benign and malignant ovarian tumors or normal ovarian tissue.

In a typical embodiment of the present invention, ovary-associated terminal HexNAcβsequences are detected and their presence indicates normal ovary tissue or benign growth of the ovary. However, in a more preferred embodiment the ovary-associated terminal HexNAcβsequences are quantitated and their increased amount, compared to other human tissues or normal ovary, indicates presence of normal ovary tissue or benign growth of the ovary. In contrast, malignant tumors of the ovary do not show similar increased amounts of HexNAcβsequences. In an even more preferred embodiment of the present invention, ovary-associated oligosaccharide sequences are profiled according to the present invention, and the relative amounts of terminal HexNAcβsequences are compared to the other oligosaccharide sequences present in the sample. Guidelines for recognition of terminal HexNAcβ sequences and oligosaccharide sequences for comparison are described below. In another embodiment of the present invention, experimental analysis signals corresponding to terminal HexNAcβoligosaccharide sequences as such, or other cancer-associated oligosaccharide sequences recognized in the present invention, for example mass spectrometric signals, are used for evaluation of the cancerous status of a sample.

### 1) Ovary-Associated HexNAcβ Oligosaccharide Sequences

According to the present invention, ovary tissue and tumor samples contain HexNAcβoligosaccharide sequences as defined by the formula above, more specifically terminal HexNAcβHexNAcβ structures. Typically, HexNAcβHexNAcβstructures include oligosaccharide sequences containing the motifs HexₘHexNAcₘ₊₁, HexₘHexNAcₘ₊₃, or HexₘHexNAcₘ₊₅ in their monosaccharide compositions. Another useful group definition of HexNAcβoligosaccharide sequences according to the present invention includes glycans that are susceptible to the action of β-hexosaminidase, but not to β-glucosaminidase, as described in the Examples. Typical mass spectrometric signals, monosaccharide compositions, and corresponding oligosaccharide sequences indicative of the cancerous status of a patient sample are further described in the Examples, and the present invention is specifically directed to using these signals, monosaccharide compositions, and the corresponding oligosaccharide sequences for the evaluation of the cancerous status of a sample. For practical reasons, the amounts of the HexNAcβHexNAcβoligosaccharide sequences can be approximated and/or extrapolated from the monosaccharide compositions and experimental evidence from previous analyses of similar tissues, and also these approximations are suitable for effective diagnostic results, as shown in the Examples.

### 2) Oligosaccharide Sequences Useful for Comparison

In approximate order of increasing specificity, oligosaccharide sequences useful for comparison include total glycans present in the sample, (1) sialylated or (2) neutral glycans, total N-glycans, total complex-type glycans, (1) sialylated or (2) neutral complex-type glycans, total complex-type N-glycans, (1) sialylated or (2) neutral complex-type N-glycans, and normal glycans corresponding to HexNAcβ glycans. In the present list, (1) and (2) indicate glycan groups useful for comparison of (1) sialylated and (2) neutral HexNAcβoligosaccharide sequences, respectively. The normal glycans in the latter definition have Hex substituted for HexNAc in their monosaccharide compositions, and may be defined for example as oligosaccharide sequences containing the motifs Hexₘ₊₁HexNAcₘ, Hexₘ₊₂HexNAcₘ₊₁, and Hexₘ₊₃HexNAcₘ₊₂ in their monosaccharide compositions, when present in the same sample as HexNAcβ oligosaccharide sequences containing the motifs HexₘHexNAcₘ₊₁, HexₘHexNAcₘ₊₃, and HexₘHexNAcₘ₊₅ in their monosaccharide compositions, respectively. For example, the normal monosaccharide composition motif Hex₅HexNAc₄ corresponds to the HexNAcβ composition Hex₃HexNAc₆. Another useful group of oligosaccharide sequences for comparison include those that are susceptible to the action of O-glucosaminidase, as described in the Examples of the present invention.

Practical procedures for comparison of samples and analysis results with regard to HexNAcβstructures and ovarian tissue and tumor patient samples as well as methods for detection and quantitation of oligosaccharide sequences are described in the present invention.

### Group 5) Terminal β-Linked GlcNAc Glycans

In glycan profiling analyses where monosaccharide compositions can be assigned to analysed glycans, these glycans are indicated among the resulting glycan signals by the formula:

n(HexNAc)>n(Hex)≧2,

wherein n(component) in the amount of the monosaccharide component in a glycan molecular formula. Oligosaccharide sequences that fulfil the formula can be used to distinguish between normal and cancerous tissue materials according to the present invention. Preferentially, the presence or amount of these oligosaccharide sequences is determined, and optionally compared with the presence or amount of other types of oligosaccharide sequences in the sample and/or specifically chosen oligosaccharide sequences groups according to the present invention. In general, terminal GlcNAcβoligosaccharide sequences are susceptible to α-glucosaminidase as well as α-hexosaminidase and other enzymes such as specific β1,4-galactosyltransferase, which together with glycan profiling according to the present invention can be used to distinguish between cancerous and healthy tissue samples. However, as described in the present invention, glycosylation is tissue specific and cancer type specific, and information about GlcNAcβstructures can be extrapolated from the reference information of glycosylation described in the present invention, and additional glycosylation information produced by the methods of the present invention.

The inventors have previously characterized GlcNAcβstructures in human tumors and described novel methods and reagents for the detection and modification of GlcNAcβ structures as well as harnessing immune responses against GlcNAcβstructures. However, the detection method of GlcNAcβstructures by approximation through the formula above in glycan profiling of tissue materials, methods for specific comparison with other oligosaccharide sequence groups present in the sample, and especially the combination of data about HexNAcβoligosaccharide sequences with the other cancer-associated oligosaccharide sequences described in the present invention to increase resolution power of the method are novel. It is realized that the present invention represents further uses also for the previously described methods and reagents, and the present invention is specifically directed to using GlcNAcβ-specific reagents and methods for the detection of cancer, especially in conduction with the other oligosaccharide sequence groups described above.

### General Structures Representing Oligosaccharide Sequences

The cancer related oligosaccharide sequences described herein can be a part of a glycolipid, a part of a glycoprotein, and/or a part of a N-acetyllactosamine chain. The cancer specific oligosaccharide sequences can also be a part of glycolipids, a part of N-linked glycans or O-linked glycans of glycoproteins, free oligosaccharides, or glycans such as glycopeptides. Defects or changes in biosynthetic and/or biodegradative pathways of tumors lead to the synthesis of the cancer related oligosaccharide sequences both on glycolipids and glycoproteins.

The term “oligosaccharide sequence” indicates that the monosaccharide residue/residues in the sequence are part of a larger glycoconjugate, which contains other monosaccharide residues in a chain, which may be branched, or may have natural substituted modifications of oligosaccharide chains. The oligosaccharide chain is normally conjugated to a lipid anchor or to a protein. In a preferred embodiment the oligosaccharide sequences according to the present invention are non-reducing terminal oligosaccharide sequences, which means here that the oligosaccharide sequences are not linked to other monosaccharide or oligosaccharide structures except optionally from the reducing end of the oligosaccharide sequence. The oligosaccharide sequence when present as conjugate is preferably conjugated from the reducing end of the oligosaccharide sequence, though other linkage positions which are tolerated by the antibody/binding substance binding can also be used. In a more specific embodiment the oligosaccharide sequence according to the present invention means the corresponding oligosaccharide residue which is not linked by natural glycosidic linkages to other monosaccharide or oligosaccharide structures. The oligosaccharide residue is preferably a free oligosaccharide or a conjugate or derivative from the reducing end of the oligosaccharide residue.

In one embodiment of the invention the cancer specific oligosaccharides are detected for the diagnostics of cancer or tumor.

Preferably the tumor specific oligosaccharide sequence is detected by a specific binding substance which can be an aptamer, lectin, peptide, or protein, such as an antibody, a fragment thereof or genetically engineered variants thereof. More preferably the specific binding substance is divalent, oligovalent or polyvalent. Most preferably the binding substance is a lectin or an antibody.

Specific binding combinatorial chemistry libraries can be used to search for the binding molecules. Saccharide binding proteins, antibodies or lectins can be engineered, for example, by phage display methods to produce specific binders for the structures of the invention. Labelled bacteria or cells or other polymeric surfaces containing molecules recognizing the structures can be used for the detection. Oligosaccharide sequences can also be released from cancer or tumor cells by endoglycosidase enzymes. Alternatively oligosaccharides can be released by protease enzymes, resulting in glycopeptides. Chemical methods to release oligosaccharides or derivatives thereof include, e.g., ozonolysis of glycolipids and beta-elimination or hydrazinolysis methods to release oligosaccharides from glycoproteins.

Alternatively the glycolipid fraction can be isolated. A substance specifically binding to the cancer specific oligosaccharide sequences can also be used for the analysis of the same sequences on cell surfaces. Said sequences can be detected e.g. as glycoconjugates or as released and/or isolated oligosaccharide fractions. The possible methods for the analysis of said sequences in various forms also include NMR spectroscopy, mass spectrometry and glycosidase degradation methods. Preferably at least two analysis methods are used, especially when methods of limited specificity are used.

Analysis of Multiple Cancer Specific Structures Simultaneously from Mass Spectrometric Profiles

The present invention is especially directed to the analysis and/or comparison of several analytical signals, preferably mass spectrometry signals produced from a sample comprising total fraction of oligosaccharides released from a cancer or a tumor sample. A single mass spectrum of an oligosaccharide fraction comprise a profile of glycosylation and multiple peaks indicating the potential presence of the oligosaccharide sequences and potential presence of cancer specific oligosaccharide sequences and altered levels thereof in comparison to normal tissue sample or a benign tumour sample. The profiles are determined preferably by MALDI-TOF mass spectrometry as described in the Examples. The total oligosaccharide fraction corresponds preferably to the total fraction of protein oligosaccharides, preferably comprising at least one cancer or tumor specific oligosaccharide sequence according to the invention. In another preferred embodiment the total oligosaccharide fraction comprises at least one cancer or tumor specific O-glycosidic and one N-glycosidic oligosaccharide according to the invention. The present invention is further directed to analysis of the multiple mass spectrometric signals after the total oligosaccharide fraction is released from a cancer or tumor sample is subjected to an enzymatic or a chemical digestion step. The enzymatic digestion is preferably performed by a glycosidase enzyme, preferably selected from the group: galactosidase, sialidase, N-acetylhexosaminidase, N-acetylglucosaminidase, facosidase, or mannosidase.

The present invention is also directed to the use of the tumor specific oligosaccharide sequences or analogs or derivatives thereof to produce polyclonal or monoclonal antibodies recognizing said structures using following process: 1) producing synthetically or biosynthetically a polyvalent conjugate of an oligosaccharide sequence of the invention or analogue or derivative thereof, the polyvalent conjugate being, for instance, according to the following structure: position C1 of the reducing end terminal of an oligosaccharide sequence (OS) comprising the cancer specific sequence described in the present invention is linked (-L-) to an oligovalent or a polyvalent carrier (Z), via a spacer group (Y) and optionally via a monosaccharide or oligosaccharide residue (X), forming the following structure

[OS—(X)ₙ-L-Y]ₘ-Z

wherein integer m has values m>1 and n is independently 0 or 1; L can be oxygen, nitrogen, sulfur, or a carbon atom; X is preferably lactosyl-, galactosyl-, poly-N-acetyl-lactosaminyl, or part of an O-glycan or an N-glycan oligosaccharide sequence, Y is a spacer group or a terminal conjugate such as a ceramide lipid moiety or a linkage to Z; 2) immunizing an animal or human with polyvalent conjugate together with an immune response activating substance. Preferably the oligosaccharide sequence is polyvalently conjugated to an immune response activating substance and the conjugate is used for immunization alone or together with an additional immune response activating substance. In a preferred embodiment the oligosaccharide conjugate is injected or administered mucosally to an antibody-producing organism with an adjuvant molecule or adjuvant molecules. For antibody production the oligosaccharide or analogs or derivatives thereof can be polyvalently conjugated to a protein such as bovine serum albumin, keyhole limpet hemocyanin, a lipopeptide, a peptide, a bacterial toxin, a part of peptidoglycan or immunoactive polysaccharide or to another antibody production activating molecule. The polyvalent conjugates can be injected to an animal with adjuvant molecules to induce antibodies by routine antibody production methods known in the art.

Antibody production or vaccination can also be achieved by analogs or derivatives of the cancer specific oligosaccharide sequences. Simple analogs of the N-acetyl-group containing oligosaccharide sequences include compounds with modified N-acetyl groups, for example, N-alkyls, such as N-propanyl.

According to the invention it is possible to use the tumor specific oligosaccharide sequences for the purification of antibodies from serum, preferably from human serum. The cancer specific oligosaccharides or derivatives or analogs, such as a close isomer, can also be immobilized for the purification of antibodies from serum, preferably from human serum. The present invention is directed to natural human antibodies that bind strongly to the cancer specific oligosaccharide sequences described in the present invention.

The cancer specific oligosaccharide sequences can also be used for detection and/or quantitation of the human antibodies binding to the cancer specific oligosaccharide sequences, for example, in enzyme-linked immunosorbent assay (ELISA) or affinity chromatography type assay formats. The detection of human antibodies binding to the cancer specific oligosaccharide sequences is preferably aimed for diagnostics of cancer, development of cancer therapies, especially cancer vaccines against the oligosaccharide sequences described in the present invention, and search for blood donors which have high amounts of the antibodies or one type of the antibody.

Furthermore, it is possible to use human antibodies or humanized antibodies against the cancer specific oligosaccharide sequences to reduce the growth of or to destroy a tumor or cancer. Human antibodies can also be tolerated analogs of natural human antibodies against the cancer specific oligosaccharide sequences; the analogs can be produced by recombinant gene technologies and/or by biotechnology and they may be fragments or optimized derivatives of human antibodies. Purified natural anti-tumor antibodies can be administered to a human patient without any expected side effect as such antibodies are transferred during regular blood transfusions. This is true under conditions that the cancer specific structures are not present on normal tissues or cells and do not vary between individuals as blood group antigens do. In another embodiment of the invention species specific animal antibodies are used against a tumor or cancer of the specific animal. The production of specific humanized antibodies by gene engineering and biotechnology is also possible: the production of humanized antibodies has been described in U.S. Pat. Nos. 5,874,060 and 6,025,481, for example. The humanized antibodies are designed to mimic the sequences of human antibodies and therefore they are not rejected by immune system as animal antibodies are, if administered to a human patient. It is realized that the method to reduce the growth of or to destroy cancer applies both to solid tumors and to cancer cells in general. It is also realized that the purified natural human antibodies recognizing any human cancer specific antigen, preferably an oligosaccharide antigen, can be used to reduce the growth of or to destroy a tumor or cancer. In another embodiment species specific animal antibodies are used against a tumor or cancer of the specific animal.

According to the invention human antibodies or humanized antibodies against the cancer specific oligosaccharides, or other tolerated substances binding the tumor specific oligosaccharides, are useful to target toxic agents to tumor or to cancer cells. The toxic agent could be, for example, a cell killing chemotherapeutics medicine, such as doxorubicin (Arap et al., 1998), a toxin protein, or a radiochemistry reagent useful for tumor destruction. Such therapies have been demonstrated in the art. The toxic agent may also cause apoptosis or regulate differentiation or potentiate defense reactions against the cancer cells or tumor. In another embodiment of the invention species specific animal antibodies are used against a tumor or cancer of the specific animal. The cancer or tumor binding antibodies according to the present invention can be also used for targeting prodrugs active against tumor or enzymes or other substances converting prodrugs to active toxic agents which can destroy or inhibit tumor or cancer, for example in so called ADEPT-approaches.

The therapeutic antibodies described above can be used in pharmaceutical compositions for the treatment or prevention of cancer or tumor. The method of treatment of the invention can also be used when patient is under immunosuppressive medication or he/she is suffering from immunodeficiency.

### Other Methods for Therapeutic Targeting of Tumors

It is realized that numerous other agents besides antibodies, antibody fragments, humanized antibodies and the like can be used for therapeutic targeting of cancer or tumors similarity with the diagnostic substances. It is specifically preferred to use non-immunogenic and tolerable substances to target cancer or tumor. The targeting substances binding to the cancer or tumor comprise also specific toxic or cytolytic or cell regulating agents which leads to destruction or inhibition of cancer or tumor. Preferably the non-antibody molecules used for cancer or tumor targeting therapies comprise molecules specifically binding to the cancer or tumor specific oligosaccharide sequences according to the present invention are aptamers, lectins, genetically engineered lectins, glycosidases and glycosyltransferase and genetically engineered variants thereof. Labelled bacteria, viruses or cells or other polymeric surfaces containing molecules recognizing the structures can be used for the cancer or tumor targeting therapies. The cancer or tumor binding non-antibody substances according to the present invention can also be used for targeting prodrugs active against cancer or tumor or for targeting enzymes or other substances converting prodrugs to active toxic agents that can destroy or inhibit cancer or tumor.

### Detection and Diagnostics

Furthermore the present invention is directed to methods for the detection of the pathogenic entities or activities by the invention. The specific transfer of modified monosaccharides to the pathogenic entities allows the detection of the pathogenic entities. For this purpose the modification of the monosaccharide need not to be toxic. The monosaccharide is modified by a label substance like a tag substance including for example an antigen detectable by an antibody, biotin, digotoxigenin, digitoxin or a directly detectable substance with examples of fluorescent substance like rhodamine or fluorescein or substance with chemiluminescence activity or phosphoresce substance or a specific molecular mass marker detectable by mass spectrometry.

In a preferred embodiment the modified monosaccharide is labeled with two label compounds, which are more preferentially a tag substance and a directly detectable substance and most preferentially a tag substance like biotin and a mass spectrometry label. The label substance is preferentially linked through a spacer to the modified monosaccharide. The invention is also directed to the use of said carbohydrate for diagnostics of the pathogenic entities and diseases related to them. The invention is specifically directed to the use of said carbohydrate/carbohydrates for diagnostics of infections, cancer and malignancies. The invention is especially directed to the use of immunologically active or toxic carbohydrate for the treatment diseases like infections, cancers and malignancies. Preferentially the cell surface carbohydrates are labeled by the modified monosaccharide. Modified monosaccharides aimed for detection are useful for detection of certain congenital disorders of glycosylation and under-sialylated LDL. Especially useful are labeled nucleotide sugars.

### Cancer Vaccines

Furthermore, according to the invention the cancer specific oligosaccharide sequences or analogs or derivatives thereof can be used as cancer or tumor vaccines in man to stimulate immune response to inhibit or eliminate cancer or tumor cells. The treatment may not necessarily cure cancer or tumor but it can reduce tumor burden or stabilize a cancer condition and lower the metastatic potential of cancers. For the use as vaccines the oligosaccharides or analogs or derivatives thereof can be conjugated, for example, to proteins such as bovine serum albumin or keyhole limpet hemocyanin, lipids or lipopeptides, bacterial toxins such as cholera toxin or heat labile toxin, peptidoglycans, immunoreactive polysaccharides, or to other molecules activating immune reactions against a vaccine molecule. A cancer or tumor vaccine may also comprise a pharmaceutically acceptable carrier and optionally an adjuvant. Suitable carriers or adjuvants are, e.g., lipids known to stimulate the immune response. The saccharides or derivatives or analogs thereof, preferably conjugates of the saccharides, can be injected or administered mucosally, such as orally or nasally, to a cancer patient with tolerated adjuvant molecule or adjuvant molecules. The cancer or tumor vaccine can be used as a medicine in a method of treatment against cancer or tumor. Preferably the method is used for the treatment of a human patient. Preferably the method of treatment is used for the treatment of cancer or tumor of a patient, who is under immunosuppressive medication or the patient is suffering from immunodeficiency.

Furthermore it is possible to produce a pharmaceutical composition comprising the cancer specific oligosaccharide sequences or analogs or derivatives thereof for the treatment of cancer or tumor. Preferably the pharmaceutical composition is used for the treatment of a human patient. Preferably the pharmaceutical composition is used for the treatment of cancer or tumor, when patient is under immunosuppressive medication or he/she is suffering from immunodeficiency. The methods of treatment or the pharmaceutical compositions described above are especially preferred for the treatment of cancer or tumor diagnosed to express the cancer specific oligosaccharide sequences of the invention. The methods of treatment or the pharmaceutical compositions can be used together with other methods of treatment or pharmaceutical compositions for the treatment of cancer or tumor. Preferably the other methods or pharmaceutical compositions comprise cytostatics, anti-angiogenic pharmaceuticals, anti-cancer proteins, such as interferons or interleukins, or use of radioactivity.

Use of antibodies for the diagnostics of cancer or tumor and for the targeting of drugs to cancer has been described with other antigens and oligosaccharide structures (U.S. Pat. No. 4,851,511; U.S. Pat. No. 4,904,596; U.S. Pat. No. 5,874,060; U.S. Pat. No. 6,025,481; U.S. Pat. No. 5,795,961; U.S. Pat. No. 4,725,557; U.S. Pat. No. 5,059,520; U.S. Pat. No. 5,171,667; U.S. Pat. No. 5,173,292; U.S. Pat. No. 6,090,789; U.S. Pat. No. 5,708,163; U.S. Pat. No. 5,902,725 and U.S. Pat. No. 6,203,999). Use of cancer specific oligosaccharides as cancer vaccines has also been demonstrated with other oligosaccharide sequences (U.S. Pat. No. 5,102,663; U.S. Pat. No. 5,660,834; U.S. Pat. No. 5,747,048; U.S. Pat. No. 5,229,289 and U.S. Pat. No. 6,083,929).

### Combination of the Therapeutic and Diagnostic Methods

The present invention is specifically directed to analysis of abnormal and normal glycosylation structures from human tumors and cancers and use of the analytical information for the production of therapeutic antibodies or cancer vaccines according to the invention. To achieve effective therapeutic response, it is preferred that the specific cancer type in the patients to be treated expresses cancer-associated glycans according to the present invention. The present invention is specifically directed to individually targeted treatment of cancer including following steps:
1. analysis of glycosylation of tumor or cancer tissue of a patient
2. analysis of normal glycosylation of the tissue containing the cancer
3. use of the therapies according to the present invention if the patient has cancer specific oligosaccharide sequences according to the present invention in cancer.

The data in the Examples shows the usefulness of the combination of analysis of the cancer specific structures according to the invention, because there are individual variations in glycosylation of tumors and normal tissues. The normal tissue close to tumor may also be partially contaminated by materials secreted by tumor that may be taken to consideration when analyzing the normal tissue data.

The substance according to the invention can be attached to a carrier. Methods for the linking of oligosaccharide sequences to a monovalent or multivalent carrier are known in the art. Preferably the conjugation is performed by linking the cancer specific oligosaccharide sequences or analogs or derivatives thereof from the reducing end to a carrier molecule. When using a carrier molecule, a number of molecules of a substance according to the invention can be attached to one carrier increasing the stimulation of immune response and the efficiency of the antibody binding. To achieve an optimal antibody production, conjugates larger than 10 kDa carrying typically more than 10 oligosaccharide sequences are preferably used.

The oligosaccharide sequences according to the invention can be synthesized, for example, enzymatically by glycosyltransferases, or by transglycosylation catalyzed by a glycosidase enzyme or a transglycosidase enzyme, for review see Ernst et al. (2000). Specificities of the enzymes and their use of co-factors such as nucleotide sugar donors, can be engineered. Specific modified enzymes can be used to obtain more effective synthesis, for example, glycosynthase is modified to achieve transglycosylation but not glycosidase reactions. Organic synthesis of the saccharides and conjugates of the invention or compounds similar to these are known (Ernst et al., 2000). Carbohydrate materials can be isolated from natural sources and be modified chemically or enzymatically into compounds according to the invention. Natural oligosaccharides can be isolated from milks of various ruminants and other animals. Transgenic organisms, such as cows or microbes, expressing glycosylating enzymes can be used for the production of saccharides.

It is possible to incorporate an oligosaccharide sequence according to the invention, optionally with a carrier, in a pharmaceutical composition, which is suitable for the treatment of cancer or tumor in a patient. Examples of conditions treatable according to the invention are cancers in which the tumor expresses one or more of the tumor specific oligosaccharides described in the invention. The treatable cancer cases can be discovered by detecting the presence of the tumor specific oligosaccharide sequences in a biological sample taken from a patient. Said sample can be a biopsy or a blood sample.

The pharmaceutical composition according to the invention may also comprise other substances, such as an inert vehicle, or pharmaceutically acceptable carriers, preservatives etc., which are well known to persons skilled in the art.

The substance or pharmaceutical composition according to the invention may be administered in any suitable way. Methods for the administration of therapeutic antibodies or vaccines are well known in the art.

The term “treatment” used herein relates to both treatment in order to cure or alleviate a disease or a condition, and to treatment in order to prevent the development of a disease or a condition. The treatment may be either performed in an acute or in a chronic way.

The term “patient”, as used herein, relates to any mammal in need of treatment according to the invention.

When a cancer specific oligosaccharide or compound specifically recognizing cancer specific oligosaccharides of the invention is used for diagnosis or typing, it may be included e.g. in a probe or a test stick, optionally in a test kit. When this probe or test stick is brought into contact with a sample containing antibodies from a cancer patient or cancer cells or tissue of a patient, components of a cancer positive sample will bind the probe or test stick and can be thus removed from the sample and further analyzed.

In the present invention the term “tumor” means solid multicellular tumor tissues. Furthermore the term “tumor” means herein premalignant tissue, which is developing to a solid tumor and has tumor specific characteristics. The present invention is preferably directed to primary human cancer samples. It is well known that glycosylations in cultivated cancer cells vary and are not in general relevant with regard to cancer. It is also known that transfections, cell culture media and dividing solid tumor to single cells may have dramatics effects for glycosylations. When referring to therapies tumor specific oligosaccharides or oligosaccharide sequences (possibly occasionally referred as cancer specific oligosaccharides/oligosaccharide sequences) are targeted for treatment of all kinds of cancers and tumors. The term cancer includes tumors.

The present invention is specifically directed to the treatment of all types of cancer or tumors expressing the tumor specific oligosaccharide sequences according to the present invention. Examples of preferred cancer types includes cancers of larynx, colon cancer, stomach cancer, breast cancer, lung cancer, kidney cancer, pancreas cancer, and ovarian cancer.

Glycolipid and carbohydrate nomenclature is according to recommendations by the IUPAC-IUB Commission on Biochemical Nomenclature (Carbohydr. Res. 1998, 322:167; Carbohydr. Res. 1997, 297:1; Eur. J. Biochem. 1998, 257:29).

It is assumed that Gal, Glc, Man, GlcNAc, GalNAc, and NeuNAc are of the D-configuration, Fuc of the L-configuration, and that all monosaccharide units are in the pyranose form. Glucosamine is referred as GlcN and galactosamine as GalN. Glycosidic linkages are shown partly in shorter and partly in longer nomenclature, the linkages α3 and α6 of the NeuNAc-residues mean the same as α2-3 and α2-6, respectively, and β1-3, β1-4, and β1-6 can be shortened as β3, β4, and β6, respectively. Lactosamine or N-acetyllactosamine or Galβ3/4GlcNAc means either type one structure residue Galβ3GlcNAc or type two structure residue Galβ1-4GlcNAc, and SA is sialic acid, NeuAc or NeuGc, preferentially Neu5Ac, Lac refers to lactose and Cer is ceramide. Hex is any hexose, preferably Man, Gal, or Glc; HexNAc is any N-acetylhexosamine, preferably GlcNAc or GalNAc; and dHex is preferably Fuc.

### Description of Preferred Glycan Methods

### Tissue Derived Glycomes

Glycomes—Novel Glycan Mixtures from Tissue Samples

The present invention reveals novel methods for producing novel carbohydrate compositions, glycomes from animal tissues, preferably from vertebrates, more preferably human and mammalian tissues. The tissue substrate materials can be total tissue samples and fractionated tissue parts, such as serums, secretions and isolated differentiated cells from the tissues, or artificial models of tissues such as cultivated cell lines.

The invention revealed that the glycan structures on cell surfaces vary between the various tissues and same tissues under changing conditions, especially cancer.

The glycan structures on cell surfaces in general have been known to have numerous biological roles. Thus the knowledge about exact glycan mixtures from cell or tissue surfaces is important for knowledge about the status of cells. The invention revealed that multiple conditions affect the cells and cause changes in their glycomes.

### Molecular Weight Distribution and Structure Groups of the Glycomes

### General Compositions

The inventors were able to release or isolate various glycan fractions from tissue materials, which are useful for the characterization of the cancer cellular material. The glycans or major part thereof are released preferably from glycoproteins or glycolipids of tissue samples. The invention is specifically directed to such glycan fractions. The glycan fractions of tissue samples comprise typically multiple, at least about 10 “glycan mass components” typically corresponding at least ten glycans and in most cases clearly more than 10 glycan structures.

### Glycan Mass Components and Corresponding Monosaccharide Compositions

The glycan mass components correspond to certain molecular weights observable by mass spectrometry and further correspond to specific monosaccharide composition or monosaccharide compositions. Each monosaccharide component is normally present in a glycan as glycosidically linked monosaccharide residue in the nonreducing end part of glycan and the reducing end monosaccharide may be in free alditol form or modified for example by reduction or conjugated to an reducing end modifying reagent well known in the art or to one, two or several amino acids in case of glycopeptides. Monosaccharide composition can be obtained from molecular mass in a mass spectrum (glycan mass component) after correcting potential effect of the ion forms observable by the specific mass spectrometry technology such as protonation/deprotonation, Na⁺, K⁺, Li⁺, or other adduct combinations, or isotope pattern derived effects. The monosaccharide compositions are calculated by fitting mixtures of individual monosaccharide (residue) masses and modification groups to corrected molecular mass of glycan mass component. Typically the molecular mass of fitting composition and the experimental mass correspond to each other very closely with similar first and even second decimals with optimal calibration.

The fitting may be further checked by measuring the experimental mass difference from the smaller and/or larger glycan mass component next in the putative biosynthetic series of a glycan type and comparing the difference with the exact molecular mass of corresponding monosaccharide unit (residue), typically the mass differences of fitting components in a good quality mass spectrum and with correct marking of peaks in decimals, preferably in second or third decimal of the mass number depending on the resolution of the specific mass spectrometric method. For optimal mass accuracy, an internal calibration may be used, where two or more known component's mass peaks are used to re-calculate masses for each component in the spectrum. Such calibration components are preferably selected among the most abundant glycan signals present in the glycan profiles, in the case of human or other animal cell derived glycan profiles most preferably selected among the most abundant glycan signals present e.g. in Figures described in the present invention.

The monosaccharide composition includes monosaccharide component names and number, typically as subscript, indicating how many of the individual mass components are present in the monosaccharide composition; and names of assigned modifying groups and numbers indicating their abundance.

It is further realized that the masses of glycan mass component may be obtained as exact monoisotopic mass of usually smallest isotope of the glycan mass component or as an average mass of the isotope distribution of the glycan mass component. Exact mass is calculated form exact masses of individual mass components and average from masses average masses of individual mass components. Person skilled in art can recognize from the peak shapes (i.e. by the resolution obtained) in the mass spectrum whether to use monoisotopic or average masses to interpret the spectra. It is further realized that average and exact masses can be converted to each other when isotope abundances of molecules are known, typically natural abundance without enrichment of isotopes can be assumed, unless the material is deliberately labelled with radioactive or stable isotopes.

It is further realized that specific rounded mass numbers can be used as names for glycan mass components. The present invention uses preferably mass numbers rounded down from the exact mass of the monosaccharide composition (and usually observable or observed mass) to closest integer as names of glycan mass components.

The masses of glycan mass components are obtained by calculating molecular mass of individual monosaccharide components (Hex, HexNAc, dHex, NeuAc) from the known atom compositions (for example hexose corresponds to C₆H₁₂O₆) and subtracting for water in case of monosaccharide residue, followed by calculating the sum of the monosaccharide components (and possible modifications such as SO₃ or PO₃H). It is further realized that molecular masses of glycans may be calculated from atomic compositions or any other suitable mass units corresponding molecular masses of these. The molecular masses and calculation thereof are known in the art and masses of monosaccharide components/residues are available in tables with multiple decimals from various sources.

It is further realized that many of the individual monosaccharide compositions described in the present invention further correspond to several isomeric individual glycans. In addition, there exist also monosaccharide compositions that have nearly equal masses, for example dHex2 and NeuAc monosaccharide residues that have nearly equal masses, and other examples can be presented by a person skilled in the art. It is realized that the ability to differentiate compositions with nearly equal masses depends on instrumentation, and the present method is especially directed to a possibility to select also such compositions in place of proposed compositions.

The preferred glycans in glycomes comprise at least two of following monosaccharide component residues selected from group: Hexoses (Hex) which are Gal, Glc and Man; N-acetylhexosamines (HexNAc) which are GlcNAc and GalNAc; pentose, which is Xyl; Hexuronic acids which are GlcA and IdoA; deoxyhexoses (dHex), which is fucose and sialic acids which are NeuAc and/or NeuGc; and further modification groups such as acetate (Ac), sulphate and phosphate forming esters with the glycans. The monosaccharide residues are further grouped as major backbone monosaccharides including GlcNAc, HexA, Man and Gal; and specific terminal modifying monosaccharide units Glc, GalNAc, Xyl and sialic acids.

### Detection of Glycan Modifications

The present invention is directed to analyzing glycan components from biological samples, preferably as mass spectrometric signals. Specific glycan modifications can be detected among the detected signals by determined indicative signals as exemplified below. Modifications can also be detected by more specific methods such as chemical or physical methods, for example mass spectrometric fragmentation or glycosidase detection as disclosed in the present invention. In a preferred form of the present method, glycan signals are assigned to monosaccharide compositions based on the detected m/z ratios of the glycan signals, and the specific glycan modifications can be detected among the detected monosaccharide compositions.

In a further aspect of the present invention, relative molar abundances of glycan components are assigned based on their relative signal intensities detected in mass spectrometry as described in the Examples, which allows for quantification of glycan components with specific modifications in relation to other glycan components. The present method is also directed to detecting changes in relative amounts of specific modifications in cells at different time points to detect changes in cell glycan compositions.

Glycome Glycan Fraction Further Comprising Monosaccharides

The invention is specifically directed to glycan compositions, which further comprise at least one monosaccharide component in free form, preferably a preferred monosaccharide component described above. The monosaccharide comprising compositions are in a preferred embodiment derived from a cell material or released glycomes, which has been in contact with monosaccharide releasing chemicals or enzymes, preferably with exoglycosidase enzymes or chemicals such as oxidizing reagents and/or acid or base, more preferably with a glycosidase enzyme. The invention is further directed to compositions comprising a specific preferred monosaccharide according to the invention, an exoglycosidase enzyme capable releasing all or part of the specific monosaccharide and an glycan composition according to the invention from which at least part of the terminal specific monosaccharide has been released.

### Limit of Detection for Glycome Components

It is further realized that by increasing the sensitivity of detection the number of glycan mass components in a given sample can be increased. The analysis according to the invention can in most cases be performed from major or significant components in the glycome mixture. The present invention is preferably directed to detection of glycan mass components from a high quality glycan preparation with optimised experimental condition, when the glycan mass components have abundance at least higher than 0.01% of total amount of glycan mass components, more preferably of glycan mass components of abundance at least higher than 0.05%, and most preferably at least higher than 0.10% are detected. The invention is further directed to practical quality glycome compositions and analytic process directed to it, when glycan mass components of at least about 0.5%, of total amount of glycan mass components, more preferably of glycan mass components of abundance at least higher than 1.0%, even more preferably at least higher than 2.0%, most preferably at least higher than 4.0% (presenting lower range practical quality glycome), are detected. The invention is further directed to glycomes comprising preferred number of glycan mass components of at least the abundance of observable in high quality glycomes, and in another embodiment glycomes comprising preferred number of glycan mass components of at least the abundance of observable in practical quality glycomes.

### Subglycomes Obtainable by Purification or Specific Release Method

It is further realized that fractionation or differential specific release methods of glycans from glycoconjugates can be applied to produce subglycomes containing part of glycome.

The subglycomes produced by fractionation of glycomes are called “fractionated subglycomes”.

The glycomes produced by specific release methods are “linkage-subglycomes”. The invention is further directed to combinations of linkage-subglycomes and fractionated subglycomes to produce “fractionated linkage-subglycomes”, for example preferred fractionated linkage-subglycomes include neutral O-glycans, neutral N-glycans, acidic O-glycans, and acidic N-glycans, which were found very practical in characterising target materials according to the invention.

The fractionation can be used to enrich components of low abundance. It is realized that enrichment would enhance the detection of rare components. The fractionation methods may be used for larger amounts of cell material. In a preferred embodiment the glycome is fractionated based on the molecular weight, charge or binding to carbohydrate binding agents.

These methods have been found useful for specific analysis of specific subglycomes and enrichment of more rare components. The present invention is in a preferred embodiment directed to charge based separation of neutral and acidic glycans. This method gives rise for an analysis method, preferably mass spectroscopy material of reduced complexity and it is useful for analysis as neutral molecules in positive mode mass spectrometry and negative mode mass spectrometry for acidic glycans.

Differential release methods may be applied to get separately linkage specific subglycomes such as O-glycan, N-glycan, glycolipid or proteoglycan comprising fractions or combinations thereof. Chemical and enzymatic methods are known for release of specific fractions, furthermore there are methods for simultaneous release of O-glycans and N-glycans.

### Novel Complete Compositions

It is realized that at least part of the glycomes have novelty as novel compositions of very large amount of components. The glycomes comprising very broad range substances are referred as complete glycomes.

Preferably the composition is a complete composition comprising essentially all degrees of polymerisation in general from at least about disaccharides, more preferably from trisaccharides to at least about 25-mers in a high resolution case and at least to about 20-mers or at least about 15-mer in case of medium and practical quality preparations. It is realized that especially the lower limit, but also upper limit of a subglycome depend on the type of subglycome and/or method used for its production. Different complete ranges may be produced in scope of general glycomes by fractionation, especially based on size of the molecules.

Novel Compositions with New Combinations of Subglycomes and Preferred Glycan Groups

It is realized that several glycan types are present as novel glycome compositions produced from the tissue samples. The invention is specifically directed to novel mixture compositions comprising different subglycomes and preferred glycan groups.

### Novel Quantitative Glycome Compositions

It is realised that the glycome compositions as described in the Examples represent quantitatively new data about glycomes from the preferred tissue sample types. The proportions of various components cannot be derived from background data and are very useful for the analysis methods according to the invention. The invention is specifically directed to glycome compositions according to the Examples when the glycan mass components are present in essentially similar relative amounts.

### Preferred Composition Formulas

The present invention is specifically directed to glycomes of tissue samples according to the invention comprising glycan material with monosaccharide composition for each of glycan mass components according to the Formula I:

NeuAcₘNeuGcₙHexₒHexNAcₚdHex_{q}HexAᵣPenₛAcₜModXₓ,

wherein m, n, o, p, q, r, s, t, and x are independent integers with values ≧0 and less than about 100,
with the proviso that for each glycan mass components at least two of the backbone monosaccharide variables o, p, or r is greater than 0, and

ModX represents a modification (or N different modifications Mod1, Mod2, . . . , ModN), present in the composition in an amount of x (or in independent amounts of x1, x2, . . . , xN), Preferably examples of such modifications (Mod) including for example SO₃ or PO₃H indicating esters of sulfate and phosphate, respectively and the glycan composition is preferably derived from isolated human tissue samples or preferred subpopulations thereof according to the invention.

It is realized that usually glycomes contain glycan material for which the variables are less much less than 100, but large figures may be obtained for polymeric material comprising glycomes with repeating polymer structures, for example ones comprising glycosaminoglycan type materials. It is farther realized that abundance of the glycan mass components with variables more than 10 or 15 is in general very low and observation of the glycome components may require purification and enrichment of larger glycome components from large amounts of samples.

### Broad Mass Range Glycomes

In a preferred embodiment the invention is directed to broad mass range glycomes comprising polymeric materials and rare individual components as indicated above. Observation of large molecular weight components may require enrichment of large molecular weight molecules comprising fraction. The broad general compositions according to the Formula I are as described above,

with the proviso that

m, n, o, p, q, r, s, t, and x are independent integers with preferable values between 0 and 50, with the proviso that for each glycan mass components at least two of o, p, or r is at least 1, and the sum of the monosaccharide variables; m, n, o, p, q, r, and s, indicating the degree of polymerization or oligomerization, for each glycan mass component is less than about 100 and the glycome comprises at least about 20 different glycans of at least disaccharides.

### Practical Mass Range Glycomes

In a preferred embodiment the invention is directed to practical mass range and high quality glycomes comprising lower molecular weight ranges of polymeric material. The lower molecular weight materials at least in part and for preferred uses are observable by mass spectrometry without enrichment.

In a more preferred general composition according to the Formula I as described above, m, n, o, p, q, r, s, t, and x are independent integers with preferable values between 0 and about 20, more preferably between 0 and about 15, even more preferably between 0 and about 10, with the proviso that at least two of o, p, or r is at least 1,

and the sum of the monosaccharide variables; m, n, o, p, q, r, and s, indicating the degree of polymerization or oligomerization, for each glycan mass component is less than about 50 and more preferably less than about 30,
and the glycome comprises at least about 50 different glycans of at least trisaccharides.

In a preferred embodiment the invention is directed to practical mass range high quality glycomes which may comprise some lower molecular weight ranges of polymeric material. The lower molecular weight materials at least in part and for preferred uses are observable by mass spectrometry without enrichment.

In a more preferred general composition according to the Formula I as described above, m, n, o, p, q, r, s, t, and x are independent integers with preferable values between 0 and about 10, more preferably between 0 and about 9, even more preferably, between 0 and about 8, with the proviso that at least two of o, p, or r is at least 1,

and the sum of the monosaccharide variables; m, n, o, p, q, r, and s, indicating the degree of polymerization or oligomerization, for each glycan mass component is less than about 30 and more preferably less than about 25,
and the glycome comprises at least about 50 different glycans of at least trisaccharides.

The practical mass range glycomes may typically comprise tens of components, for example in positive ion mode MALDI-TOF mass spectrometry for neutral subglycomes it is usually possible to observe even more than 50 molecular mass components, even more than 100 mass components corresponding to much larger number of potentially isomeric glycans. The number of components detected depends on sample size and detection method.

### Preferred Subglycomes

The present invention is specifically directed to subglycomes of tissue sample glycomes according to the invention comprising glycan material with monosaccharide compositions for each of glycan mass components according to the Formula I and as defined for broad and practical mass range glycomes. Each subglycome has additional characteristics based on glycan core structures of linkage-glycomes or fractionation method used for the fractionated glycomes. The preferred linkage glycomes include N-glycans, O-glycans, glycolipid glycans, and neutral and acidic subglycomes.

### N-Glycan Subglycome

Protein N-glycosidase releases N-glycans comprising typically two N-acetylglycosamine units in the core, optionally a core linked fucose unit and typically then 2-3 hexoses (core mannoses), after which the structures may further comprise hexoses being mannose or in complex-type N-glycans further N-acetylglycosamines and optionally hexoses and sialic acids.

N-glycan subglycomes released by protein N-glycosidase comprise N-glycans containing N-glycan core structure and are releasable by protein N-glycosidase from cells. The N-glycan core structure is Manβ4GlcNAcβ4(Fucα6)ₙGlcNAc, wherein n is 0 or 1 and the N-glycan structures can be elongated from the Manβ4 with additional mannosylresidues. The protein N-glycosidase cleaves the reducing end GlcNAc from Asn in proteins. N-glycan subglycomes released by endo-type N-glycosidases cleaving between GlcNAc units contain Manβ4GlcNAc-core, and the N-glycan structures can be elongated from the Manβ4 with additional mannosylresidues.

In case the Subglycome and analysis representing it as Glycan profile is formed from N-glycans liberated by N-glycosidase enzyme, the preferred additional constraints for Formula I are:

p>0, more preferably 1≦p≦100, typically p is between 2 and about 20, but polymeric structures containing glycomes may comprise larger amounts of HexNAc and it is realized that in typical core of N-glycans indicating presence of at least partially complex type structure, when p≧3 it follows that o≧1.

### Glycolipid Subglycome

In case the Subglycome and analysis representing it as Glycan profile is formed from lipid-linked glycans liberated by endoglycoceramidase enzyme, the preferred additional constraints for Formula I are:

o>0, more preferably 1≦o≦100, and when p≧1 it follows that o≧2.

Typically glycolipids comprise two hexoses (a lactosylresidue) at the core. The degree of oligomerization in a usual practical glycome from glycolipids is under about 20 and more preferably under 10. Very large structures comprising glycolipids, polyglycosylceramides, may need enrichment for effective detection.

### Neutral and Acidic Subglycomes

Most preferred fractionated Subglycomes includes 1) subglycome of neutral glycans and 2) subglycome of acidic glycans. The major acidic monosaccharide unit is in most cases a sialic acid, the acidic fraction may further comprise natural negatively charged structure/structures such as sulphate(s) and/or phosphate(s).

In case the Subglycome and analysis representing it as Glycan profile is formed from sialylated glycans, the preferred additional constraints for Formula I are: (m+n)>0, more preferably 1≦(m+n)≦100.

Large amounts of sialic acid in a glycan mass component would indicate presence of polysialic acid type structures. Practical and high resolutions acidic glycomraes usually have m+n values for individual major glycan mass components with preferred abundance between 1 and 10, more preferably between 1 and 5 and most preferably between 1 and 4 for usual glycomes according to the invention. For neutral glycans, (m+n)=0, and they do not contain negatively charged groups as above. However, glycans with negatively charged groups can be eluted together with the neutral glycans into the neutral glycan fraction, and they may optionally be analyzed in the neutral glycan fraction according to the present invention.

### Preferred Structure Groups Observable in Glycome Profiles

The present invention is specifically directed to the glycomes of tissue samples according to the invention comprising as major components at least one of structure groups selected from the groups described below.

### Glycan Groups

According to the present invention, the glycan signals are optionally organized into glycan groups and glycan group profiles based on analysis and classification of the assigned monosaccharide and modification compositions and the relative amounts of monosaccharide and modification units in the compositions, according to the following classification rules:

1° The glycan structures are described by the formulae:

HexₘHexNAcₙdHexₒNeuAcₚNeuGc_{q}PenᵣMod1_{sMod1}Mod2_{sMod2} . . . MOdX_{sModX},
wherein m, n, o, p, q, individual sMod, and X, are each independent variables, and Mod is a functional group covalently linked to the glycan structure.

2° Glycan structures in general are classified as follows:
a. Structures (p,q=0) are classified as “non-sialylated”,
b. Structures (p,q>0) are classified as “sialylated”,
c. Structures (q>0) are classified as “NeuGc-containing”,
d. Relation [2 (p+q): (m+n)] describes the general sialylation degree of a glycan structure,
e. In the case of mammalian glycans, structures (o=0) are classified as “non-fucosylated”,
f. In the case of mammalian glycans, structures (o>0) are classified as “fucosylated”,
g. Structures (Mod=Ac and sAc>0) are classified as ‘acetylated’,
h. Structures (Mod=SO₃ and sSO₃>0) are classified as ‘sulfated’, and
i. Structures (Mod=PO₃H and sPO₃H>0) are classified as ‘phosphorylated’.

3° N-glycan glycan structures, generated e.g. by the action of peptide-N-glycosidases, are classified as follows:
a. Structures (n=2 and m>0 and p,q=0) are classified as “mannose-terminated N-glycans”,
b. Structures (n=2 and m≧5 and o,p,q=0) are classified as “high-mannose N-glycans”,
c. Structures (n=2 and m≧5 and o>0 and p,q=0) are classified as “flicosylated high-mannose N-glycans”,
d. Structures (n=2 and 4≧m≧1 and p,q=0) are classified as “low-mannose N-glycans”,
e. Structures (n=2 and 5≧m≧1 and o>0 and p,q=0) are classified as “fucosylated low-mannose N-glycans”,
f. Structures (n=3 and m≧2) are classified as “hybrid-type or monoantennary N-glycans”,
g. Structures (n≧4 and m≧3) are classified as “complex-type N-glycans”,
h. Structures (n>m≧2) are classified as “N-glycans containing non-reducing terminal N-acetylhexosamine”,
i. Structures (n=m≧5) are classified as “N-glycans potentially containing bisecting N-acetylglucosamine”,
j. In the case of mammalian N-glycans, structures (o≧2) are classified as “N-glycans containing α2-, α3-, or α4-linked fucose”,
k. Relation [2 (p+q): (m+n−5)] describes the “overall sialylation degree” of a sialylated N-glycan structure, and
l. Specifically, sum (p+q) describes the “sialylation degree” of a sialylated hybrid-type or monoantennary N-glycan structure.

4° Mucin-type O-glycan structures, generated e.g. by alkaline O-elimination, are classified as follows:
a. Structures (n=m), with (N=n=m), are classified as “Type N O-glycans”,
b. More specifically, structures (n=m=1) are classified as “Type 1 O-glycans”,
c. More specifically, structures (n=m=2) are classified as “Type 2 O-glycans”,
d. More specifically, structures (n=m=3) are classified as “Type 3 O-glycans”,
e. Relation [2 (p+q): (m+n)] describes the overall sialylation degree of a sialylated O-glycan structure, and
f. Specifically, relation [(p+q): N] describes the sialylation degree of a sialylated Type N O-glycan structure.

Lipid-linked can also be classified into structural groups based on their monosaccharide compositions, as adopted from the classifications above according to the invention.

For example, glycan signal corresponding to a tissue sample N-glycan structure:

Hex₅HexNAc₄dHex₂NeuAc₁Ac₁,
is classified as belonging to the following Glycan Groups:
   sialylated (general sialylation degree: 2/9),
   fucosylated,
   acetylated,
   complex-type N-glycans (overall sialylation degree: 0.5),
   N-glycans containing α2-, α3-, or α4-linked fucose.

### Glycomes Comprising Novel Glycan Types

The present invention revealed novel unexpected components among in the glycomes studied. The present invention is especially directed to glycomes comprising such unusual materials.

### Preferred Glycome Types

### Derivatized Glycomes

It is further realized that the glycans may be derivatized chemically during the process of release and isolation. Preferred modifications include modifications of the reducing end and/or modifications directed especially to the hydroxyls- and/or N-atoms of the molecules. The reducing end modifications include modifications of reducing end of glycans involving known derivatization reactions, preferably reduction, glycosylamine, glycosylamide, oxime (aminooxy-) and reductive amination modifications. Most preferred modifications include modification of the reducing end. The derivatization of hydroxyl- and/or amine groups, such as produced by methylation or acetylation methods including permethylation and peracetylation has been found especially detrimental to the quantitative relation between natural glycome and the released glycome.

### Non-Derivatized Released Glycomes

In a preferred embodiment the invention is directed to non-derivatized released glycomes. The benefit of the non-derivatized glycomes is that less processing is needed for the production. The non-derivatized released glycomes correspond more exactly to the natural glycomes from which these are released. The present invention is further directed to quantitative purification according to the invention for the non-derivatized released glycomes and analysis thereof.

The present invention is especially directed to released glycomes when the released glycome is not a permodified glycome such as permethylated glycome or peracetyated glycome. The released glycome is more preferably reducing end derivatized glycome or a non-derivatized glycome, most preferably non-derivatized glycome.

### Novel Cell Surface Glycomes and Released Glycomes of the Target Material

The present invention is further directed to novel total compositions of glycans or oligosaccharides referred as glycomes and in a more specific embodiment as released glycomes observed from or produced from the target material according to the invention. The released glycome indicates the total released glycans or total specific glycan subfractions released from the target material according to the invention. The present invention is specifically directed to released glycomes meaning glycans released from the target material according to the invention and to the methods according to the invention directed to the glycomes.

The present invention is preferably directed to the glycomes released as truncated and/or non-truncated glycans and/or derivatized according to the invention.

The invention is especially directed to N-linked and/or O-linked and/or lipid-linked released glycomes from the target material according to the invention. The invention is more preferably directed to released glycomes comprising glycan structures according to the invention, preferably glycan structures as defined in Formula I. The invention is more preferably directed to N-linked released glycomes comprising glycan structures according to the invention, preferably glycan structures as defined in Formula I.

### Non-Derivatized Released Cell Surface Glycomes and Production

In a preferred embodiment the invention is directed to non-derivatized released cell surface glycomes. The non-derivatized released cell surface glycomes correspond more exactly to the fractions of glycomes that are localized on the cell surfaces, and thus available for biological interactions. These cell surface localized glycans are of especial importance due to their availability for biological interactions as well as targets for reagents (e.g. antibodies, lectins, etc.) targeting the cells or tissues of interest. The invention is further directed to release of the cell surface glycomes, preferably from intact cells by hydrolytic enzymes such as proteolytic enzymes, including proteinases and proteases, and/or glycan releasing enzymes, including endo-glycosidases or protein N-glycosidases. Preferably the surface glycoproteins are cleaved by proteinase such as trypsin and then glycans are analysed as glycopeptides or preferably released further by glycan releasing enzyme.

### Analysis of the Glycomes

The present invention is especially directed to analysis of glycan mixtures present in tissue samples by chemical, biochemical, or physical means, preferably by mass spectrometry, as described below.

### Quantitative and Qualitative Analysis of Glycan Profile Data

The invention is directed to novel methods for qualitative analysis of glycome data. The inventors noticed that there are specific components in glycomes according to the invention, the presence or absence of which are connected or associated with specific cell type or cell status. It is realized that qualitative comparison about the presence of absence of such signals are useful for glycome analysis. It is further realized that signals either present or absent that are derived from a general glycome analysis may be selected to more directed assay measuring only the qualitatively changing component or components optionally with a more common component or components useful for verification of data about the presence or absence of the qualitative signal.

The present invention is further specifically directed to quantitative analysis of glycan data from tissue samples. The inventors noted that quantitative comparisons of the relative abundances of the glycome components reveal substantial differences about the glycomes useful for the analysis according to the invention.

### Essential Steps of the Glycome Analysis

The process contains essential key steps which should be included in every process according to the present invention.

The essential key steps of the analysis are:
1. Release of total glycans or total glycan groups from a tissue sample
2. Purification of the glycan fraction/fractions from contaminating biological material of the sample, preferably by a small scale column array or an array of solid-phase extraction steps
3. Analysis of the composition of the released glycans, preferably by mass spectrometry

In most cases it is useful to compare the data with control sample data. The control sample may be for example from a healthy tissue or cell type and the sample from same tissue altered by cancer or another disease. It is preferable to compare samples from same individual organism, preferably from the same human individual.

### Specific Types of the Glycome Analysis

### Comparative Analysis

The steps of a comparative analysis are:
1. Release of total glycans or total glycan groups from tissue sample
2. Purification of the glycan fraction/fractions from biological material of the sample, preferably by a small scale column array or an array of solid-phase extraction steps
3. Analysis of the composition of the released glycans, preferably by mass spectrometry
4. Comparing data about the released glycans quantitatively or qualitatively with data produced from another tissue sample

It may be useful to analyse the glycan structural motifs present in the sample, as well as their relative abundances. The ability to elucidate structural motifs results from the quantitative nature of the present analysis procedure, comparison of the data to data from previously analyzed samples, and knowledge of glycan biosynthesis.

### Analysis Including Characterization of Structural Motives

The glycome analysis may include characterization of structural motives of released glycans. The structural motif analysis may be performed in combination with structural analysis.

Preferred methods to reveal specific structural motifs include
a) direct analysis of specific structural modifications of the treatment of glycans preferably by exo- or endoglycosidases and/or chemical modification or
b) indirect analysis by analysis of correlating factors for the structural motives for such as mRNA-expression levels of glycosyltransferases or enzymes producing sugar donor molecules for glycosyltransferases.

The direct analyses are preferred as they are in general more effective and usually more quantitative methods, which can be combined to glycome analysis.

In a preferred embodiment the invention is directed to combination of analysis of structural motifs and glycome analysis.

### The Steps of a Structural Motif Analysis are:

1. Release of total glycans or total glycan groups from a tissue sample
2. Purification of the glycan fraction/fractions from biological material of the sample, preferably by a small scale column array or an array of solid-phase extraction steps
3. Analysis of the composition of the released glycans, preferably by mass spectrometry
4. Analysis of structural motifs present in of the glycan mixture, and optionally their relative abundancies
5. Optionally, comparing data about the glycan structural motifs with data produced from another tissue sample

The steps 3 and 4 may be combined or performed in order first 4 and then 3.

### Preferred Detailed Glycome Analysis Including Quantitative Data Analysis

More detailed preferred analysis method include following analysis steps:
1. Preparing a tissue sample containing glycans for the analysis
2. Release total glycans or total glycan groups from a tissue sample
3. Optionally modifying glycans or part of the glycans.
4. Purification of the glycan fraction/fractions from biological material and reagents of the sample by a small scale column array
5. Optionally modifying glycans and optionally purifying modified glycans
6. Analysis of the composition of the released glycans preferably by mass spectrometry using at least one mass spectrometric analysis method
7. a) Optionally presenting the data about released glycans quantitatively and
7. b) Comparing the quantitative data set with another data set from another tissue sample and/or alternatively to 7a) and 7b)
8. Comparing data about the released glycans quantitatively or qualitatively with data produced from another tissue sample

The present methods further allow the possibility to use part of the non-modified material or material modified in step 3 or 5 for additional modification step or step and optionally purified after modification step or steps, optionally combining modified samples, and analysis of additionally modified samples, and comparing results from differentially modified samples.

As mentioned above, it is realized that many of the individual monosaccharide compositions in a given glycome further corresponds to several isomeric individual glycans. The present methods allow for generation of modified glycomes. This is of particular use when modifications are used to reveal such information about glycomes of interest that is not directly available from a glycan profile alone (or glycome profiles to compare). Modifications can include selective removal of particular monosaccharides bound to the glycome by a defined glycosidic bond, by degradation by specific exoglycosidases or selective chemical degradation steps such as e.g. periodic acid oxidation. Modifications can also be introduced by using selective glycosyltransferase reactions to label the free acceptor structures in glycomes and thereby introduction of a specific mass label to such structures that can act as acceptors for the given enzyme. In preferred embodiment several of such modifications steps are combined and used to glycomes to be compared to gain further insights of glycomes and to facilitate their comparison.

Such modifications may also include non-covalent modification, such as ion-pairing of charged groups. Sulphate esters may be ion-paired with cationic moiety, which enhances the ionization of sulphated glycans in positive-ion mode mass spectrometry. Such cationic moieties include e.g. lysine or arginine tripeptide (KKK or RRR), as described previously for glycopeptides. The present invention is specifically directed to using ion-pairing of free oligosaccharides to enhance detection of glycans with charged groups such as sulphate or phosphate. According to the present invention, glycans containing charged groups may also be identified by analysis as differential adduct and/or ion-pairing ions. For example, comparison of spectra obtained from the same sample in the presence of sodium or lithium ions gives information about the presence of charged groups without covalent modification.

### Quantitative Presentation of Glycome Analysis

The present invention is specifically directed to quantitative presentation of glycome data.

### Two-Dimensional Presentation by Quantitation and Component Indicators

The quantitative presentation means presenting quantitative signals of components of the glycome, preferably all major components of the glycome, as a two-dimensional presentation including preferably a single quantitative indicator presented together with component identifier. A preferred purpose of this operation is to allow reliable comparison between data obtained from different samples or to identify glycan structures present in the analyzed sample. Any given glycan can exist as multiple ions in mass spectrometry or multiple modified forms in mass spectrometry and chromatography. For example, a glycan can exist as adduct ions with e.g. sodium or potassium. This may divide the signal to e.g. three or four components in the case of sialylated glycans, and these signals have to be summed up to get the correct signal intensity value for the glycan component. Another example is related to comparison of samples with different sample quality, and normalization is required to be able to reliably compare these samples. The prior art describes comparison between glycan samples without proper correction and normalization of the data.

The preferred two-dimensional presentations includes tables and graphs presenting the two dimensional data. The preferred tables list quantitative indicators in connection with, preferably beside or under or above the component identifiers, most preferably beside the identifier because in this format the data comprising usually large number of component identifier—quantitation indicator pairs.

### Quantitation Indicator

The quantitation indicator is a value indicating the relative abundance of the single glycome component with regard to other components of total glycome or subglycome. The quantitation indicator can be directly derived from quantitative experimental data, or experimental data corrected to be quantitative.

### Normalized Quantitation Indicator

The quantitation indicator is preferably a normalized quantitation indicator. The normalized quantitation indicator is defined as the experimental value of a single experimental quantitation indicator divided by total sum of quantitation indicators multiplied by a constant quantitation factor.

Preferred quantitation factors includes integer numbers from 1-1000 0000 000, more preferably integer numbers 1, 10 or 100, and more preferably 1 or 100, most preferably 100.

The quantitation number one is preferred as commonly understandable portion from 1 concept and the most preferred quantitation factor 100 corresponds to common concept of percent values.

The quantitation indicators in tables are preferably rounded to correspond to practical accuracy of the measurements from which the values are derived from. Preferred rounding includes 2-5 meaningful accuracy numbers, more preferably 2-4 numbers and most preferably 2-3 numbers.

### Component Indicators

The preferred component indicators may be experimentally derived component indicators. Preferred components indicators in the context of mass spectrometric analysis includes mass numbers of the glycome components, monosaccharide or other chemical compositions of the components and abbreviation corresponding to thereof, names of the molecules preferably selected from the group: descriptive names and abbreviations; chemical names, abbreviations and codes; and molecular formulas including graphic representations of the formulas. It is further realized that molecular mass based component indicators may include multiple isomeric structures. The invention is in a preferred embodiment directed to practical analysis using molecular mass based component indicators. In more specific embodiment the invention is further directed to chemical or enzymatic modification methods or indirect methods according to the invention in order to resolve all or part of the isomeric components corresponding to a molecular mass based component indicators.

### Glycan Signals

The present invention is directed to a method of accurately defining the molecular masses of glycans present in a sample, and assigning monosaccharide compositions to the detected glycan signals.

The glycan signals according to the present invention are glycan components characterized by:

1° mass-to-charge ratio (m/z) of the detected glycan ion,
2° molecular mass of the detected glycan component, and/or
3° monosaccharide composition proposed for the glycan component.

### Glycan Profiles

The present invention is further directed to a method of describing mass spectrometric raw data of glycan signals as two-dimensional tables of:

1° monosaccharide composition, and
2° relative abundance,

which form the glycan profiles according to the invention. Monosaccharide compositions are as described above. For obtaining relative abundance values for each glycan signal, the raw data is recorded in such manner that the relative signal intensities of the glycan signals represent their relative molar proportions in the sample. Methods for relative quantitation in MALDI-TOF mass spectrometry of glycans are known in the art (Naven & Harvey, 1996; Papac et al., 1996) and are described in the present invention. However, the relative signal intensities of each glycan signal are preferably corrected by taking into account the potential artefacts caused by e.g. isotopic overlapping, alkali metal adduct overlapping, and other disturbances in the raw data, as described below.

By forming these glycan profiles and using them instead of the raw data, analysis of the biological data carried by the glycan profiles is improved, including for example the following operations:

1° identification of glycan signals present in the glycan profile,
2° comparison of glycan profiles obtained from different samples,
3° comparison of relative intensities of glycan signals within the glycan profile, and
4° organizing the glycan signals present in the glycan profile into subgroups or subprofiles.

### Analysis of Associated Signals to Produce Single Quantitative Signal (Quantitation Indicator)

### Analysis of Associated Signals: Isotope Correction

Glycan signals and their associated signals may have overlapping isotope patterns. Overlapping of isotope patterns is corrected by calculating the experimental isotope patterns and subtracting overlapping isotope signals from the processed data.

### Analysis of Associated Signals: Adduct Ion Correction in Positive Ion Mode

Glycan signals may be associated with signals arising from multiple adduct ions in positive ion mode, e.g. different alkali metal adduct ions. Different glycan signals may give rise to adduct ions with similar m/z ratios: as an example, the adduct ions [Hex+Na]⁺ and [dHex+K]⁺ have m/z ratios of 203.05 and 203.03, respectively. Overlapping of adduct ions is corrected by calculating the experimental alkali metal adduct ion ratios in the sample and using them to correct the relative intensities of those glycan signals that have overlapping adduct ions in the experimental data. Preferably, the major adduct ion type is used for comparison of relative signal intensities of the glycan signals, and the minor adduct ion types are removed from the processed data. The calculated proportions of minor adduct ion types are subtracted from the processed data.

### Analysis of Associated Signals: Adduct Ion Correction in Negative Ion Mode

Also in negative ion mode mass spectrometry, glycan signals may be associated with signals arising from multiple adduct ions. Typically, this occurs with glycan signals that correspond to multiple acidic group containing glycan structures. As an example, the adduct ions [NeuAc₂-H+Na]⁻ at m/z 621.2 and [NeuAc₂-H+K]⁻ at m/z 637.1, are associated with the glycan signal [NeuAc₂-H]⁻ at m/z 599.2. These adduct ion signals are added to the glycan signal and thereafter removed from the processed data. In cases where different glycan signals and adduct ion signals overlap, this is corrected by calculating the experimental alkali metal adduct ion ratios in the sample and using them to correct the relative intensities of those glycan signals that have overlapping adduct ions in the experimental data.

### Analysis of Associated Signals: Removal of Elimination Products

Glycan signals may be associated with signals, e.g. elimination of water (loss of H₂O), or lack of methyl ether or ester groups (effective loss of CH₂), resulting in experimental m/z values 18 or 14 mass units smaller than the glycan signal, respectively. These signals are not treated as individual glycan signals, but are instead treated as associated signals and removed from the processed data.

Classification of Glycan Signals into Glycan Groups

According to the present invention, the glycan signals are optionally organized into glycan groups and glycan group profiles based on analysis and classification of the assigned monosaccharide and modification compositions and the relative amounts of monosaccharide and modification units in the compositions, according to the classification rules described above.

### Generation of Glycan Group Profiles

To generate glycan group profiles, the proportions of individual glycan signals belonging to each glycan group are summed. The proportion of each glycan group of the total glycan signals equals its prevalence in the glycan profile. The glycan group profiles of two or more samples can be compared. The glycan group profiles can be further analyzed by arranging glycan groups into subprofiles, and analyzing the relative proportions of different glycan groups in the subprofiles. Similarly formed subprofiles of two or more samples can be compared.

### Specific Technical Aspects of Tissue Glycome Analysis

### Preferred Sample Sizes

The present invention is especially useful when low sample amounts are available. Practical cellular or tissue material may be available for example for diagnostic only in very small amounts.

Sample sizes for preferred pico-scale preparation methods

The inventors found surprisingly that glycan fraction could be produced and analysed effectively from samples containing low amount of material, for example 100 000-1 000 000 cells or a cubic millimetre (microliter) of the cells.

The combination of very challenging biological samples and very low amounts of samples forms another challenge for the present analytic method. The yield of the purification process must be very high. The estimated yields of the glycan fractions of the analytical processes according to the present invention varies between about 50% and 99%. Combined with effective removal of the contaminating various biological materials even more effectively over the wide preferred mass ranges according to the present invention show the ultimate performance of the method according to the present invention.

### Isolation of Glycans and Glycan Fractions

The present invention is directed to a method of preparing an essentially unmodified glycan sample for analysis from the glycans present in a given sample.

A preferred glycan preparation process consists of the following steps:

1° isolating a glycan-containing fraction from the sample,
2° optionally purification the fraction to useful purity for glycome analysis

The preferred isolation method is chosen according to the desired glycan fraction to be analyzed. The isolation method may be either one or a combination of the following methods, or other fractionation methods that yield fractions of the original sample:

1° extraction with water or other hydrophilic solvent, yielding water-soluble glycans or glycoconjugates such as free oligosaccharides or glycopeptides,
2° extraction with hydrophobic solvent, yielding hydrophilic glycoconjugates such as glycolipids,
3° N-glycosidase treatment, especially F. meningosepticum N-glycosidase F treatment, yielding N-glycans,
4° alkaline treatment, such as mild (e.g. 0.1 M) sodium hydroxide or concentrated ammonia treatment, either with or without a reductive agent such as borohydride, in the former case in the presence of a protecting agent such as carbonate, yielding 3-elimination products such as O-glycans and/or other elimination products such as N-glycans,
5° endoglycosidase treatment, such as endo-β-galactosidase treatment, especially Escherichia freundii endo-β-galactosidase treatment, yielding fragments from poly-N-acetyllactosamine glycan chains, or similar products according to the enzyme specificity, and/or
6° protease treatment, such as broad-range or specific protease treatment, especially trypsin treatment, yielding proteolytic fragments such as glycopeptides.

The released glycans are optionally divided into sialylated and non-sialylated subfractions and analyzed separately. According to the present invention, this is preferred for improved detection of neutral glycan components, especially when they are rare in the sample to be analyzed, and/or the amount or quality of the sample is low. Preferably, this glycan fractionation is accomplished by graphite chromatography and/or ion-exchange chromatography.

According to the present invention, sialylated glycans are optionally modified in such manner that they are isolated together with the non-sialylated glycan fraction in the non-sialylated glycan specific isolation procedure described above, resulting in improved detection simultaneously to both non-sialylated and sialylated glycan components. Preferably, the modification is done before the non-sialylated glycan specific isolation procedure. Preferred modification processes include neuraminidase treatment and derivatization of the sialic acid carboxyl group, while preferred derivatization processes include amidation and esterification of the carboxyl group.

### Glycan Release Methods

The preferred glycan release methods include, but are not limited to, the following methods:

Free glycans—extraction of free glycans with for example water or suitable water-solvent mixtures.

Protein-linked glycans including O- and N-linked glycans-alkaline elimination of protein-linked glycans, optionally with subsequent reduction of the liberated glycans.

Mucin-type and other Ser/Thr O-linked glycans-alkaline β-elimination of glycans, optionally with subsequent reduction of the liberated glycans.

N-glycans—enzymatic liberation, optionally with N-glycosidase enzymes including for example N-glycosidase F from C. meningosepticum, Endoglycosidase H from Streptomyces, or N-glycosidase A from almonds.

Lipid-linked glycans including glycosphingolipids-enzymatic liberation with endoglycoceramidase enzyme; chemical liberation; ozonolytic liberation.

Glycosaminoglycans—treatment with endo-glycosidase cleaving glycosaminoglycans such as chondroinases, chondroitin lyases, hyalurondases, heparanases, heparatinases, or keratanases/endo-beta-galactosidases; or use of O-glycan release methods for O-glycosidic Glycosaminoglycans; or N-glycan release methods for N-glycosidic glycosaminoglycans or use of enzymes cleaving specific glycosaminoglycan core structures; or specific chemical nitrous acid cleavage methods especially for amine/N-sulphate comprising glycosaminoglycans Glycan fragments-specific exo- or endoglycosidase enzymes including for example keratanase, endo-β-galactosidase, hyaluronidase, sialidase, or other exo- and endoglycosidase enzyme; chemical cleavage methods; physical methods

### Effective Purification Process

The invention describes special purification methods for glycan mixtures from tissue samples. Previous glycan sample purification methods have required large amounts of material and involved often numerous chromatographic steps and even purification of specific proteins. It is known that protein glycosylation varies protein specifically and single protein specific data can thus not indicate the total tissue level glycosylation. Purification of single protein is a totally different task than purifying the glycan fraction according to the present invention.

When the purification starts from a tissue or cells, the old processes of prior art involve often laborious homogenisation steps affecting the quality of the material produced. The present purification directly from a biological sample such as cell or tissue material, involves only a few steps and allows quick purification directly from the biological material to analysis preferably by mass spectrometry.

Purification from Cellular Materials of Cells and/or Tissues

The cellular material contains various membranes, small metabolites, various ionic materials, lipids, peptides, proteins etc. All of the materials can prevent glycan analysis by mass spectrometry if these cannot be separated from the glycan fraction. Moreover, for example peptide or lipid materials may give rise to mass spectrometric signals within the preferred mass range within which glycans are analysed. Many mass spectrometric methods, including preferred MALDI-mass spectrometry for free glycan fractions, are more sensitive for peptides than glycans. With the MALDI method peptides in the sample may be analysed with approximately 1000-fold higher sensitivity in comparison to methods for glycans. Therefore the method according to the present invention should be able to remove for example potential peptide contaminations from free glycan fractions most effectively. The method should remove essential peptide contaminations from the whole preferred mass range to be analysed.

### Purification Suitable for Mass Spectrometry, Especially MALDI-TOF Mass Spectrometry

The inventors discovered that the simple purification methods would separate released glycans from all possible cell materials so that

1) The sample is technically suitable for mass spectrometric analysis.
This includes two major properties,
a) the samples is soluble for preparation of mass spectrometry sample and
b) does not have negative interactions with chemicals involved in the mass spectrometric method, preferably the sample dries or crystallizes properly with matrix chemical used in MALDI-TOF mass spectrometry
   When using MALDI-technologies, the sample does not dry or crystallize properly if the sample contains harmful impurity material in a significant amount.

2) The purity allows production of mass spectrum of suitable quality.
a) the sample has so low level impurities that it gives mass spectrometric signals. Especially when using MALDI-TOF mass spectrometry, signals can be suppressed by background so that multiple components/peaks cannot be obtained.
b) the sample is purified so that there is no major impurity signals in the preferred mass ranges to be measured.

Preferably the present invention is directed to analysis of unusually small sample amounts. This provides a clear benefit over prior art, when there is small amount of sample available from a small region of diseased tissue or diagnostic sample such as tissue slice produced for microscopy or biopsy sample. Methods to achieve such purity (purity being a requirement for the sensitivity needed for such small sample amounts) from tissue or cell samples (or any other complex biological matrices e.g. serum, saliva) has not been described in the prior art.

In a preferred embodiment the method includes use of non-derived glycans and avoiding general derived glycans. There are methods of producing glycan profiles including modification of all hydroxyl groups in the sample such as permethylation. Such processes require large sample amounts and produces chemical artefacts such as undermethylated molecules lowering the effectively of the method. These artifact peaks cover all minor signals in the spectra, and they can be misinterpreted as glycan structures. It is of importance to note that in glycome analyses the important profile-to profile differences often reside in the minor signals. In a specific embodiment the present invention is directed to site specific modification of the glycans with effective chemical or enzyme reaction, preferably a quantitative reaction.

### Preferred Analytical Technologies for Glycome Analysis

### Mass Spectrometric Analysis of Glycomes

The present invention is specifically directed to quantitative mass spectrometric methods for the analysis of glycomes. Most preferred mass spectrometric methods are MALDI-TOF mass spectrometry methods.

### MALDI-TOF Analysis

The inventors were able to optimise MALDI-TOF mass spectrometry for glycome analysis. The preferred mass spectrometric analysis process is MALDI-TOF mass spectrometry, where the relative signal intensities of the unmodified glycan signals represent their relative molar proportions in the sample, allowing relative quantification of both neutral (Naven & Harvey, 1996) and sialylated (Papac et al., 1996) glycan signals. Preferred experimental conditions according to the present invention are described under Experimental procedures of Examples listed below.

### Preferred Mass Ranges for MALDI-TOF Analysis and Released Non-Modified Glycomes

For MALDI-TOF mass spectrometry of unmodified glycans in positive ion mode, optimal mass spectrometric data recording range according to the present invention is over m/z 200, more preferentially between m/z 200-10000, or even more preferably between m/z 200-4000 for improved data quality. In the most preferred form according to the present invention, the data is recorded between m/z 700-4000 for accurate relative quantification of glycan signals.

For MALDI-TOF mass spectrometry of unmodified glycans in negative ion mode, optimal mass spectrometric data recording range according to the present invention is over m/z 300, more preferentially between m/z 300-10000, or even more preferably between m/z 300-4000 for improved data quality. In the most preferred forms according to the present invention, the data is recorded between m/z 700-4000 or most preferably between m/z 800-4000 for accurate relative quantification of glycan signals.

Practical m/z-Ranges

The practical ranges comprising most of the important signals, as observed by the present invention may be more limited than these. Preferred practical ranges includes lower limit of about m/z 400, more preferably about m/z 500, and even more preferably about m/z 600, and most preferably m/z about 700 and upper limits of about m/z 4000, more preferably m/z about 3500 (especially for negative ion mode), even more preferably m/z about 3000 (especially for negative ion mode), and in particular at least about 2500 (negative or positive ion mode) and for positive ion mode to about m/z 2000 (for positive ion mode analysis). The preferred range depends on the sizes of the sample glycans, samples with high branching or polysaccharide content or high sialylation levels are preferably analysed in ranges containing higher upper limits as described for negative ion mode. The limits are preferably combined to form ranges of maximum and minimum sizes or lowest lower limit with lowest higher limit, and the other limits analogously in order of increasing size.

### Preferred Analysis Modes for MALDI-TOF for Effective Glycome Analysis

The inventors were able to show effective quantitative analysis in both negative and positive mode mass spectrometry.

### Sample Handling

The inventors developed optimised sample handling process for preparation of the samples for MALDI-TOF mass spectrometry.

### Glycan Purification

The glycan purification method according to the present invention consists of at least one of purification options, preferably in specific combinations described below, including the following purification options:

### 1) Precipitation-extraction;

### 2) Ion-exchange;

3) Hydrophobic interaction;
4) Hydrophilic interaction; and
5) Affinity to graphitized carbon.

1) Precipitation-extraction may include precipitation of glycans or precipitation of contaminants away from the glycans. Preferred precipitation methods include:
1. Glycan material precipitation, for example acetone precipitation of glycoproteins, oligosaccharides, glycopeptides, and glycans in aqueous acetone, preferentially ice-cold over 80% (v/v) aqueous acetone; optionally combined with extraction of glycans from the precipitate, and/or extraction of contaminating materials from the precipitate;
2. Protein precipitation, for example by organic solvents or trichloroacetic acid, optionally combined with extraction of glycans from the precipitate, and/or extraction of contaminating materials from the precipitate;
3. Precipitation of contaminating materials, for example precipitation with trichloroacetic acid or organic solvents such as aqueous methanol, preferentially about ⅔ aqueous methanol for selective precipitation of proteins and other non-soluble materials while leaving glycans in solution;

2) Ion-exchange may include ion-exchange purification or enrichment of glycans or removal of contaminants away from the glycans. Preferred ion-exchange methods include:
1. Cation exchange, preferably for removal of contaminants such as salts, polypeptides, or other cationizable molecules from the glycans; and
2. Anion exchange, preferably either for enrichment of acidic glycans such as sialylated glycans or removal of charged contaminants from neutral glycans, and also preferably for separation of acidic and neutral glycans into different fractions.

3) Hydrophilic interaction may include purification or enrichment of glycans due to their hydrophilicity or specific adsorption to hydrophilic materials, or removal of contaminants such as salts away from the glycans. Preferred hydrophilic interaction methods include:
1. Hydrophilic interaction chromatography, preferably for purification or enrichment of glycans and/or glycopeptides;
2. Adsorption of glycans to cellulose in hydrophobic solvents for their purification or enrichment, preferably to microcrystalline cellulose, and even more preferably using an n-butanol:methanol:water or similar solvent system for adsorption and washing the adsorbed glycans, in most preferred system n-butanol:methanol:water in relative volumes of 10:1:2, and water or water: ethanol or similar solvent system for elution of purified glycans from cellulose.

4) Affinity to graphitized carbon may include purification or enrichment of glycans due to their affinity or specific adsorption to graphitized carbon, or removal of contaminants away from the glycans. Preferred graphitized carbon affinity methods include porous graphitized carbon chromatography.

Preferred purification methods according to the invention include combinations of one or more purification options. Examples of the most preferred combinations include the following combinations:

1) For neutral underivatized glycan purification: 1. cation exchange of contaminants, 2. hydrophobic adsorption of contaminants, and 3. graphitized carbon affinity purification of glycans.
1) For sialylated underivatized glycan purification: 1. cation exchange of contaminants, 2. hydrophobic adsorption of contaminants, 3. optionally adsorption of glycans to cellulose, and 4. graphitized carbon affinity purification of glycans.

### NMR-Analysis of Glycomes

The present invention is directed to analysis of released glycomes by spectrometric method useful for characterization of the glycomes. The invention is directed to NMR spectroscopic analysis of the mixtures of released glycans. The inventors showed that it is possible to produce a released glycome from tissue samples in large scale enough and useful purity for NMR-analysis of the glycome. In a preferred embodiment the NMR-analysis of the tissue glycome is one dimensional proton NMR-analysis showing structural reporter groups of the major components in the glycome. The present invention is further directed to combination of the mass spectrometric and NMR analysis of small scale tissue samples.

### Analysis of Changes Related to Animal Individuals, Animal Species and Animal Status

The inventors further realized major glycome differences between samples from the same species. The invention is specifically directed to analysis of individual differences between animals. The invention is further directed to the use of the information in breeding of animals, especially production animals, preferentially in context of increased susceptibility to cancer, especially genetic susceptibility to cancer.

The inventors further realized major glycome differences between samples from animals related to the cancer status of the animal. The invention is especially directed to the analysis of biological status related changes of animal.

The inventors further noticed major species specific differences in the total released glycomes analysed. It is realized that species specific glycome differences are useful for analysis of effects of glycosylations in animal materials from different species in context of cancer.

### Preferred Target Species, Especially Animals for Tissue Analysis

The invention revealed that glycome oligosaccharide mixtures can be produced effectively from eukaryotic species especially animal tissues.

The invention is in a preferred embodiment directed to analysis of human type primates such as monkeys especially apes (examples include chimpanzee, pygmy chimpanzee, gorilla, orangutan) and human, the preference is based on close similarity of primates and human on genetic and cell biological level, providing similarity for samples to be analysed and scientifically important evolution based glycosylation changes between similar species.

The invention is further directed to analysis of animals useful for development of pharmaceutical and therapeutic materials in context of cancer. The preferred animals include rodents (such as mouse, hamster, rat) and human type primates.

### Targets of Analysis—Tissue Materials

The present invention refers as “tissue materials” all preferred target tissue related material including for example tissues, secretions and cultivated differentiated cells

### Preferred Tissue Type

The present invention is preferably directed to specific tissue types for the analysis according to the invention. The tissue type are found to be very suitable and feasible for the analysis according to the invention. The analysis is especially directed to analysis of

1) tissues of gastrointestinal track, preferably mouth, larynx, stomach, large and small intestine
2) internal organs such as ovarian tissue, liver, lungs, or kidney
3) tissues of circulatory system, especially blood
4) cultivated cell line models of the differentiated tissues

### Preferred Tissue Parts

The present invention is preferably directed to specific parts of tissue for the analysis according to the invention. The inventors realized that it is possible perform glycomics analysis of specific parts of tissues and reveal differences useful for studies of diseases and disease induced changes and other changes or presence of receptor structures on specific subtissues. Preferred subtissues includes

1) tissues surfaces, especially epithelia of gastrointestinal tract and cell surfaces and
2) components of circulatory system, preferably serum/plasma, and blood cells, especially red cells and white blood cells

### Preferred Tissue Derivatives to be Analysed Including Liquid Secretions

The invention is further directed to material produced by tissues.

Preferably the invention is directed to the analysis of secretions of tissues, preferably liquid secretions of tissues, preferably milk, saliva or urine. It is realized that liquid secretions form a specific group of tissue derived materials found especially useful for the glycome analysis methods according to the invention. Milk is especially preferred as a food material consumed by animals and human and analysis with regard to each of individual specific, animal status specific and species specific differences.

The invention is under separate preferred embodiment directed to the analysis of specific conjugated glycomes such as protein or lipid derived glycomes, from the secretions and in another preferred embodiment free soluble glycomes of the secretions.

Soluble Glycome Materials: Tissue and/or Secretion Materials, Especially with High Protein Content

The invention is in a preferred embodiment directed to specific methods developed for the analysis of soluble glycome material from tissues and secretions. This group includes background for purification different from solid tissue and cell derived materials. The group includes tissue solutions such as blood serum/plasma and liquid secretions such as milk, saliva and urine.

Subcomponents of Glycomes, Especially from Secreted Proteins

The invention is further directed to methods for selecting specific components of glycomes and searching enriched fractions such as specific protein fraction comprising the specific glycome components. Examples of such preferred methods include search of “cancer specific”? oligosaccharide structures according to the invention from serum, saliva or urine. The “cancer specific” oligosaccharide can exist as free secreted oligosaccharides or as conjugates to other biomolecules such as proteins or lipids.

### Tissue Surface Glycomes

In a preferred embodiment the invention is directed to special methods for the analysis of the surfaces of tissues.

The preferred tissue surfaces includes

1) epithelia or endothelia of the preferred cancer tissues and
2) surfaces of cells according to cells on surface of tissues or separable homogeneously from tissue, such as blood cells and
3) surfaces of cultivated cells which may be used as models for differentiated tissues.

### Non-Derivatized Released Target Material Surface Glycomes and Production

In a preferred embodiment the invention is directed to non-derivatized released cell surface glycomes. The non-derivatized released cell surface glycomes correspond more exactly to the fractions of glycomes that are localized on the cell surfaces, and thus available for biological interactions. These cell surface localized glycans are of especial importance due to their availability for biological interactions as well as targets for reagents (e.g. antibodies, lectins, etc.) targeting the cells or tissues of interest. The invention is further directed to release of the cell surface glycomes, preferably from intact cells by hydrolytic enzymes such as proteolytic enzymes, including proteinases and proteases, and/or glycan releasing enzymes, including endo-glycosidases or protein N-glycosidases. Preferably the surface glycoproteins are cleaveed by proteinase such as trypsin and then glycans are analysed as glycopeptides or preferably released further by glycan releasing enzyme.

### Cell Models of Differentiated Tissues

The invention is further directed to cultured cells corresponding to cancer cells. Such cells may be used as models for cancer. The cancer cells include cell models of cancer.

### The Glycome Compositions

The invention is further directed to the compositions and compositions produced by the methods according to the invention. The invention further represent preferred methods for analysis of the glycomes, especially mass spectrometric methods.

The invention is specifically directed to released glycomes derived from conjugated glycans from preferred tissue materials and cell models of differentiated tissues.

### Purification Method

The invention represents effective methods for purification of oligosaccharide fractions from tissues, especially in very low scale. The prior art has shown analysis of separate glycome components from tissues, but not total glycomes. It is further realized that the methods according to the invention are useful for analysis of glycans from isolated proteins or peptides.

### Analysis of Glycomes

The invention is further directed to novel quantitative analysis methods for glycomes. The glycome analysis produces large amounts of data. The invention reveals methods for the analysis of such data quantitatively and comparison of the data between different samples.

The invention is especially directed to quantitative two-dimensional representation of the data.

### Integrated Glycome Analysis

The invention is further directed to integrated glycomics or glycome analysis process including
1) Optional release of glycans from tissues
2) isolation/purification of glycans from sample,
3) analysis of the glycome
4) quantitative presentation of the data
   The first step is optional as the method is further directed to analysis of known and novel secretion derivable soluble glycomes.

### Application of the Methods for Analysis of Proteins

The invention represents effective methods for the practical analysis of glycans from isolated proteins especially from very small amounts of samples. The invention is especially directed to the application of the methods for the analysis of proteins using the purification method, analysis methods and/or integrated glycome analysis. In a specific embodiment the invention is especially directed in analysis of separated cancer associated proteins for their glycome analysis.

### Product by Process

The present invention is specifically directed to the glycan fraction produced according to the present invention from the pico scale tissue material sample according to the present invention. The preferred glycan fraction is essentially devoid of signals of contaminating molecules within the preferred mass range when analysed by MALDI mass spectrometry according to the present invention.

Preferred Uses of Glycomes and Analysis Thereof with Regard to Status of Cells

In the present invention the word cell refer to cells of tissue material according to the invention, especially cancer cells.

### Product by Process

The present invention is specifically directed to the glycan fraction produced according to the present invention from the pico scale tissue material sample according to the present invention. The preferred glycan fraction is essentially devoid of signals of contaminating molecules within the preferred mass range when analysed by MALDI mass spectrometry according to the present invention.

The glycome products from tissue samples according to present invention are produced preferably directly from complete tissue material cells or membrane fractions thereof, more preferably directly from intact cells as effectively shown in examples. In another preferred embodiment the glycome fractions are cell surface glycomes and produced directly from surfaces of complete tissue material cells, preferably intact or essentially intact cells of tissue materials or surfaces of intact tissues according to the invention. In another embodiment the glycome products according to the invention are produced directly from membrane fraction

Preferred Uses of Glycomes and Analysis Thereof with Regard to Status of Cells

### Search of Novel of Novel Carbohydrate Marker Structures

It is further realized that the analysis of glycome is useful for search of most effectively altering glycan structures in the tissue materials for analysis by other methods. The glycome component identified by glycome analysis according to the invention can be further analysed/verified by known methods such as chemical and/or glycosidase enzymatic degradation(s) and further mass spectrometric analysis and by fragmentation mass spectrometry, the glycan component can be produced in larger scale by known chromatographic methods and structure can be verified by NMR spectroscopy.

The other methods would preferably include binding assay using specific labelled carbohydrate binding agents including especially carbohydrate binding proteins (lectins, antibodies, enzymes and engineered proteins with carbohydrate binding activity) and other chemicals such as peptides or aptamers aimed for carbohydrate binding. It is realized that the novel marker structure can be used for analysis of cells, cell status and possible effects of contaminants to cell with similar indicative value as specific signals of the glycan mass components in glycome analysis by mass spectrometry according to the invention.

The invention is especially directed to search of novel carbohydrate marker structures from cell/tissue surfaces, preferably by using cell surface profiling methods. The cell surface carbohydrate marker structures would be further preferred for the analysis and/or sorting of cells.

### Identification and Classification of Differences in Glycan Datasets

The present invention is specifically directed to analyzing glycan datasets and glycan profiles for comparison and characterization of different tissue materials. In one embodiment of the invention, glycan signals or signal groups associated with given tissue material are selected from the whole glycan datasets or profiles and indifferent glycan signals are removed. The resulting selected signal groups have reduced background and less observation points, but the glycan signals most important to the resolving power are included in the selection. Such selected signal groups and their patterns in different sample types serve as a signature for the identification of the cell type and/or glycan types or biosynthetic groups that are typical to it. By evaluating multiple samples from the same tissue material, glycan signals that have individual i.e. cell line specific variation can be excluded from the selection. Moreover, glycan signals can be identified that do not differ between tissue materials, including major glycans that can be considered as housekeeping glycans.

To systematically analyze the data and to find the major glycan signals associated with given tissue material according to the invention, difference-indicating variables can be calculated for the comparison of glycan signals in the glycan datasets. Preferential variables between two samples include variables for absolute and relative difference of given glycan signal between the datasets from two tissue materials. Most preferential variables according to the invention are:

1. absolute difference A=(S2-S1), and
2. relative difference R=A/S1,
wherein S1 and S2 are relative abundances of a given glycan signal in cell types 1 and 2, respectively.

It is realized that other mathematical solutions exist to express the idea of absolute and relative difference between glycan datasets, and the above equations do not limit the scope of the present invention. According to the present invention, after A and R are calculated for the glycan profile datasets of the two tissue materials, the glycan signals are thereafter sorted according to the values of A and R to identify the most significant differing glycan signals. High value of A or R indicates association with tissue material 2, and vice versa. In the list of glycan data sorted independently by R and A, the tissue material specific glycans occur at the top and the bottom of the lists. More preferentially, if a given signal has high values of both A and R, it is more significant.

Preferred Representation of the Dataset when Comparing Two Tissue Materials

The present invention is specifically directed to the comparative presentation of the quantitative glycome dataset as multidimensional graphs comparing the paraller data or as other three dimensional presentations or for example as two dimensional matrix showing the quantities with a quantitative code, preferably by a quantitative color code.

### Specific Recognition Between Preferred Tissue Materials and Contaminating Materials

The invention is further directed to methods of recognizing different tissue materials, preferably human tissues and more preferably human excretions or serum. It is further realized, that the present reagents can be used for purification of tissue materials by any fractionation method using the specific binding reagents.

Preferred fractionation methods includes fluorescence activated cell sorting (FACS), affinity chromatography methods, and bead methods such as magnetic bead methods.

The invention is further directed to positive selection methods including specific binding to the tissue material but not to contaminating tissue materials. The invention is further directed to target selection methods including specific binding to the contaminating tissue material but not to the target tissue materials. In yet another embodiment of recognition of tissue materials the tissue material is recognized together with a homogenous reference sample, preferably when separation of other materials is needed. It is realized that a reagent for positive selection can be selected so that it binds tissue materials as in the present invention and not to the contaminating tissue materials and a reagent for negative selection by selecting opposite specificity. In case of tissue material type according to the invention is to be selected amongst novel tissue materials not studied in the present invention, the binding molecules according to the invention maybe used when verified to have suitable specificity with regard to the novel tissue material (binding or not binding). The invention is specifically directed to analysis of such binding specificity for development of a new binding or selection method according to the invention.

The preferred specificities according to the invention include recognition of:
i) mannose type structures, especially alpha-Man structures like lectin PAA
ii) sialylated structures similarity as by MAA-lectin
iii) Gal/GalNAc binding specificity, preferably Gal1-3/GalNAc1-3 binding specificity, more preferably Galβ1-3/GalNAcβ1-3 binding specificity similar to PNA

### Specific Characteristic Marker Structures and Glycome Marker Components/Compositions

The N-glycan analysis of total profiles of released N-glycans revealed beside the glycans above, which were verified to comprise

1) complex biantennary N-glycans, such as Galβ4GlcNAcβ2Manα3 (Galβ4GlcNAc β2Manα6)Manβ4GlcNAcβ4(Fucα6)₀₋₁GlcNAcβ-,

wherein the reminal N-acetyllactosamines can be elongated from Gal with NeuNAcα3 and/or NeuNAcα6 and
2) terminal mannose containing N-glycans such as High-mannose glycans with formula Hex₅₋₉HexNAc₂ and degradation products thereof comprising low number of mannose residues (Low mannose glycans) Hex₁₋₄HexNAc₂.

### The Specific N-Glycan Core Marker Structure

The glycan share common core structure according to the Formula:

[Manα3]ₙ₁(Manα6)ₙ₂Manβ4GlcNAcβ4(Fucα6)₀₋₁GlcNAcβAsn,

wherein n1 and n2 are integers 0 or 1, independently indicating the presence or absence of the terminal Man-residue, and
wherein the non-reducing end terminal Manα3/Manα6-residues can be elongated to the complex type, especially biantennary structures or to mannose type (high-Man and/or low Man) or to hybrid type structures as described in examples.

It was further analyzed that the N-glycan compositions contained only very minor amounts of glycans with additional HexNAx in comparison to monosaccharide compositions of the complex type glycan above, which could indicate presence of no or very low amounts of the N-glycan core linked GlcNAc-residues described by Stanley P M and Raju T S (JBC-(1998) 273 (23) 14090-8; JBC (1996) 271 (13) 7484-93) and/or bisecting GlcNAc. is realized that part of the terminal HexNAc-type structures appear to represent bisecting GlcNAc-type type glycans, and quite low or non-existent amounts of the GlcNAcα6-branching and also low amounts of GlcNAcβ2-branch of Manβ4 described by Stanley and colleagues. Here, essentially devoid of indicates less than 10% of all the protein linked N-glycans, more preferably the additional HexNAc units are present in less than 8% of the tissue material N-glycans by mass spectrometric analysis.

The invention thus describes the major core structure of N-glycans in human tissue materials verified by NMR-spectroscopy and by specific glycosidase digestions and was further quantitated to comprise a characteristic smaller structural group glycans comprising specific terminal HexNAc group and/or bisecting GlcNAc-type structures, which additionally modify part of the core structure. The invention further reveals that the core structure is a useful target structure for analysis of tissue materials.

The characteristic monosaccharide composition of the core structure will allow recognition of the major types of N-glycan structure groups present as additional modification of the core structure. Furthermore composition of the core structure is characteristic in mass spectrometric analysis of N-glycan and allow immediate recognition for example from HexₓHexNAc₁-type (preferentially ManₓGlcNAc₁) glycans also present in total glycome composition.

### Low-Molecular Weight Glycan Marker Structures and Tissue Material Glycome Components

The invention describes novel low-molecular weight acidic glycan components within the acidic N-glycan and/or soluble glycan fractions with characteristic monosaccharide compositions SAₓHex₁₋₂HexNAc₁₋₂, wherein x indicates that the corresponding glycans are preferentially sialylated with one or more sialic acid residues. The inventors realized that such glycans are novel and unusual with respect to N-glycan biosynthesis and described mammalian cell glycan components, as reveal also by the fact that they are classified as “other (N-)glycan types” in N-glycan classification scheme of the present invention. The invention is directed to analyzing, isolating, modifying, and/or binding to these novel glycan components according to the methods and uses of the present invention, and further to other uses of specific marker glycans as described here. As demonstrated in the Examples of the present invention, such glycan components were specific parts of total glycomes of certain tissue materials and preferentially to certain tissue material types, making their analysis and use beneficial with regard to tissue materials. The invention is further directed to tissue material glycomes and subglycomes containing these glycan components.

### Preferred Glycomes

The present invention is specifically directed to tissue material glycomes, which are essentially pure glycan mixtures comprising various glycans as described in the invention preferably in proportions shown by the invention. The essentially pure glycan mixtures comprise the key glycan components in proportions which are characteristics to tissue material glycomes. The preferred glycomes are obtained from human tissue materials according to the invention.

The invention is further directed to glycomes as products of purification process and variations thereof according to the invention. The products purified from tissue materials by the simple, quantitative and effective methods according to the invention are essentially pure. The essentially pure means that the mixtures are essentially devoid of contaminations disturbing analysis by MALDI mass spectrometry, preferably by MALDI-TOF mass spectrometry. The mass spectra produced by the present methods from the essentially pure glycomes reveal that there is essentially no non-carbohydrate impurities with weight larger than trisaccharide and very low amount of lower molecular weight impurities so that crystallization of MALDI matrix is possible and the glycan signals can be observed for broad glycomes with large variations of monosaccharide compositions and ranges of molecular weight as described by the invention. It is realized that the purification of the materials from low amounts of tissue materials comprising very broad range of cellular materials is very challenging task and the present invention has accomplished this.

Combination Compositions of the Preferred Glycome Mixtures with Matrix for Analysis

### Mass Spectrometric Matrix

The invention further revealed that it is possible to combine the glycomes with matrix useful for a mass spectrometric analysis and to obtain combination mixture useful for spectrometric analysis. The preferred mass spectrometric matrix is matrix for MALDI (matrix assisted laser desorption ionization mass spectrometry) with mass spectrometric analysis (abbreviated as MALDI matrix), MALDI is preferably performed with TOF (time of flight) detection.

Preferred MALDI matrices include aromatic preferably benzene ring structure comprising molecules with following characteristic. The benzene ring structure molecules preferably comprises 1-4 substituents such as hydroxyl, carboxylic acid or ketone groups. Known MALDI matrixes have been reviewed in Harvey, Mass. Spec. Rev. 18, 349 (1999). The present invention is especially and separately directed to specific matrixes for analysis in negative ion mode of MALDI mass spectrometry, preferred for analysis of negatively charged (acidic, such as sialylated and/or sulfated and/or phosphorylated) subglycome, and in positive ion mode of MALDI mass spectrometry (preferred for analysis of neutral glycomes). It is realized that the matrices can be optimized for negative ion mode and positive ion mode.

The present invention is especially directed to glycome matrix composition optimized for the use in positive ion mode, and to the use of the MALDI-TOF matrix and matrix glycome composition, that is optimized for the use in the analysis in positive ion mode, for the analysis of glycome, preferably neutral glycome. The preferred matrices for positive ion mode are aromatic matrices, e.g. 2,5-dihydroxybenzoic acid, 2,5-dihydroxybenzoic acid/2-hydroxy-5-methoxybenzoic acid, 2,4,6-trihydroxyacetophenone or 6-aza-2-thiothymine, more preferably 2,5-dihydroxybenzoic acid. The present invention is especially directed to glycome matrix composition optimized for the use in negative ion mode, and to the use of the MALDI-TOF matrix and the matrix glycome compositions, that is optimized for the negative ion mode, for the analysis of glycome, preferably acidic glycome. The preferred matrices for negative ion mode are aromatic matrices, e.g. 2,4,6-trihydroxyacetophenone, 3-hydroxypicolinic acid, 2,5-dihydroxybenzoic acid, 2,5-dihydroxybenzoic acid/2-hydroxy-5-methoxybenzoic acid, or 6-aza-2-thiothymine, more preferably 2,4,6-trihydroxyacetophenone. The invention is further directed to analysis method and glycome-matrix composition for the analysis of glycome compositions, wherein the glycome composition comprises both negative and neutral glycome components. Preferred matrices for analysis of negative and neutral glycome components comprising glycome are aromatic matrices, e.g. 2,4,6-trihydroxyacetophenone, 3-hydroxypicolinic acid, 2,5-dihydroxybenzoic acid, 2,5-dihydroxybenzoic acid/2-hydroxy-5-methoxybenzoic acid, or 6-aza-2-thiothymine, more preferably 2,4,6-trihydroxyacetophenone.

The MALDI-matrix is a molecule capable of

1) Specifically and effectively co-crystallizing with glycome composition with the matrix, crystallizing meaning here forming a solid mixture composition allowing analysis of glycome involving two steps below
2) absorbing UV-light typically provided by a laser in MALDI-TOF instrument, preferred wavelength of the light is 337 nm as defined by the manuals of MALDI-TOF methods
3) transferring energy to the glycome composition so that these will ionize and be analyzable by the MALDI-TOF mass spectrometry. The present invention is especially directed to compositions of glycomes in complex with MALDI mass spectrometry matrix.

The present invention is specifically directed to methods of searching novel MALDI-matrixes with the above characteristic, preferably useful for analysis by the method below. The method for searching novel MALDI-matrixes using the method in the next paragraph.

The present invention is specifically directed to methods of analysis of glycomes by MALDI-TOF including the steps:

1) Specifically and effectively co-crystallizing the glycome composition with the MALDI-TOF-matrix, crystallizing meaning here forming a solid mixture composition allowing analysis of glycome involving two steps below
2) Providing UV light to crystalline sample by a laser in MALDI-TOF instrument allowing the ionization of sample

3) Analysis of the ions produced by the MALDI mass spectrometer, preferably by TOF analysis. The invention is further directed to the combination of glycome purification methods and/or quantitative and qualitative data analysis methods according to the invention.

### Crystalline Compositions of Glycomes

The present invention is further directed to essentially pure glycome compositions in solid co-crystalline form with MALDI matrix. The invention is preferably a neutral and/or acidic glycome as complex with a matrix optimized for analysis of the specific glycome type, preferably analysis in negative ion mode with a matrix such as 2,4,6-trihydroxyacetophenone. The invention is preferably a neutral (or non-acidic) glycome as complex with a matrix optimized for analysis in positive ion mode such as 2,5-dihydroxybenzoic acid.

The invention revealed that it is possible to analyze glycomes using very low amount of sample. The preferred crystalline glycome composition comprises between 0.1-100 pmol, more preferably 0.5-10 pmol, more preferably 0.5-5 pmol and more preferably about 0.5-3 pmol, more preferably about 0.5-2 pmol of sample co-crystallized with optimized amount of matrix preferably about 10-200 nmol, more preferably 30-150 nmol, and more preferably about 50-120 nmol and most preferably between 60-90 nmols of the matrix, preferably when the matrix is 2,5-dihydroxybenzoic acid. The matrix and analyte amounts are optimized for a round analysis spot with radius of about 1 mm and area of about 0.8 mm². It is realized that the amount of materials can be changed in proportion of the area of the spot, smaller amount for smaller spot. Examples of preferred amounts per area of spot are 0.1-100 pmol/0.8 mm² and 10-200 pmol/3 mm². Preferred molar excess of matrix is about 5000-1000000 fold, more preferably about 10000-500000 fold and more preferably about 15000 to 200 000 fold and most preferably about 20000 to 100000 fold excess when the matrix is 2,5-dihydroxybenzoic acid.

It is realized that the amount and relative amount of new matrix is optimized based on forming suitable crystals and depend on chemical structure of the matrix. The formation of crystals is observed by microscope and further tested by performing test analysis by MALDI mass spectrometry.

The invention is further directed to specific methods for crystallizing MALDI-matrix with glycome. Preferred method for crystallization in positive ion mode includes steps: (1) optionally, elimination of impurities, like salts and detergents, which interfere with the crystallization, (2) providing solution of glycome in H₂O or other suitable solvent in the preferred concentration, (3) mixing the glycome with the matrix in solution or depositing the glycome in solution on a precrystallized matrix layer and (4) drying the solution preferably by a gentle stream of air.

Preferred method for crystallization in negative ion mode includes steps: (1) optionally, elimination of impurities, like salts and detergents, which interfere with the crystallization, (2) providing solution of glycome in H₂O or other suitable solvent in the preferred concentration, (3) mixing the glycome with the matrix in solution or depositing the glycome in solution on a precrystallized matrix layer and (4) drying the solution preferably by vacuum.

### Other Preferred Glycome Analysis Compostions

### Binder Glycome Compositions

The invention is further directed to compositions of essentially pure glycome composition with specific glycan binding molecules such as lectins, glycosidases or glycosyltransferases and other glycosyl modifying enzymes such as sulfateses and/or phosphatases and antibodies. It is realized that these composition are especially useful for analysis of glycomes.

The present invention revealed that the complex glycome compositions can be effectively and even quantitatively modified by glycosidases even in very low amounts. It was revealed that the numerous glycan structures similar to target structures of the enzymes do not prevent the degradation by competitive inhibition, especially by the enzymes used. The invention is specifically directed to preferred amounts directed to MALDI analysis for use in composition with a glycosyl modifying enzyme, preferably present in low amounts. Preferred enzymes suitable for analysis include enzymes according to the Examples.

The invention is further directed to binding of specific component of glycome in solution with a specific binder. The preferred method further includes affinity chromatography step for purification of the bound component or analysis of the non-bound fraction and comparing it to the glycome solution without the binding substance. Preferred binders include lectins engineered to be lectins by removal of catalytic amino acids (methods published by Roger Laine, Anomeric, Inc., USA, and Prof. Jukka Finne, Turku, Finland), lectins and antibodies or antibody fragments or minimal binding domains of the proteins.

### Additional Data Analysis and Related Methods

The present invention is especially directed to the use of glycome data for production of mathematical formulas, or algorithms, for specific recognition or identification of specific tissue materials. Data analysis methods are presented in Examples.

The invention is especially directed to selecting specific “structural features” such as mass spectrometric signals (such as individual mass spectrometric signal corresponding to one or several monosaccharide compositions and/or glycan structures), or signal groups or subglycomes or signals corresponding to specific glycan classes, which are preferably according to the invention, preferably the signal groups (preferably defined as specific structure group by the invention), from quantitative glycome data, preferably from quantitative glycome data according to the invention, for the analysis of status of tissue materials. The invention is furthermore directed to the methods of analysis of the tissue materials by the methods involving the use of the specific signals or signal groups and a mathematical algorithm for analysis of the status of a tissue material.

Preferred algorithm includes use of proportion (such as %-proportion) of the specific signals from total signals as specific values (structural features) and creating a “glycan score”, which is algorithm showing characteristics/status of a tissue material based on the specific proportional signal intensities (or quantitative presence of glycan structures measured by any quantitation method such as specific binding proteins or quantitative chromatographic or electrophoresis analysis such as HPLC analysis). Preferably signals which are, preferably most specifically, upregulated in specific tissue materials and signals which are, preferably most specifically, downregulated in the tissue material in comparison to control tissue materials are selected to for the glycan score. In a preferred embodiment value(s) of downregulated signals are subtracted from upregulated signals when glycan score is calculated. The method yields largest score values for a specific tissue material type or types selected to be differentiated from other tissue materials.

The invention is specifically directed to methods for searching characteristic structural features (values) from glycome profiling data, preferably quantitative or qualitative glycome profiling data. The preferred methods include methods for comparing the glycome data sets obtained from different samples, or from average data sets obtained from a group of similar samples such as paraller samples from same or similar tissue material preparations. Methods for searching characteristic features are briefly described in the section: identification and classification of differences in glycan datasets. The comparison of datasets of the glycome data according to the invention preferably includes calculation of relative and/or absolute differences of signals, preferably each signal between two data sets, and in another preferred embodiment between three or more datasets. The method preferably further includes step of selecting the differing signals, or part thereof, for calculating glycan score.

It is further realized that the analyzed glycome data has other uses preferred by the invention such as use of the selected characteristic signals and corresponding glycan material:

1) for targets for structural analysis of glycans (preferably chemically by glycosidases, fragmentation mass spectrometry and/or NMR spectroscopy as shown by the present invention and/or structural analysis based on the presence of other signals and knowledge of biosynthesis of glycans). The preferred use for targets includes estimation of chemical characteristics of potential corresponding glycans for complete or partial purification/separation of the specific glycan(s). The preferred chemical characteristics to be analysed preferably include one or several of following properties: a) acidity (e.g. by presence of acidic residues such as sialic acid and/or sulfate and/or phosphate) for charge based separation, b) molecular weight or hydrodynamic volume affecting chromatographic separation, e.g. estimation of the elution volume in gel filtration methods (the effect of acidic residue can be estimated from effects of similar structures and the “size” of HexNAc (GalNAc/GlcNAc) is in general twice the size of Hex (such as Gal, Man or Glc), c) estimation (e.g. based on composition and biosynthetic knowledge of glycans) of presence of epitopes for specific binding reagents for labelling identification in a mixture or for affinity purification, d) estimation of presence of target epitopes for specific glycosylmodifying enzymes including glycosidases and/or glycosyltransferases (types of binding reagents) or for specific chemical modification reagents (such as periodate for specific oxidation or acid for specific acid hydrolysis), for modification of glycans and recognition of the modification by potential chemical change such as incorporation of radioactive label or by change of mass spectrometric signal of the glycan for labelling identification in a mixture.
2) use of the signals or partially or fully analysed glycan structures corresponding to the signals for searching specific binding reagents for recognition of tissue materials which are preferably selected as described by the present invention (especially as described above) and in the methods for identification and classification of differences in glycan datasets and/or signals selected and/or tested by glycan score methods, are preferably selected for targets for structural analysis of glycans (preferably by glycosidases, fragmentation mass spectrometry and/or NMR spectroscopy as shown by the present invention) and/or for use of the signals or partially or fully analysed glycan structures corresponding to the signals for searching specific binding reagents for recognition of tissue materials.

The preferred method includes the step of comparing the values, and preferably presenting the score values in graphs such as ones shown in FIG. 36 (example 23), and preferably evaluating the statistic significance of the result by a statistic analysis methods such as a mathematical test for statistic significance, tissue material type refers here to tissue materials with specific status and/or identity, e.g. malignancy, with possible individual variability, e.g. between individual patients.

It is realized that to differentiate a tissue materials type from other(s) different characteristic signals may be selected than for another tissue material type. The invention however revealed that for tissue materials and especially for human cancer patients preferred characteristic signals include ones selected in the Examples as described above. It is realized that a glycan score can be also created with less characteristic signals or with only part of signals and still relevant results can be obtained. The invention is further directed to methods for optimisation of glycan score algorithms and methods for selecting signals for glycan scores.

In case the specific proportion (value) of a characteristic signal is low in comparison to other values a specific factor can be selected for increase the relative “weight” of the value in the glycan scores to be calculated for the cell populations.

The preferred statuses of tissue materials, to be analysed by mathematical methods such as algorithms using quantitative glycome profiling data according to the invention include differentiation status, individual characteristics and mutation, cell culture or storage conditions related status, effects of chemicals or biochemicals on cells, and other statuses described by the invention.

### Preferred Structures of O-Glycan Glycomes of Tissue Materials

The present invention is especially directed to following O-glycan marker structures of tissue materials:

Core 1 type O-glycan structures following the marker composition NeuAc₂Hex₁HexNAc₁, preferably including structures SAα3Galβ3GalNAc and/or SAα3Galβ3(Saα6)GalNAc; and Core 2 type O-glycan structures following the marker composition NeuAc₀₋₂Hex₂HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1; more specifically preferably including R₁Galβ4(R₃)GlcNAcβ6(R₂Galβ3)GalNAc, wherein R₁ and R₂ are independently either nothing or sialic acid residue, preferably α-2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and

R₃ is independently either nothing or fucose residue, preferably α1,3-linked fucose residue. It is realized that these structures correlate with expression of β6GlcNAc-transferases synthesizing core 2 structures.

### Preferred Qualitative and Quantitative Complete N-Glycomes of Tissue Materials

### High-Mannose Type and Glycosylated N-Glycans

The present invention is especially directed to glycan compositions (structures) and analysis of high-mannose type and glycosylated N-glycans according to the formula:

Hexₙ₃HexNAcₙ₄,

wherein n3 is 5, 6, 7, 8, 9, 10, 11, or 12, and n4=2.

According to the present invention, within total N-glycomes of tissue materials the major high-mannose type and glycosylated N-glycan signals preferentially include the compositions with 5≦n3≦10: Hex5HexNAc2 (1257), Hex6HexNAc2 (1419), Hex7HexNAc2 (1581), Hex8HexNAc2 (1743), Hex9HexNAc2 (1905), and Hex10HexNAc2 (2067); and more preferably with 5≦n3≦9: Hex5HexNAc2 (1257), Hex6HexNAc2 (1419), Hex7HexNAc2 (1581), Hex8HexNAc2 (1743), and Hex9HexNAc2 (1905).

### Low-Mannose Type N-Glycans

The present invention is especially directed to glycan compositions (structures) and analysis of low-mannose type N-glycans according to the formula:

Hexₙ₃HexNAcₙ₄dHexₙ₅,

wherein n3 is 1, 2, 3, or 4, n4=2, and n5 is 0 or 1.

According to the present invention, within total N-glycomes of tissue materials the major low-mannose type N-glycan signals preferably include the compositions with 2≦n3≦4: Hex2HexNAc2 (771), Hex3HexNAc2 (933), Hex4HexNAc2 (1095), Hex2HexNAc2dHex (917), Hex3HexNAc2dHex (1079), and Hex4HexNAc2dHex (1241); and more preferably when n5 is 0: Hex2HexNAc2 (771), Hex3HexNAc2 (933), and Hex4HexNAc2 (1095).

As demonstrated in the present invention by glycan structure analysis of tissue materials, preferably this glycan group in tissue materials includes the molecular structures: (Manα)1-3Manβ4GlcNAcβ4(Fucα6)₀₋₁GlcNAc within the glycan signals 771, 917, 933, 1079, 1095, and 1095, and

the preferred low-Man structures includes structures common all tissue material types, tri-Man and tetra-Man structures according to the Examples, (Manα)ₒ— Manα6(ManαG)Manβ4GlcNAcβ4(Fucα6)₀₋₁GlcNAc, more preferably the tri-Man structures:

### Manα6(Manα3)Man 4GlcNAeβ4(Fucα6)₀₋₁GlcNAc

even more preferably the abundant molecular structure:
Manα6(Manα3)Manβ4GlcNAcβ4GlcNAc within the glycan signal 933.

### Quantitative Analysis Directed to the Low-Man Components

Beside the qualitative variations the low-Man glycans have specific value in quantitative analysis of tissue materials. The present invention revealed that the low-Man glycans are especially useful for the analysis of status of the cells. For example the analysis in the Examples revealed that the amounts of the glycans vary between total tissue profiles and specific organelles, preferably lysosomes.

The group of low-Man glycans form a characteristic group among glycome compositions. The relative total amount of neutral glycans is notable in average human tissues. The glycan group was revealed also to be characteristic in cancerous tissues and tumors a with total relative amount of neutral glycomes increased. The difference is more pronounced within lysosomal organelle-specific glycome, wherein low-Man structures accounted nearly 50% of the neutral protein-linked glycome. Glycome analysis of tissue materials is especially useful for methods for development of binder reagents for separation of different tissue materials.

The invention is directed to analysis of relative amounts of low-Man glycans, and to the specific quantitative glycome compositions, especially neutral glycan compositions, comprising about 0 to 50% of low-Man glycans, more preferably between about 1 to 50% of solid tissue glycomes, for the analysis of tissue materials according to the invention, and use of the composition for the analysis of tissue materials.

### Fucosylated High-Mannose Type N-Glycans

The present invention is especially directed to glycan compositions (structures) and analysis of fucosylated high-mannose type N-glycans according to the formula:

Hexₙ₃HexNAcₙ₄dHexₙ₅,

wherein n3 is 5, 6, 7, 8, or 9, n4=2, and n5=1.

According to the present invention, within total N-glycomes of tissue materials the major fucosylated high-mannose type N-glycan signal preferentially is the composition Hex5HexNAc2dHex (1403).

### Soluble Glycans

The present invention is especially directed to glycan compositions (structures) and analysis of neutral soluble N-glycan type glycans according to the formula:

Hexₙ₃HexNAcₙ₄,

wherein n3 is 1, 2, 3, 4, 5, 6, 7, 8, or 9, and n4=1.

Within total N-glycomes of tissue materials the major soluble N-glycan signals include the compositions with 4≦n3≦8, more preferably 4≦n3≦7: Hex4HexNAc (892), Hex5HexNAc (1054), Hex6HexNAc (1216), Hex7HexNAc (1378). In the most preferred embodiment of the present invention, the major glycan signal in this group within total neutral glycomes of tissue materials is Hex5HexNAc (1054).

### Neutral Monoantennary or Hybrid-Type N-Glycans

The present invention is especially directed to glycan compositions (structures) and analysis of neutral monoantennary or hybrid-type N-glycans according to the formula:

Hexₙ₃HexNAcₙ₄dHexₙ₅,

wherein n3 is an integer greater or equal to 2, n4=3, and n5 is an integer greater or equal to 0.

According to the present invention, within total N-glycomes of tissue materials the major neutral monoantennary or hybrid-type N-glycan signals preferentially include the compositions with 2≦n3≦8 and 0≦n5≦2, more preferentially compositions with 3≦n3≦6 and 0≦n5≦1, with the proviso that when n3=6 also n5=0: preferentially Hex4HexNAc3 (1298), Hex4HexNAc3dHex (1444), Hex5HexNAc3 (1460), and Hex6HexNAc3 (1622).

### Neutral Complex-Type N-Glycans

The present invention is especially directed to glycan compositions (structures) and analysis of neutral complex-type N-glycans according to the formula:

Hexₙ₃HexNAcₙ₄dHexₙ₅,

wherein n3 is an integer greater or equal to 3, n4 is an integer greater or equal to 4, and n5 is an integer greater or equal to 0.

Within the total N-glycomes of tissue materials the major neutral complex-type N-glycan signals preferentially include the compositions with 3≦n3≦8, 4≦n4≦7, and 0≦n5≦4, more preferentially the compositions with 3≦n3≦5, n4=4, and 0≦n5≦1, with the proviso that when n3 is 3 or 4, then n5=1: Hex3HexNAc4dHex (1485), Hex4HexNAc4dHex (1647), Hex5HexNAc4 (1663), Hex5HexNAc4dHex (1809); and even more preferentially also including the composition Hex3HexNAc5dHex (1688).

In another embodiment of the present invention, the N-glycan signal Hex3HexNAc4dHex (1485) contains non-reducing terminal GlcNAcβ, and more preferentially the total N-glycome includes the structure:

### GlcNAc β2Manα3(GlcNAcβ2Manα6)Manβ4GlcNAcβ4(Fucα6)GlcNAc (1485).

In yet another embodiment of the present invention, within the total N-glycome of tissue materials, the N-glycan signal Hex5HexNAc4dHex (1809), more preferentially also Hex5HexNAc4 (1663), contain non-reducing terminal β1,4-Gal. Even more preferentially the total N-glycome includes the structure:

Galβ4GlcNAcβ2Manα3 (Galβ4GlcNAcβ2Manα6)Manβ4GlcNAcβ4GlcNAc (1663); and in a further preferred embodiment the total N-glycome includes the structure:

Galβ4GlcNAcβ2Manα3 (Galβ34GlcNAcβ2Manα6)Manβ4GlcNAcβ4(Fucα6)GlcNAc (1809).

### Neutral Fucosylated N-Glycans

The present invention is especially directed to glycan compositions (structures) and analysis of neutral fucosylated N-glycans according to the formula:

Hexₙ₃HexNAcₙ₄dHexₙ₅,

wherein n5 is an integer greater than or equal to 1.

Within the total N-glycomes of tissue materials the major neutral fucosylated N-glycan signals preferentially include glycan compositions wherein 1≦n5≦4, more preferentially 1≦n5≦3, even more preferentially 1≦n5≦2, and further more preferentially compositions Hex3HexNAc2dHex (1079), more preferentially also Hex2HexNAc2dHex (917), and even more preferentially also Hex5HexNAc4dHex (1809).

The inventors further found that within the total N-glycomes of tissue materials a major fucosylation form is N-glycan core α1,6-fucosylation. In a preferred embodiment of the present invention, major fucosylated N-glycan signals contain GlcNAcβ4(Fucα6)GlcNAc reducing end sequence.

Neutral N-Glycans with Non-Reducing Terminal HexNac

The present invention is especially directed to glycan compositions (structures) and analysis of neutral N-glycans with non-reducing terminal HexNAc according to the formula:

Hexₙ₃HexNAcₙ₄dHexₙ₅,

wherein n4≧n3.

Preferably these glycan signals include Hex3HexNAc4dHex (1485) in all tissue materials.

### Acidic Hybrid-Type or Monoantennary N-Glycans

The present invention is especially directed to glycan compositions (structures) and analysis of acidic hybrid-type or monoantennary N-glycans according to the formula:

NeuAcₙ₁NeuGcₙ₂Hexₙ₃HexNAcₙ₄dHexₙ₅SPₙ₆,

wherein n1 and n2 are either independently 1, 2, or 3; n3 is an integer between 3-9; n4 is 3; n5 is an integer between 0-3; and n6 is an integer between 0-2; with the proviso that the sum n1+n2+n6 is at least 1.

Within the total N-glycomes of tissue materials the major acidic hybrid-type or monoantennary N-glycan signals preferentially include glycan compositions wherein 3≦n3≦6, more preferentially 3≦n5≦5, and further more preferentially composition NeuAcHex4HexNAc3dHex (1711).

### Acidic Complex-Type N-Glycans

The present invention is especially directed to glycan compositions (structures) and analysis of acidic complex-type N-glycans according to the formula:

NeuAcₙ₁NeuGcₙ₂Hexₙ₃HexNAcₙ₄dHexₙ₅SPₙ₆,

wherein n1 and n2 are either independently 1, 2, 3, or 4; n3 is an integer between 3-10; n4 is an integer between 4-9; n5 is an integer between 0-5; and n6 is an integer between 0-2; with the proviso that the sum n1+n2+n6 is at least 1.

Within the total N-glycomes of tissue materials the major acidic complex-type N-glycan signals preferentially include glycan compositions wherein 4≦n4≦8, more preferentially 4≦n4≦6, more preferentially 4≦n4≦5, and further more preferentially compositions NeuAcHex5HexNAc4 (1930), NeuAcHex5HexNAc4dHex (2076), NeuAc2Hex5HexNAc4 (2221), NeuAcHex5HexNAc4dHex2 (2222), and NeuAc2Hex5HexNAc4dHex (2367).

### Modified Glycan Types

The inventors found that tissue material total N-glycomes; and soluble+N-glycomes further contain characteristic modified glycan signals, including sialylated fucosylated N-glycans, multifucosylated glycans, sialylated N-glycans with terminal HexNAc (the N>H and N═H subclasses), and sulphated or phosphorylated N-glycans, which are subclasses of the abovementioned glycan classes. According to the present invention, their quantitative proportions in different tissue materials have characteristic values as described in Tables 8 and 13.

### Phosphorylated and Sulphated Glycans

Specifically, major phosphorylated glycans typical to tissue materials, more preferentially to lysosomal organelle glycomes, include Hex5HexNAc2(HPO₃) (1313), Hex6HexNAc2(HPO₃) (1475), and Hex7HexNAc2(HPO₃) (1637). Preferred combinations of glycan types in complete glycomes

The preferred complete glycomes of tissue materials include low-mannose type, hybrid-type or monoantennary, hybrid, and complex-type N-glycans,

which more preferentially contain fucosylated glycans, even more preferentially also sialylated glycans, and further more preferentially also sulphated and/or phosphorylated glycans;
and most preferentially also including soluble glycans as described in the present invention.

In a preferred embodiment of the present invention the tissue material total N-glycome contains the three glycan types: 1) high-mannose type, 2) hybrid-type or monoantennary, and 3) complex-type N-glycans; and more preferably, in the case of solid tissues or cells also 4) low-mannose type N-glycans; and further more preferably, in the case of solid tissues or cells additionally 5) soluble glycans.

In a preferred embodiment of the preferred glycan type combinations within the tissue material complete glycomes, their relative abundances are as described in Tables of Examples.

### More Detailed Structure and Method Descriptions

### Structures of N-Linked Glycomes

### Common Core Structure of N-Linked Glycomes

The inventors revealed that the N-glycans released by specific N-glycan release methods from the cells according to the invention, and preferred cells according to the invention, comprise mostly a specific type of N-glycan core structure.

The preferred N-glycan structure of each cell type is characterised and recognized by treating cells with a N-glycan releasing enzyme releasing practically all N-glycans with core type according to the invention. The N-glycan releasing enzyme is preferably protein N-glycosidase enzyme, preferably by protein N-glycosidase releasing effectively the N-glycomes according to the invention, more preferably protein N-glycosidase with similar specificity as protein N-glycosidase F, and in a specifically preferred embodiment the enzyme is protein N-glycosidase F from F. meningosepticum. Alternative chemical N-glycan release method was used for controlling the effective release of the N-glycomes by the N-glycan releasing enzyme.

The inventors used the NMR glycome analysis according to the invention for further characterization of released N-glycomes from small cell samples available. NMR spectroscopy revealed the N-glycan core signals of the preferred N-glycan core type of the cells according to the invention.

### The Minimum Formula

The present invention is directed to glycomes derived from cells and comprising a common N-glycosidic core structures. The invention is specifically directed to minimum formulas covering both GN₁-glycomes and GN₂-glycomes with difference in reducing end structures.

The minimum core structure includes glycans from which reducing end GlcNAc or Fucα6GlcNAc has been released. These are referred as GN₁-glycomes and the components thereof as GN₁-glycans. The present invention is specifically directed to natural N-glycomes from cells comprising GN₁-glycans. In a preferred embodiment the invention is directed to purified or isolated practically pure natural GN₁-glycome from human cells. The release of the reducing end GlcNAc-unit completely or partially may be included in the production of the N-glycome or N-glycans from cells for analysis. The invention is specifically directed to soluble high/low mannose glycome of GN₁-type.

The glycomes including the reducing end GlcNAc or Fucα6GlcNAc are referred as GN₂-glycomes and the components thereof as GN₂-glycans. The present invention is also specifically directed to natural N-glycomes from cells and tissues comprising GN₂-glycans. In a preferred embodiment the invention is directed to purified or isolated practically pure natural GN₂-glycome from cells.

The preferred N-glycan core structure(s) and/or N-glycomes from cells according to the invention comprise structure(s) according to the formula NC1:

R₁Mβ4GNXyR₂,

Wherein X is glycosidically linked disaccharide epitope β4(Fucα6)ₙGN, wherein n is 0 or 1, or X is nothing and
y is anomeric linkage structure α and/or β or linkage from derivatized anomeric carbon, and R₁ indicates 1-4, preferably 1-3, natural type carbohydrate substituents linked to the core structures,
R₂ is reducing end hydroxyl, chemical reducing end derivative or natural asparagine N-glycoside derivative such as asparagine N-glycosides including asparagines N-glycoside aminoacids and/or peptides derived from protein.

It is realized that when the invention is directed to a glycome, the formula indicates mixture of several or typically more than ten or even higher number of different structures according to the Formulas describing the glycomes according to the invention.

The possible carbohydrate substituents R₁ comprise at least one mannose (Man) residue, and optionally one or several GlcNAc, Gal, Fuc, SA and/GalNAc residues, with possible sulphate and or phosphate modifications.

When the glycome is released by N-glycosidase the free N-glycome saccharides comprise in a preferred embodiment reducing end hydroxyl with anomeric linkage A having structure cc and/or β, preferably both β and β. In another embodiment the glycome is derivatized by a molecular structure which can be reacted with the free reducing end of a released glycome, such as amine, aminooxy or hydrazine or thiol structures. The derivatizing groups comprise typically 3 to 30 atoms in aliphatic or aromatic structures or can form terminal group spacers and link the glycomes to carriers such as solid phases or microparticles, polymeric carries such as oligosaccharides and/or polysaccharide, peptides, dendrimer, proteins, organic polymers such as plastics, polyethyleneglycol and derivatives, polyamines such as polylysines.

When the glycome comprises asparagine N-glycosides, A is preferably beta and R is linked asparagine or asparagine peptide. The peptide part may comprise multiple different aminoacid residues and typically multiple forms of peptide with different sequences derived from natural proteins carrying the N-glycans in cell materials according to the invention. It is realized that for example proteolytic release of glycans may produce mixture of glycopeptides. Preferably the peptide parts of the glycopeptides comprises mainly a low number of amino acid residues, preferably two to ten residues, more preferably two to seven amino acid residues and even more preferably two to five aminoacid residues and most preferably two to four amino acid residues when “mainly” indicates preferably at least 60% of the peptide part, more preferably at least 75% and most preferably at least 90% of the peptide part comprising the peptide of desired low number of aminoacid residues.

### The Preferred GN₂-N-Glycan Core Structure(s)

The preferred GN₂-N-glycan core structure(s) and/or N-glycomes from cells according to the invention comprise structure(s) according to the formula NC2:

R₁Mβ4GNβ4(Fucα6)ₙGNyR₂,

wherein n is 0 or 1 and
wherein y is anomeric linkage structure α and/or β or linkage from derivatized anomeric carbon and
R₁ indicates 1-4, preferably 1-3, natural type carbohydrate substituents linked to the core structures,
R₂ is reducing end hydroxyl, chemical reducing end derivative or natural asparagine N-glycoside derivative such as asparagine N-glycosides including asparagines N-glycoside aminoacid and/or peptides derived from protein.

The preferred compositions thus include one or several of the following structures
NC2a: Mα3{Mα6}Mβ4GNβ4{Fucα6}ₙ₁GNyR₂
NC2b: Mα6Mβ4GNβ4{Fucα6}ₙ₁GNyR₂
NC2c: Mα3Mβ4GNβ4{Fucα6}ₙ₁GNyR₂

More preferably compositions comprise at least 3 of the structures or most preferably both structures according to the formula NC2a and at least both fucosylated and non-fucosylated with core structure(s) NC2b and/or NC2c.

### The Preferred GN₁-N-Glycan Core Structure(s)

The preferred GN₁-N-glycan core structure(s) and/or N-glycomes from cells according to the invention comprise structure(s) according to the formula NC3:

R₁Mβ4GNyR₂,

wherein y is anomeric linkage structure cc and/or P or linkage from derivatized anomeric carbon and
R₁ indicates 1-4, preferably 1-3, natural type carbohydrate substituents linked to the core structures,
R₂ is reducing end hydroxyl, chemical reducing end derivative or natural asparagine N-glycoside derivative such as asparagine N-glycosides including asparagine N-glycoside aminoacids and/or peptides derived from protein.

### Multi-Mannose GN₁—N-Glycan Core Structure(s)

The invention is specifically directed glycans and/or glycomes derived from preferred cells according to the present invention when the natural glycome or glycan comprises Multi-mannose GN₁-N-glycan core structure(s) structure(s) according to the formula NC4:

[R₁Mα3]ₙ₃{R₃Mα6}ₙ₂Mβ4GNXyR₂,

R₁ and R₃ indicate nothing or one or two, natural type carbohydrate substituents linked to the core structures, when the substituents are α-linked mannose monosaccharide and/or oligosaccharides and the other variables are as described above.

Furthermore common elongated GN₂-N-glycan core structures are preferred types of glycomes according to the invention

THE preferred N-Glycan Core Structures Further Include Differently Elongated GN₂-N-Glycan Core Structures According to the formula NC5:

[R₁Mα3]ₙ₃{R₃Mα6}ₙ₂Mβ4GNβ4{Fucα6}ₙ₁GNyR₂,

wherein n1, n2 and n3 are either 0 or 1 and wherein y is anomeric linkage structure a and/or D or linkage from derivatized anomeric carbon and
R₁ and R₃ indicate nothing or 1-4, preferably 1-3, most preferably one or two, natural type carbohydrate substituents linked to the core structures,
R₂ is reducing end hydroxyl, chemical reducing end derivative or natural asparagine N-glycoside derivative such as asparagine N-glycosides including asparagine N-glycoside aminoacids and/or peptides derived from protein,
GN is GlcNAc, M is mannosyl-, [ ] indicate groups either present or absent in a linear sequence.
{ }indicates branching which may be also present or absent.
with the provision that at least n2 or n3 is 1. Preferably the invention is directed to compositions comprising with all possible values of n2 and n3 and all saccharide types when R1 and/or are R3 are oligosaccharide sequences or nothing.

Preferred N-Glycan Types in Glycomes Comprising N-Glycans

The present invention is preferably directed to N-glycan glycomes comprising one or several of the preferred N-glycan core types according to the invention. The present invention is specifically directed to specific N-glycan core types when the compositions comprise N-glycan or N-glycans from one or several of the groups Low mannose glycans, High mannose glycans, Hybrid glycans, and Complex glycans, in a preferred embodiment the glycome comprise substantial amounts of glycans from at least three groups, more preferably from all four groups.

### Major Subtypes of N-Glycans in N-Linked Glycomes

The invention revealed certain structural groups present in N-linked glycomes. The grouping is based on structural features of glycan groups obtained by classification based on the monosaccharide compositions and structural analysis of the structural groups. The glycans were analysed by NMR, specific binding reagents including lectins and antibodies and specific glycosidases releasing monosaccharide residues from glycans. The glycomes are preferably analysed as neutral and acidic glycomes

### The Major Neutral Glycan Types

The neutral glycomes mean glycomes comprising no acidic monosaccharide residues such as sialic acids (especially NeuNAc and NeuGc), HexA (especially GlcA, glucuronic acid) and acid modification groups such as phosphate and/or sulphate esters. There are four major types of neutral N-linked glycomes which all share the common N-glycan core structure: High-mannose N-glycans, low-mannose N-glycans, hydrid type and complex type N-glycans. These have characteristic monosaccharide compositions and specific substructures. The complex and hybrid type glycans may include certain glycans comprising monoantennary glycans.

The groups of complex and hybrid type glycans can be further analysed with regard to the presence of one or more fucose residues. Glycans containing at least one fucose units are classified as fucosylated. Glycans containing at least two fucose residues are considered as glycans with complex fucosylation indicating that other fucose linkages, in addition to the α1,6-linkage in the N-glycan core, are present in the structure. Such linkages include α1,2-, α1,3-, and α1,4-linkage.

Furthermore the complex type N-glycans may be classified based on the relations of HexNAc (typically GlcNAc or GalNAc) and Hex residues (typically Man, Gal). Terminal HexNAc glycans comprise at least three HexNAc units and at least two Hexose units so that the number of Hex Nac residues is at least larger or equal to the number of hexose units, with the provision that for non branched, monoantennary glycans the number of HexNAcs is larger than number of hexoses.

This consideration is based on presence of two GlcNAc units in the core of N-glycan and need of at least two Mannose units to for a single complex type N-glycan branch and three mannose to form a trimannosyl core structure for most complex type structures. A specific group of HexNAc N-Glycans contains the same number of HexNAcs and Hex units, when the number is at least 5.

### Preferred Mannose Type Structures

The invention is further directed to glycans comprising terminal Mannose such as Mα6-residue or both Manα6- and Manα3-residues, respectively, can additionally substitute other Mα2/3/6 units to form a Mannose-type structures including hydrid, low-Man and High-Man structures according to the invention.

Preferred high- and low mannose type structures with GN2-core structure are according to the Formula M2:

[Mα2]ₙ₁[Mα3]ₙ₂{[Mα2]ₙ₃[Mα6)]ₙ₄}[Mα6]ₙ₅{[Mα2]ₙ₆[Mα2]ₙ₇[Mα3]ₙ₈}Mβ4GNβ4[{Fucα6}]ₘ GNyR₂

wherein p, n1, n2, n3, n4, n5, n6, n7, n8, and m are either independently 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0;
y is anomeric linkage structure cc and/or P or linkage from derivatized anomeric carbon, and
R₂ is reducing end hydroxyl, chemical reducing end derivative or natural asparagine N-glyco side derivative such as asparagine N-glycosides including asparagines N-glyco side aminoacid and/or peptides derived from protein;
[ ] indicates determinant either being present or absent depending on the value of n1, n2, n3, n4, n5, n6, n7, n8, and m; and
{ } indicates a branch in the structure.

Preferred yR₂-structures include [β-N-Asn]ₚ, wherein p is either 0 or 1.

### Preferred Mannose Type Glycomes Comprising GN1-Core Structures

As described above a preferred variant of N-glycomes comprising only single GlcNAc-residue in the core. Such structures are especially preferred as glycomes produced by endo-N-acetylglucosaminidase enzymes and Soluble glycomes. Preferred Mannose type glycomes include structures according to the Formula M2

[Mα2]ₙ₁[Mα3]ₙ₂{[Mα2]ₙ₃[Mα6]ₙ₄}[Mα6]ₙ₅{[Mα2]ₙ₆[Mβ2]ₙ₇[Mα3]ₙ₈}Mβ4GNyR₂

Fucosylated high-mannose N-glycans according to the invention have molecular compositions Man₅₋₉GlcNAc₂Fuc₁. For the fucosylated high-mannose glycans according to the formula, the sum of n1, n2, n3, n4, n5, n6, n7, and n8 is an integer from 4 to 8 and m is 0.

The low-mannose structures have molecular compositions Man₁₋₄GlcNAc₂Fuc₀₋₁. They consist of two subgroups based on the number of Fuc residues: 1) nonfucosylated low-mannose structures have molecular compositions Man₁₋₄GlcNAc₂ and 2) fucosylated low-mannose structures have molecular compositions Man₁₋₄GlcNAc₂Fuc₁. For the low mannose glycans the sum of n1, n2, n3, n4, n5, n6, n7, and n8 is less than or equal to (m+3); and preferably n1, n3, n6, and n7 are 0 when m is 0.

### Low Mannose Glycans

The invention revealed a very unusual group glycans in N-glycomes of invention defined here as low mannose N-glycans. These are not clearly linked to regular biosynthesis of N-glycans, but may represent unusual biosynthetic midproducts or degradation products. The low mannose glycans are especially characteristics changing during the changes of cell status, the differentiation and other changes according to the invention, for examples changes associated with differentiation status of cells and their differentiated products and control cell materials. The invention is especially directed to recognizing low amounts of low-mannose type glycans in cell types, such as with low degree of differentiation.

The invention revealed large differences between the low mannose glycan expression in the cell and tissue glycomes and material from tissue secretions such as human serum.

The invention is especially directed to the use of specific low mannose glycan comprising glycomes for analysis of tissues and cells, preferably cultivated cells.

The invention further revealed specific mannose directed recognition methods useful for recognizing the preferred glycomes according to the invention. The invention is especially directed to combination of glycome analysis and recognition by specific binding agents, most preferred binding agent include enzymes and theis derivatives. The invention further revealed that specific low mannose glycans of the low mannose part of the glycomes can be recognized by degradation by specific α-mannosidase (Man₂₋₄GlcNAc₂Fuc₀₋₁) or β-mannosidase (Man, GlcNAc₂Fuc₀₋₁) enzymes and optionally further recognition of small low mannose structures, even more preferably low mannose structures comprising terminal Manβ4-structures according to the invention.

The low mannose N-glycans, and preferred subgroups and individual structures thereof, are especially preferred as markers of the novel glycome compositions of the cells according to the invention useful for characterization of the cell types.

The low-mannose type glycans includes a specific group of α3- and/or α6-linked mannose type structures according to the invention including a preferred terminal and core structure types according to the invention.

The inventions further revealed that low mannose N-glycans comprise a unique individual structural markers useful for characterization of the cells according to the invention by specific binding agents according to the invention or by combinations of specific binding agents according to the invention.

Neutral low-mannose type N-glycans comprise one to four or five terminal Man-residues, preferentially Manαt structures; for example Manα3Manβ34GlcNAcβ4GlcNAc(β-N-Asn) or Manα₀₋₄Manβ4GlcNAcβ4(Fucα6)GlcNAc(β-N-Asn).

Low-mannose N-glycans are smaller and more rare than the common high-mannose N-glycans (Man₅₋₉GlcNAc₂). The low-mannose N-glycans detected in cell samples fall into two subgroups: 1) non-fucosylated, with composition ManₙGlcNAc₂, where 1≦n≦4, and 2) core-fucosylated, with composition ManₙGlcNAc₂Fuc₁, where 1≦n≦5. The largest of the detected low-mannose structure structures is Man₅GlcNAc₂Fuc₁ (m/z 1403 for the sodium adduct ion), which due to biosynthetic reasons most likely includes the structure below (in the figure the glycan is free oligosaccharide and β-anomer; in glycoproteins in tissues the glycan is N-glycan and H-anomer):

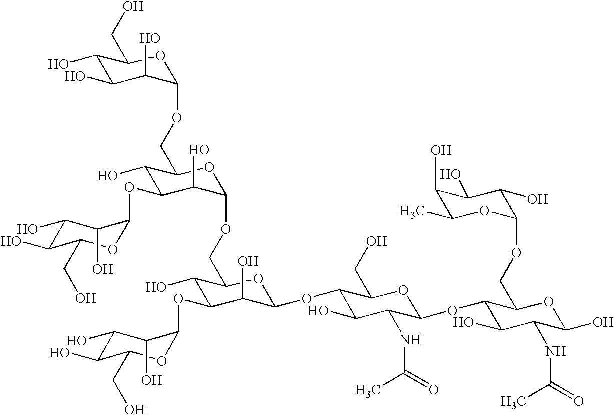

### Preferred General Molecular Structural Features of Low Alan Glycans

According to the present invention, low-mannose structures are preferentially identified by mass spectrometry, preferentially based on characteristic Hex₁₋₄HexNAc₂dHex₀₋₁ monosaccharide composition. The low-mannose structures are further preferentially identified by sensitivity to exoglycosidase digestion, preferentially α-mannosidase (Hex₂₋₄HexNAc₂dHex₀₋₁) or β-mannosidase (Hex₁HexNAc₂dHex₀₋₁) enzymes, and/or to endoglycosidase digestion, preferentially N-glycosidase F detachment from glycoproteins, Endoglycosidase H detachment from glycoproteins (only Hex₁₋₄HexNAc₂ liberated as Hex₂₋₄HexNAc₁), and/or Endoglycosidase F2 digestion (only Hex₁₋₄HexNAc₂dHex, digested to Hex₁₋₄HexNAc₁). The low-mannose structures are further preferentially identified in NMR spectroscopy based on characteristic resonances of the Manβ4GlcNAcβ4GlcNAc N-glycan core structure and Manαresidues attached to the Manβ4 residue.

Several preferred low Man glycans described above can be presented in a single Formula:

[Mα3]ₙ₂{[Mα6)]ₙ₄}[Mα6]ₙ₅{[Mα3]ₙ₈}Mβ4GNβ4[{Fucα6}]ₘGNyR₂

wherein p, n12, n4, n5, n8, and m are either independently 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; the sum of n1, n2, n3, n4, n5, n6, n7, and n8 is less than or equal to (m+3); [ ] indicates determinant either being present or absent depending on the value of n2, n4, n5, n8, and m; and
{ } indicates a branch in the structure;
y and R2 are as indicated above.

Preferred non-fucosylated low-mannose glycans are according to the formula:

[Mα3]ₙ₂([Mα6)]ₙ₄)[Mα6]ₙ₅{[Mα3]ₙ₈}Mβ4GNβ4GNyR₂

wherein p, n2, n4, n5, n8, and m are either independently 0 or 1,
with the provisio that when n5 is 0, also n2 and n4 are 0, and preferably either n2 or n4 is 0,
[ ] indicates determinant either being present or absent depending on the value of, n2, n4, n5, n8,
{ } and 0 indicates a branch in the structure,
y and R₂ are as indicated above.

### Preferred Individual Structures of Nonfucosylated Low-Mannose Glycans

### Special Small Structures

Small non-fucosylated low-mannose structures are especially unusual among known N-linked glycans and characteristic glycans group useful for separation of cells according to the present invention. These include:
Mβ4GNβ4GNyR₂
Mα6Mβ4GNβ4GNyR₂
Mα3Mβ4GNβ4GNyR₂ and
Mα6{Mα3}Mβ4GNβ4GNyR₂.
   Mβ4GNβ4GNyR₂ trisaccharide epitope is a preferred common structure alone and together with its mono-mannose derivatives Mα6Mβ4GNβ4GNyR₂ and/or Mα3Mβ4GNβ4GNyR₂, because these are characteristic structures commonly present in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

### Special Large Structures

The invention further revealed large non-fucosylated low-mannose structures that are unusual among known N-linked glycans and have special characteristic expression features among the preferred cells according to the invention. The preferred large structures include
[Mα3]ₙ₂([Mα6]ₙ₄)Mα6{Mα3}Mβ4GNβ4GNyR₂
   more specifically
Mα6Mα6{Mα3}M4GNβ4GNyR₂
Mα3Mα6{Mα3}M4GNβ4GNyR₂ and
Mα3(Mα6)Mα61Mα31M4GNβ4GNyR₂.

The hexasaccharide epitopes are preferred in a specific embodiment as rare and characteristic structures in preferred cell types and as structures with preferred terminal epitopes. The heptasaccharide is also preferred as structure comprising a preferred unusual terminal epitope Mα3(Mα6)Ma useful for analysis of cells according to the invention.

Preferred fucosylated low-mannose glycans are derived according to the formula:

[Mα3]ₙ₂{[Mα6]ₙ₄}[Mα6]ₙ₅{[Mα3]ₙ₈}Mβ34GNβ4(Fucα6)GNyR₂

wherein p, n2, n4, n5, n8, and m are either independently 0 or 1, with the provisio that when n5 is 0, also n2 and n4 are 0, [ ] indicates determinant either being present or absent depending on the value of n1, n2, n3, n4, ( ) indicates a branch in the structure; and wherein n1, n2, n3, n4 and m are either independently 0 or 1, with the provision that when n3 is 0, also n1 and n2 are 0,
[ ] indicates determinant either being present or absent depending on the value of n1, n2, n3, n4 and m,
{ } and ( ) indicate a branch in the structure.

### Preferred Individual Structures of Fucosylated Low-Mannose Glycans

Small fucosylated low-mannose structures are especially unusual among known N-linked glycans and form a characteristic glycan group useful for separation of cells according to the present invention. These include:
Mβ4GNβ4(Fucα6)GNyR₂
Mα6Mβ4GNβ4(Fucα6)GNyR₂
Mα3Mβ4GNβ4(Fucα6)GNyR₂ and
Mα6{Mα3}Mβ4GNβ4(Fucα6)GNyR₂.
   Mβ4GNβ4(Fucα6)GNyR₂ tetrasaccharide epitope is a preferred common structure alone and together with its mono-mannose derivatives Mα6Mβ4GNβ4(Fucα6)GNyR₂ and/or Mα3Mβ4GNβ4(Fucα6)GNyR₂, because these are commonly present characteristics structures in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

### Special Large Structures

The invention further revealed large fucosylated low-mannose structures are unusual among known N-linked glycans and have special characteristic expression features among the preferred cells according to the invention. The preferred large structure includes
[Mα3]ₙ₂([Mα6]ₙ₄)Mα6{Mα3}Mβ4GNβ4(Fucα6)GNyR₂
   more specifically
Mα6Mα6{Mα3}Mβ4GNβ4(Fucα6)GNyR₂
Mα3Mα6{Mα6}Mβ4GNβ4(Fucα6)GNyR₂ and
Mα3(Mα6)Mα6{Mα3}Mβ4GNβ4(Fucα6)GNyR₂.

The heptasaccharide epitopes are preferred in a specific embodiment as rare and characteristic structures in preferred cell types and as structures with preferred terminal epitopes. The octasaccharide is also preferred as structure comprising a preferred unusual terminal epitope Mα3(Mα6)Mα useful for analysis of cells according to the invention.

### Preferred Non-Reducing End Terminal Mannose-Epitopes

The inventors revealed that mannose-structures can be labeled and/or otherwise specifically recognized on cell surfaces or cell derived fractions/materials of specific cell types. The present invention is directed to the recognition of specific mannose epitopes on cell surfaces by reagents binding to specific mannose structures from cell surfaces.

The preferred reagents for recognition of any structures according to the invention include specific antibodies and other carbohydrate recognizing binding molecules. It is known that antibodies can be produced for the specific structures by various immunization and/or library technologies such as phage display methods representing variable domains of antibodies. Similarly with antibody library technologies, including aptamer technologies and including phage display for peptides, exist for synthesis of library molecules such as polyamide molecules including peptides, especially cyclic peptides, or nucleotide type molecules such as aptamer molecules.

The invention is specifically directed to specific recognition high-mannose and low-mannose structures according to the invention. The invention is specifically directed to recognition of non-reducing end terminal Manα-epitopes, preferably at least disaccharide epitopes, according to the formula:

[Mα2]ₘ₁[Mαx]ₘ₂[Mα6]ₘ₃{{[Mα2]ₘ₉[Mα2]ₘ₈[Mα3]ₘ₇}ₘ₁₀(Mβ4[GN]ₘ₄)ₘ₅}ₘ₆yR₂

wherein m1, m2, m3, m4, m5, m6, m7, m8, m9 and m10 are independently either 0 or 1; with the proviso that when m3 is 0, then m1 is 0 and, when m7 is 0 then either m1-5 are 0 and m8 and m9 are 1 forming Mα2Mα2 disaccharide or both m8 and m9 are 0
y is anomeric linkage structure α and/or β or linkage from derivatized anomeric carbon, and
R₂ is reducing end hydroxyl, chemical reducing end derivative
and x is linkage position 3 or 6 or both 3 and 6 forming branched structure,
{ } indicates a branch in the structure.

The invention is further directed to terminal Mα2-containing glycans containing at least one Mα2-group and preferably Mα2-group on each, branch so that m1 and at least one of m8 or m9 is 1. The invention is further directed to terminal Mα3 and/or Mα6-epitopes without terminal Mα2-groups, when all m1, m8 and m9 are 1.

The invention is further directed in a preferred embodiment to the terminal epitopes linked to a Mβ-residue and for application directed to larger epitopes. The invention is especially directed to Mβ4GN-comprising reducing end terminal epitopes.

The preferred terminal epitopes comprise typically 2-5 monosaccharide residues in a linear chain. According to the invention short epitopes comprising at least 2 monosaccharide residues can be recognized under suitable background conditions and the invention is specifically directed to epitopes comprising 2 to 4 monosaccharide units and more preferably 2-3 monosaccharide units, even more preferred epitopes include linear disaccharide units and/or branched trisaccharide non-reducing residue with natural anomeric linkage structures at reducing end. The shorter epitopes may be preferred for specific applications due to practical reasons including effective production of control molecules for potential binding reagents aimed for recognition of the structures.

The shorter epitopes such as Mα2M-may is often more abundant on target cell surface as it is present on multiple arms of several common structures according to the invention.

### Preferred Disaccharide Epitopes Includes

Manα2Man, Manα3Man, Manα6Man, and more preferred anomeric forms Manα2Manα, Manα3Manβ, Manα6Manα, Manα3Manα and Manα6Manα.

Preferred branched trisaccharides includes Manα3(Manα6)Man, Manα3(Manα6)Manβ, and Manα3(Manα6)Manα.

The invention is specifically directed to the specific recognition of non-reducing terminal Manα2-structures especially in context of high-mannose structures.

The invention is specifically directed to following linear terminal mannose epitopes:

a) preferred terminal Manα2-epitopes including following oligosaccharide sequences:
Manα2Man,
Manα2Manα,
Manα2Manα2Man, Manα2Manα3Man, Manα2Manα6Man,
Manα2Manα2Man, Manα2Manα3Manβ, Manα2Manα6Manα, Manα2Manα2Manα3Man, Manα2Manα3Manα6Man, Manα2Manα6Manα6Man
Manα2Manα2Manα3Manβ, Manα2Manα3Manα6Manβ, Manα2Manα6Manα6Manβ;

The invention is further directed to recognition of and methods directed to non-reducing end terminal Manα3- and/or Manα6-comprising target structures, which are characteristic features of specifically important low-mannose glycans according to the invention. The preferred structural groups includes linear epitopes according to b) and branched epitopes according to the c3) especially depending on the status of the target material.

b) preferred terminal Manα3- and/or Manα6-epitopes including following oligosaccharide sequences:
Manα3Man, Manα6Man, Manα3Manβ, Manα6Manβ, Manα3Manα, Manα6Manα,
Manβ3Manα6Man, Manβ6Manα6Man, Manα3Manα6Manβ, Manα6Manα6Manβand to following
   c) branched terminal mannose epitopes, are preferred as characteristic structures of especially high mannose structures (c1 and c2) and low-mannose structures (c3), The preferred branched epitopes include:
   c1) branched terminal Manα2-epitopes
Manα2Manβ3(Manα2Manα6)Man, Manα2Manα3 (Manβ2Manα6)Manα,
Manα2Manβ3(Manα2Manα6)Manα6Man, Manα2Manα3(Manα2Manα6)Manα6Manβ,
Manβ2Manα3 (Manα2Manα6)Manα6(Manβ2Manα3)Man,
Manα2Manα3 (Manα2Manα6)Manβ6(Manβ2Manα2Manα3)Man,
Manα2Manα3 (Manα2Manα6)Manα6(Manα2Manα3)Manβ
Manα2Manα3(Manα2Manα6)Manα6(ManαManα2Manα3)Manβ
   c2) branched terminal Manα2- and Manα3 or Manα6-epitopes
   according to formula when m1 and/or m8 and/m9 is 1 and the molecule comprise at least one nonreducing end terminal Manα3 or Manα6-epitope
   c3) branched terminal Manβ3 or Manα6-epitopes
Manα3(Manα6)Man, Manα3(Manα6)Manβ, Manα3(Manβ6)Manβ,
Manα3(Manα6)Manα6Man, Manα3(Manα6)Manα6Manβ,
Manα3 (Manβ6)Manα6(Manβ3)Man, Manα3 (Manα6)Manα6(Manβ3)Manβ

The present invention is further directed to increase of selectivity and sensitivity in recognition of

Target glycans by combining recognition methods for terminal Manα2 and Manα3 and/or Manα6-comprising structures. Such methods would be especially useful in context of cell material according to the invention comprising both high-mannose and low-mannose glycans.

### Complex Type N-Glycans

According to the present invention, complex-type structures are preferentially identified by mass spectrometry, preferentially based on characteristic monosaccharide compositions, wherein HexNAc≧4 and Hex≧3. In a more preferred embodiment of the present invention, 4≦HexNAc≦20 and 3≦Hex≦21, and in an even more preferred embodiment of the present invention, 4≦HexNAc≦10 and 3≦Hex≦11. The complex-type structures are further preferentially identified by sensitivity to endoglycosidase digestion, preferentially N-glycosidase F detachment from glycoproteins. The complex-type structures are further preferentially identified in NMR spectroscopy based on characteristic resonances of the Manα3(Manα6)Manβ4GlcNAcβ4GlcNAc N-glycan core structure and GlcNAc residues attached to the Manα3 and/or Manα6 residues.

### Beside Mannose-Type Glycans the Preferred N-Linked Glycomes Include GlcNAcβ2-Type Glycans INCLUDING COMPLEX TYPE GLYCANS Comprising Only GlcNAcβ2-Branches and Hydrid Type Glycan Comprising Both Mannose-Type Branch and GlcNAcβ2-Branch.

GlcNAcβ2-Type glycans

The invention revealed GlcNAcβ2Man structures in the glycomes according to the invention. Preferably GlcNAcβ2Man-structures comprise one or several of GlcNAcβ2Manα-structures, more preferably GlcNAcβ2Manα3 or GlcNAcβ2Manα6-structure.

The Complex type glycans of the invention comprise preferably two GlcNAcβ2Manαstructures, which are preferably GlcNAcβ2Manα3 and GlcNAcβ2Manα6-. The Hybrid type glycans comprise preferably GlcNAcβ2Manα3-structure.

The present invention is directed to at least one of natural oligosaccharide sequence structures and structures truncated from the reducing end of the N-glycan according to the Formula GNβ2

[R₁GNβ2]ₙ₁[Mα3]ₙ₂{[R₃]ₙ₃[GNβ2]ₙ₄Mα6}ₙ₅Mβ4GNXyR₂,

with optionally one or two or three additional branches according to formula [RₓGNβz]ₙₓ linked to Mα6-, Mα3-, or Mβ4 and Rₓ may be different in each branch
wherein n1, n2, n3, n4, n5 and nx, are either 0 or 1, independently,
with the proviso that when n2 is 0 then n1 is 0 and when n3 is 1 or/and n4 is 1 then n5 is also 1, and at least n1 or n4 is 1, or n3 is 1,
when n4 is 0 and n3 is 1 then R₃ is a mannose type substituent or nothing and
wherein X is glycosidically linked disaccharide epitope β4(Fucα6)ₙGN, wherein n is 0 or 1, or X is nothing and
y is anomeric linkage structure cc and/or β or linkage from derivatized anomeric carbon, and R₁, Rₓ and R₃ indicate independently one, two or three, natural substituents linked to the core structure,
R₂ is reducing end hydroxyl, chemical reducing end derivative or natural asparagine N-glycoside derivative such as asparagine N-glycosides including asparagines N-glycoside aminoacids and/or peptides derived from protein.
[ ] indicate groups either present or absent in a linear sequence. { }indicates branching which may be also present or absent.

### Elongation of GlcNAcβ2-Type Structures, Complex/Hydrid Type Structures

The substituents R₁, Rₓ and R₃ may form elongated structures. In the elongated structures R₁, and Rₓ represent substituents of GlcNAc (GN) and R₃ is either substituent of GlcNAc or when n4 is 0 and n3 is 1 then R₃ is a mannose type substituent linked to mannose a 6-branch forming a Hybrid type structure. The substituents of GN are monosaccharide Gal, GalNAc, or Fuc or and acidic residue such as sialic acid or sulfate or phosphate ester.

GlcNAc or GN may be elongated to N-acetyllactosaminyl also marked as GalβGN or di-N-acetyllactosdiaminyl GalNAcβGlcNAc preferably GalNAcβ4GlcNAc. LNβ2M can be further elongated and/or branched with one or several other monosaccharide residues such as by galactose, fucose, SA or LN-unit(s) which may be further substituted by SAα-structures, and/or Mα6 residue and/or Mα3 residues can be further substituted one or two β6-, and/or β4-linked additional branches according to the formula,

and/or either of Mα6 residue or Mα3 residue may be absent
and/or Mα6-residue can be additionally substitutes other Manα units to form a hybrid type structures
and/or Manβ4 can be further substituted by GNβ4,
and/or SA may include natural substituents of sialic acid and/or it may be substituted by other SA-residues preferably by α8- or α9-linkages.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof.

### Hybrid Type Structures

According to the present invention, hybrid-type or monoantennary structures are preferentially identified by mass spectrometry, preferentially based on characteristic monosaccharide compositions, wherein HexNAc=3 and Hex≧2. In a more preferred embodiment of the present invention 2≦Hex≦11, and in an even more preferred embodiment of the present invention 2≦Hex≦9. The hybrid-type structures are further preferentially identified by sensitivity to exoglycosidases digestion, preferentially α-mannosidase digestion when the structures contain non-reducing terminal α-mannose residues and Hex≧3, or even more preferably when Hex≧4, and to endoglycosidase digestion, preferentially N-glycosidase F detachment from glycoproteins. The hybrid-type structures are further preferentially identified in NMR spectroscopy based on characteristic resonances of the Manα3(Manα6)Manβ4GlcNAcβ4GlcNAc N-glycan core structure, a GlcNAcβ residue attached to a Manα residue in the N-glycan core, and the presence of characteristic resonances of non-reducing terminal α-mannose residue or residues.

The monoantennary structures are further preferentially identified by insensitivity to α-mannosidase digestion and by sensitivity to endoglycosidase digestion, preferentially N-glycosidase F detachment from glycoproteins. The monoantennary structures are further preferentially identified in NMR spectroscopy based on characteristic resonances of the Manα3Manβ4GlcNAcβ4GlcNAc N-glycan core structure, a GlcNAcβresidue attached to a Manαresidue in the N-glycan core, and the absence of characteristic resonances of further non-reducing terminal α-mannose residues apart from those arising from a terminal α-mannose residue present in a ManαManβsequence of the N-glycan core.

The present invention is directed to at least one of natural oligosaccharide sequence structures and structures truncated from the reducing end of the N-glycan according to the Formula HY1

R₁GNβ2Mα3{[R₃]ₙ₃Mα6}Mβ4GNXyR₂,

wherein n3, is either 0 or 1, independently,
and
wherein X is glycosidically linked disaccharide epitope β4(Fucα6)ₙGN, wherein n is 0 or 1, or X is nothing and
y is anomeric linkage structure α and/or β or linkage from derivatized anomeric carbon, and
R₁ indicate nothing or substituent or substituents linked to GlcNAc, R₃ indicates nothing or Mannose-substituent(s) linked to mannose residue, so that each of R₁, and R₃ may correspond to one, two or three, more preferably one or two, and most preferably at least one natural substituents linked to the core structure,
R₂ is reducing end hydroxyl, chemical reducing end derivative or natural asparagine N-glycoside derivative such as asparagine N-glycosides including asparagines N-glycoside aminoacids and/or peptides derived from protein.
[ ] indicate groups either present or absent in a linear sequence. { }indicates branching which may be also present or absent.

### Preferred Hybrid Type Structures

The preferred hydrid type structures include one or two additional mannose residues on the preferred core structure.

R₁GNβ2Mα3{[Mα3]ₘ₁([Mα6])ₘ₂Mα6}Mβ4GNXyR₂, Formula HY2

wherein n3, is either 0 or 1, and m1 and m2 are either 0 or 1, independently,
{ } and ( ) indicates branching which may be also present or absent,
other variables are as described in Formula HY1.

Furthermore the invention is directed to structures comprising additional lactosamine type structures on GNβ2-branch. The preferred lactosamine type elongation structures includes N-acetyllactosamines and derivatives, galactose, GalNAc, GlcNAc, sialic acid and fucose.

Preferred structures according to the formula HY2 include:

Structures containing non-reducing end terminal GlcNAc

As a specific preferred group of glycans
GNβ2Mα3{Mα3Mα6}Mβ4GNXyR₂,
GNβ2Mα3{M6Mα6}Mβ4GNXyR₂,
GNβ2Mα3{Mα3(Mα6)Mα6}Mβ4GNXyR₂,
   and/or elongated variants thereof
R₁GNβ2Mα3{M3Mα6}Mβ4GNXyR₂,
R₁GNβ2Mα3{M6Mα6}Mβ4GNXyR₂,
R₁GNβ2Mα3{Mα3(Mα6)Mα6}M134GNXyR₂,

### Formula HY3

[R₁Gal[NAc]ₒ₂βz]ₒ₁GNβ2Mα3{[Mα3]ₘ₁[(Mα6)]ₘ₂Mα6}ₙ₅Mβ4GNXyR₂,

wherein n1, n2, n3, n5, m1, m2, 01 and o2 are either 0 or 1, independently,
z is linkage position to GN being 3 or 4, ? in a preferred embodiment 4,
R₁ indicates on or two a N-acetyllactosamine type elongation groups or nothing,
{ } and ( ) indicates branching which may be also present or absent,
other variables are as described in Formula HY1.

Preferred structures according to the formula HY3 include especially

structures containing non-reducing end terminal Galβ, preferably Galβ3/4 forming a terminal N-acetyllactosamine structure. These are preferred as a special group of Hybrid type structures, preferred as a group of specific value in characterization of balance of Complex N-glycan glycome and High mannose glycome:
GalβzGNβ2Mα3{Mα3Mα6}Mβ4GNXyR₂, GalβzGNβ2Mα3{Mα6Mα6}Mβ4GNXyR₂,
GalβzGNβ2Mα3{Mα3 (Mα6)Mα6}Mβ4GNXyR₂,
   and/or elongated variants thereof preferred for carrying additional characteristic terminal structures useful for characterization of glycan materials
R₁GalβzGNβ2Mα3{Mα3Mα6}Mβ4GNXyR₂,
R₁GalβzGNβ2Mα3{Mα6Mα6}Mβ4GNXyR₂,
   R₁GalβzGNβ2Mα3{Mα3 (Mα6)Mα6}Mβ4GNXyR₂. Preferred elongated materials include structures wherein R₁ is a sialic acid, more preferably NeuNAc or NeuGc.

### Complex N-Glycan Structures

The present invention is directed to at least one of natural oligosaccharide sequence structures and structures truncated from the reducing end of the N-glycan according to the Formula CO1

[R₁GNβ2]ₙ₁[Mα3]ₙ₂{[R₃GNβ2]ₙ₄Mα6}ₙ₅Mβ4GNXyR₂

with optionally one or two or three additional branches according to formula [RₓGNβz]ₙₓ linked to Mα6-, Mα3-, or Mβ4 and Rₓ may be different in each branch
wherein n1, n2, n4, n5 and nx, are either 0 or 1, independently, with the proviso that when n2 is 0 then n1 is 0 and when n4 is 1 then n5 is also 1, and at least
n1 is 1 or n4 is 1, and at least either of n1 and n4 is 1 and
wherein X is glycosidically linked disaccharide epitope β4(Fucα6)ₙGN, wherein n is 0 or 1,
or X is nothing and
y is anomeric linkage structure cc and/or β or linkage from derivatized anomeric carbon, and
R₁, Rₓ, and R₃ indicate independently one, two or three, natural substituents linked to the core structure,

R₂ is reducing end hydroxyl, chemical reducing end derivative or natural asparagine N-glycoside derivative such as asparagine N-glycosides including asparagines N-glycoside aminoacids and/or peptides derived from protein.

[ ] indicate groups either present or absent in a linear sequence. { }indicates branching which may be also present or absent.

### Preferred Complex Type Structures

### Incomplete Monoantennary N-Glycans

The present invention revealed incomplete Complex monoantennary N-glycans, which are unusual and useful for characterization of glycomes according to the invention. The most of the in complete monoantennary structures indicate potential degradation of biantennary N-glycan structures and are thus preferred as indicators of cellular status. The incomplete Complex type monoantennary glycans comprise only one GNβ2-structure.

The invention is specifically directed to structures are according to the Formula CO1 above when only n1 is 1 or n4 is one and mixtures of such structures.

The preferred mixtures comprise at least one monoantennary complex type glycans

A) with single branches from a likely degradative biosynthetic process:
R₁GNβ2Mα3β4GNXyR₂
R₃GNβ2Mα6Mβ4GNXyR₂ and
   B) with two branches comprising mannose branches
   B1) R₁GNβ2Mα3{Mα6}ₙ₅Mβ4GNXyR₂
   B2) Mα3{R₃GNβ2Mα6}ₙ₅M4GNXyR₂

The structure B2 is preferred with A structures as product of degradative biosynthesis, it is especially preferred in context of lower degradation of Manα3-structures. The structure B1 is useful for indication of either degradative biosynthesis or delay of biosynthetic process

### Biantennary and Multiantennary Structures

The inventor revealed a major group of biantennary and multiantennary N-glycans from cells according to the invention, the preferred biantennary and multiantennary structures comprise two GNβ2 structures.

These are preferred as an additional characteristics group of glycomes according to the invention and are represented according to the Formula CO2:

R₁GNβ2Mα3{R₃GNβ2Mα6}Mβ4GNXyR₂

with optionally one or two or three additional branches according to formula [RₓGNβz]ₙₓ linked to Mα6-, Mα3-, or Mβ4 and Rₓ may be different in each branch
wherein nx is either 0 or 1,
and other variables are according to the Formula CO1.

### Preferred Biantennary Structure

A biantennary structure comprising two terminal GNP-epitopes is preferred as a potential indicator of degradative biosynthesis and/or delay of biosynthetic process. The more preferred structures are according to the Formula CO2 when R₁ and R₃ are nothing.

### Elongated Structures

The invention revealed specific elongated complex type glycans comprising Gal and/or GalNAc-structures and elongated variants thereof. Such structures are especially preferred as informative structures because the terminal epitopes include multiple informative modifications of lactosamine type, which characterize cell types according to the invention. The present invention is directed to at least one of natural oligosaccharide sequence structure or group of structures and corresponding structure(s) truncated from the reducing end of the N-glycan according to the Formula CO3

[R₁Gal[NAc]ₒ₂βz2]ₒ₁GNβ2Mα3{[R₁Gal[NAc]ₒ₄βz2]ₒ₃GNβ2Mα6}Mβ4GNXyR₂,

with optionally one or two or three additional branches according to formula [RₓGNβz1]ₙₓ linked to Mα6-, Mα3-, or Mβ4 and Rₓ may be different in each branch
wherein nx, o1, o2, o3, and o4 are either 0 or 1, independently,
with the provisio that at least o1 or o3 is 1, in a preferred embodiment both are 1
z2 is linkage position to GN being 3 or 4, ? in a preferred embodiment 4,
z1 is linkage position of the additional branches.
R₁, Rₓ and R₃ indicate on or two a N-acetyllactosamine type elongation groups or nothing,
{ } and ( ) indicates branching which may be also present or absent,
other variables are as described in Formula CO1.

### Galactosylated Structures

The inventors characterized especially directed to digalactosylated structure
GalβzGNβ2Mα3{GalβzGNβ2Mα6}Mβ4GNXyR₂,
   and monogalactosylated structures:
GalβzGNβ2Mα3{GNβ2Mα6}Mβ4GNXyR₂,
GNβ2Mα3{GalβzGNβ2Mα6}Mβ4GNXyR₂,
   and/or elongated variants thereof preferred for carrying additional characteristic terminal structures useful for characterization of glycan materials
R₁GalβzGNβ2Mα3{R₃GalβzGNβ2Mα6}Mβ4GNXyR₂
R₁GalβzGNβ2Mα3{GNβ2Mα6}Mβ4GNXyR₂, and
GNβ2Mα3{R₃GalβzGNβ2Mα6}Mβ4GNXyR₂.

Preferred elongated materials include structures wherein R₁ is a sialic acid, more preferably NeuNAc or NeuGc.

### LacdiNAc-Structure Comprising N-Glycans

The present invention revealed for the first time LacdiNAc, GalNacbGlcNAc structures from the cell according to the invention. Preferred N-glycan lacdiNAc structures are included in structures according to the Formula CO1, when at least one the variable o2 and o4 is 1.

### The Major Acidic Glycan Types

The acidic glycomes mean glycomes comprising at least one acidic monosaccharide residue such as sialic acids (especially NeuNAc and NeuGc) forming sialylated glycome, HexA (especially GlcA, glucuronic acid) and/or acid modification groups such as phosphate and/or sulphate esters.

According to the present invention, presence of phosphate and/or sulphate ester (SP) groups in acidic glycan structures is preferentially indicated by characteristic monosaccharide compositions containing one or more SP groups. The preferred compositions containing SP groups include those formed by adding one or more SP groups into non-SP group containing glycan compositions, while the most preferential compositions containing SP groups according to the present invention are selected from the compositions described in the acidic N-glycan fraction glycan group tables. The presence of phosphate and/or sulphate ester groups in acidic glycan structures is preferentially further indicated by the characteristic fragments observed in fragmentation mass spectrometry corresponding to loss of one or more SP groups, the insensitivity of the glycans carrying SP groups to sialidase digestion. The presence of phosphate and/or sulphate ester groups in acidic glycan structures is preferentially also indicated in positive ion mode mass spectrometry by the tendency of such glycans to form salts such as sodium salts as described in the Examples of the present invention. Sulphate and phosphate ester groups are further preferentially identified based on their sensitivity to specific sulphatase and phosphatase enzyme treatments, respectively, and/or specific complexes they form with cationic probes in analytical techniques such as mass spectrometry.

### Complex N-Glycan Glycomes, Sialylated

The present invention is directed to at least one of natural oligosaccharide sequence structures and structures truncated from the reducing end of the N-glycan according to the Formula

[{SAα3/6}ₛ₁LNβ2]ᵣ₁Mα3{({SAα3/6}ₛ₂LNβ2)ᵣ₂Mα6}ᵣ₈{M[β4GN[β4{Fucα6}ᵣ₃GN]_{r4]}ᵣ₅}ᵣ₆ (I)

with optionally one or two or three additional branches according to formula

{SAα3/6}ₛ₃LNβ, (IIb)

wherein r1, r2, r3, r4, r5, r6, r7 and r8 are either 0 or 1, independently,
wherein s1, s2 and s3 are either 0 or 1, independently,
with the proviso that at least r1 is 1 or r2 is 1, and at least one of s1, s2 or s3 is 1. LN is N-acetyllactosaminyl also marked as GalβGN or di-N-acetyllactosdiaminyl GalNAcβGlcNAc preferably GalNAcβ4GlcNAc, GN is GlcNAc, M is mannosyl-, with the proviso LNβ2M or GNβ2M can be further elongated and/or branched with one or several other monosaccharide residues such as by galactose, fucose, SA or LN-unit(s) which may be further substituted by SAα-structures,
and/or one LNβ can be truncated to GNβ
and/or Mα6 residue and/or Mα3 residues can be further substituted one or two β6-, and/or β4-linked additional branches according to the formula,
and/or either of Mα6 residue or Mα3 residue may be absent and/or Mα6-residue can be additionally substitutes other Manα units to form a hybrid type structures
and/or Manβ4 can be further substituted by GNβ4,
and/or SA may include natural substituents of sialic acid and/or it may be substituted by other SA-residues preferably by α8- or α9-linkages.
( ), { }, [ ] and [ ] indicate groups either present or absent in a linear sequence. { }indicates branching which may be also present or absent.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof. In a preferred embodiment the invention is directed to glycans wherein r6 is 1 and r5 is 0, corresponding to N-glycans lacking the reducing end GlcNAc structure.

The LN unit with its various substituents can in a preferred general embodiment represented by the formula:

[Gal(NAc)ₙ₁α3]ₙ₂{Fucα2}ₙ₃Gal(NAc)ₙ₄β3/4{Fucα4/3}ₙ₅GlcNAcβ

wherein n1, n2, n3, n4, and n5 are independently either 1 or 0,
with the provisio that
the substituents defined by n2 and n3 are alternative to presence of SA at the non-reducing end terminal
the reducing end GlcNAc-unit can be further β3- and/or β6-linked to another similar LN-structure forming a poly-N-acetyllactosamine structure
with the provision that for this LN-unit n2, n3 and n4 are 0,
the Gal(NAc)ₚ and GlcNAcβunits can be ester linked a sulphate ester group,
( ), and [ ] indicate groups either present or absent in a linear sequence; { }indicates branching which may be also present or absent.

LN unit is preferably Galβ4GN and/or Galβ3GN.

### Uses of Glycan Structure Grouping and Analysis

In the present invention, glycan signals with preferential monosaccharide compositions can be grouped into structure groups based on classification rules described in the present invention.

The present invention includes parallel and overlapping classification systems that are used for the classification of the glycan structure groups.

Glycan signals isolated from the N-glycan fractions from the tissue material types studied in the present invention are grouped into glycan structure groups based on their preferential monosaccharide compositions according to the invention, in glycan group Tables of Examples for neutral glycan fractions and for acidic glycan fractions. Taken together, the analyses revealed that all the structure groups according to the invention are present in the studied tissue material types. In another aspect of the present invention, the glycan structure grouping is used to compare different tissue materials and characterize their specific glycosylation features. According to the present invention the discovered and analyzed differences between the glycan signals within the glycan signal groups between different tissue material samples are used for comparison and characterization.

The quantitative glycan profiling combined with glycan structural classification is used according to the present invention to characterize and identify glycosylation features occurring in tissue materials, glycosylation features specific for certain tissue materials as well as differences between different tissue materials. According to the present invention, the classification is used to characterize and compare glycosylation features of different tissues, of normal and diseased tissues, preferentially cancerous tissues, and solid tissues such as lung tissue and fluid tissues such as blood and/or serum. In another aspect of the present invention, the glycan structure grouping is used to compare different tissue materials and characterize their specific glycosylation features. According to the present invention differences between relative proportions of glycan signal structure groups are used to compare different tissue material samples.

In a further aspect of the present invention, analysis of the glycan structure groups, preferentially including terminal HexNAc and/or low-mannose and optionally other groups separately or in combination, is used to differentiate between different tissue materials or different stages of tissue materials, preferentially to identify human disease and more preferentially human cancer. In a further preferred form the present method is used to differentiate between benign and malignant tumors. According to the present invention analysis of human serum glycan groups or combinations thereof according to the present invention can be used to identify the presence of other tissue materials in blood or serum samples, more preferably to identify disease and preferably malignant cancer.

### Integrated Glycome Analysis Technology

The invention is directed to analysis of present cell materials based on single or several glycans (glycome profile) of cell materials according to the invention. The analysis of multiple glycans is preferably performed by physical analysis methods such as mass spectrometry and/or NMR.

The invention is specifically directed to integrated analysis process for glycomes, such as total glycomes and cell surface glycomes. The integrated process represent various novel aspects in each part of the process. The methods are especially directed to analysis of low amounts of cells. The integrated analysis process includes

A) preferred preparation of substrate cell materials for analysis, including one or several of the methods: use of a chemical buffer solution, use of detergents, chemical reagents and/or enzymes.
B) release of glycome(s), including various subglycome type based on glycan core, charge and other structural features, use of controlled reagents in the process
C) purification of glycomes and various subglycomes from complex mixtures
D) preferred glycome analysis, including profiling methods such as mass spectrometry and/or NMR spectroscopy
E) data processing and analysis, especially comparative methods between different sample types and quantitative analysis of the glycome data.

### A. Preparation of Cell Materials

Cell substrate material and its preparation for total and cell surface glycome analysis. The integrated glycome analysis includes preferably a cell preparation step to increase the availability of cell surface glycans. The cell preparation step preferably degrades either total cell materials or cell surface to yield a glycome for more effective glycan release. The degradation step preferably includes methods of physical degradation and/or chemical degradation. In a preferred embodiment at least one physical and one chemical degradation methods are combined, more preferably at least one physical method is combined with at least two chemical methods, even more preferably with at least three chemical methods.

The physical degration include degration by energy including thermal and/or mechanical energy directed to the cells to degrade cell structures such as heating, freezing, sonication, and pressure. The chemical degradation include use of chemicals and specific concentrations of chemicals for disruption of cells preferably detergents including ionic and neutral detergents, chaotropic salts, denaturing chemicals such as urea, and non-physiological salt concentrations for disruption of the cells.

The glycome analysis according to the invention is divided to two methods including Total cell glycomes, and Cell surface glycomes. The production of Total cell glycomes involves degradation of cells by physical and/or chemical degradation methods, preferably at least by chemical methods, more preferably by physical and chemical methods. The Cell surface glycomes is preferably released from cell surface preserving cell membranes intact or as intact as possible, such methods involve preferably at least one chemical method, preferably enzymatic method. The cell surface glycomes may be alternatively released from isolated cell membranes, this method involves typically chemical and/or physical methods similarity as production of total cell glycomes, preferably at least use of detergents.

a. Total Cell Glycomes

The present invention revealed special methods for effective purification of released glycans from total cell derived materials so that free oligosaccharides can be obtained. In a preferred embodiment a total glycome is produced from a cell sample, which is degraded to form more available for release of glycans. A preferred degraded form of cells is detergent lysed cells optionally involving physical disruption of cell materials.

Preferred detergents and reaction conditions include,

a1) ionic detergents, preferably SDS type anionic detergent comprising an anionic group such as sulfate and an alkyl chain of 8-16 carbon atoms, more preferably the anionic detergent comprise 10-14 carbon atoms and it is most preferably sodium dodecyl sulfate (SDS), and/or
a2) non-ionic detergents such as alkylglycosides comprising a hexose and 4-12 carbon alkyl chain more preferably the alkyl chain comprises a hexoses being galactose, glucose, and/or mannose, more preferably glucose and/or mannose and the alkyl comprises 6-10 carbon atoms, preferably the non-ionic detergent is octylglucoside

It is realized that various detergent combinations may be produced and optimized. The combined use of an ionic, preferably anionic, and non-ionic detergents according to the invention is especially preferred.

### Preferred Cell Preparation Methods for Production of Total Cell Glycome

The preferred methods of cell degration for Total cell glycomes include physical degration including at least heat treatment heat and chemical degration by a detergent method or by a non-detergent method preferably enzymatic degradation, preferably heat treatment. Preferably two physical degradation methods are included.

### A Preferred Non-Detergent Method Includes

A non-detergent method is preferred for avoiding detergent in later purification. The preferred non-detergent method involves physical degradation of cells preferably pressure and or by heat and a chemical degradation by protease. A preferred non-detergent method includes:

i) cell degradation by physical methods, for example by pressure methods such as by French press.

The treatment is preferably performed quickly in cold temperatures, preferably at 0-2 degrees of Celsius, and more preferably at about 0 or 1 degree of celcius and/or in the presence of glycosidase inhibitors.

ii) The degraded cells are further treated with chemical degradation, preferably by effective general protease, more preferably trypsin is used for the treatment. Preferred trypsin preparation according to the invention does not cause glycan contamination to the sample/does not contain glycans releasable under the reaction conditions.
iii) optionally the physical degradation and chemical degradation are repeated.
iv) At the end of protease treatment the sample is boiled for further denaturing the sample and the protease. The boiling is performed at temperature denaturing/degrading further the sample and the protease activity (conditions thus depend on the protease used) preferably about 100 degrees Celsius for time enough for denaturing protease activity preferably about 10-20 minutes for trypsin, more preferably about 15 minutes.

### Preferred Detergent Method for Production of Total Glycomes

The invention is in another preferred embodiment directed to detergent based method for lysing cells. The invention includes effective methods for removal of detergents in later purification steps. The detergent methods are especially preferred for denaturing proteins, which may bind or degrade glycans, and for degrading cell membranes to increase the accessibility of intracellular glycans.

For the detergent method the cell sample is preferably a cell pellet produced at cold temperature by centrifuging cells but avoiding disruption of the cells, optionally stored frozen and melted on ice. Optionally glycosidase inhibitors are used during the process.

The method includes following steps:

i) production of cell pellet preferably by centrifugation,
ii) lysis by detergent on ice, the detergent is preferably an anionic detergent according to the invention, more preferably SDS. The concentration of the detergent is preferably between about 0.1% and 5%, more preferably between 0.5%-3%, even more preferably between 0.5-1.5% and most preferably about 1% and the detergent is SDS (or between 0.9-1.1%). the solution is preferably produced in ultrapure water,
iii) mixing by effective degradation of cells, preferably mixing by a Vortex-mixer as physical degradation step,
iv) boiling on water bath, preferably for 3-10 min, most preferably about 5 min (4-6 min) as second physical degradation step, it is realized that even longer boiling may be performed for example up to 30 min or 15 min, but it is not optimal because of evaporation sample
v) adding one volume of non-ionic detergent, preferably alkyl-glycoside detergent according to the invention, most preferably n-octyl-β-D-glucoside, the preferred amount of the detergent is about 5-15% as water solution, preferably about 10% of octyl-glucoside. The non-ionic detergent is especially preferred in case an enzyme sensitive to SDS, such as a N-glycosidase, is to be used in the next reaction step.
and
vi) incubation at room temperature for about 5 min to about 1-4 hours, more preferably less than half an hour, and most preferably about 15 min.

### Preferred Amount of Detergents in the Detergent Method

Preferably the anionic detergent and cationic detergent solutions are used in equal volumes. Preferably the solutions are about 1% SDS and about 10% octyl-glucoside. The preferred amounts of the solutions are preferably from 0.1 μl to about 2 μl, more preferably 0.15 μl to about 1.5 μl per and most preferably from 0.16 μl to 1 pt per 100 000 cells of each solution.

Lower amounts of the detergents are preferred if possible for reduction of the amount of detergent in later purification, highest amounts in relation to the cell amounts are used for practical reasons with lowest volumes. It is further realized that corresponding weight amounts of the detergents may be used in volumes of about 10% to about 1000%, or from about 20% to about 500% and even more effectively in volumes from 30% to about 300% and most preferably in volumes of range from 50% to about 150% of that described. It is realized that critical micellar concentration based effects may reduce the effect of detergents at lowest concentrations.

In a preferred embodiment a practical methods using tip columns as described in the invention uses about 1-3 μl of each detergent solution, more preferably 1.5-2.5 μl, and most preferably about 2 μl of the preferred detergent solutions or corresponding detergent amounts are used for about 200 000 or less cells (preferably between 2000 and about 250 000 cells, more preferably from 50 000 to about 250 000 cells and most preferably from 100 000 to about 200 000 cells). Another practical method uses about 2-10 μl of each detergent solution, more preferably 4-8 μl, and most preferably about 5 μl (preferably between 4 and 6 μl and more preferably between 4.5 and 5.5 μl) of detergent solutions or corresponding amount of the detergents for lysis of cell of a cell amount from about 200 000-3 million cells (preferred more exact ranges include 200 000-3.5 million, 200 000 to 3 million and 200 000 to 2.5 million cells), preferably a fixed amount (specific amount of microliters preferably with the accuracy of at least 0.1 microliter) in a preferred range such as of 5.0 μl is used for the wider range of cells 200 000-3 million. It was invented that is possible to handle similarity wider range of materials. It is further realized that the method can be optimized so that exact amount of detergent, preferably within the ranges described, is used for exact amount of cells, such method is preferably an atomized when there is possible variation in amounts of sample cells.

b. Cell Surface Glycomes

In another preferred embodiment the invention is directed to release of glycans from intact cells and analysis of released cell surface glycomes. The present invention is directed to specific buffer and enzymatic cell pre-modification conditions that would allow the efficient use of enzymes for release and optionally modification and release of glycans.

### B. The Glycan Release Methods

The invention is directed to various enzymatic and chemical methods to release glycomes. The release step is not needed for soluble glycomes according to the invention. The invention further revealed soluble glycome components which can be isolated from the cells using methods according to the invention.

C. Purification of glycans from cell derived materials The purification of glycome materials form cell derived molecules is a difficult task. It is especially difficult to purify glycomes to obtain picomol or low nanomol samples for glycome profiling by mass spectrometry or NMR-spectrometry. The invention is especially directed to production of material allowing quantitative analysis over a wide mass range. The invention is specifically directed to the purification of non-derivatized or reducing end derivatized glycomes according to the invention and glycomes containing specific structural characteristics according to the invention. The structural characteristics were evaluated by the preferred methods according to the invention to produce reproducible and quantitative purified glycomes.

### Glycan Purification Process Steps

The glycan purification method according to the present invention consists of at least one of purification options, preferably in specific combinations described below, including one or several of following the following purification process steps in varying order:

### 6) Precipitation-extraction;

### 7) Ion-exchange;

8) Hydrophobic interaction;
9) Hydrophilic interaction; and
10) Affinity to carbon materials especially graphitized carbon.

### Prepurification and Purification Process Steps

In general the purification steps may be divided to two major categories:

Prepurification steps to remove major contaminations and purification steps usually directed to specific binding and optionally fractionation og glycomes

### a) Prepurification to Remove Non-Carbohydrate Impurities

The need for prepurification depends on the type and amounts of the samples and the amounts of impurities present. Certain samples it is possible to omit all or part of the prepurification steps. The prepurification steps are aimed for removal of major non-carbohydrate impurities by separating the impurity and the glycome fraction(s) to be purified to different phases by precipitation/extraction or binding to chromatography matrix and the separating the impurities from the glycome fraction(s).

The prepurification steps include one, two or three of following major steps:

Precipitation-extraction, Ion-exchange, Hydrophobic interaction.

The precipitation and/or extraction is based on the high hydrophilic nature of glycome compositions and components, which is useful for separation from different cellular components and chemicals. The prepurification ion exchange chromatography is directed to removal of classes molecules with different charge than the preferred glycome or glycome fraction to be studied. This includes removal of salt ions and aminoacids, and peptides etc. The glycome may comprise only negative charges or in more rare case also only positive charges and the same charge is selected for the chromatography matrix for removal of the impurities for the same charge without binding the glycome at prepurification.

In a preferred embodiment the invention is directed to removal of cationic impurities from glycomes glycomes containing neutral and/or negatively charged glycans. The invention is further directed to use both anion and cation exchange for removal of charged impurities from non-charged glycomes. The preferred ion exchange and cation exchange materials includes polystyrene resins such as Dowex resins.

The hydrophilic chromatography is preferably aimed for removal of hydrophobic materials such as lipids detergents and hydrophobic protein materials. The preferred hydrophobic chromatography materials includes.

It is realized that different combinations of the prepurification are useful depending on the cell preparation and sample type. Preferred combinations of the prepurification steps include: Precipitation-extraction and Ion-exchange; Precipitation-extraction and Hydrophobic interaction; and Ion-exchange and Hydrophobic interaction. The two prepurification steps are preferably performed in the given order.

### Purification Steps Including Binding and Optionally Fractionation of Glycomes

The purification steps utilize two major concepts for binding to carbohydrates and combinations thereof: a) Hydrophilic interactions and b) Ion exchange

### a) Hydrophilic Interactions

The present invention is specifically directed to use of matrices with repeating polar groups with affinity for carbohydrates for purification of glycome materials according to the invention in processes according to the invention. The hydrophilic interaction material may include additional ion exchange properties.

The preferred hydrophilic interaction materials includes carbohydrate materials such as carbohydrate polymers in presence of non-polar organic solvents. A especially preferred hydrophilic interaction chromatography matrix is cellulose.

A specific hydrophilic interaction material includes graphitized carbon. The graphitized carbon separates non-charged carbohydrate materials based mainly on the size on the glycan. There is also possible ion exchange effects. In a preferred embodiment the invention is directed to graphitized carbon chromatography of prepurified samples after desalting and removal of detergents.

The invention is specifically directed to purification of non-derivatized glycomes and neutral glycomes by cellulose chromatography. The invention is further directed to purification of non-derivatized glycomes and neutral glycomes by graphitized carbon chromatography. In a preferred embodiment the purification according to the invention includes both cellulose and graphitized carbon chromatography.

### b) Ion Exchange

The glycome may comprise only negative charges or in more rare case also only positive charges. At purification stage the ion exchange material is selected to contain opposite charge than the glycome or glycome fraction for binding the glycome. The invention is especially directed to the use of anion exchange materials for binding of negatively charged Preferred ion exchange materials includes ion exchange and especially anion exchange materials includes polystyrene resins such as Dowex-resins, preferably quaternary amine resins anion exchange or sulfonic acid cation exchange resins

It was further revealed that even graphitized carbon can be used for binding of negatively charged glycomes and the materials can be eluted from the carbon separately from the neutral glycomes or glycome fractions according to the invention.

The invention is specifically directed to purification of anionic glycomes by anion exchange chromatography.

The invention is specifically directed to purification of anionic glycomes by anion exchange chromatography.

The invention is further directed to purification of anionic glycomes by cellulose chromatography. The preferred anionic glycomes comprise sialic acid and/or sulfo/fosfo esters, more preferably both sialic acid and sulfo/fosfo esters. A preferred class of sulfo/fosfoester glycomes are complex type N-glycans comprising sulfate esters.

### Prepurification and Purification Steps in Detail

1) Precipitation-extraction may include precipitation of glycans or precipitation of contaminants away from the glycans. Preferred precipitation methods include:
4. Glycan material precipitation, for example acetone precipitation of glycoproteins, oligosaccharides, glycopeptides, and glycans in aqueous acetone, preferentially ice-cold over 80% (v/v) aqueous acetone; optionally combined with extraction of glycans from the precipitate, and/or extraction of contaminating materials from the precipitate;
5. Protein precipitation, for example by organic solvents or trichloroacetic acid, optionally combined with extraction of glycans from the precipitate, and/or extraction of contaminating materials from the precipitate;
6. Precipitation of contaminating materials, for example precipitation with trichloroacetic acid or organic solvents such as aqueous methanol, preferentially about ⅔ aqueous methanol for selective precipitation of proteins and other non-soluble materials while leaving glycans in solution;

2) Ion-exchange may include ion-exchange purification or enrichment of glycans or removal of contaminants away from the glycans. Preferred ion-exchange methods include:
3. Cation exchange, preferably for removal of contaminants such as salts, polypeptides, or other cationizable molecules from the glycans; and
4. Anion exchange, preferably either for enrichment of acidic glycans such as sialylated glycans or removal of charged contaminants from neutral glycans, and also preferably for separation of acidic and neutral glycans into different fractions.

3) Hydrophilic interaction may include purification or enrichment of glycans due to their hydrophilicity or specific adsorption to hydrophilic materials, or removal of contaminants such as salts away from the glycans. Preferred hydrophilic interaction methods include:
3. Hydrophilic interaction chromatography with specific organic or inorganic polar interaction materials, preferably for purification or enrichment of glycans and/or glycopeptides;
4. Preferably adsorption of glycans to carbohydrate materials, preferably to cellulose in hydrophobic solvents for their purification or enrichment, preferably to microcrystalline cellulose, and elution by polar solvents such as water and or alcohol, which is preferably ethanol or methanol. The solvent system for absorption comprise preferably
   i) a hydrophobic alcohol comprising about three to five carbon atoms, including propanols, butanols, and pentanols, more preferably being n-butanol;
   ii) a hydrophilic alcohol such as methanol or ethanol, more preferably methanol, or a hydrophilic weak organic acid, preferably acetic acid and;
   iii) water. The hydrophobic alcohol being the major constituent of the mixture with multifold excess to other components. The absorption composition is preferably using an n-butanol:methanol:water or similar solvent system for adsorption and washing the adsorbed glycans, in most preferred system n-butanol:methanol:water in relative volumes of 10:1:2. The preferred polar solvents for elution of the glycomes are water or water:ethanol or similar solvent system for elution of purified glycans from cellulose. Fractionation is possible by using first less polar elution solvent to elute a fraction of glycome compositions and the eluting rest by more polar solvent such as water
      3. Affinity to carbon may include purification or enrichment of glycans due to their affinity or specific adsorption to specific carbon materials preferably graphitized carbon, or removal of contaminants away from the glycans. Preferred graphitized carbon affinity methods includes porous graphitized carbon chromatography.

Preferred purification methods according to the invention include combinations of one or more prepurification and/or purification steps. The preferred method include preferably at least two and more preferably at least three prepurification steps according to the invention. The preferred method include preferably at least one and more preferably at least two purification steps according to the invention. It is further realized that one prepurification step may be performed after a purification step or one purification step may be performed after a prepurification step. The method is preferably adjusted based on the amount of sample and impurities present in samples. Examples of the preferred combinations include the following combinations:

For neutral underivatized glycan purification:

A. 1. precipitation and/or extraction 2. cation exchange of contaminants, 3. hydrophobic adsorption of contaminants, and 4. hydrophilic purification, preferably by carbon, preferably graphitized carbon, and/or carbohydrate affinity purification of glycans.

B. 1. precipitation and/or extraction, 2. hydrophobic adsorption of contaminants 3. cation exchange of contaminants, 4. hydrophilic purification by carbon, preferably graphitized carbon, and/or carbohydrate affinity purification of glycans

The preferred method variants further includes preferred variants when
1. both carbon and carbohydrate chromatography is performed in step 4,
2. only carbon chromatography is performed in step 4
3. only carbohydrate chromatography is performed in step 4
4. order steps three and four is exchanged
5. both precipitation and extraction are performed in prepurification step

2) For sialylated/acidic underivatized glycan purification: The same methods are preferred but preferably both carbon and carbohydrate chromatography is performed in step 4. The carbohydrate affinity chromatography is especially preferred for acidic and/sialylated glycans. In a preferred embodiment for additional purification one or two last chromatography methods are repeated.

### D. Analysis of the Glycomes

The present invention is specifically directed to detection various component in glycomes by specific methods for recognition of such components. The methods includes binding of the glycome components by specific binding agents according to the invention such as antibodies and/or enzymes, these methods preferably include labeling or immobilization of the glycomes. For effective analysis of the glycome a large panel of the binding agents are needed.

The invention is specifically directed to physicochemical profiling methods for exact analysis of glycomes. The preferred methods includes mass spectrometry and NMR-spectroscopy, which give simultaneously information of numerous components of the glycomes. In a preferred embodiment the mass spectrometry and NMR-spectroscopy methods are used in a combination.

### E. Quantitative and Qualitative Analysis of Glycome Data

The invention revealed methods to create reproducible and quantitative profiles of glycomes over large mass ranges and degrees of polymerization of glycans. The invention further reveals novel methods for quantitative analysis of the glycomics data produced by mass spectrometry. The invention is specifically directed to the analysis of non-derivatized or reducing end derivatized glycomes according to the invention and the glycomes containing specific structural characteristics according to the invention.

The invention revealed effective means of comparison of glycome profiles from different cell types or tissue materials with difference in cell status or cell types. The invention is especially directed to the quantitative comparison of relative amount of individual glycan signal or groups of glycan signals described by the invention.

The invention is especially directed to

i) calculating average value and variance values of signal or signals, which have obtained from several experiments/samples and which correspond to an individual glycan or glycan group according to the invention for a first cell sample and for a second cell sample
ii) comparing these with values derived for the corresponding signal(s)
iii) optionally calculating statistic value for testing the probability of similarity of difference of the values obtained for the cell types or estimating the similarity or difference based on the difference of the average and optionally also based on the variance values.
iv) preferably repeating the comparison one or more signals or signal groups, and further preferably performing combined statistical analysis to estimate the similarity and/or differences between the data set or estimating the difference or similarity
v) preferably use of the data for estimating the differences between the first and second cell samples indicating difference in cell status and/or cell type

The invention is further directed to combining information of several quantitative comparisons of between corresponding signals by method of

i) calculating differences between quantitative values of corresponding most different glycan signals or glycan group signals, changing negative values to corresponding positive values, optionally multiplying selected signals by selected factors to adjust the weight of the signals in the calculation
ii) adding the positive difference values to a sum value
iii) comparing the sum values as indicators of cell status or type.

It was further revealed that there is characteric signals that are present in certain cell types according to the invention but absent or practically absent in other cell types. The invention is therefore directed to the qualitative comparison of relative amount of individual glycan signal or groups of glycan signals described by the invention and observing signals present or absent/practically absent in a cell type. The invention is further directed to selection of a cut off value used for selecting absent or practically absent signals from a mass spectrometric data, for example the preferred cut off value may be selected in range of 0-3% of relative amount, preferably the cut off value is less than 2%, or less than 1% or less than 0.5%. In a preferred embodiment the cut off value is adjusted or defined based on quality of the mass spectrum obtained, preferably based on the signal intensities and/or based on the number of signals observable.

The invention is further directed to atomized qualitative and/or quantitative comparisons of data from corresponding signals from different samples by computer and computer programs processing glycome data produced according to the invention. The invention is further directed to raw data based analysis and neural network based learning system analysis as methods for revealing differences between the glycome data according to the invention.

### Methods for Low Sample Amounts

The present invention is specifically directed to methods for analysis of low amounts of samples.

The invention further revealed that it is possible to use the methods according to the invention for analysis of low sample amounts. It is realized that the cell materials are scarce and difficult to obtain from natural sources. The effective analysis methods would spare important cell materials. Under certain circumstances such as in context of cell culture the materials may be available from large scale. The required sample scale depends on the relative abundancy of the characteristic components of glycome in comparison to total amount of carbohydrates. It is further realized that the amount of glycans to be measured depend on the size and glycan content of the cell type to be measured and analysis including multiple enzymatic digestions of the samples would likely require more material. The present invention revealed especially effective methods for free released glycans.

The picoscale samples comprise preferably at least about 1000 cells, more preferably at least about 50 000 cells, even more preferably at least 100 000 cells, and most preferably at least about 500 000 cells. The invention is further directed to analysis of about 1 000 000 cells. The preferred picoscale samples contain from at least about 1 000 cells to about 10 000 000 cells according to the invention. The useful range of amounts of cells is between 50 000 and 5 000 000, even more preferred range of cells is between 100 000 and 3 000 000 cells. A preferred practical range for free oligosaccharide glycoomes is between about 500 000 and about 2 000 000 cells. It is realized that cell counting may have variation of less than 20%, more preferably 10% and most preferably 5%, depending on cell counting methods and cell sample, these variations may be used instead of term about. It is further understood that the methods according to the present invention can be upscaled to much larger amounts of material and the pico/nanoscale analysis is a specific application of the technology.

The invention is specifically directed to use of microcolumn technologies according to the invention for the analysis of the preferred picoscale and low amount samples according to the invention,

The invention is specifically directed to purification to level, which would allow production of high quality mass spectrum covering the broad size range of glycans of glycome compositions according to the invention.

### Glycan Preparation and Purification for Glycome Analysis of Cell Materials According to the Invention, Especially for Mass Spectrometric Methods

Use of microfluidistic methods including microcolumn chromatography The present invention is especially directed to use microfluidistic methods involving low sample volumes in handling of the glycomes in low volume cell preparation, low volume glycan release and various chromatographic steps. The invention is farther directed to integrated cell preparation, glycan release, and purification and analysis steps to reduce loss of material and material based contaminations. It is further realized that special cleaning of materials is required for optimal results.

### Low Volume Reaction in Cell Preparation and Glycan Release

The invention is directed to reactions of volume of 1-100 microliters, preferably about 2-50 microliters and even more preferably 3-20 microliters, most preferably 4-10 microliter. The most preferred reaction volumes includes 5-8 microliters+/−1 microliters. The minimum volumes are preferred to get optimally concentrated sample for purification. The amount of material depend on number of experiment in analysis and larger amounts may be produced preferably when multiple structural analysis experiments are needed.

It is realized that numerous low volume chromatographic technologies may be applied, such low volume column and for example disc based microfluidistic systems.

The inventors found that the most effective methods are microcolumns. Small column can be produced with desired volume. Preferred volumes of microcolumns are from about 2 Microliters to about 500 microliters, more preferably for routine sample sizes from about 5 microliter to about 100 microliters depending on the matrix and size of the sample. Preferred microcolumn volumes for graphitized carbon, cellulose chromatography and other tip-columns are from 2 to 20 μl, more preferably from 3 to 15 μl, even more preferably from 4 to 10 μl, For the microcolumn technologies in general the samples are from about 10 000 to about million cells. The methods are useful for production of picomol amounts of total glycome mixtures from tissue materials according to the invention.

In a preferred embodiment microcolumns are produced in regular disposable usually plastic pipette tips used for example in regular “Finnpipette”-type air-piston pipettes. The pipette-tip microcolumn contain the preferred chromatographic matrix. In a preferred embodiment the microcolumn contains two chromatographic matrixes such as an anion and cation exchange matrix or a hydrophilic and hydrophobic chromatography matrix.

The pipette tips may be chosen to be a commercial tip contain a filter. In a preferred embodiment the microcolumn is produced by narrowing a thin tip from lower half so that the preferred matrix is retained in the tip. The narrowed tip is useful as the volume of filter can be omitted from washing steps

The invention is especially directed to plastic pipette tips containing the cellulose matrix, and in an other embodiment to the pipette tip microcolumns when the matrix is graphitised carbon matrix. The invention is further directed to the preferred tip columns when the columns are narrowed tips, more preferably with column volumes of 1 microliter to 100 microliters.

The invention is further directed to the use of the tip columns containing any of the preferred chromatographic matrixes according to the invention for the purification of glycomes according to the invention, more preferably matrixes for ion exchange, especially polystyrene anion exchangers and cation exchangers according to the invention; hydrophilic chromatographic matrixes according to the invention, especially carbohydrate matrixes and most cellulose matrixes.

The Binding Methods for Recognition of Structures from Cell Surfaces

### Antibodies

Information about useful lectin and antibody specificities useful according to the invention and for reducing end elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds. Sharon, Nathan and L is, Halina) Kluwer Academic publishers Dordrecht, The Neatherlands and internet databases such as pubmed/espacenet or antibody databases such as www.glyco.is.ritsumei.ac.jp/epitope/, which list monoclonal antibody specificities).

Various procedures known in the art may be used for the production of polyclonal antibodies to glycan motifs or oligosaccharides and regions or fragments thereof. For the production of antibodies, any suitable host animal (including but not limited to rabbits, mice, rats, or hamsters) are immunized by injection with a glycan, glycan fragment or oligosaccharide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corγnebacterium parvum.

A monoclonal antibody to a glycan motif(s)/oligosaccharide may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-I1, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce glycan, glycan fragment, or oligosaccharide specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the condensed framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Preferably, the antibody is any antibody specific for a glycan structure of Formulas of the present invention, oligosaccharides, glycan epitopes or a fragment thereof, preferably a low-mannose glycan, LacNAc O-glycan, or sialyl-LacNAc O-glycan, more preferably a Core 2 type O-glycan, as described in the present invention. The antibody used in the present invention encompasses any antibody or fragment thereof, native or recombinant, synthetic or naturally-derived, monoclonal or polyclonal which retains sufficient specificity to bind specifically to a glycan structure according to the present invention, without gaining affinity to other glycan sequences. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments. The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, I-125 and amino acids comprising any radionuclides, including, but not limited to C-14, H-3 and S-35.

Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to two methods:
i) Recognition by enzymes involving binding and alteration of structures.
This method alters specific glycan structures by enzymes capable of altering the glycan structures. The preferred enzymes includes
   a) glycosidase-type enzymes capable of releasing monosaccharide units from glycans
   b) glycosyltransferring enzymes, including transglycosylating enzymes and glycosyltransferases
   c) glycan modifying enzymes including sulfate and or phosphate modifying enzymes
ii) Recognition by molecules binding glycans referred as the binders
These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group monoclonal antibody, glycosidase, glycosyl transferring enzyme, plant lectin, animal lectin or a peptide mimetic thereof, and wherein the binder includes a detectable label structure.

### Preferred Binder Molecules

The present invention revealed various types of binder molecules useful for characterization of tissue materials according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

### Target Structures for Specific Binders and Examples of the Binding Molecules

Combination of Terminal Structures in Combination with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition terminal epitopes.

### Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount.

### Specific Preferred Structural Groups

The present invention is directed to recognition of oligosaccharide sequences comprising specific terminal monosaccharide types, optionally further including a specific core structure. The preferred oligosaccharide sequences classified based on the terminal monosaccharide structures.

1. Structures with Terminal Mannose Monosaccharide

Preferred mannose-type target structures have been specifically classified by the invention. These include various types of high and low-mannose structures and hybrid type structures according to the invention.

### Low or Uncharacterised Specificity Binders

preferred for recognition of terminal mannose structures includes mannose-monosaccharide binding plant lectins.

Preferred High Specific High Specificity Binders include
i) Specific mannose residue releasing enzymes such as linkage specific mannosidases, more preferably an x-mannosidase or β-mannosidase.
Preferred α-mannosidases includes linkage specific α-mannosidases such as α-Mannosidases cleaving preferably non-reducing end terminal
α2-linked mannose residues specifically or more effectively than other linkages, more preferably cleaving specifically Manα2-structures; or
α6-linked mannose residues specifically or more effectively than other linkages, more preferably cleaving specifically Manα6-structures;
Preferred β-mannosidases includes β-mannosidases capable of cleaving β4-linked mannose from non-reducing end terminal of N-glycan core Manβ4GlcNAc-structure without cleaving other β-linked monosaccharides in the glycomes.
ii) Specific binding proteins recognizing preferred mannose structures according to the invention. The preferred reagents include antibodies and binding domains of antibodies (Fab-fragments and like), and other engineered carbohydrate binding proteins. The invention is directed to antibodies recognizing MS2B1 and more preferably MS3B2-structures
2. Structures with Terminal Gal-Monosaccharide

Preferred galactose-type target structures have been specifically classified by the invention. These include various types of N-acetyllactosamine structures according to the invention.

### Low or Uncharacterised Specificity Binders for Terminal Gal

Preferred for recognition of terminal galactose structures includes plant lectins such as ricin lectin (ricinus communis agglutinin RCA), and peanut lectin(/agglutinin PNA).

### Preferred High Specific High Specificity Binders Include

i) Specific galactose residue releasing enzymes such as linkage specific galactosidases, more preferably α-galactosidase or β-galactosidase.
Preferred α-galactosidases include linkage galactosidases capable of cleaving Galα3Gal-structures revealed from specific cell preparations
Preferred β-galactosidases includes O— galactosidases capable of cleaving
β4-linked galactose from non-reducing end terminal Galβ4GlcNAc-structure without cleaving other β-linked monosaccharides in the glycomes and
β3-linked galactose from non-reducing end terminal Galβ3GlcNAc-structure without cleaving other β-linked monosaccharides in the glycomes
ii) Specific binding proteins recognizing preferred galactose structures according to the invention. The preferred reagents include antibodies and binding domains of antibodies (Fab-fragments and like), and other engineered carbohydrate binding proteins and animal lectins such as galectins.
3. Structures with Terminal GalNAc-Monosaccharide

Preferred GalNAc-type target structures have been specifically revealed by the invention. These include especially LacdiNAc, GalNAcβGlcNAc-type structures according to the invention.

### Low or Uncharacterised Specificity Binders for Terminal GalNAc

Several plant lectins has been reported for recognition of terminal GalNAc. It is realized that some GalNAc-recognizing lectins may be selected for low specificity recognition of the preferred LacdiNAc-structures.

### Preferred High Specific High Specificity Binders Include

i) The invention revealed that β-linked GalNAc can be recognized by specific β-N-acetylhexosaminidase enzyme in combination with β-N-acetylhexosaminidase enzyme. This combination indicates the terminal monosaccharide and at least part of the linkage structure.

Preferred β-N-acetylhexosaminidase, includes enzyme capable of cleaving β-linked GalNAc from non-reducing end terminal GalNAcβ4/3-structures without cleaving α-linked HexNAc in the glycomes; preferred N-acetylglucosaminidase include enzyme capable of cleaving-linked GlcNAc but not GalNAc.

ii) Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins, and a special plant lectin WFA (Wisteria floribunda agglutinin).
4. Structures with Terminal GlcNAc-Monosaccharide

Preferred GlcNAc-type target structures have been specifically revealed by the invention. These include especially GlcNAcβ-type structures according to the invention.

### Low or Uncharacterised Specificity Binders for Terminal GlcNAc

Several plant lectins has been reported for recognition of terminal GlcNAc. It is realized that some GlcNAc-recognizing lectins may be selected for low specificity recognition of the preferred GlcNAc-structures.

### Preferred High Specific High Specificity Binders Include

i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving-linked GalNAc or α-linked HexNAc in the glycomes;

ii) Specific binding proteins recognizing preferred GlcNAcβ2/3/6, more preferably GlcNAcβ2Manα, structures according to the invention. The preferred reagents include antibodies and binding domains of antibodies (Fab-fragments and like), and other engineered carbohydrate binding proteins.
5. Structures with Terminal Fucose-Monosaccharide

Preferred fucose-type target structures have been specifically classified by the invention. These include various types of N-acetyllactosamine structures according to the invention.

### Low or Uncharacterised Specificity Binders for Terminal Fuc

Preferred for recognition of terminal fucose structures includes fucose monosaccharide binding plant lectins. Lectins of Ulex eutropeaus and Lotus tetragonolobus has been reported to recognize for example terminal Fucoses with some specificity binding for α2-linked structures, and branching α3-fucose, respectively.

### Preferred High Specific High Specificity Binders Include

i) Specific fucose residue releasing enzymes such as linkage fucosidases, more preferably α-fucosidase.

Preferred α-fucosidases include linkage fucosidases capable of cleaving Fucα2Gal-, and Galβ4/3(Fucα3/4)GlcNAc-structures revealed from specific cell preparations.

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

6. Structures with Terminal Sialic Acid-Monosaccharide

Preferred sialic acid-type target structures have been specifically classified by the invention.

### Low or Uncharacterised Specificity Binders for Terminal Fuc

Preferred for recognition of terminal sialic acid structures includes sialic acid monosaccharide binding plant lectins.

### Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal -structures revealed from specific cell preparations by the invention.

Preferred lectins, with linkage specificity include the lectins, that are specific for SAα3Gal-structures, preferably being Maackia amurensis lectin and/or lectins specific for SAa6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins. The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

### Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells or tissue materials according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

### Use of Binder and Labelled Binder-Conjugates for Cell Sorting

The invention is specifically directed to use of the binders and their labelled conjugates for sorting or selecting cells from biological materials or samples including cell materials comprising other cell types. The preferred cell types includes cultivated cells and associated cells such as feeder cells. The labels can be used for sorting cell types according to invention from other similar cells. In another embodiment the cells are sorted from different cell types such as blood cells or in context of cultured cells preferably feeder cells, for example in context of complex cell cultures corresponding feeder cells such as human or mouse feeder cells. A preferred cell sorting method is FACS sorting. Another sorting methods utilized immobilized binder structures and removal of unbound cells for separation of bound and unbound cells.

### Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials

mRNA Corresponding to Glycosylation Enzymes

The present invention is further directed to correlation of specific messenger mRNA molecules with the preferred glycan structures according to the present invention. It is realized that glycosylation can be controlled in multiple levels and one of them is transcription. The presence of glycosylated structures may in some case correlate with mRNAs involved in the synthesis of the structures.

The present invention is especially directed to analysis of mRNA-species having correlation with expressed fucosylated glycan structures and “terminal HexNAc” containing structures preferred according to the present invention. The preferred mRNA-species includes mRNA corresponding to fucosyltransferases and N-acetylglucosaminyltransferases.

### NMR-Analysis of Glycomes

The present invention is directed to analysis of released glycomes by spectrometric method useful for characterization of the glycomes from tissue specimens or cells. The invention is directed to NMR spectroscopic analysis of the mixtures of released glycans.

The invention is especially directed to methods of producing NMR from specific subglycomes, preferably N-linked glycome, O-linked glycome, glycosaminoglycan glycome and/or glycolipid glycome. The NMR-profiling according to the invention is further directed to the analysis of the novel and rare structure groups revealed from cell glycomes according to the invention. The general information about complex cell glycome material directed NMR-methods are limited.

Preferably the NMR-analysis is performed from an isolated subglycome. The preferred isolated subglycomes include acidic glycomes and neutral glycomes.

### NMR-Glycome Analysis of Larger Tissue Specimens or Larger Amounts of Cells

It is realized that numerous methods have been described for purification of oligosaccharide mixtures useful for NMR from various materials, including usually purified individual proteins. It is realized that present methods are useful for NMR-profiling even for larger tissue specimens or higher amounts of cells according to the invention, especially in combination with NMR-profiling according to the invention and/or when directed to the analysis specific and preferred structure groups according to the invention. The preferred purification methods are effective and form an optimised process for purification of glycomes from even larger amounts of cells and tissues than described for nanoscale methods below. The methods are preferred also for any larger amount of cells.

### Purification Method for Low Amount Nanoscale NMR-Profiling Of Samples

Moreover, when purification methods for larger amounts of carbohydrate materials exists, but very low and complex carbohydrate materials with very complex impurities such as cell-derived materials have been less studied as low amounts, especially when purity useful for NMR-analysis is needed.

### Preferred Sample Amounts Allowing Effective NMR Analysis of Cell Glycomes

The invention is directed to analysis of NMR-samples that can be produced from very low amounts of cells according to the invention. Preferred sample amounts of cells or corresponding amount of tissue material for a one-dimensional proton-NMR profiling are from about 2 million to 100 million cells, more preferably 10-50 million cells. It is further realized that good quality NMR data can be obtained from samples containing at least about 10-20 million cells.

The preferred analysis methods is directed to high resolution NMR observing oligosaccharide/saccharide conjugate mixture from an amount of at least 4 nmol, more preferably at least 1 nmol and the cell amount yielding the preferred amount of saccharide mixture. For nanoscale analysis according to the invention cell material is selected so that it would yield at least about 50 nmol of oligosaccharide mixture, more preferably at least about 5 nmol and most preferably at least about 1 nmol of oligosaccharide mixture. Preferred amounts of major components in glycomes to be observed effectively by the methods according to the invention include yield at least about 10 nmol of oligosaccharide component, more preferably at least about 1 nmol and most preferably at least about 0.2 nmol of oligosaccharide component.

The preferred cell amount for analysis of a subglycome from a cell type is preferably optimised by measuring the amounts of glycans produced from the cell amounts of preferred ranges.

It is realized that depending on the cell and subglycome type the required yield of glycans and the heterogeneity of the materials vary yielding different amounts of major components.

### Preferred Purification Methods

For the production of sample for nanoscale NMR, the methods described for preparation of cell samples and release of oligosaccharides for mass spectrometric profiling according to the invention may be applied.

For the purification of sample for nanoscale NMR the methods described for purification mass spectrometry profiling samples according to the invention may be applied.

The preferred purification method for nanoscale NMR-profiling according to the invention include following general purification process steps:
a. Precipitation/extraction;
b. Hydrophobic interaction;
c. Affinity to carbon material, especially graphitized carbon.
d. Gel filtration chromatography

The more preferred purification process includes precipitation/extraction aimed for removal of major non-carbohydrate impurities by separating the impurity and the glycome fraction(s) to be purified to different phases. Hydrophobic interaction step aims to purify the glycome components from more hydrophobic impurities as these are bound to hydrophobic chromatography matrix and the glycome components are not retained. Chromatography on graphitized carbon may include purification or enrichment of glycans due to their affinity or specific adsorption to graphitized carbon, or removal of contaminants from the glycans. The glycome components obtained by the aforementioned steps are then subjected to gel filtration chromatography, separating molecules according to their hydrodynamic volume, i.e. size in solution. The gel filtration chromatography step allows detection and quantitation of glycome components by absorption at low wavelengths (205-214 nm).

The most preferred purification process includes precipitation/extraction and hydrophobic interaction steps aimed for removal of major non-carbohydrate impurities and more hydrophobic impurities. Chromatography on graphitized carbon is used for removal of contaminants from the glycans, and to devide the glycome components to fractions of neutral glycome components and acidic glycome components. The neutral and acidic glycome component fractions are then subjected to gel filtration chromatography, which separates molecules according to their size. Preferably, a high-performance liquid chromatography (HPLC) type gel filtration column is used. The neutral glycome component fraction is preferably chromatographed in water and the acidic glycome component fraction is chromatographed in 50-200 mM aqueous ammonium bicarbonate solution. Fractions are collected and evaporated prior to further analyses. The gel filtration chromatography step allows detection and quantitation of glycome components by absorption at low wavelengths (205-214 nm). Quantitation is performed against external standards. The standards are preferably N-acetylglucosamine, N-acetylneuraminic acid, or oligosaccharides containing the same. Fractions showing absorbance are subjected to MALDI-TOF mass spectrometry. Preferably, the neutral glycome components are analyzed in the positive-ion mode and the acidic glycome components in the negative-ion mode in a delayed-extraction MALDI-TOF mass spectrometer.

### Preferred Methods for Producing Enriched Glycome Fractions for NMR

The fractionation can be used to enrich components of low abundance. It is realized that enrichment would enhance the detection of rare components. The fractionation methods may be used for larger amounts of cell material. In a preferred embodiment the glycome is fractionated based on the molecular weight, charge or binding to carbohydrate binding agents such as lectins and/or other binding agents according to the invention.

These methods have been found useful for specific analysis of specific subglycomes and enrichment more rare components. The present invention is in a preferred embodiment directed to charge based separation of neutral and acidic glycans. This method gives for analysis method, preferably mass spectroscopy material of reduced complexity and it is useful for analysis as neutral molecules in positive mode mass spectrometry and negative mode mass spectrometry for acidic glycans.

It is realized that preferred amounts of enriched glycome oligosaccharide mixtures and major component comprising fractions can be produced from larger glycome preparations.

In a preferred embodiment the invention is directed to size based fractionation methods for effective analysis of preferred classes of glycans in glycomes. The invention is especially directed to analysis of lower abundance components with lower and higher molecular weight than the glycomes according to the invention. The preferred method for size based fractionation is gel filtration. The invention is especially directed to analysis of enriched group glycans of N-linked glycomes preferably including lower molecular weight fraction including low-mannose glycans, and one or several preferred low mannose glycan groups according to the invention.

### Preferred NMR-Methods

In a preferred embodiment the NMR-analysis of the glycome is one-dimensional proton-NMR analysis showing structural reporter groups of the major components in the glycome. The invention is further directed to specific two- and multidimensional NMR-experiments of the glycomes when enough sample is available. It is realized that two-dimensional NMR-experiments require about a ten-fold increase in sample amount compared to proton-NMR analyses.

### Combination of NMR- and Mass Spectrometry for Glycome Analysis

The present invention is further directed to combination of the mass spectrometric and NMR glycome analyses. The preferred method include production of any mass spectrometric profile from any glycome according to the invention from a cell sample according to the invention, optionally characterizing the glycome by other methods like glycosidase digestion, fragmentation mass spectrometry, specific binding agents, and production of NMR-profile from the same sample glycome or glycomes to compare these profiles.

### Production of Cancer Antigens and Vaccines

As described in the invention the over expressed glycan structures in cancers are useful therapeutic targets, especially when the structures are not present in normal tissues. The invention is directed to production of therapeutic cancer binding antibodies and anti-cancer immune response inducing cancer vaccines. It is realized that the glycoconjugates of invention for cancer vaccines and/or antigens can be produced by multiple synthetic and or biosynthetic methods known in the art.

### Biotechnic Production of Cancer Vaccines

The invention is especially directed to the biotechnical production of cancer vaccine/antigen comprising target cancer glycans. Preferred biotechnical methods for the production of the vaccine by purification from a natural source. Preferred sources of cancer vaccines includes natural glycoproteins obtainable from pharmaceutically acceptable source, preferably the glycoprotein is an eucaryotic glycoprotein.

### KLH-Cancer Vaccine

A preferred example of a natural glycoprotein is KLH (keyhole limpet hemocyanin). In a preferred embodiment the invention is directed to alteration of the KLH to more cancer vaccine like structure by removing non-human type β(6)-Gal-structures from KLH. The KLH thus obtained contains increased amount of low mannose antigen/vaccine structures according to the invention. It is further realized that the preferred antigenicity is more effective in the low-mannose enriched KLH-materials as the non-human structure would otherwise direct immune system to non-useful reactions. In another preferred embodiment the amount of N-glycand core fucose is specifically reduced (preferably by fucosidase or acid hydrolysis) or increased by enzymatic fucosylation. It is well-known protein for immunization. Suitable amounts of KLH for use as an immunoadjuvant in cancers are well-known in the art and the invention is especially directed to the use of similar amounts of glycan antigen enriched molecules for cancer treatment.

Recombinant Proteins from Lower Eukayots

It is further realized that multiple recombinant protein host cell systems such as yeast and fungal cells can be engineered for production specific low-Mannose and/or high-Mannose structures (described e.g. in glycoprotein production patents of Kirin Bier Japan, Glycofi US and prof Roland Contreras Belgium) and other structures preferably core II type O-glycans, for O-glycans mammalian expression systems are currently more feasible.

The present invention is directed to the methods of clinical evaluation of effect of immunization by glycoproteins/peptides enriched with the preferred cancer antigens according to the invention for patients with one or several types of cancers containing the structures.

### Analysis of Serum and Other Biological Fluids,

### Preferred Analysis of Soluble Markes Such as CA15-3 Antigen

In a specific embodiment the invention is directed to analysis of secretory proteins at least partially derived from cancer such as serum proteins from cancer. As an example the inventors analyzed glycosylation of a commercial CA15-3 cancer antigen sample (Calbiochem, USA, prod number 209915, cancer associated breast tumor antigen from tumor fluids). The invention revealed the preferred neutral and sialylated core II glycans, especially fucosylated core II glycans, with substantial expression sialyl-Lewis x core II structure and even polylactosame elongated sialylated and fucosylated structures according to the invention.

The data indicates that the common and useful cancer marker CA15-3 for breast cancer is actually a core II O-glycan structure, more preferably sialylated and/or fucosylated, preferably sialylated and at least partially fucosylated core two O-glycan epitope, when the CA15-3 antigen is derived from fluids of human breast tumors. The current antigen preparations are heterogenous human derived materials which are very difficult to standardize due to heterogeneity of the material further yielding heterogeneity in the characterization of heterogenous natural antibodies. The present invention is directed to novel synthetic CA15-3 standard, which comprise all or part of the preferred core II CA15-3 glycans, preferably linked to a polypeptide corresponding a part of CA15-3 protein (which has been considered as MUC1 mucin). The present invention is directed standardization methods, assaying the synthetic structures with regard to binding of known CA15-3 antibodies and analysis of the binding specificities of the antibodies with regard to the glycan structures. Based on the analysis one or several glycan structures according to the invention, preferably in a peptide linked form, is/are selected as synthetic CA15-3 antigen(s) or antigen types to be used for more exact cancer analysis, especially breast cancer analysis. The invention is further directed to production of recombinant CA15.3 antibody or antibody mixtures with specificities characterized with the standard glycan structures, a preferred such antibody composition would comprise. The invention is preferably directed to a novel recombinant CA15-3 antibody compositions comprising a core II slex-binding antibody, such as preferred antibody according to the invention. It is realized that the synthetic antigen can be adjusted with regard to individual cancer type specific variations of the glycan antigen structures.

The invention is further directed to analysis of the preferred glycan structures in context of cancer sample according to the invention, preferably a core II glycan on serum mucin, when the carrier protein is first purified or bound by an antibody specific for the carrier protein and then the complex is analysed by the specific glycan expression levels by other antibody or antibodies recognizing the glycan structures. The analysis may be performed by well-known sandwich ELISA type assays in which the first antibody is preferably immobilized and the second antibody is linked with a detectable label such as a fluorescent label or radiolabel; or for example by a fluorescence energy transfer analysis (FRET) when both antibodies (or other corresponding binders) are labeled with suitable fluorescent labels.

### Quantitative Synthesis and Analysis Method for Oxidation and Reduction of a Protein Linked Glycan

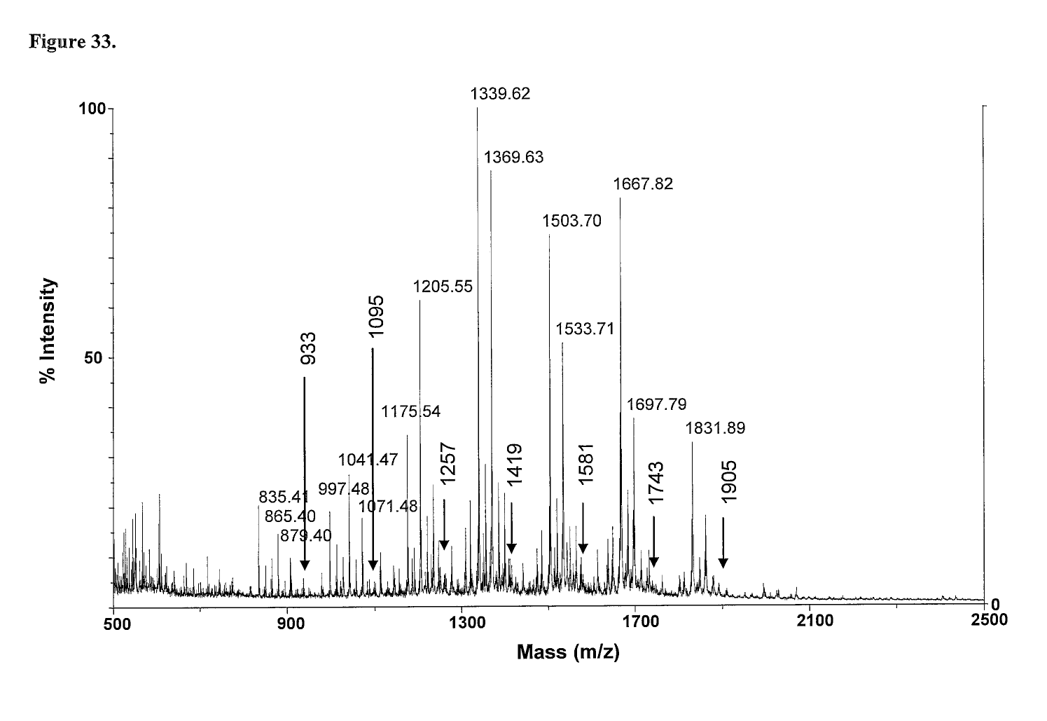

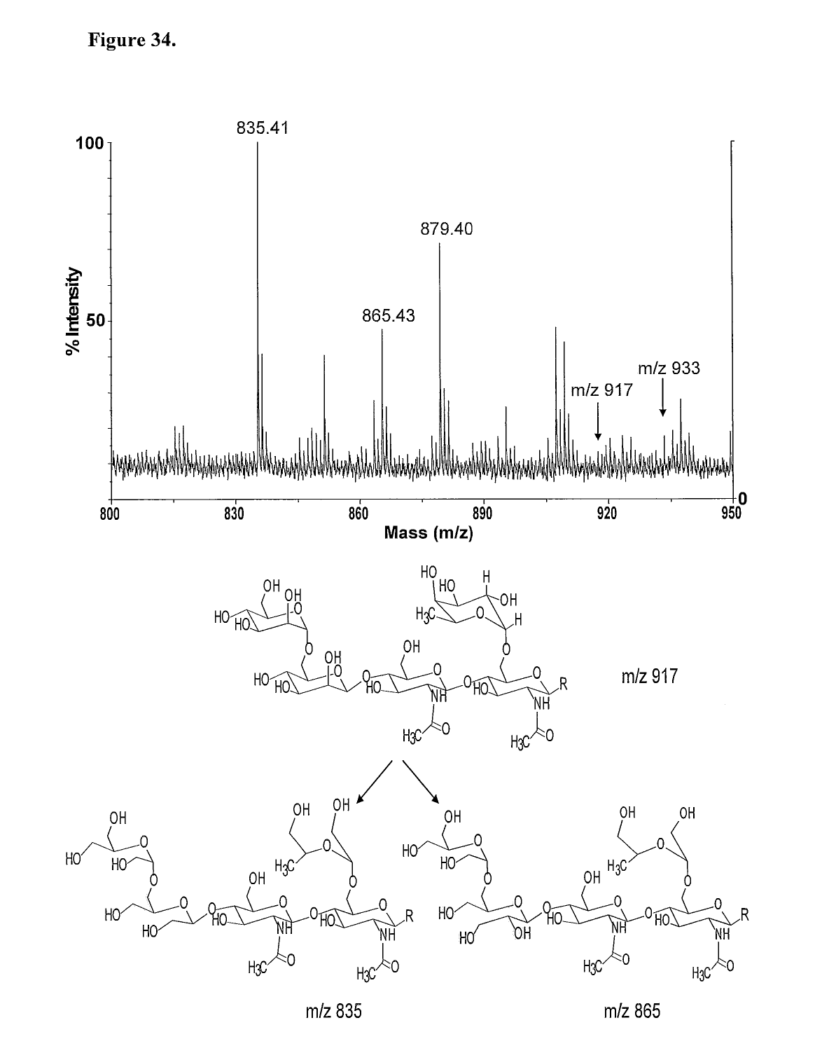

The present invention revealed a novel quantitative synthesis and analysis method for quantitative periodate(/vicinal hydroxyl) oxidation and reduction of a protein linked glycan. Under the preferred low temperature conditions and elongated reaction time as shown in Example illustrated by FIGS. 33 and 34. The preferred glycans to be oxidized and analyzed are terminal-Man glycans according to the invention, more preferably lower high-man and/or low-Man N-glycans according to the invention, more preferably low-Man glycans, including fucosylated and/or non-fucosylated glycans, which were revealed to be processable/analyzable according to the invention, in preferred embodiment the glycans are non-fucosylated. It is realized that the glycan structures have effect on the oxidation potential. The invention further revealed a quantitative analysis of reaction products of the oxidation and reduction method by MALDI-TOF mass spectrometry, preferably in positive ion mode according to the example. The mass spectra revealed that novel highly oxidized and reduced molecules ionize at least semiquantitatively similarity from oligosaccharides with various degrees of oligomerization, and the signals correlated to the quantitative amounts of corresponding raw material oligosaccharides with different lengths. The example further includes useful purification method for the glycans preserving the quantities of the samples. Before the invention there was not no quantitative, effective and quick method for this analysis of such reaction especially from low sample amounts such as lower picomol level of samples. It is realized that the method is useful for effective modification and characterization of all types glycans, especially terminal-Man glycans.

### Preferred Cancer Types According to the Present Invention

As described in the Examples, especially in Example 20, the inventors detected the presence and/or altered expression levels of the glycans widely in human cancerous tissues, e.g. in following cancer types: lung cancer, both small cell lung adenocarcinoma and non-small cell lung adenocarcinoma, and lung carcinoma liver metastases; breast cancer; ductale type breast adenocarcinoma and lymph node metastases thereof; lobulare type breast adenocarcinoma and lymph node metastases thereof; ovarian cystadenocarcinoma; colon cancer/carcinoma, carcinoma adenomatosum, and liver metastases thereof; kidney cancer/carcinoma, and kidney hypemephroma; gastric cancer/carcinoma, and lymph node metastases thereof, liver cancer/carcinoma; larynx cancer/carcinoma; pancreas cancer/carcinoma; melanoma and liver metastases thereof; gall bladder cancer/carcinoma, and liver metastases thereof; salivary gland cancer/carcinoma, and skin metastases thereof; and lymph node cancer/carcinoma (lymphoma). The present invention is especially directed to uses of identified glycan structures according to the present invention in these preferred cancer types.

Uses of Metastasis-Associated Glycans

The present invention is specifically directed to methods studying metastasis-associated, metastasis-specific, metastasis-enriched, and/or metastasis-inducing glycans according to the invention. As described in Examples 20-23, the inventors identified metastasis-associated and metastasis-enriched glycans in various human cancer types, including mannosylated glycans, preferentially low-mannose type glycans, O-glycans, non-reducing terminal HexNAc glycans, preferentially non-reducing terminal GlcNAc glycans, blood group antigen related glycans, and glycans with +80 Da units in their monosaccharide compositions, preferentially sulphated and/or phosphorylated glycans; in different specific combinations according to both primary cancer type and site of metastasis.

The present invention is especially directed to methods for identifying metastasis-associated glycans according to the invention. The present invention is further directed to methods for identifying primary cancer type and/or site of metastasis based on identifying the glycans according to the invention. In a further embodiment, the present invention is directed to methods for studying metastasis growth and/or initiation based on identifying the glycans according to the invention.

The present invention is also directed to methods for studying the biological function(s) of the metastasis-associated glycans according to the invention, preferentially in context of metastasis formation and cancer malignancy, more preferentially mechanisms of cancer spreading and migration. It is realized that metastasis-associated glycans are potentially immunologically active, and the present invention is further directed to methods for studying immunological properties of cancer, more preferentially metastasizing cancer, based on the glycans according to the invention. The invention is also directed to methods for using biological models to study cancer, more preferentially metastasizing cancer, in context of using metastasis-associated glycans according to the present invention, more preferentially including modulation of the glycans or using inhibiting glycans or their analogs.

It is further realized that cancer metastasis is directly related to cancer malignancy. The present invention is specifically directed to methods for analyzing cancer malignancy based on use of the metastasis-associated glycans according to the present invention.

The present invention is further illustrated in Examples, which in no way are intended to limit the scope of the invention.

Preferred N-Glycans According to Structural Subgroups with Terminal HexNAc

The inventors found that there are cancer-specific differences with regard to terminal HexNAc containing N-glycans characterized by either the formulae: n_{HexNAc}=n_{Hex}≧5 and n_{dHex}≧1 (group I), or the formulae: n_{HexNAc}=n_{Hex}≧5 and n_{dHex}=0 (group II). The present data demonstrated that group I glycans were 1) detected in various protein-linked glycan samples isolated from malignant tumor samples and metastases, 2) overexpressed when compared to corresponding normal tissues, 3) expressed independently of group II that was not expressed in specific studied examples of malignant tumors. Therefore, the N-glycan structure group determined by the formula n_{HexNAc}=n_{Hex}≧5 is divided into two independently expressed subgroups I and II as described above. It is realized that both of these groups preferably correspond to bisecting GlcNAc type N-glycans and other terminal HexNAc containing N-glycans.

Based on the known specificities of the biosynthetic enzymes synthesizing N-glycan core α1,6-linked fucose and β1,4-linked bisecting GlcNAc, group II preferably corresponds to bisecting GlcNAc type N-glycans while group I preferentially corresponds to other terminal HexNAc containing N-glycans, preferentially with a branching HexNAc in the N-glycan core structure, more preferentially including structures with a branching GlcNAc in the N-glycan core structure. In a specific embodiment the glycan structures of this group includes core fucosylated bisecting GlcNAc comprising N-glycan, wherein the additional GlcNAc is GlcNAcβ4 linked to Manβ4GlcNAc epitope forming epitope structure GlcNAcβ4Manβ4GlcNAc preferably between the complex type N-glycan branches.

In a preferred embodiment of the present invention, such structures include GlcNAc linked to the 2-position of the β1,4-linked mannose. In a further preferred embodiment of the present invention, such structures include GlcNAc linked to the 2-position of the β1,4-linked mannose as described for LEC14 structure (Raju and Stanley J. Biol Chem (1996) 271, 7484-93). In a further preferred embodiment of the present invention, such structures include GlcNAc linked to the 6-position of the β1,4-linked GlcNAc of the N-glycan core as described for LEC14 structure (Raju, Ray and Stanley J. Biol Chem (1995) 270, 30294-302).

The invention is specifically directed to further analysis of the subtypes of the group one glycans comprising structures according to the group I. The invention is further directed to production of specific binding reagents against the N-glycan core marker structures and use of these for analysis of the preferred cancer marker structures. The invention is further directed to the analysis of LEc 14 and/or 18 structures by negative recognition by lectins PSA (pisum sativum) or Intil (Lens culinaris) lectin or core Fuc specific monoclonal antibodies, which binding is prevented by the GlcNAcs.

Invention is specifically directed to N-glycan core marker structure, wherein the disaccharide epitope is Manβ4GlcNAc structure in the core structure of N-linked glycan according to the Formula CGN:

[Manα3]ₙ₁(Manα6)ₙ₂Manβ4GlcNAcβ4(Fucα6)ₙ₃GlcNAcxR,
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures for the analysis of the status of stem cells and/or manipulation of the stem cells, wherein xR indicates reducing end structure of N-glycan linked to protein or peptide such as βAsn or βAsn-peptide or βAsn-protein, or free reducing end of N-glycan or chemical derivative of the reducing produced for analysis.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises the GlcNAc substitutions.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises between 1-8% of the GlcNAc substitutions.

The invention is further directed to the N-glycan core marker structure and marker glycan compositions comprising structures of Formula CGN, wherein the structure is selected from the group

[GlcNAcβ2Manα3](GlcNAcβ2Manα6) Manβ4GlcNAcβ4(Fucα6)ₙ₃GlcNAcxR
[Galβ4GlcNAcβ2Manα3](Galβ4GlcNAcβ2Manα6) Manβ4GlcNAcβ4(Fucα6)ₙ₃GlcNAcxR And sialylated variants thereof when SA is α3 and or α6-linked to one or two Gal residues and Manβ4 or GlcNAcβ4 is substituted by GlcNAc.

The invention is further directed to the N-glycan core marker structure and marker glycan compositions comprising of Formula CGN, wherein the Manβ4GlcNAc-epitope comprises and the GlcNAc residue is β2-linked to Manβ4 forming epitope GlcNAcβ2Manβ4.

The invention is further directed to the N-glycan core marker structure and marker glycan compositions comprising of Formula CGN, wherein the Manβ4GlcNAc-epitope comprises and the GlcNAc residue is 2-linked to GlcNAc of the epitope forming epitope Manβ4(GlcNAc6)GlcNAc.

The invention is further directed to the N-glycan core marker structure and marker glycan compositions comprising of Formula CGN, wherein the Manβ4GlcNAc-epitope comprises and the GlcNAc residue is 4-linked to GlcNAc of the epitope forming epitope GlcNAcβ4Manβ4GlcNAc.

### Preferred Low-Mannose Type N-Glycan Binding Antibodies

Antibodies suitable for low-mannose type N-glycan recognition according to the invention include: Manα3 (Manα6)Manβ4(GlcNAcβ4GlcNAc) low-mannose type N-glycan specific antibody clone 100-4G11-A (mouse IgM, van Remoortere et al., 2003. Glycobiology 13, 217-225); Manα6(Manα3)Manα6(Manα3)Manβ4(GlcNAcβ4GlcNAc) low-mannose type N-glycan specific antibody clones L3 and L4 (Schmitz, B., et al., 1993. Glycobiology 3, 609-617); and Manα3Manβ4GlcNAc2 low-mannose type N-glycan specific antibody clone OMR5 and Manα6Manβ4GlcNAc2 low-mannose type N-glycan specific antibody clone OMR6 (Ozawa et al., 1997. Arch. Biochem. Biophys. 342, 48-57). Preferably, clone 100-4G11-A or antibody with similar binding specificity is used to recognize cancer types with low-mannose type N-glycan expression according to the invention,

The invention is in specific preferred embodiment directed to recognition of the epitopes by the antibodies (specifically preferably by Manα3(Manα6)Manβ4(GlcNAcβ4GlcNAe) low-mannose type N-glycan specific antibody clone) from a major cancer type including colon, ovarian, lung cancer or breast cancer, more preferably ovarian, lung cancer or breast cancer, most preferably lung cancer or breast cancer, and in specific embodiment or breast cancer.

### Preferred LacdiNAc Binding Antibodies

Antibodies suitable for LacdiNAc recognition according to the invention include: clone 259-2A1 (IgG3, against LacdiNAc); clones 273-3F2, 114-2H12-C, 100-2H5-A, and 99-2A5-B (IgM, against LacdiNAc); and clones 204-6A1, 290-2E6, and 294-2A1 (IgM, against α3Fuc-LacdiNAc; van Remoortere et al., 2000. Glycobiology 10, 601-609). Preferably, clone 259-2A1 or antibody with similar binding specificity is used to recognize cancer types with LacdiNAc expression according to the invention, more preferably ovarian cancer.

### Preferred Terminal GlcNAcβBinding Antibodies

Antibodies suitable for terminal GlcNAcβ recognition according to the invention include preferably clone HGAC85, recognizing β-linked GlcNAc in oligo/polysaccharides and in O-GlcNAc (Nahm, M. H., et al., 1982. J. Immunol. 129, 1513-1518).

### Preferred Lex/sLex Antibody Binders

The inventors found that specific cell types carry Lex/sLex epitopes on different glycan backbones according to the invention. The invention is specifically directed to use of one or more of listed antibodies (Table 21) for structure-specific recognition of Lex/sLex epitopes in different cell types and on different glycan backbones.

In preferred embodiment a core two slex antibody is used, more preferably specific for larger epitope according to the Formulas, in another preferred embodiment another preferred embodiment sLexβ3Gal epitope is recognized. The list is ordered according to preferred glycan backbone specificities. Suitable binders against Lex and/or sLex on each backbone can be selected according to the present invention.

### LacdiNAc-Expressing Tumor Metastasis

The inventors found that novel LacdiNAc (GalNAcβ4GlcNAc and fucosylated/sialylated variants thereof according to the invention)-carrying glycans are expressed in malignancy-specific manner in human tumors, especially ovarian cancer tumors. Further, the inventors found suitable antibodies for detecting these specific glycan groups. It is realized that such binding reagents are useful to detection, binding to, and imaging of malignant tumors and metastases in animals. Relevant detection and imaging procedures suitable for cancer cell binding reagents, preferably antibodies, are known to a person skilled in the art.

The present invention is especially directed to detecting cancer, tumors, and metastases by use of antibodies and other binding reagents recognizing present cancer antigens/target glycans, in a preferred embodiment from metastasis, more preferably antibodies recognizing LacdiNAc structures. The invention is further directed to imaging ovarian tumors in living animals, preferably human patients. In a further preferred embodiment, the invention is directed to detection of cancer, preferably metastasis, more preferably ovarian tumor metastases, preferably by in vivo imaging.

### Example 1

### Structure Analysis of Glycans that are Expressed in Various Human Cancer Types

### Experimental Procedures

Isolation of glycans from formalin-fixed and paraffin-embedded tissue samples. Prior to glycan isolation from formalin-fixed and paraffin-embedded samples, the samples were deparaffinised. Glycans were detached from sample glycoproteins by non-reductive β-elimination essentially as described previously (Huang et al., 2001) and purified and analyzed essentially as described in Examples 11 and 12.

MALDI-TOF MS. MALDI-TOF mass spectrometry was performed with a Voyager-DE STR BioSpectrometry Workstation, essentially as described previously (Saarinen et al., 1999; Harvey et al., 1993).

### 1.1 Neutral Low-Mannose Type N-Glycans

Exoglycosidase digestions. All exoglycosidase reactions were performed essentially as described previously (Nyman et al, 1998; Saarinen et al, 1999) and analysed by MALDI-TOF MS. The enzymes and their specific control reactions with characterised oligosaccharides were (R denotes reducing end oligosaccharide sequences in the following examples): β1,4-galactosidase (Streptococcus pneumoniae, recombinant, E. coli; Calbiochem, USA) digested Galβ1-4GlcNAc-R but not Galβ1-3GlcNAc-R; β-N-acetylglucosaminidase (Streptococcus pneumoniae, recombinant, E. coli; Calbiochem, USA) digested GlcNAcβ-6Gal-R in β1,4-galactosidase treated lacto-N-hexaose but not GalNAcβ1-4GlcNAcβ1-3/6Gal-R in a synthetic oligosaccharide; α-mannosidase (Jack bean; Glyko, UK) transformed a mixture of high-mannose N-glycans to the Man, GlcNAc₂ N-glycan core trisaccharide; β-mannosidase (Helix pomatia; Calbiochem, USA) digested the β1,4-linked mannose residue from the N-glycan core trisaccharide Manβ4GlcNAcβ4GlcNAc, without affecting the α-linked mannose residues of high-mannose N-glycans. Control digestions were performed in parallel and analysed similarly to the analytical exoglycosidase reactions. Endoglycosidase digestions were performed essentially as described previously (Plummer & Tarentino, 1991), and the reaction products were analyzed by MALDI-TOF MS after purification. The specific N-glycosidase F1 control oligosaccharides and the control reactions were as follows: the enzyme transformed all Hex₅₋₉HexNAc₂ oligosaccharides in a sample of high-mannose N-glycans into Hex₅₋₉HexNAc, oligosaccharides; in contrast, the enzyme was not able to digest a core-fucosylated N-glycan, namely the hexasaccharide

### Manα6(Manα3)Manβ4GlcNAcβ4(Fucα6)GlcNAc.

### Results

Glycan isolation and analysis. Detached and purified glycans from paraffin-embedded formalin-fixed tissue samples from cancer patients were analysed by MALDI-TOF mass spectrometry after isolation of the neutral glycan fraction. Relative quantification of the glycans were done by comparing relative MALDI-TOF MS signal intensities, which is accurate for the obtained mixtures of purified glycans (Saarinen et al., 1999; Harvey et al., 1993).

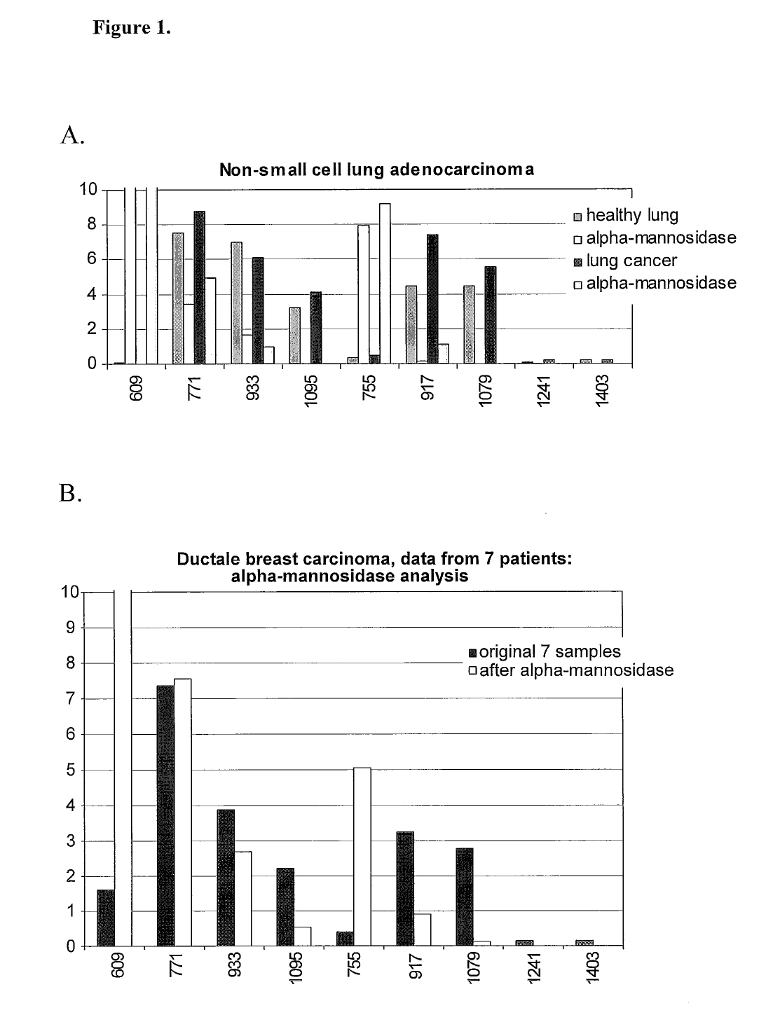

Specific mannosidase digestion analyses. The proportions of the non-reducing terminal α-mannose containing glycans were determined by their sensitivity towards hydrolysis with α-mannosidase from Jack beans. After the specific exoglycosidase digestion, the presence of high-mannose and low-mannose type glycans in the original sample can be deduced by their disappearance from the recorded mass spectra, and the simultaneous increase in signal intensities of the expected reaction products at m/z 609 and 755, which correspond to the sodium adduct ions [Hex₁HexNAc₂+Na]⁺ (calc. m/z 609.21) and [Hex₁HexNAc₂dHex₁+Na]⁺ (calc. m/z 755.27), respectively. An example of the reaction scheme is presented in FIG. 1. The results are summarized in Table 1. According to the digestion results, the majority of the detectable signals in the original samples with proposed compositions Hex₄₋₉HexNAc₂ and Hex₃₋₅HexNAc₂dHex₁, correspond to glycans that are sensitive to α-mannosidase and contain non-reducing terminal α-mannose residues, whereas signals with proposed compositions of Hex₂₋₃HexNAc₂ and Hex₂HexNAc₂dHex, most likely partially correspond to other glycan types, although they also contain variable amounts of non-reducing terminal α-mannose residues.

The Hex₁HexNAc₂dHex₀₋₁ components were studied with specific β-mannosidase digestion both before and after α-mannosidase digestion. The results are summarized in Table 1. The results indicate that 1) the original components contain variable amounts of non-reducing terminal 13-mannose containing oligosaccharides with the compositions Hex₁HexNAc₂dHex₀₋₁, and 2) the major α-mannosidase digestion products with the compositions Hex₁HexNAc₂dHex₀₋₁ have non-reducing terminal β-mannose residues, as they are susceptible towards digestion with β-mannosidase enzyme.

N-glycosidase digestion analyses. The assignment of the majority of the Hex₁HexNAc₂ and Hex₁HexNAc₂dHex₀₋₁ glycan components as low-mannose and high-mannose type N-glycans was confirmed by their isolation and digestion analysis by specific endoglycosidase enzymes, namely N-glycosidase F and N-glycosidase F1 from Chryseobacterium meningosepticum. The first series of experiments was done with glycan samples isolated from a lung tumor of a patient with non-small cell lung adenocarcinoma. In addition to chemical detachment, the glycans in question could also be isolated by N-glycosidase F digestion, indicating that they are N-glycans. However, all Hex₁₋₉HexNAc₂ components, but not any of the Hex₁₋₅HexNAc₂dHex₁ components, could be digested with N-glycosidase F1, resulting in transformation of the first glycan group into peaks with masses of one less HexNAc residue with monosaccharide compositions Hex₁₋₉HexNAc₁. In combination with the α- and β-mannosidase digestion results, these experiments indicate that all the components are N-glycans that have the chitobiose disaccharide sequence in their reducing end, and that the latter components have a dHex residue linked to the reducing terminal GlcNAc. Furthermore, as the latter components are susceptible to digestion with N-glycosidase F, they must have a reducing terminal sequence dHex-6(GlcNAcβ1-4)GlcNAc.

Chemical analyses. In individual samples, the Hex₁₋₉HexNAc₂ and Hex₁₋₅HexNAc₂dHex₁ components were also studied with periodate oxidation, subsequent reduction with alkaline sodium borohydride, and MALDI-TOF mass spectrometry. Also post-source decay (PSD) MALDI-TOF mass spectrometric analyses were performed to specific components of the structure group. The results from these analyses support the structural features deduced from the experiments described above. Periodate reaction cleaves structures with vicinal hydroxyl groups including non-reducing terminal monosaccharides, and isomeric structures differ from each other in this reaction. The data is in accordance with low-mannose and high-mannose type N-glycans produced by regular biosynthesis.

More specifically, cancer cell N-glycans with the compositions Hex₂HexNAc₂ and Hex₂HexNAc₂dHex₁, were studied with periodate oxidation, subsequent borohydride reduction, and MALDI-TOF MS. From the Hex₂HexNAc₂ component at m/z 771.59 (calc. m/z 771.26 for the ion [Hex₂HexNAc₂+Na]⁺), two product ions were observed after the reaction at m/z 717.34 (calc. m/z 717.29 for the ion [Hex₂HexNAc₂-C₂O₂+H₂+Na]⁺) and 745.34 (calc. m/z 745.29 for the ion [Hex₂HexNAc₂-C₁O₁+H₂+Na]⁺), which can result from Manα6Manβ4GlcNAcβ4GlcNAc and Manα3Manβ4GlcNAcβ4GlcNAc N-glycan oligosaccharide isomers, respectively. These products occurred in respective relative amounts of approximately 60% and 40%, indicating that the original sample contained nearly equal amounts of the both α-mannose linkage isomers. From the Hex₂HexNAc₂dHex₁ component at m/z 917.63 (calc. m/z 917.32 for the ion [Hex₂HexNAc₂dHex₁+Na]⁺), two product ions were observed after the reaction at m/z 835.41 (calc. m/z 835.35 for the ion [Hex₂HexNAc₂dHex₁-C₃O₃+H₂+Na]⁺) and 863.42 (calc. m/z 863.35 for the ion [Hex₂HexNAc₂dHex₁-C₂O₂+H₂+Na]⁺), which can result from Manα6Manβ4GlcNAcβ4(Fucα6)GlcNAc and Manα3Manα4GlcNAcβ4(Fucα6)GlcNAc N-glycan oligosaccharide isomers, respectively. These components occurred in relative amounts of approximately 80% and 20%, respectively, indicating that the original sample contained significantly more of the isomer containing α6-linked mannose, but that the both isomers were present in the sample.

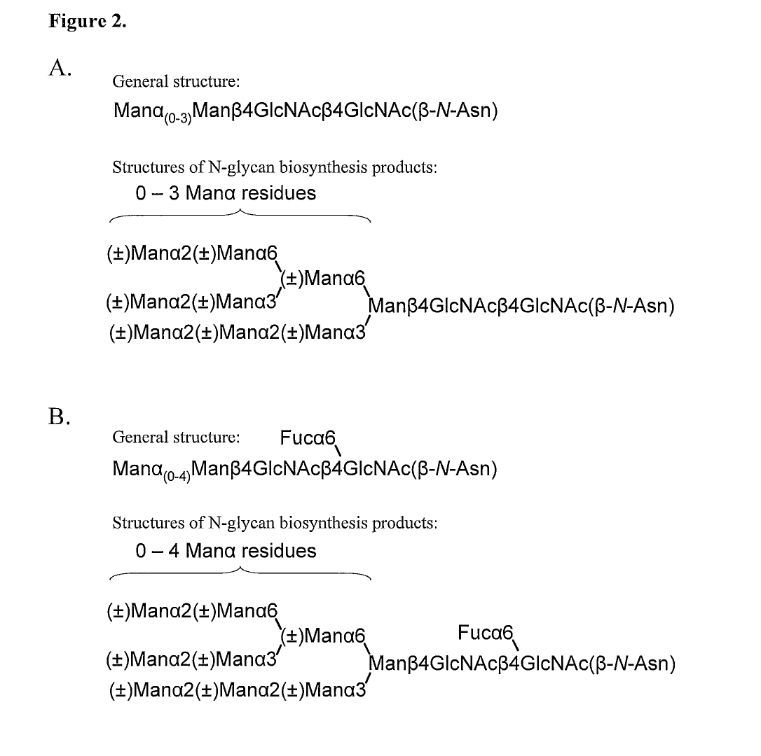

Taken together, all the experiments suggest that the terminal mannose-containing oligosaccharides, which have monosaccharide compositions Hex₁₋₉HexNAc₂ and Hex₁₋₅HexNAc₂dHex₁, include the structures presented in FIG. 2.

### 1.2 Neutral O-Glycans

Exoglycosidase digestions. All exoglycosidase reactions were performed essentially as described previously (Nyman et al., 1998; Saarinen et al., 1999) and analysed by MALDI-TOF MS. The enzymes and their specific control reactions with characterised oligosaccharides were (R denotes reducing end oligosaccharide sequences in the following examples): β1,4-galactosidase (Streptococcus pneumoniae, recombinant, E. coli; Calbiochem, USA) digested Galβ1-4GlcNAc-R but not Galβ1-3GlcNAc-R; α-mannosidase (Jack bean; Glyko, UK) transformed a mixture of high-mannose N-glycans to the Man, GlcNAc₂ N-glycan core trisaccharide; recombinant β1,3-galactosidase (Calbiochem, USA) digested Galβ3GlcNAc-R but not Galβ34GlcNAc-R; α3/4-fucosidase (Xanthomonas sp.; Calbiochem, USA) digested Galβ4(Fucα3)GlcNAcβ3Galβ4Glc but not Fucα2Galβ3GlcNAcβ3(Galβ4GlcNAcβ6)Galβ4Glc. Control digestions were performed in parallel and analysed similarly to the analytical exoglycosidase reactions.

### Results

Glycan isolation and analysis. Detached and purified glycans from paraffin-embedded formalin-fixed tissue samples from cancer patients were analysed by MALDI-TOF mass spectrometry after isolation of the neutral glycan fraction. Relative quantification of the glycans were done by comparing relative MALDI-TOF MS signal intensities, which is accurate for the obtained mixtures of purified glycans (Saarinen et al., 1999; Harvey et al., 1993).

Specific mannosidase digestion analyses. The proportions of the non-reducing terminal α-mannose containing glycans in the samples were determined by their sensitivity towards hydrolysis with α-mannosidase from Jack beans. After the specific exoglycosidase digestion, the presence of terminal α-mannose residues containing glycans in the original samples could be deduced by their disappearance from the recorded mass spectra, and the simultaneous increase in signal intensities of the expected reaction products at m/z 609 and 755, which correspond to the sodium adduct ions [Hex₁HexNAc₂+Na]⁺ (calc. m/z 609.21) and [Hex₁HexNAc₂dHex₁+Na]⁺ (calc. m/z 755.27), respectively. The glycans that resisted digestion with mannosidases, were studied further.

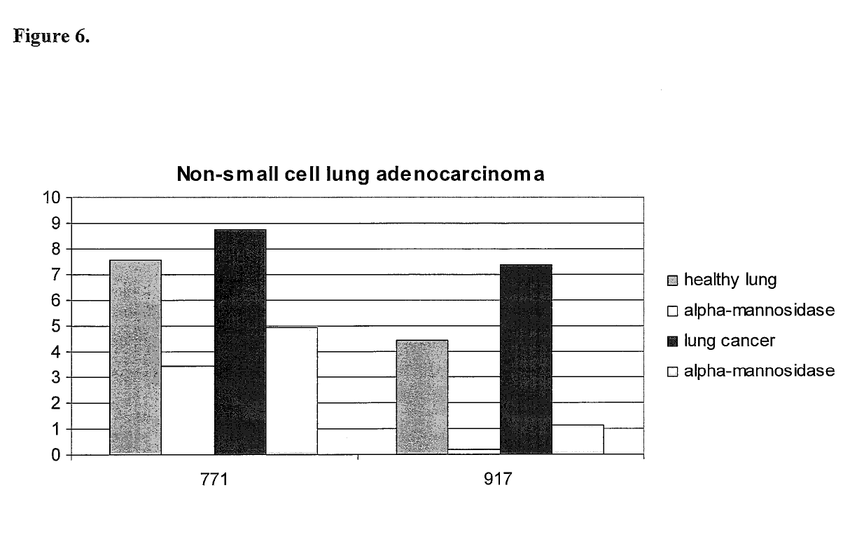

Specific galactosidase and fucosidase digestion analyses. The structural features of the mannosidase-resistant glycans in the samples were investigated by digestion with S. pneumoniae β1,4-galactosidase, recombinant β1,3-galactosidase, and α3/4-fucosidase. An example of the reaction scheme is presented in FIG. 6 and the results are summarized in Table 3. According to the digestion results, a significant proportion of signals in the original samples, with proposed compositions Hex₂HexNAc₂ and Hex₂HexNAc₂dHex₁, correspond to glycans that contain non-reducing terminal β1,4-linked galactose residues and/or α1,3-fucose residues, respectively.

N-glycosidase digestion analyses. The assignment of the majority of the Hex₁₋₉HexNAc₂ and Hex₁₋₅HexNAc₂dHex₁ glycan components as low-mannose and high-mannose type N-glycans was confirmed by their isolation and digestion analysis by a specific endoglycosidase enzyme, namely N-glycosidase F from Chryseobacterium meningosepticum. In combination with the α- and β-mannosidase digestion results, these experiments indicate that the mannosidase-resistant components at m/z 771 and 917, contain glycan species that are not N-glycans.

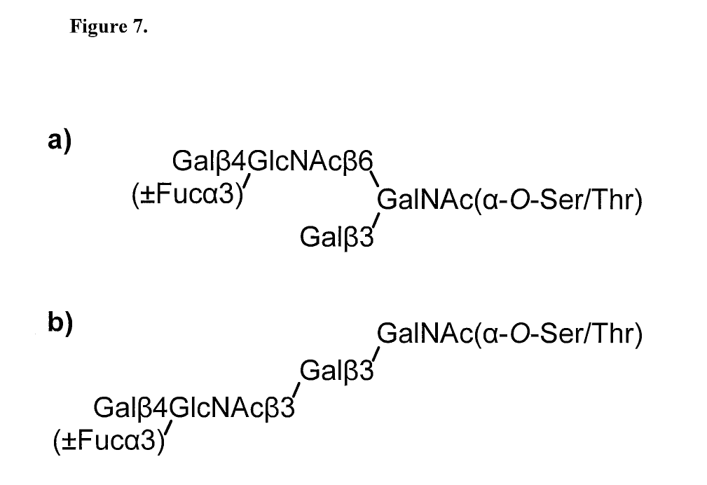

The presence of a glycan fragment at m/z 608 in the tissue samples corresponds to a structure, in which a Hex₁HexNAc₁ unit is linked to the 6-position of an O-glycan core GalNAc fragment. The presence of the m/z 608 peaks in the tissue samples indicates that part of the O-glycan structures may contain the Core 2 O-glycan structure. However, Core 1 O-glycan structures may also be present in the samples. Specific β1,3-galactosidase experiments can be used to reveal the relative proportions of these structures in the samples. Taken together, all the experiments suggest that the mannosidase-resistant oligosaccharides that have monosaccharide compositions Hex₂HexNAc₂ and Hex₂HexNAc₂dHex₁, include the structures presented in FIG. 7.

### 1.3 Sialylated Core 2 Type O-Glycans

Exoglycosidase digestions. All exoglycosidase reactions were performed essentially as described previously (Saarinen et al., 1999) and analysed by MALDI-TOF MS. The enzymes and their specific control reactions with characterised oligosaccharides were (R denotes reducing end oligosaccharide sequences in the following examples): β1,4-galactosidase (Streptococcus pneumoniae, recombinant, E. coli; Calbiochem, USA) digested Galβ4GlcNAc-R but not Galβ3GlcNAc-R; Arthrobacter ureafaciens neuraminidase (Calbiochem, USA) digested both Neu5Acα3Galβ4GlcNAc-R and Neu5Acα6Galβ4GlcNAc-R; Streptococcus pneumoniae α2,3-sialidase (Calbiochem, USA) digested Neu5Acα3Galβ4GlcNAc-R but not Neu5Acα6Galβ4GlcNAc-R. Control digestions were performed in parallel and analysed similarly to the analytical exoglycosidase reactions.

Chemical modification reactions. Mild acid hydrolysis of sialic acid residues was performed with 50 mM trifluoroacetic acid in water at 60° C. for 5 hours. After the reaction, the acid was eliminated by evaporation. Mild periodate oxidation, alkaline reduction with borohydride, mild alkaline hydrolysis for cleavage of carboxylic acid esters, and permethylation for fragmentation analyses were performed essentially as described previously (Ylönen et al., 2001). Methylation with iodomethane was performed essentially as described previously (Powell & Harvey, 1996).

### Results

Glycan isolation and analysis. Detached and purified glycans from paraffin-embedded formalin-fixed tissue samples from cancer patients were analysed by MALDI-TOF mass spectrometry after isolation of the neutral glycan fraction. Relative quantification of the glycans were done by comparing relative MALDI-TOF MS signal intensities, which is accurate for the obtained mixtures of purified glycans (Papac et al., 1996; Harvey, 1993; Saarinen et al., 1999).

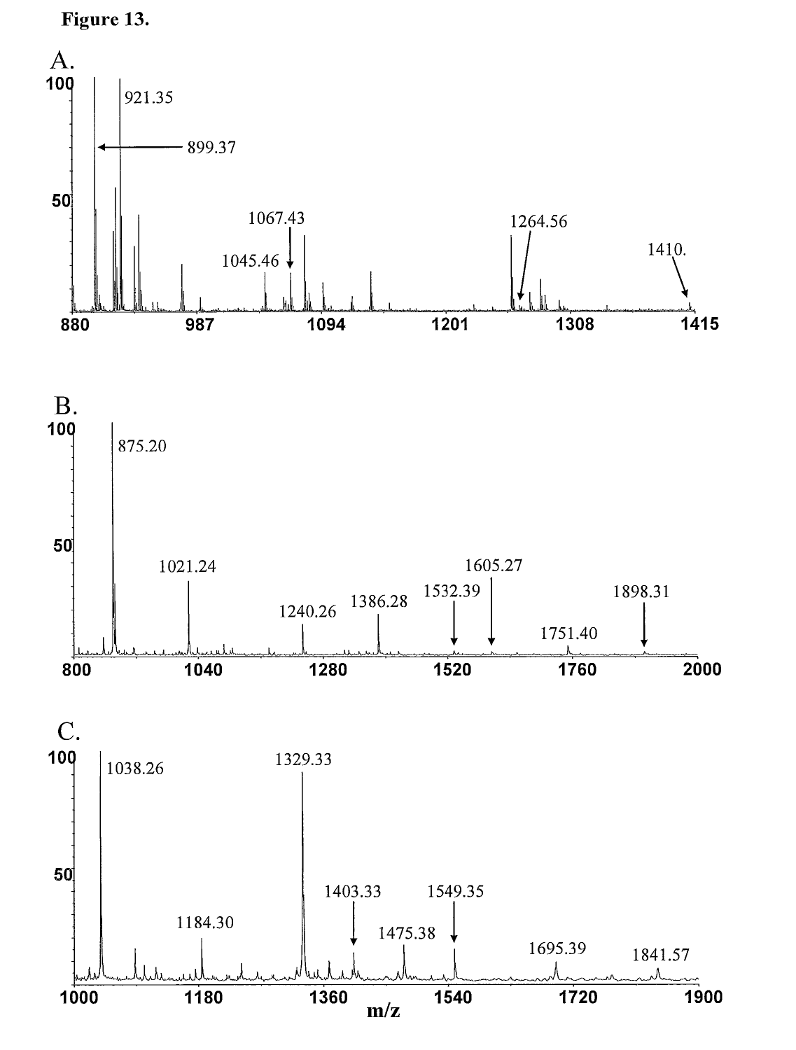

Indicative mass spectrometric signals of the structure group. The indicative mass spectrometric signals of the structure group, in both positive and negative ion mode MALDI-TOF MS, are presented in Table 5. Examples of the glycan antigen signals present in lung cancer tumor samples from a patient with non-small cell lung adenocarcinoma, are presented in FIG. 13.

General features of the structure group. The indicative glycan signals of the structure group in the non-sialylated glycan fraction, include O-glycan fragments that share in common the presence of an unusual reducing end terminal monosaccharide (2-acetamido-3-amino-2,3-dideoxyhexose, or deoxyamino-HexNAc, in which hexose is either D-galactose, D-gulose, D-allose, or D-glucose), which results from the strong alkaline conditions in the glycan isolation method, as discussed below. The major structure present at m/z 899 in various glycan samples from cancer patients is presented in FIG. 11, together with the principal biochemical evidence that supports the proposed structure. The more complex components in the structure group contain 1-2 additional HexHexNAc units and/or 1-2 dHex units, forming either linear or branched structures.

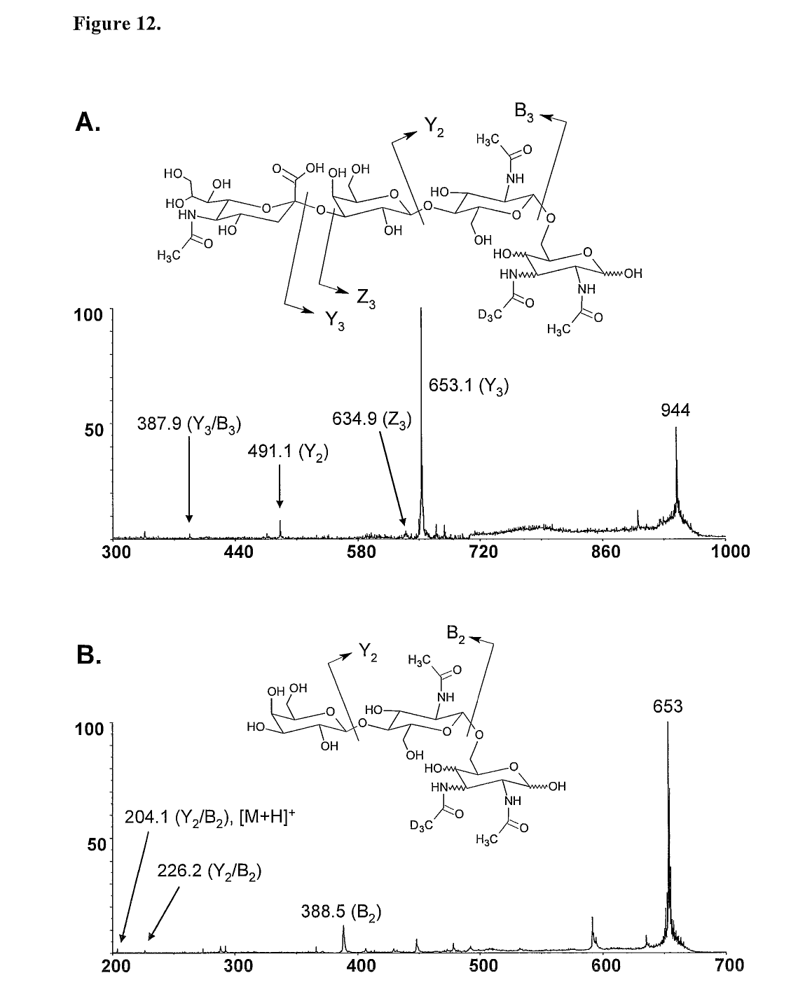

Nature of the acidic group. The acidic group of the m/z 899 peak was recognized as N-acetylneuraminic acid, based on the following experiments. Mild acid hydrolysis destroyed the m/z 899 peak without affecting the other peaks in the profile. Simultaneously, this resulted in increase of the signal at m/z 608, which has 291 mass units smaller m/z value, corresponding to a mass difference of an acetylneuraminic acid residue. Mild periodic acid oxidation and subsequent borohydride reduction, resulted in the destroying of the m/z 899 peak. However, the m/z 608 peak together with the neutral glycan peaks in the glycan profile, were not affected by the periodic acid treatment. This corresponds to a cleavage between the C7 and C8 carbons of the glycerol tail, namely removal of 60 mass units (C₂H₄O₂) from a neuraminic acid residue, and addition of 2 mass units (due to reduction of the reducing end of the oligosaccharide). The cleavage site was further shown to reside in the acid-labile sialic acid residue, because the neutral fragment at m/z 608 was not affected by mild periodate, but was instead reduced and transformed into m/z 610 during the reaction. Furthermore, both Arthrobacter ureafaciens neuraminidase and recombinant Streptococcus pneumoniae α2,3-sialidase hydrolyzed the m/z 899 peak from the spectrum, and transformed it into the peak at m/z 608. This suggests that the sialic acid linkage is α2→3, and not either α2→6, α2→8, or α2→9, to the next monosaccharide residue in the sequence. In addition, cleavage of a 291 mass unit fragment was shown to be the major cleavage route of the m/z 899 glycan peak, in post-source decay (PSD) MIALDI-TOF mass spectrometric fragmentation experiments (FIG. 12). The identification of the sialic acid residue as N-acetylneuraminic acid is based on the strongly alkaline conditions of the glycan isolation procedure. In these conditions, any O-acetyl groups would have been removed from the glycans.

Oligosaccharide sequence of the m/z 899 glycan. After removal of the sialic acid residue, the oligosaccharide sequence of the remaining glycan at m/z 608 was studied by specific exoglycosidases. Streptococcus pneumoniae β1,4-galactosidase, but not a recombinant β1,3-galactosidase, transformed the m/z 608 peak into a peak at m/z 446, corresponding to the removal of one hexose residue. Together with the known specificity of the enzymes and the general biosynthetic routes of human O-glycan structures (Brockhausen, 1999), this suggests that the major component at m/z 899 in the glycan profiles contains the non-reducing terminal oligosaccharide sequence NeuNAcα2-3Galβ1-4GlcNAcβ-R, where R is the reducing end component of 243 mass units that corresponds to the sodium adduct ion of acetamido-amino-dideoxyhexose [C₈H₁₆N₂O₅+Na]⁺.

Nature of the reducing terminal monosaccharide. The m/z 899 glycan had an intact reducing terminal, as evidenced by the transformation of the peak into a peak at m/z 901 upon reduction with alkaline sodium borohydride. The reducing terminal monosaccharide also contained a free primary amino group, as evidenced by the following experiments. Upon N-acetylation by acetic anhydride, the glycan peak was transformed into a peak at m/z 941, corresponding to an addition of 42 mass units, typical for acetylation. The +42 Da modification was resistant to mild alkaline hydrolysis, which is in accordance with the suggested acetamido linkage. Furthermore, the glycan peaks at m/z 899 and m/z 608 are usually accompanied by peaks with 22 mass units lesser mass in the mass spectra of the present experiments, namely at m/z 877 and m/z 586 respectively, corresponding to proton adduct ions of the same molecule ([M+H]⁺). In contrast, neither the normal neutral oligosaccharides present in the same glycan profile, nor the acetylated counterparts of the same glycan peaks have the accompanying proton adduct peaks. This suggests that the non-acetylated glycans at m/z 899 and m/z 608 have an unusual basic functional group, which is in accordance with the presence of the suggested free primary amine group. The reducing terminal monosaccharide could also be efficiently methylated by iodomethane in alkaline dimethylsulfoxide. In this reaction, the parent glycan peak at m/z 899 was transformed into a peak at m/z 933 for [M+C₄H₉]⁺, corresponding to the formation of a quarternary amine group and a molecular ion, and with the sialic acid residue transformed into a methyl ester. A carboxylic acid methyl ester is alkali-labile, and accordingly, upon mild alkali hydrolysis, this group was transformed into a free carboxylic acid. The resulting ion that corresponds to the formation of a quaternary amine strongly suggests for the presence of a primary amine in the original molecule.

The formation of the m/z 899 fragment was further studied by using bovine fetuin as a model glycoprotein. Fetuin contains both Core 1 and Core 2 branched O-glycans with structures NeuNAcα2-3Galβ1-3GalNAc(α-O-Ser/Thr), NeuNAcα2-3Gal 1-3(NeuNAcα2-6)GalNAc(α-O-Ser/Thr), and NeuNAcα2-3Galβ1-4GlcNAcβ1-6([±NeuNAcα2-3]Galβ1-3)GalNAc(α-O-Ser/Thr), respectively. The non-reductive β-elimination glycan isolation procedure that was used in glycan isolation from cancer patient tissues, produced abundant glycans at m/z 899. This glycan fragment was similar in its biochemical properties to its counterpart in human tissues. The only parent molecules available in the fetuin glycoprotein for the formation of the m/z 899 fragment, are the O-glycans that contain the substructure NeuNAcα2-3Galβ1-4GlcNAβ1-6(R-3)GalNAc(α-O-Ser/Thr), where R are [±NeuNAcα2-3]Gal(p).

Based on known susceptibility of the 3-position substituent of GalNAc to β-emulation in alkaline conditions, the structure of the m/z 899 glycan peak could be assigned as arising from elimination of the 3-substituent from the O-glycan, and subsequent addition of ammonia into the unsaturated glycan ring, which forms a primary amine functional group into the reducing end monosaccharide that arises from the GalNAc residue. Furthermore, as the fragmentation starts with elimination from the 3-position of GalNAc, the amine modification will reside in the 3-position of the monosaccharide. In conclusion, the evidence suggests that the reducing terminal monosaccharide is 2-acetamido-3-amino-2,3-dideoxyhexose, to which the rest of the glycan sequence is attached at 6-position. The sequence of the major oligosaccharide present at m/z 899 is therefore NeuNAcα2-3Galβ1-4GlcNAcβ1-6(2-acetamido-3-amino-2,3-dideoxy)hexose. As the fragment formation starts from N-acetylgalactosamine, the most likely hexose isomers in the fragment are D-galactose, D-gulose, D-allose, and D-glucose.

Mass spectrometric fragmentation analyses. The fragments obtained in post-source decay MALDI-TOF mass spectrometry, from native glycan peaks at m/z 899 and m/z 608, and their acetylated as well as deuteroacetylated forms, showed the presence and sequence of the acetylneuraminic acid, hexose, and N-acetylhexosamine residues in the m/z 899 glycan, thus confirming the structural features described above (FIG. 12).

Analysis of sialylated glycans. In negative ion mode MALDI-TOF MS of the isolated sialylated glycan fraction of lung tumor and healthy control tissues, more specifically patients with non-small cell lung adenocarcinoma and ovarian cystadenocarcinoma, when tumor samples were compared to the corresponding healthy lung and ovary samples, respectively, sialylated glycan peaks were elevated at m/z 1038, corresponding to NeuNAc₁Hex₂HexNAc₂ (calc. m/z 1038.36 for the ion [M−H]⁻), at m/z 1329, corresponding to NeuNAc₁Hex₂HexNAc₂ (calc. m/z 1329.46 for the ion [M−H]⁻), and at m/z 1475, corresponding to NeuNAc₂Hex₂HexNAc₂dHex₁ (calc. m/z 1475.52 for the ion [M−H]⁻). This indicates that these glycan components are major parent glycans from which originates the m/z 899 glycan peak present in the positive ion mode mass spectra.

In contrast, sialylated glycan peaks were decreased at m/z 673, corresponding to NeuNAc₁Hex₁HexNAc₁ (calc. m/z 673.23 for the ion [M−H]⁻), and at m/z 964, corresponding to NeuNAc₂Hex₁HexNAc₁ (calc. m/z 964.33 for the ion [M−H]⁻), when compared to the larger glycan peaks mentioned above. This indicates that the increase in the amounts of the larger glycans happens in conjunction with the decrease in the amounts of the smaller glycans at m/z 673 and m/z 964. Furthermore, this suggests a change from Core 1 type O-glycans to Core 2 type O-glycans associated with malignant tumor samples.

Furthermore, in the healthy control samples from the lung and the ovary, no detectable peaks were present at m/z at m/z 1081, corresponding to NeuNAc₁Hex₁HexNAc₃ (calc. m/z 1081 for the ion [M−H]⁻), at m/z 1370, corresponding to NeuNAc₂Hex₁HexNAc₃ (calc. m/z 1329.46 for the ion [M−H]⁻), or at m/z 1516, corresponding to NeuNAc₂Hex₁HexNAc₃dHex₁ (calc. m/z 1475.52 for the ion [M−H]⁻). This indicates that the major 3-position substituents of the m/z 899 component present in the positive ion mode mass spectra, may be either a hexose monosaccharide, or a neuraminic acid-hexose disaccharide, in the original sample.

Samples from benign tumors of the ovary, namely benign ovarian cystadenoma, were similar to the healthy ovary sample in respect of their specific glycan structures, indicating that the described changes in the relative amounts of the glycan peaks, reflect a change associated with malignant transformation of cancer, or at least ovarian adenocarcinoma.

### Example 2

### Expression of Glycans in Tissue Samples of Various Cancer Patients

### Experimental Procedures

Statistical calculations. Statistical analyses were performed with the SAS Software (SAS System, version 8.2, SAS Institute Inc., Cary, N.C., USA), using SAS/STAT and SAS/BASE modules. All tests were performed as two-sided. The distributions of the experimental data were evaluated as 1) normal and symmetric, 2) only symmetric, or 3) non-symmetric and not normal, and the statistical test used was accordingly chosen as 1) Student's t Test, 2) Wilcoxon Signed Rank Test, or 3) Sign Test. A p value of less than 0.05 was considered statistically significant.

### Results

### 2.1 Neutral Low-Mannose Type N-Glycans

Neutral low-mannose type N-glycans are more abundant in tumor tissue samples than in healthy control tissue samples from cancer patients. Formalin-fixed samples, from tumor and surrounding healthy tissue, were obtained from patients with various types of cancer. The studied cancer types included non-small cell lung adenocarcinoma, ductale breast carcinoma, lobulare breast carcinoma, stomach cancer, colon cancer, kidney cancer, ovarian carcinoma, pancreatic cancer, and cancers of the lymph nodes and the larynx. There were significant differences between the neutral low-mannose type N-glycans isolated from tumor samples and healthy tissue samples (Table 1), more specifically the Hex₂₋₄HexNAc₂ and Hex₂₋₅HexNAc₂dHex, neutral glycans, as described above. Neutral low-mannose type N-glycans were shown to be expressed in statistically significant manner in lung cancer and in two types of breast cancer (Table 2). In the examples below, it must be taken into account that at least m/z 609, 755, 771, and 917, glycan peaks may contain multiple oligosaccharide structures.

### 2.2 Neutral O-Glycans

The neutral O-glycans Hex₂HexNAc₂dHex₀₋₁ are more abundant in tumor tissue samples than in healthy control tissue samples from cancer patients. Formalin-fixed samples, from tumor and surrounding healthy tissue, were obtained from patients with various types of cancer. The studied cancer types included non-small cell lung adenocarcinoma, ductale breast carcinoma, lobulare breast carcinoma, stomach cancer, colon cancer, kidney cancer, ovarian carcinoma, pancreatic cancer, and cancers of the lymph nodes and the larynx. There were significant differences between the neutral O-glycans isolated from tumor samples and healthy tissue samples (Table 3), more specifically the Hex₂HexNAc₂ and Hex₂HexNAc₂dHex₁ neutral glycans, as described above. O-glycans were shown to be expressed in statistically significant manner in lung cancer and in two types of breast cancer (Table 4). In the examples below, it must be taken into account that the m/z 771 and 917 glycan peaks may contain multiple oligosaccharide structures. However, while the m/z 917 glycan peak contains significant amounts of mannosidase-sensitive glycans, the vast majority of the glycans in the glycan peak at m/z 771, corresponding to Hex₂HexNAc₂, are mannosidase-resistant.

### 2.3 m/z 899 Series Glycans

The 899 series glycans are more abundant in tumor tissue samples than in healthy control tissue samples from cancer patients. Formalin-fixed samples, from tumor and surrounding healthy tissue, were obtained from patients with various types of cancer. The studied cancer types included non-small cell lung adenocarcinoma, ductale breast carcinoma, lobulare breast carcinoma, stomach cancer, colon cancer, kidney cancer, ovarian carcinoma, pancreatic cancer, and cancers of the lymph nodes and the larynx. There were significant differences between the m/z 899 series glycans isolated from tumor samples and healthy tissue samples (FIG. 13), and the results are summarized in Table 6. The m/z 899 glycan fragment was shown to be expressed in statistically significant manner in comparison to normal tissue in populations of patients, in lung cancer and in two types of breast cancer (Table 7).

### Example 3

### Expression of Glycans in Lung Cancer Patients

### 3.1 Neutral Low-Mannose Type N-Glycans

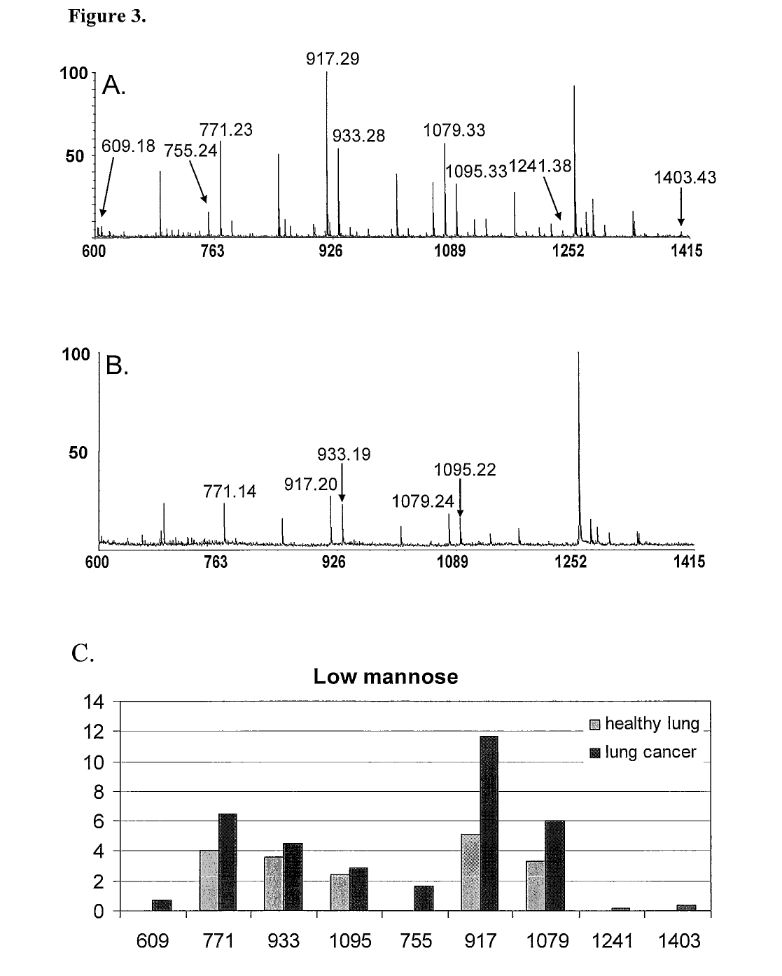

In a group of lung cancer patients, more specifically non-small cell lung adenocarcinoma, the glycan peaks at m/z 609, 755, 771, 917, 1079, 1095, 1241, and/or 1403 were expressed in significantly elevated amounts in the tissue samples from the tumor (Table 1), when compared to healthy control tissues from the same patients. These glycan peaks correspond to Hex₁HexNAc₂, Hex₁HexNAc₂dHex₁, Hex₂HexNAc₂, Hex₂HexNAc₂dHex₁, Hex₃HexNAc₂dHex₁, Hex4HexNAc₂, Hex4HexNAc₂dHex₁, and Hex₅HexNAc₂dHex₁ glycan epitopes, respectively. An example pair of mass spectra from a lung cancer patient is presented in FIG. 3.

### 3.2 Neutral O-Glycans

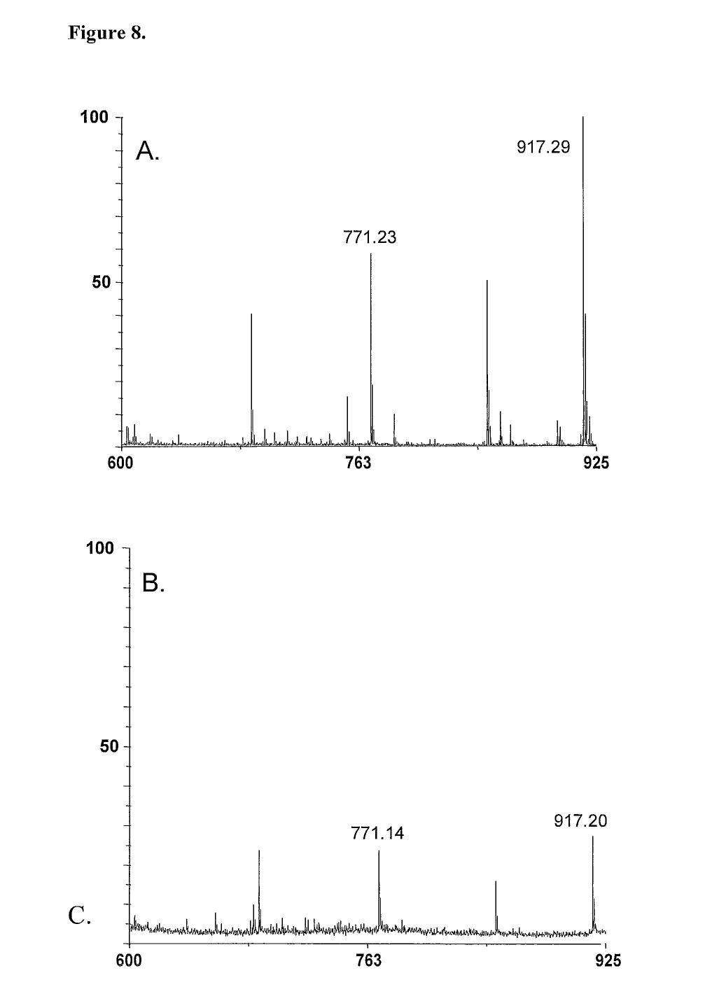

In a group of lung cancer patients, more specifically non-small cell lung adenocarcinoma, the glycan peaks at m/z 771 and 917 were expressed in significantly elevated amounts in the tissue samples from the tumor (Table 3), when compared to healthy control tissues from the same patients. These glycan peaks correspond to Hex₂HexNAc₂ and Hex₂HexNAc₂dHex, glycan epitopes, respectively. This difference was shown to be statistically significant (Table 4). As stated above, the glycan peak at m/z 771 is practically mannosidase-resistant, while the peak m/z 917 consists of multiple structures. An example pair of mass spectra from a lung cancer patient is presented in FIG. 8.

### 3.3 m/z 899 Series Glycans

In a group of lung cancer patients, more specifically non-small cell lung adenocarcinoma, the glycan peaks at m/z 899 and m/z 1045 were expressed in significantly elevated amounts in the tissue samples from the tumor (Table 6 and FIG. 13), when compared to healthy control tissues from the same patients. These glycan peaks correspond to NeuNAc₁Hex₁HexNAc₁ and NeuNAc₁Hex₁HexNAc₁dHex₁ glycan epitopes linked to the 6-position of the O-glycan core GalNAc residue, respectively, when the GalNAc had been originally substituted to the 3-position in the intact tissue. This difference was shown to be statistically significant (Table 7).

### Example 4

### Expression of Glycans in Ductale Breast Cancer Patients

### 4.1 Neutral Low-Mannose Type N-Glycans

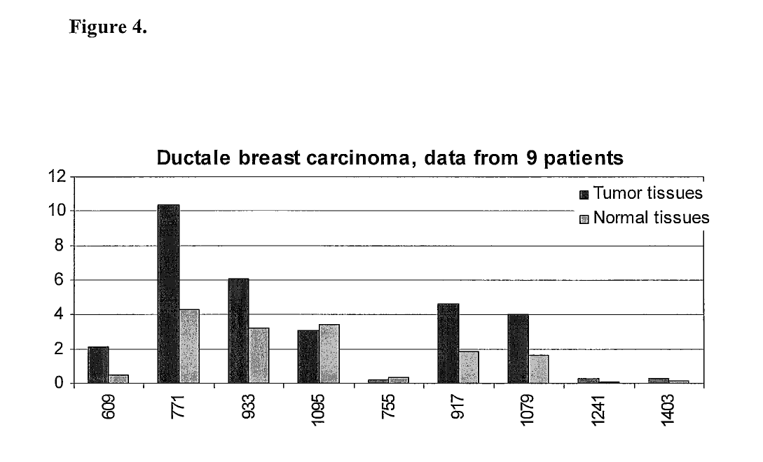

In a group of breast cancer patients, more specifically ductale breast carcinoma, the glycan peaks at m/z 609, 771, 917, 933, 1079, 1095, 1241, and/or 1403 were expressed in significantly elevated amounts in the tissue samples from the tumor (Table 1), when compared to healthy control tissues from the same patients. These glycan peaks correspond to Hex₁HexNAc₂, Hex₂HexNAc₂, Hex₂HexNAc₂dHex₁, Hex₃HexNAc₂, Hex₃HexNAc₂dHex₁, Hex4HexNAc₂, Hex4HexNAc₂dHex₁, and Hex₅HexNAc₂dHex₁ glycan epitopes, respectively. This difference was shown to be statistically significant (Table 2). An example pair of mass spectra from a ductale breast cancer patient is presented in FIG. 4.

### 4.2 Neutral O-Glycans

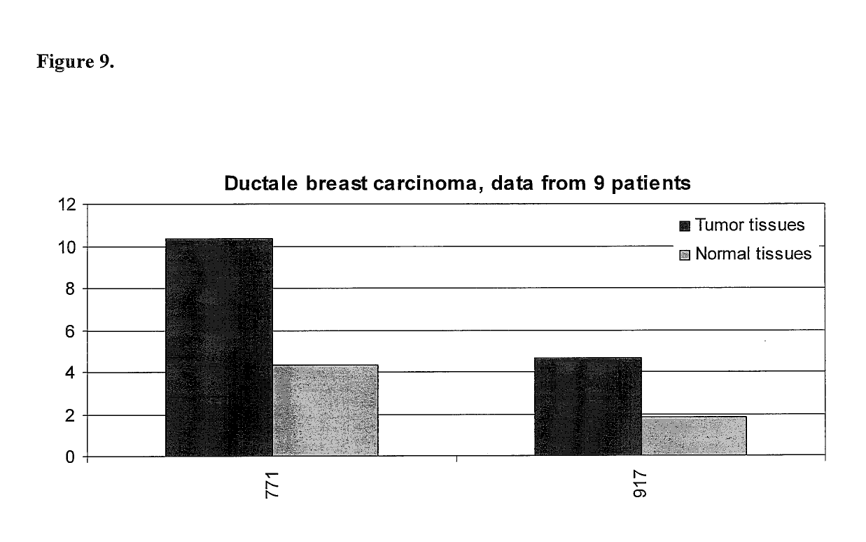

In a group of breast cancer patients, more specifically ductale breast carcinoma, the glycan peaks at m/z 771 and 917 were expressed in significantly elevated amounts in the tissue samples from the tumor (Table 3), when compared to healthy control tissues from the same patients. These glycan peaks correspond to Hex₂HexNAc₂ and Hex₂HexNAc₂dHex₁ glycan epitopes, respectively. This difference was shown to be statistically significant (Table 4). As stated above, the glycan peak at m/z 771 is practically mannosidase-resistant, while the peak m/z 917 consists of multiple structures. An example pair of mass spectra from a ductale breast cancer patient is presented in FIG. 9.

### 4.3. m/z 899 Series Glycans

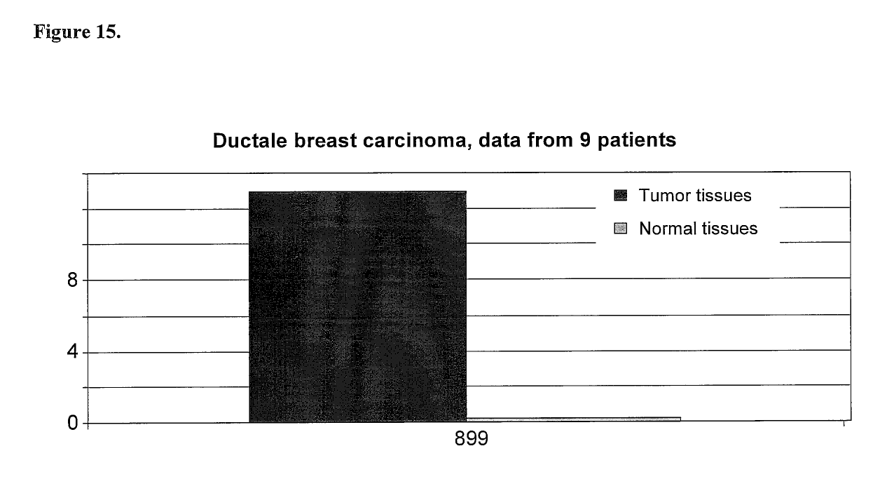

In a group of breast cancer patients, more specifically ductale breast carcinoma, the glycan peak at m/z 899 was expressed in significantly elevated amounts in the tissue samples from the tumor (Table 6), when compared to healthy control tissues from the same patients. This glycan peak corresponds to NeuNAc₁Hex₁HexNAc₁ glycan epitope linked to the 6-position of the O-glycan core GalNAc residue, when the GalNAc had been originally substituted to the 3-position in the intact tissue. This difference was shown to be statistically significant (Table 7). An example pair of mass spectra from a ductale breast cancer patient is presented in FIG. 15.

### Example 5

### Expression of Glycans in Lobulare Type Breast Cancer Patients

### 5.1 Neutral Low-Mannose Type N-Glycans

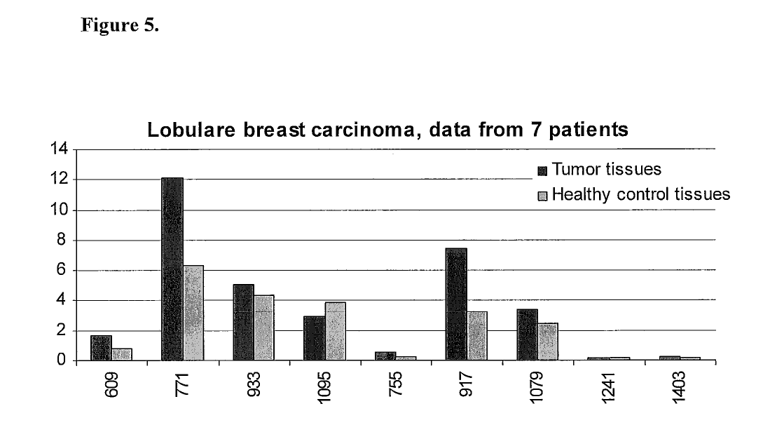

In a group of breast cancer patients, more specifically lobulare breast carcinoma, the glycan peaks at m/z 609, 755, 771, 917, 933, 1079, 1095, 1241, and/or and 1403 were expressed in significantly elevated amounts in the tissue samples from the tumor (Table 1), when compared to healthy control tissues from the same patients. These glycan peaks correspond to Hex₁HexNAc₂, Hex₁HexNAc₂dHex₁, Hex₂HexNAc₂, Hex₂HexNAc₂dHex₁, Hex₃HexNAc₂, Hex₃HexNAc₂dHex₁, Hex4HexNAc₂, Hex4HexNAc₂dHex₁, and Hex₅HexNAc₂dHex, glycan epitopes, respectively. An example pair of mass spectra from a lobulare breast cancer patient is presented in FIG. 5.

### 5.2 Neutral O-Glycans

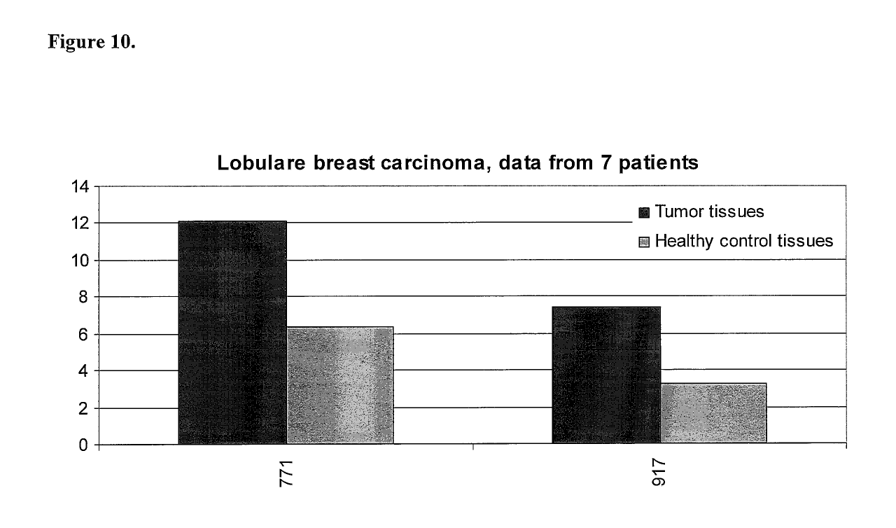

In a group of breast cancer patients, more specifically lobulare breast carcinoma, the glycan peaks at m/z 771 and 917 were expressed in significantly elevated amounts in the tissue samples from the tumor (Table 3), when compared to healthy control tissues from the same patients. These glycan peaks correspond to Hex2HexNAc2 and Hex₂HexNAc₂dHex, glycan epitopes, respectively. As stated above, the glycan peak at m/z 771 is practically mannosidase-resistant, while the peak m/z 917 consists of multiple structures. An example pair of mass spectra from a lobulare breast cancer patient is presented in FIG. 10.

### 5.3 m/z 899 Series Glycans

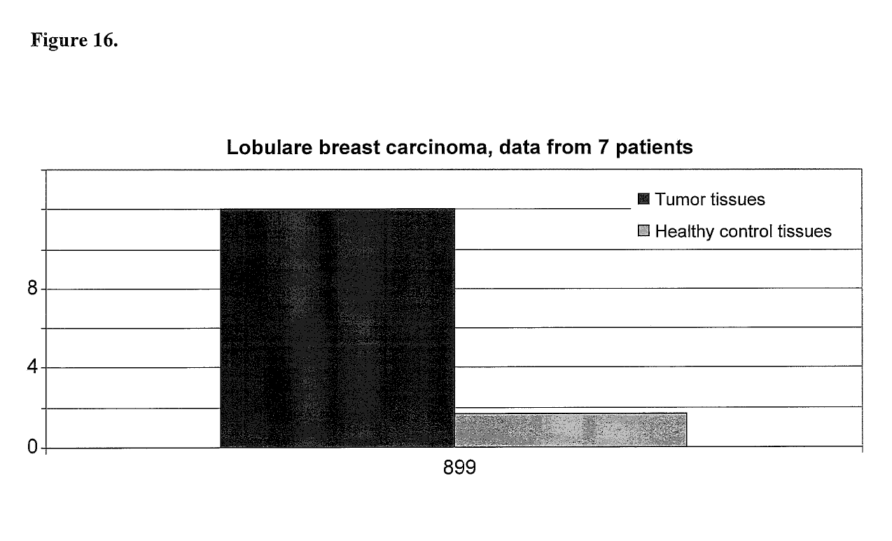

In a group of breast cancer patients, more specifically lobulare breast carcinoma, the glycan peak at m/z 899 was expressed in significantly elevated amounts in the tissue samples from the tumor (Table 6), when compared to healthy control tissues from the same patients. This glycan peak corresponds to NeuNAc₁Hex₁HexNAc₁ glycan epitope linked to the 6-position of the O-glycan core GalNAc residue, when the GalNAc had been originally substituted to the 3-position in the intact tissue. This difference was shown to be statistically significant (Table 7). An example pair of mass spectra from a lobulare breast cancer patient is presented in FIG. 16.

### Example 6

### Expression of m/z 899 Series Glycans in Patients with Malignant Ovarian Tumors

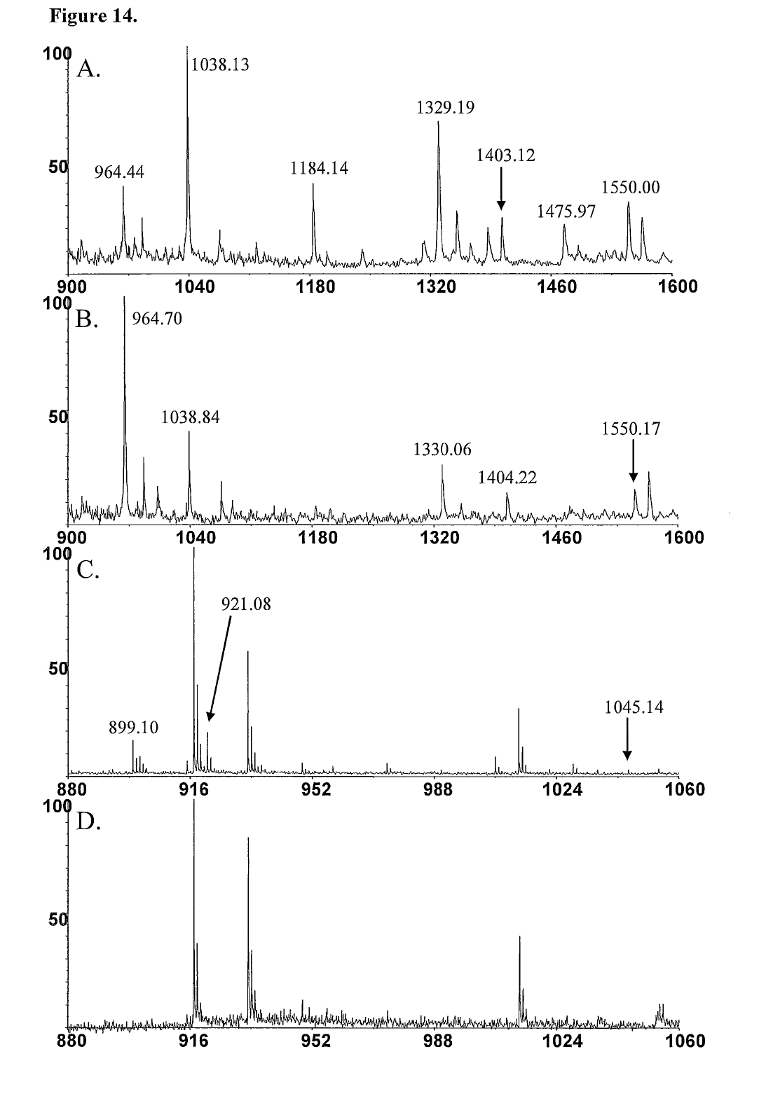

In a group of patients with ovarian tumors, more specifically malign ovarian cystadenocarcinoma or benign ovarian cystadenoma, the glycan peaks at m/z 899 and m/z 1045 were expressed in significantly elevated amounts in the tissue samples from the malignant tumors (Table 6 and FIG. 14), when compared to either the benign tumors or healthy control tissues from the same patients. These glycan peaks correspond to NeuNAc₁Hex₁HexNAc₁ and NeuNAc₁Hex₁HexNAc₁dHex₁ glycan epitopes linked to the 6-position of the O-glycan core GalNAc residue, respectively, when the GalNAc had been originally substituted to the 3-position in the intact tissue

### Example 7

### Expression of Glycans in Individual Patient Samples of Various Cancer Types

### 7.1 Neutral Low-Mannose Type N-Glycans

In addition to the statistically studied larger patient populations in lung and breast cancers, neutral low-mannose type N-glycans were expressed in many cancer types, when compared to healthy control tissues from the same patients. These results are summarized in Table 1.

7.2 Neutral O-Glycans

The neutral O-glycans at m/z 771 and 917 were expressed in many cancer types, when compared to healthy control tissues from the same patients. These results are summarized in Table 3.

### 7.3. m/z 899 Series Glycans

The m/z 899 series glycans were expressed in many cancer types, when compared to healthy control tissues from the same patients. These results are summarized in Table 6.

### Example 8

### Detection of Lung Tissue and Lung Tumor-Specific Glycan Structures

### Experimental Procedures

Tissue samples and glycan isolation. Archival paraffin-embedded and formalin-fixed tissue samples were from patients with non-small cell adenocarcinoma. After deparaffinisation, protein-linked glycans were detached from tissue sections with non-reductive alkaline β-elimination in concentrated ammonia-ammonium carbonate essentially as described previously (Huang et al., 2001). The isolated glycans were purified and divided into sialylated and non-sialylated glycan fractions as described in the other Examples of the present invention.

MALDI-TOF mass spectrometry. MALDI-TOF mass spectrometry was performed with a Voyager-DE STR BioSpectrometry Workstation, essentially as described previously (Saarinen et al., 1999; Harvey et al, 1993). Relative molar abundancies of both neutral (Naven & Harvey, 1996) and sialylated (Papac et al., 1996) glycan components were assigned based on their relative signal intensities.

### Results and Discussion

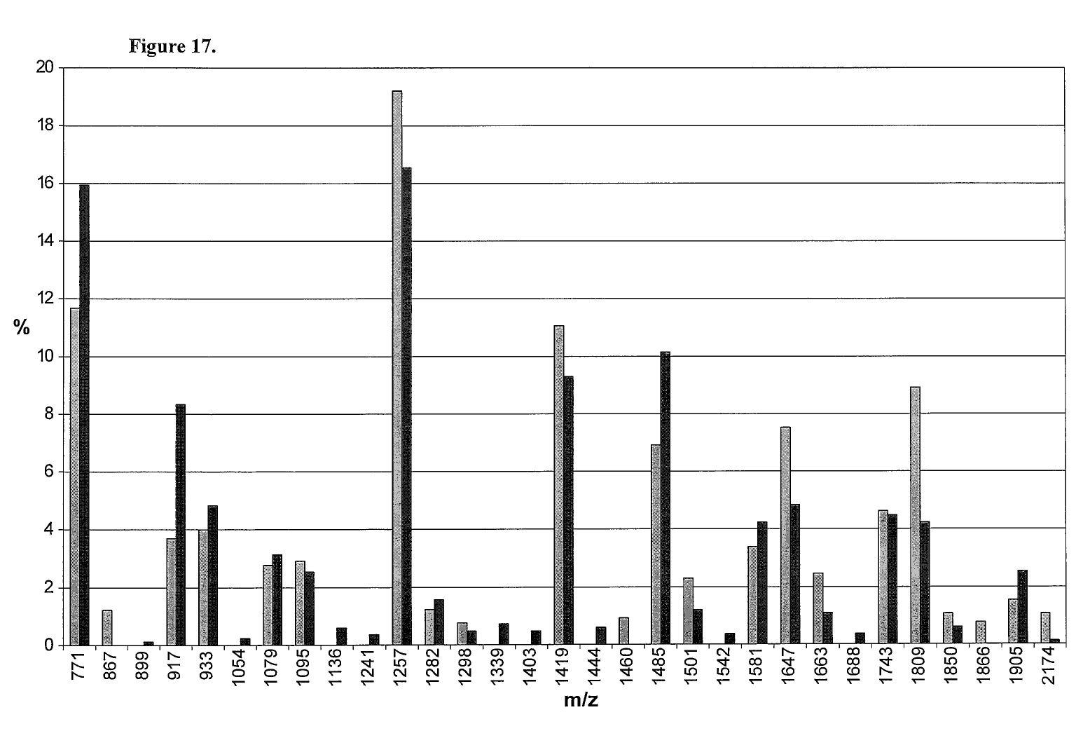

Occurrence of multiple cancer-associated protein-linked glycans in tissue glycan profiles. FIG. 17 shows the neutral glycan profiles averaged from multiple lung cancer samples. In the healthy tissue profiles, signals corresponding to neutral O-glycans (m/z 771, 917), sialylated O-glycans (m/z 899), and several signals among the low-mannose N-glycans (m/z 917, 1079, 1095, 1241, and 1403), are indicated as cancer-associated, as discussed in the preceding Examples. Also a profile change in the relative proportions of the signals at m/z 1485, 1647, and 1809 is visible: in the cancer samples the relative glycan abundancies are in the order 1485>1647>1809, whereas in the healthy samples the signals are approximately of the same abundance. The signals at m/z 1485 and 1647, corresponding to Hex₃HexNAc₄dHex₁ and Hex4HexNAc₄dHex₁, contain non-reducing terminal GlcNAcβresidues, as evidenced by S. pneumoniae β-glucosaminidase digestion. It is concluded that terminal GlcNAcβresidues are associated with lung cancer and also with the three other cancer-associated glycan groups discussed above. Furthermore, the present method can detect these and other potential cancer-associated glycosylation changes simultaneously, allowing for multiparameter cancer diagnostics.

### Example 9

### Detection of Ovarian Tissue and Ovarian Tumor-Specific Glycan Structures

### Experimental Procedures

Tissue samples and glycan isolation. Archival paraffin-embedded and formalin-fixed tissue samples were from patients with malignant ovarian cystadenocarcinoma or benign ovarian cystadenoma. After deparaffinisation, protein-linked glycans were detached from tissue sections with non-reductive alkaline β-elimination in concentrated ammonia-ammonium carbonate essentially as described previously (Huang et al., 2001). The isolated glycans were purified and divided into sialylated and non-sialylated glycan fractions as described in the other Examples of the present invention.

MALDI-TOF mass spectrometry. MALDI-TOF mass spectrometry was performed with a Voyager-DE STR BioSpectrometry Workstation, essentially as described previously (Saarinen et al, 1999; Harvey et al., 1993). Relative molar abundancies of both neutral (Naven & Harvey, 1996) and sialylated (Papac et al., 1996) glycan components were assigned based on their relative signal intensities. The mass spectrometric fragmentation analysis was done with the Voyager-DE STR BioSpectrometry Workstation according to manufacturer's instructions. For the fragmentation analysis, sialylated glycans were further purified by gel filtration HPLC and permethylated essentially as described previously (Nyman et al., 1998).

Exoglycosidase digestions. Digestions with A. ureafaciens neuraminidase (Glyko, UK), S. pneumoniae β-glucosaminidase (Calbiochem, USA), and Jack bean β-hexosaminidase (C. ensiformis; Calbiochem, USA) were performed essentially as described previously (Saarinen et al., 1999). The specificity of the two latter enzymes was controlled with synthetic oligosaccharides with terminal 1) Galβ1-4GlcNAcβ, 2) GlcNAcβ, and 3) GalNAcβ1-4GlcNAcβ epitopes: β-hexosaminidase digested the HexNAc residues in 2) and 3), but not 1), and β-glucosaminidase digested 2) but not 1) or 3).

### Results

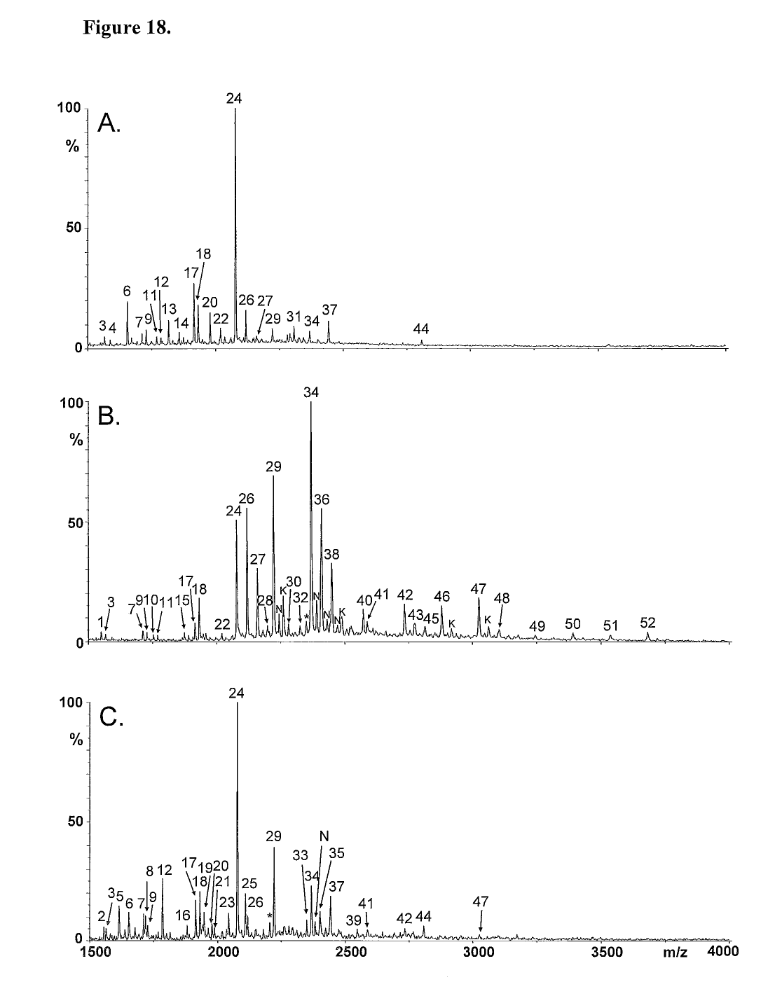

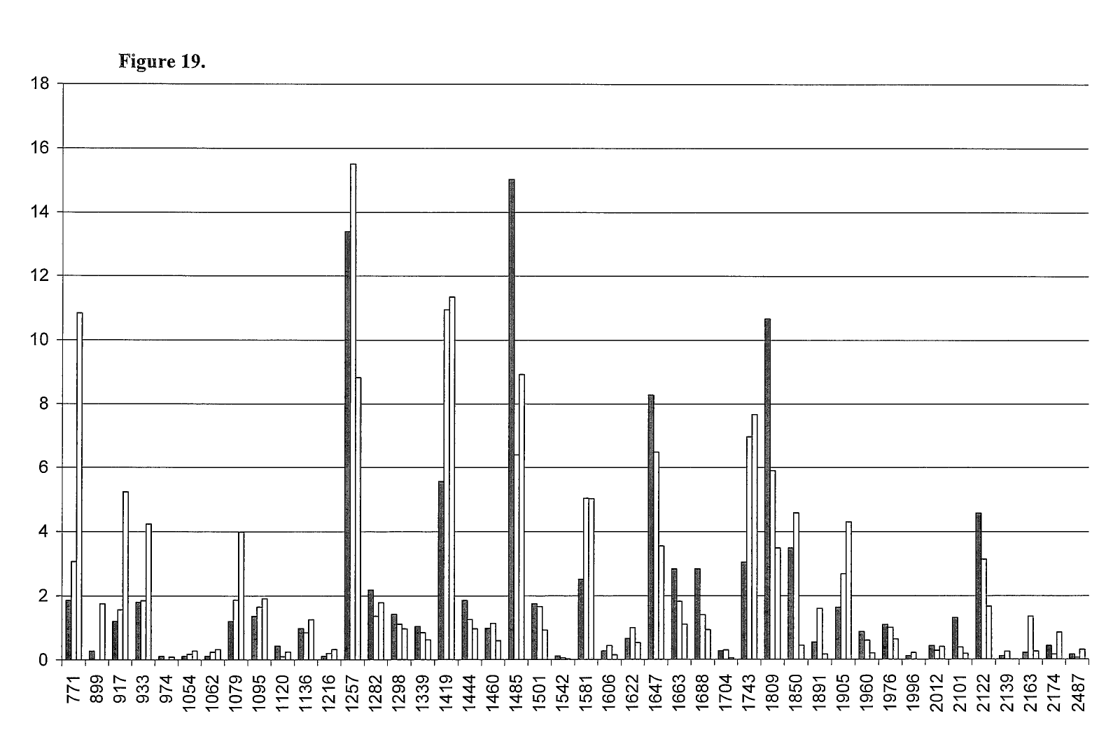

Occurrence of HexNAcβHexNAcβ sequences in sialylated and neutral protein-linked glycans from ovarian tissue samples. FIG. 18 shows MALDI-TOF mass spectra from normal ovarian tissue (FIG. 18A) and ovarian tumors. Especially in benign ovarian tumors (FIG. 18B) there occurs high amounts of sialylated glycan structures with more HexNAc than Hex units in their proposed monosaccharide compositions (Table 8). Such glycans include glycans No 26, 27, 36, and 38 (Table 8) which are major glycans in the benign tumors. However, only one of these glycans occurs in the malignant tissue and in a significantly reduced amount. In fact, in the sample from normal ovary tissue these glycan signals are more abundant than in the malignant tumour samples. In the neutral glycan fraction, the situation is similar (FIG. 19). The normal ovary sample and all the benign ovarian tumor samples resemble each other in that they contain more glycans at m/z 1850 and 1891, corresponding to Hex4HexNAc₅dHex, and Hex₃HexNAc₆dHex₁, respectively.

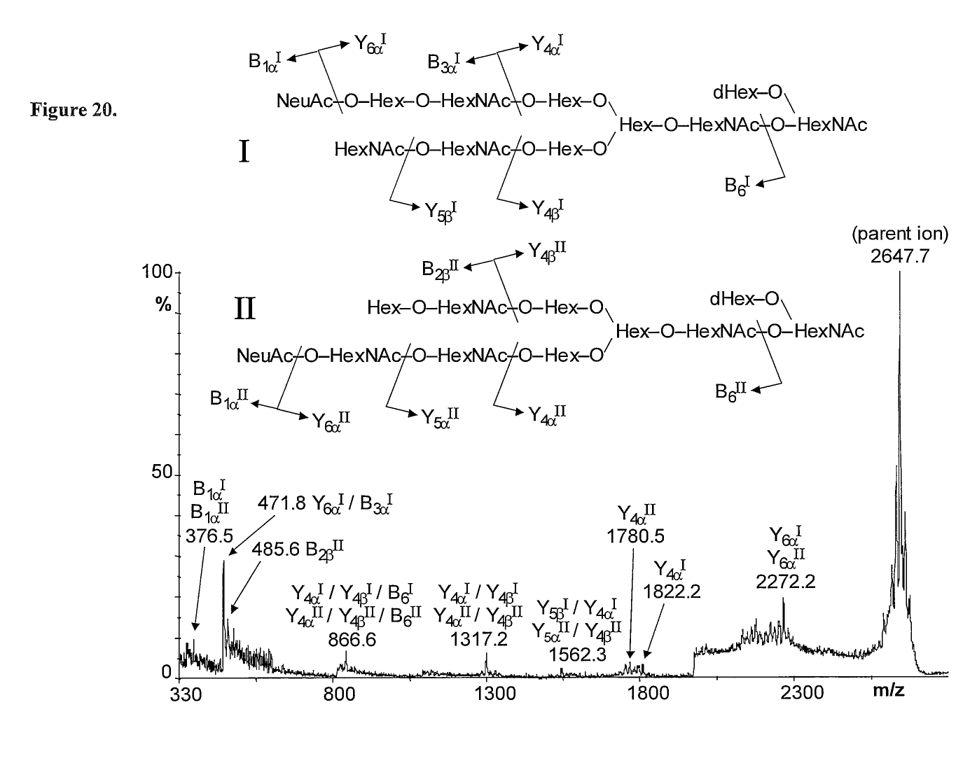

Structures of the glycans. The sialylated glycans were indicated to contain sialic acid residues upon neuraminidase digestion and subsequent analysis by MALDI-TOF mass spectrometry (data not shown). Exoglycosidase digestions of both neutral and sialylated fractions of benign ovarian tumor glycan samples showed that e.g. sialylated glycans 26 and 27, as well as neutral glycans at m/z 1850 and 1891 were resistant to the action of 3-glucosaminidase, but upon β-hexosaminidase digestion they lost either two or four HexNAc units. This indicates that the terminal residue in these glycans is not GlcNAcβ but that it may be GalNAcβ which is terminal to another HexNAcβ unit. The possible such structures include LacdiNAc (GalNAcβ4GlcNAcβ) that has been indicated previously in an ovarian glycoprotein. FIG. 20 shows a fragmentation mass spectrometric analysis of permethylated glycan 26 from benign ovarian tumor. The results indicate that glycan 26 contains the structures drawn in the Figure, which include both neutral and sialylated terminal HexNAc-HexNAc sequences. In conclusion, the present results therefore suggest that malignant transformation of the ovary is associated with diminished amounts of HexNAcβHexNAcβsequences, more specifically including neutral and sialylated LacdiNAc sequences.

Cancer-associated glycan signals and glycosylation changes. The present Example shows that multiple glycans and the glycan profiles are different between normal and malignant ovarian tissue, and that benign and malignant tumors differ from each other in multiple glycosylation features (see FIGS. 18 and 19). Significantly, also neutral O-glycan, sialylated O-glycan, and low-mannose N-glycan signals are elevated in all malignant ovarian tumor samples compared to the normal ovary and benign ovarian tumor samples (FIG. 19), as discussed in the other Examples of the present invention.

### Example 10

### Discrimination Analysis of Major Cancer Types Based on Mass Spectrometric Glycan Profiling

### Experimentation and Results

Relative abundancy profiles were obtained for the neutral protein-linked glycan fractions of cancer patient tissue samples with ductale and lobulare type breast adenocarcinoma, non-small cell lung adenocarcinoma, colon carcinoma or benign colon tumor, and ovarian cystadenocarcinoma (malignant tumor) or cystadenoma (benign tumor) as described in the other Examples.

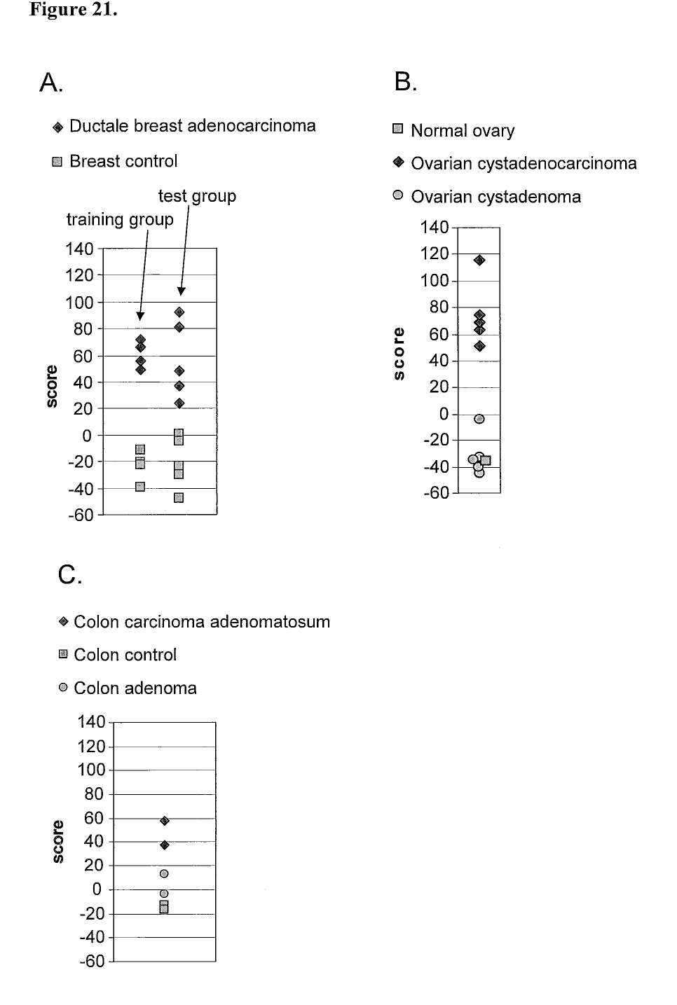

Generation of a discrimination formula. A principal component and discrimination analysis was done to the results of a randomly picked group of four cancer and four healthy tissue samples from patients with ductale type breast carcinoma. It was found that three glycan signals could resolve the samples into cancer and healthy groups (FIG. 21A, ‘training group’), and the experimental formula is:

5.36×I(m/z 771)+20.0×I(m/z 899)+8.13×I(m/z 917)−60.6,

where ‘I(glycan signal)’ refers to the relative abundance of the glycan signal marked in parenthesis, the three glycan signals correspond to sodium adduct ions of Hex₂HexNAc₂, NeuAc₁Hex₁HexNAc₁(deoxyamino)HexNAc, and Hex₂HexNAc₂dHex₁, respectively, and the resulting ‘scores’ for each sample are plotted on the y-axis in FIG. 21. As described in the preceding Examples, these glycan signals more specifically correspond to neutral O-glycans, sialylated Core 2 type O-glycans, and low-mannose N-glycans.

Testing the discrimination formula. The obtained experimental discrimination formula was first applied to neutral protein-linked glycan analysis results of a group of ductale type breast carcinoma patients. As seen in Figure xA (‘test group’), the formula could correctly discriminate 10 out of 10 samples (100%). Similarly, the formula was applied to samples from lobulare type breast carcinoma and lung cancer patients, and it discriminated correctly 12 out of 14 (86%) and 15 out of 17 (88%) samples from these patients, respectively. When applied to samples of ovarian tumors and healthy ovarian tissue (FIG. 21B), the formula correctly discriminated 11 out of 11 samples (100%) and placed the normal sample into the group of benign tumors. Similarly, when applied to samples of colon tumors and healthy colon tissue (FIG. 21C), the formula correctly discriminated 6 out of 6 samples (100%) and placed the normal samples into the group of benign tumors.

### Conclusions

The present results show that a simple experimental discrimination formula derived from a small group of breast cancer patients, applied on the results of neutral protein-linked glycan profiling, could effectively discriminate between cancerous and healthy samples of multiple cancer types. In particular, the same formula could correctly differentiate between malignant and benign tumors in both colon and ovary. In addition, it was shown that benign tumors resemble normal tissues and that malignant tumors differ significantly from both normal tissues and benign tumors with respect to the relative abundancies of glycan signals including neutral O-glycans, sialylated Core 2 type O-glycans, and low-mannose N-glycans. In conclusion, similar discrimination procedures can be used in diagnostics of human tumors and cancer. The present results indicate that there are individual differences in the expression of the cancer-associated glycan structures in both normal tissues between persons and in cancerous tissues between individual tumors. However, this did not effect the detection results of the present method.

Due to the presence of tissue-specific cancer-associated glycosylation revealed in the present invention, such as HexNAcβHexNAcβ glycans in tumors of the ovary (described in the other Examples), it is indicated that tissue-specific discrimination formulas based on the results of the present invention can differentiate between cancer and healthy samples and/or benign tumors even more efficiently than the exemplary formula of the present Example.

### Example 11

### Glycan Isolation and Analysis

### Examples of Glycan Isolation Methods

Glycan isolation. N-linked glycans are preferentially detached from cellular glycoproteins by F. meningosepticum N-glycosidase F digestion (Calbiochem, USA) essentially as described previously (Nyman et al., 1998), after which the released glycans are preferentially purified for analysis by solid-phase extraction methods, including ion exchange separation, and divided into sialylated and non-sialylated fractions. For O-glycan analysis, glycoproteins are preferentially subjected to reducing alkaline 1-elimination essentially as described previously (Nyman et al., 1998), after which sialylated and neutral glycan alditol fractions are isolated as described above. Free glycans are preferentially isolated by extracting them from the sample with water.

Example of a glycan purification method. Isolated oligosaccharides can be purified from complex biological matrices as follows, for example for MALDI-TOF mass spectrometric analysis. Optionally, contaminations are removed by precipitating glycans with 80-90% (v/v) aqueous acetone at −20° C., after which the glycans are extracted from the precipitate with 60% (v/v) ice-cold methanol. After glycan isolation, the glycan preparate is passed in water through a strong cation-exchange resin, and then through C₁₈ silica resin. The glycan preparate can be further purified by subjecting it to chromatography on graphitized carbon material, such as porous graphitized carbon (Davies, 1992). To increase purification efficiency, the column can be washed with aqueous solutions. Neutral glycans can be washed from the column and separated from sialylated glycans by elution with aqueous organic solvent, such as 25% (v/v) acetonitrile. Sialylated glycans can be eluted from the column by elution with aqueous organic solvent with added acid, such as 0.05% (v/v) trifluoroacetic acid in 25% (v/v) acetonitrile, which elutes both neutral and sialylated glycans. A glycan preparation containing sialylated glycans can be further purified by subjecting it to chromatography on microcrystalline cellulose in n-butanol:ethanol:water (10:1:2, v/v) and eluted by aqueous solvent, preferentially 50% ethanol:water (v/v). Preferentially, glycans isolated from small sample amounts are purified on miniaturized chromatography columns and small elution and handling volumes. An efficient purification method comprises most of the abovementioned purification steps. In an efficient purification sequence, neutral glycan fractions from small samples are purified with methods including carbon chromatography and separate elution of the neutral glycan fraction, and glycan fractions containing sialylated glycans are purified with methods including both carbon chromatography and cellulose chromatography.

MALDI-TOF mass spectrometry. MALDI-TOF mass spectrometry is performed with a Voyager-DE STR BioSpectrometry Workstation or a Bruker Ultraflex TOF/TOF instrument, essentially as described previously (Saarinen et al, 1999; Harvey et al., 1993). Relative molar abundancies of both neutral (Naven & Harvey, 1996) and sialylated (Papac et al., 1996) glycan components are assigned based on their relative signal intensities. The mass spectrometric fragmentation analysis is done with the Bruker Ultraflex TOF/TOF instrument according to manufacturer's instructions.

### Results

Examples of analysis sensitivity. Protein-linked and free glycans, including N- and O-glycans, are typically isolated from as little as about 5×10⁴ cells in their natural biological matrix and analyzed by MALDI-TOF mass spectrometry.

Examples ofanalysis reproducibility and accuracy. The present glycan analysis methods have been validated for example by subjecting a single biological sample, containing human cells in their natural biological matrix, to analysis by five different laboratory personnel. The results were highly comparable, especially by the terms of detection of individual glycan signals and their relative signal intensities, indicating that the reliability of the present methods in accurately describing glycan profiles of biological samples including cells is excellent. Each glycan isolation and purification phase has been controlled by its reproducibility and found to be very reproducible. The mass spectrometric analysis method has been validated by synthetic oligosaccharide mixtures to reproduce their molar proportions in a manner suitable for analysis of complex glycan mixtures and especially for accurate comparison of glycan profiles from two or more samples. The analysis method has also been successfully transferred from one mass spectrometer to another and found to reproduce the analysis results from complex glycan profiles accurately by means of calibration of the analysis.

Examples of biological samples and matrices for successful glycan analysis. The method has been successfully implied on analysis of e.g. blood cells, cell membranes, aldehyde-fixated cells, glycans isolated from glycolipids and glycoproteins, free cellular glycans, and free glycans present in biological matrices such as blood. The experience indicates that the method is especially useful for analysis of oligosaccharide and similar molecule mixtures and their optional and optimal purification into suitable form for analysis.

### Example 12

### Glycan Profiling

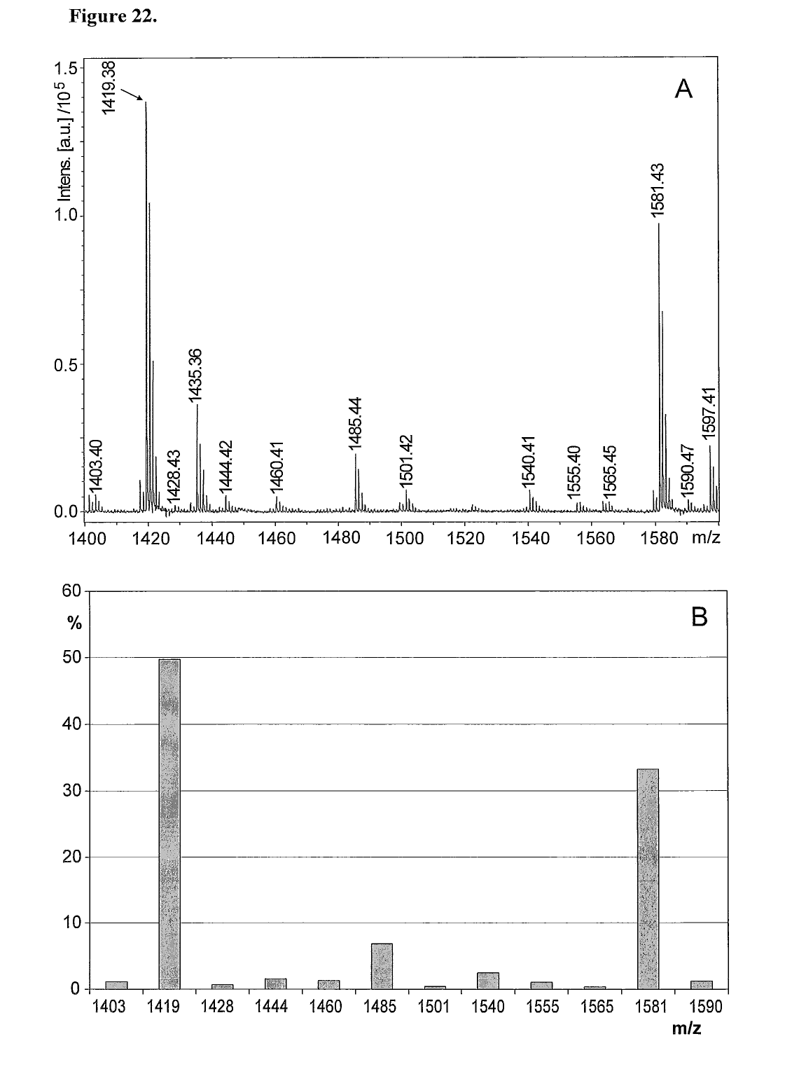

Generation of glycan profiles from mass spectrometric data. FIG. 22A shows a MALDI-TOF mass spectrum recorded in positive ion mode from a sample of neutral N-glycans. The profile includes multiple signals that interfere with the interpretation of the original sample's glycosylation, including non-glycan signals and multiple signals arising from single glycan signals. According to the present invention, the mass spectrometric data is transformed into a glycan profile (FIG. 22B), which represents better the original glycan profile of the sample.

An exemplary procedure is briefly as follows, and it includes following steps: 1) The mass spectrometric signals are first assigned to proposed monosaccharide compositions e.g. according to Table 9. 2) The mass spectrometric signals of ions in the molecular weight are of glycan signals typically show isotopic patterns, which can be calculated based on natural abundancies of the isotopes of the elements in the Earth's crust. The relative signal intensities of mass spectrometric signals near each other can be overestimated or underestimated, if their isotopic patterns are not taken into account. According to the present method, the isotopic patterns are calculated for glycan signals near each other, and relative intensities of glycan signals corrected based on the calculations. 3) Glycan ions are predominantly present as [M+Na]+ ions in positive ion mode, but also as other adduct ions such as [M+K]+. The proportion of relative signal intensities of [M+Na]+ to [M+K]+ ions is deduced from several signals in the spectrum, and the proportion is used to remove the effect of [M+K]+ adduct ions from the spectrum. 4) Other contaminating mass spectrometric signals not arising from the original glycans in the sample can optionally be removed from the profile, such as known contaminants, products of elimination of water, or in a case of permethylated oligosaccharides, undermethylated glycan signals. 5) The resulting glycan signals in the profile are normalized, for example to 100%, for allowing comparison between samples.

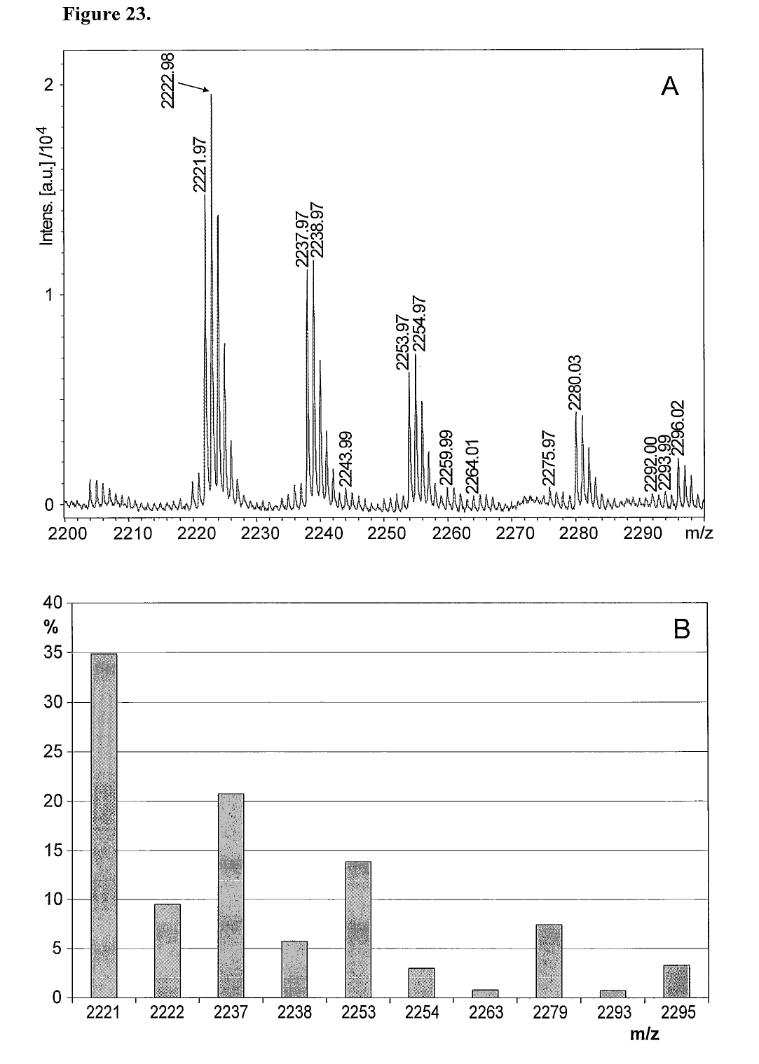

FIG. 23A shows a MALDI-TOF mass spectrum recorded in negative ion mode from a sample of neutral N-glycans. The profile includes multiple signals that interfere with the interpretation of the original sample's glycosylation, including non-glycan signals and multiple signals arising from single glycan signals. According to the present invention, the mass spectrometric data is transformed into a glycan profile (FIG. 23B), which represents better the original glycan profile of the sample. An exemplary procedure is briefly as follows, and it includes following steps: 1) The mass spectrometric signals are first assigned to proposed monosaccharide compositions e.g. according to Table 10. 2) The mass spectrometric signals of ions in the molecular weight are of glycan signals typically show isotopic patterns, which can be calculated based on natural abundancies of the isotopes of the elements in the Earth's crust. The relative signal intensities of mass spectrometric signals near each other can be overestimated or underestimated, if their isotopic patterns are not taken into account. According to the present method, the isotopic patterns are calculated for glycan signals near each other, and relative intensities of glycan signals corrected based on the calculations. 3) Glycan ions are predominantly present as [M−H]− ions in negative ion mode, but also as ions such as [M−2H+Na]− or [M−2H+ K]−. The proportion of relative signal intensities of e.g. [M−H]− to [M−2H+Na]- and [M−2H+ K]− ions is deduced from several signals in the spectrum, and the proportion is used to remove the effect of e.g. these adduct ions from the spectrum. 4) Other contaminating mass spectrometric signals not arising from the original glycans in the sample can optionally be removed from the profile, such as known contaminants or products of elimination of water. 5) The resulting glycan signals in the profile are normalized, for example to 100%, for allowing comparison between samples.

### Example 13

### Glycan Structures of Keyhole Limpet Hemocyanin (KLH) and Their Modification

### Experimentation and Results

Analysis of KLH glycosylation. Keyhole limpet hemocyanin was from Sigma (Megathura crenulata; USA). N-linked and O-linked glycans were released from KLH by N-glycosidase enzyme and alkaline β-elimination, respectively, and the released glycans were purified and analyzed essentially as described in the previous Examples. The detected N-glycan masses, as resolved by MALDI-TOF mass spectrometry, were essentially similar to those described by Kurokawa et al. (2001). However, novel glycan components could be engineered and/or identified on KLH by exoglycosidase digestions with α-mannosidase (Jack beans, C. ensiformis, Sigma) and combined β-galactosidase digestion (β1,4-galactosidase from S. pneumoniae and recombinant β1,3/6-galactosidase, Calbiochem, USA). More specifically, based on the susceptibility of the Hex₃HexNAc₂dHex, glycan to β-galactosidase (one hexose removed) and N-glycosidase F (detachment from the glycoprotein) it was found that KLH contains Manαt3(Galβ6)Manβ4GlcNAcβ4(Fucα6)GlcNAc that can be converted to Manα3Manβ4GlcNAcβ4(Fucα6)GlcNAc. The latter structure is a novel component in KLH. High-mannose and low-mannose N-glycans were similarly identified based on susceptibility to α-mannosidase. It was concluded that KLH contains at least the following human-type glycans in significant amounts: (Manα)₃Manβ4GlcNAcβ4GlcNAc, (Manα)₄Manβ4GlcNAcβ4GlcNAc, (Manα)₅Manβ4GlcNAcβ4GlcNAc, (Manα)₆Manβ4GlcNAcβ4GlcNAc, (Manα)₁Manβ4GlcNAcβ4(Fucα6)GlcNAc, (Manα)₂Manβ4GlcNAcβ4(Fucα6)GlcNAc, (Manα)₃Manβ4GlcNAcβ4(Fucα6)GlcNAc, (Manα)₄Manβ4GlcNAcβ4(Fucα6)GlcNAc, and (Manα)₅Manβ4GlcNAcβ4(Fucα6)GlcNAc. About ⅕ of the non-fucosylated and ⅓ of the fucosylated glycans with similar masses as the glycans listed above were assigned as containing β1,6-linked galactose residues based on their resistance to the action of α-mannosidase (Kurokawa et al., 2001). The latter are not human-type glycans.

Production of KLH with human-type low-mannose N-glycans. β-galactosidase was used to removal of, and the reaction result was characterized by MALDI-TOF mass spectrometry of released glycans, verifying that significant amounts of β-galactose residues, occurring in KLH glycans and capping the human-type low-mannose N-glycans, were removed. Based on the structural knowledge described previously (Kurokawa et al., 2001), it was concluded that a modified spectrum of KLH glycans was produced, containing more low-mannose N-glycans than original KLH, and especially the novel human-type low-mannose glycan Manα3Manβ4GlcNAcβ4(Fucα6)GlcNAc described above. As described in the preceding Examples, the increased glycans present in KLH are associated with malignant tumors in major human cancer types.

### Example 14

### Analyses of Human Tissue Material and Cell Protein-Linked Glycan Structures

### Experimental Procedures

Protein-linked glycans were isolated by non-reductive alkaline elimination essentially as described by Huang et al. (2000), or by N-glycosidase digestion to specifically retrieve N-glycans as described in the preceding Examples.

### Results and Discussion

Tissue-specific glycosylation analyses and comparison of glycan profiles between tissues. Human tissue protein-linked glycan profiles were analyzed from lung, breast, kidney, stomach, pancreas, lymph nodes, liver, colon, larynx, ovaries, and blood cells and serum. In addition, cultured human cells were analyzed similarly. Tables 14 and 15 show neutral and acidic protein-linked glycan signals, respectively, observed in these human tissues and cells together with their classification into glycan structure groups. However, the individual glycan signals in each structure group varied from sample type to sample type, reflecting tissue material and cell type specific glycosylation. Importantly, in analyses of multiple samples, such as 10 samples from an individual human tissue type, glycan group feature proportions remain relatively constant with respect to variation in the occurrence of individual glycan signals.

Furthermore, it was observed that each tissue demonstrated a specific glycan profile that could be distinguished from the other tissues, cells, or blood or serum samples by comparison of glycan profiles according to the methods described in the present invention. It was also found out that glycan profile difference could be quantitated by comparing the difference between two glycan profiles, for example according to the Equation (resulting in difference expressed in %):

$$difference = \frac{1}{2} {\sum }_{i = 1}^{n} \vert {p}_{i , a} - {p}_{i , b} \vert ,$$

wherein p is the relative abundance (%) of glycan signal i in profile a or b, and n is the total number of glycan signals. For example, the Equation reveals that human lung and ovary tissue protein-linked glycan profiles differ from each other significantly more than human lung and kidney tissue protein-linked glycan profiles differ from each other. Each tissue or cell type could be compared in this manner.

Comparison of glycosylation features between human tissue materials. Table 16 shows how glycan signal structural classification according to the present invention was applied to the comparison of quantitative differences in glycan structural features in glycan profiles between human tissue materials. The results show that each sample type was different from each other with respect to the quantitative glycan grouping and classification. Specifically, normal human lung and lung cancer tissues were different from each other both in the neutral glycan and sialylated glycan fractions with respect to the quantitative glycan structure grouping. In particular, lung cancer showed increased amounts of glycan signals classified into terminal HexNAc containing glycans. In analysis of individual glycan signals by β-glucosaminidase digestion, it was found that lung cancer associated glycan signals, such as Hex₃HexNAc₄dHex₁, contained terminal β-linked GlcNAc residues, correlating with the classification of these glycan signals into the terminal HexNAc (N>H and/or N═H) glycan groups. Furthermore, the human serum protein-linked glycan profile showed significantly lower amounts of high-mannose and especially low-mannose type N-glycan signals. It is concluded that the glycan grouping profile of human serum is significantly different from the corresponding profiles of solid tissues, and the present methods are suitable for identification of normal and diseased human tissue materials and blood or serum typical glycan profiles from each other.

Disease- and tissue-specific differences in glycan structure groups. FIG. 19 shows a neutral protein-linked glycan profile of human ovary with abnormal growth. As described above, there are clear differences in the overall glycan profiles of FIG. 19 and other human tissue samples. In analyses of multiple samples of ovarian tissues, it was found that benign abnormal growth of the ovary is especially characterized by increased amounts of glycan signals classified as terminal HexNAc (N>H). In structural analyses by fragmentation mass spectrometry and combined β-hexosaminidase and β-glucosaminidase digestions, the corresponding terminal HexNAc glycan signals were found to include structures with terminal and sialylated β-GalNAc, more specifically terminal and sialylated GalNAcβ4GlcNAcβ(LacdiNAc) structures. According to the glycan structure classification, the protein-linked glycan profiles of normal ovarian tissue also contain increased amounts of terminal HexNAc glycans compared to other human tissues studied in the present invention, and normal human ovary preferentially also contains higher amounts of terminal and/or sialylated LacdiNAc structures than other human tissues on average. However, in malignant transformation the proportion of LacdiNAc structures among the protein-linked glycans of the ovary are decreased, and this is also reflected in the glycan grouping classification of malignant ovarian glycan profiles.

The analysis of protein-linked glycan profiles of human tissues revealed also that tissues with abundant epithelial structures, such as stomach, colon, and pancreas, contain increased amounts of small glycan structures, preferentially mucin-type glycans, and fucosylated glycan structures compared to the other glycan structure groups in structure classification. Similarly as epithelial tissues, mucinous carcinomas were differentiated from other carcinoma types based on analysis of their protein-linked glycan profiles and structure groups according to the methods of the present invention.

### Example 15

### Proton-NMR Analysis of Glycan Fractions

### Experimental NMR Procedures

Glycan material is liberated from biological material by enzymatic or chemical means. To obtain a less complex sample, glycans are fractionated into neutral and acidic glycan fractions by chromatography on a graphitized carbon as described above. A useful purification step prior to NMR analysis is gel filtration high-performance liquid chromatography (HPLC). For glycans of glycoprotein or glycolipid origin, a Superdex Peptide HR10/300 column (Amersham Pharmacia) may be used. For larger glycans, chromatography on a Superdex 75 HR10/300 column may yield superior results. Superdex columns are eluted at a flow rate of 1 ml per minute with water or with 50-200 mM ammonium bicarbonate for the neutral and acidic glycan fractions, respectively, and absorbance at 205-214 nm is recorded. Fractions are collected (typically 0.5-1 ml) and dried. Repeated dissolving in water and evaporation may be necessary to remove residual ammonium bicarbonate salts in the fractions. The fractions are subjected to MALDI-TOF mass spectrometry and all fractions containing glycans are pooled.

Prior to NMR analysis, the pooled fractions are dissolved in deuterium oxide and evaporated. With glycan preparations containing about 100 nmol or more material, the sample is finally dissolved in 600 microliters of high-quality deuterium oxide (99.9-99.996%) and transferred to a NMR analysis tube. A roughly equimolar amount of an internal standard, e.g. acetone, is commonly added to the solution. With glycan preparations derived from small tissue specimens or from a small number of cells (5-25 million cells), the sample is preferably evaporated from very high quality deuterium oxide (99.996%) twice or more to eliminate H₂O as efficiently as possible, and then finally dissolved in 99.996% deuterium oxide. These low-material samples are preferably analyzed by more sensitive NMR techniques. For example, NMR analysis tubes of smaller volumes can be used to obtain higher concentration of glycans. This kind of tubes include e.g. nanotubes (Varian) in which sample is typically dissolved in a volume of 37 microliters. Alternatively, higher sensitivity is achieved by analyzing the sample in a cryo-NMR instrument, which increases the analysis sensitivity through low electronic noise. The latter techniques allow gathering of good quality proton-NMR data from glycan samples containing about 1-5 nmol of glycan material.

### Analysis of NMR Data

It is realized that numerous studies have shown that proton-NMR data has the ability to indicate the presence of several structural features in the glycan sample. In addition, by careful integration of the spectra, the relative abundancies of these structural features in the glycan sample can be obtained.

For example, the proton bound to monosaccharide carbon-1, i.e. H-1, yields a distinctive signal at the lower field, well separated from the other protons of sugar residues. Most monosaccharide residues e.g. in N-glycans are identified by their H-1 signals (see Tables 4 and 5 for representative examples). In addition, the H-2 signals of mannose residues are indicative of their linkages.

Sialic acids do not possess a H-1, but their H-3 signals (H-3 axial and H-3 equatorial) reside well separated from other protons of sugar residues. Moreover, differently bound sialic acids may be identified by their H-3 signals. For example, the Neu5Ac H-3 signals of Neu5Acα2-3Gal structure are found at 1.797 ppm (axial) and 2.756 ppm (equatorial). On the other hand, the Neu5Ac H-3 signals of Neu5Acα2-6Gal structure are found at 1.719 ppm (axial) and 2.668 ppm (equatorial). By comparing the integrated areas of these signals, the molar ratio of these structural features is obtained.

Other structural reporter signals are commonly known and those familiar with the art use the extensive literature for reference in glycan NMR assignments.

### Results of Malignant Tumor Glycan Analyses by NMR

Samples: N-glycan fractions were liberated from pancreas carcinoma samples, purified as described in the preceding Examples, ultimately by gel filtration HPLC, and fractionated into 1) large neutral, 2) small neutral, and 3) acidic N-glycan fractions. These samples were analyzed by cryo-probe ¹H-NMR as described above.

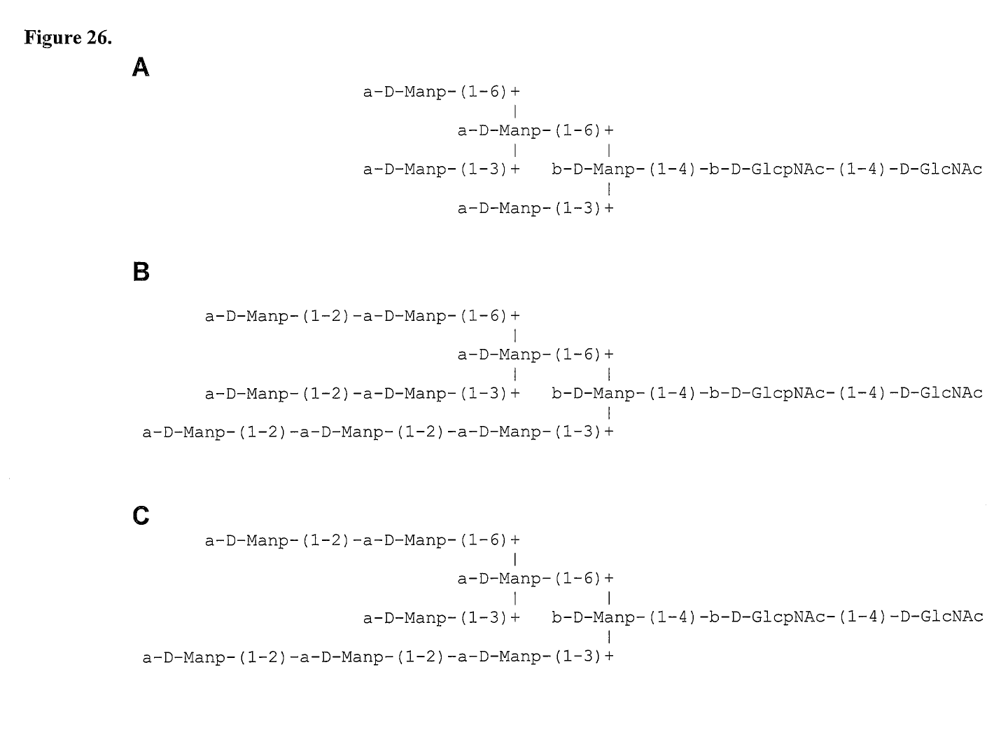

Large neutral N-glycan fraction: This fraction contained high-mannose N-glycans corresponding to the structural elements of reference structures in FIG. 26. Correlation with these structures is described in Table 11, demonstrating that such structures were the major glycan signals in the glycan fraction.

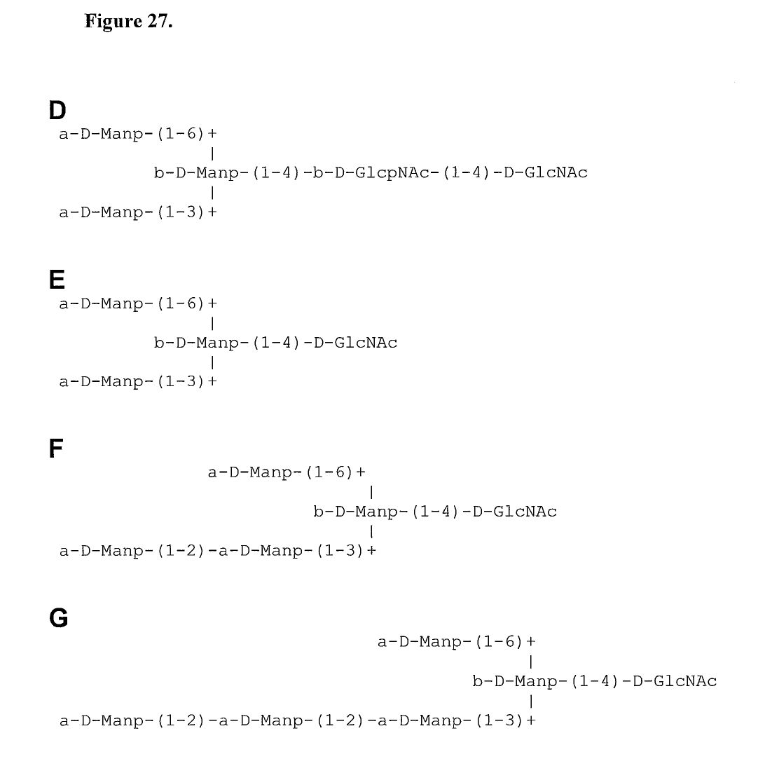

Small neutral N-glycan fraction: This fraction contained low-mannose N-glycans as well as ManₙGlcNAc₁ glycans corresponding to the structural elements of reference structures in FIG. 27. Correlation with these structures is described in Table 12, demonstrating that such structures were major glycan signals in the glycan fraction. In particular, the results demonstrated the presence of Manα3(Manα6)Manβ4GlcNAcβ4GlcNAc low-mannose N-glycan, as well as the presence of (Manα2)₀₋₁Manα3 N-glycan antenna, and the presence of (Manα2)₀₋₁Manα3(Manα6)Manβ4GlcNAe glycans in the glycan fraction.

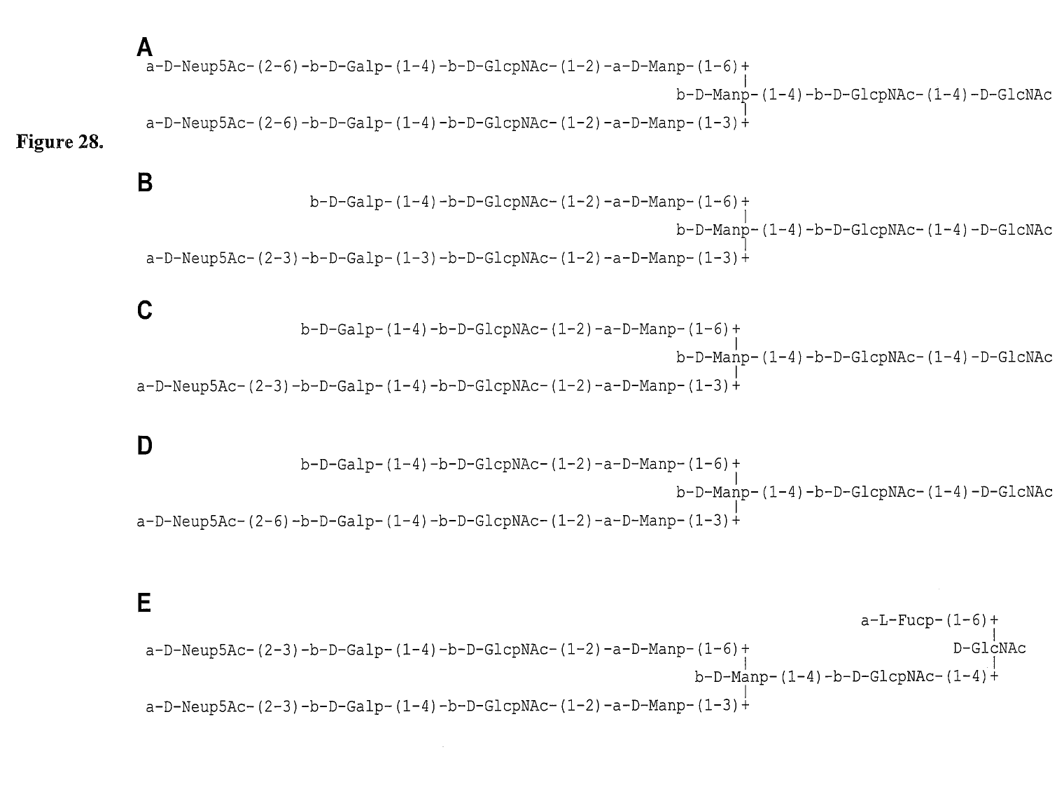

Acidic neutral N-glycan fraction: This fraction contained complex-type N-glycans corresponding to the structural elements of biantennary N-glycan reference structures in FIG. 28, as well as triantennary N-glycans with Gal 34GlcNAcβ4Manα3 antennae (not shown). Correlation with these structures is described in Table 13, demonstrating that such structures were major glycan signals in the glycan fraction. In particular, the results demonstrated the presence of Galβ4GlcNAcβ2Manα3(Galβ4GlcNAcβ2Manα6)Manβ4GlcNAcβ4(±Fucα6)GlcNAc N-glycan core structure as the major structure, elongated with different sialylation as in the antennae of the reference structures. Quantitative analysis of sialic acid linkages by integration of representative signals in the spectrum yielded β2,6- and β2,3-linked sialic acids in relative proportion 2:1 in the analyzed sample.

In addition, in malignant sample of pancreas cancer a detected N-glycan signal at 4.515 ppm was preferably from the sialylated N-glycan antenna sequence NeuAcα3Galβ3GlcNAcβ2Manα3 that was quantitated against Galβ4GlcNAc antennae, and found to be expressed in the cancer tissue in ≧10% proportion.

### NMR REFERENCES

Fu D., Chen L. and O'Neill R. A. (1994) Carbohydr. Res. 261, 173-186
Hård K., Mekking A., Kamerling J. P., Dacremont G. A. A. and Vliegenthart J. F. G. (1991) Glycoconjugate J. 8, 17-28
Hård K., Van Zadelhoff G., Moonen P., Kamerling J. P. and Vliegenthart J. F. G. (1992) Eur. J. Biochem. 209, 895-915 Helin J., Maaheimo H., Seppo A., Keane A. and Renkonen O. (1995) Carbohydr. Res. 266, 191-209

### Example 16

### Lysosomal Organelle-Specific N-Glycosylation

### Experimental Procedures

Lysosomal protein sample including human myeloperoxidase was chosen to represent lysosomal organelle glycoproteins. The sample was digested with N-glycosidase F to isolate N-glycans, and they were purified for MALDI-TOF mass spectrometric analysis as described in the preceding Examples.

Alkaline phosphatase digestion was performed essentially according to manufacturer's instructions. After the digestion glycans were purified for MALDI-TOF mass spectrometric analysis as above.

### Results and Discussion

Neutral N-glycan profiles. The neutral N-glycan profile is presented in FIG. 25 (upper panel). The profile is dominated by low-mannose type and high-mannose-type N-glycan signals, comprising 49% and 46% of the total signal intensity, respectively. Especially the high proportion of low-mannose type N-glycans is characteristic to the sample (Table 17, upper panel).

Acidic N-glycan profiles. The acidic N-glycan profile is presented in FIG. 25 (lower panel). The profile is dominated by three glycan signal groups: 1) sulphated or phosphorylated low-mannose type and high-mannose type N-glycans (Hex₃₋₈HexNAc₂SP), 2) fucosylated hybrid-type or monoantennary N-glycans (NeuAc₁Hex₃₋₄HexNAc₃dHex₁), and 3) fucosylated complex-type N-glycans (NeuAc₁Hex₄₋₅HexNAc₄dHex₁₋₂). Unusual features of the sample are the high proportion of hybrid-type or monoantennary N-glycans (Table 17, lower panel), high fucosylation rate of hybrid-type, monoantennary, and complex-type N-glycans, and the high proportion of the characteristic sulphated or phosphorylated low-mannose type and high-mannose type N-glycans.

Phosphorylated N-glycans. Major glycan signals with phosphate or sulphate ester (SP) in their monosaccharide compositions were Hex₅HexNAc₂SP (1313), Hex₆HexNAc₂SP (1475), and Hex₇HexNAc₂SP (1637). When the acidic glycan fraction was subjected to alkaline phosphatase digestion, these major signals were specifically digested and disappeared from the acidic glycan spectrum as detected by MALDI-TOF mass spectrometry (data not shown). In contrast, the major glycan signals with sialic acids in their monosaccharide compositions were not digested, including NeuAc₁Hex₃HexNAc₃dHex₁ (1549). This indicates that the three original glycan signals corresponded to phosphorylated N-glycans (PO₃H)Hex₅HexNAc₂, (PO₃H)Hex₆HexNAc₂, and (PO₃H)Hex₇HexNAc₂, respectively, wherein PO₃H denotes phosphate ester.

The data further indicated that the present organelle-specific N-glycan profile included phosphorylated low-mannose type and high-mannose type N-glycans (PO₃H)Hex₃HexNAc₂ (989), (PO₃H)Hex4HexNAc₂ (1151), (PO₃H)Hex₅HexNAc₂ (1313), (PO₃H)Hex₆HexNAc, (1475), (PO₃H)Hex₇HexNAc₂ (1637), and (PO₃H)Hex₈HexNAc₂ (1799). In this glycan profile the phosphorylated glycan residues are preferentially mannose residues, more preferentially α-mannose residues, and most preferentially 6-phospho-α-mannose residues i.e. (PO₃H-6Manα).

### Example 17

### Identification of Specific Glycosylation Signatures from Glycan Profiles of Malignant and Normal Human Tissue Samples Based on Quantitative Glycomics

### Experimental Procedures

Normal lung (Sample I) and malignant lung tumor samples (Sample II) were archival formalin-fixed and paraffin-embedded tissue sections from cancer patients with small cell lung cancer. Protein-linked glycans were isolated from the representative samples by non-reductive β-elimination, purified, and analyzed by MALDI-TOF mass spectrometry as described in the preceding Examples. In the present analysis, the total desilylated protein-linked glycomes from each sample were used.

To analyze the data and to find the major glycan signals associated with either the normal state or the disease, two variables were calculated for the comparison of glycan signals between the two samples:

1. absolute difference A=(SII−SI), and
2. relative difference R=A/SI,
wherein SI and SII are relative abundances of a given glycan signal in Sample I (normal human lung tissue) and Sample II (small cell lung cancer), respectively.

The glycan signals were further classified into structure classes by a one letter code:

a b c d,
wherein a is either N (neutral) or S (sialylated); b is either L (low-mannose type), M (high-mannose type), H (hybrid-type or monoantennary), C (complex-type), S (soluble), or O (other); c is either—(nothing), F (fucosylated), or E (multifucosylated); and d is either—(nothing), T (terminal HexNAc, N>H), or B (terminal HexNAc, N═H); as described in the present invention.

### Results

To identify protein-linked glycan signals correlating with malignant tumors in total tissue glycomes from cancer patient, major signals specific to either normal lung tissue or malignant small cell lung cancer tumors were selected based on their relative abundances. When, A and R were calculated for the glycan profile datasets of the two samples, and the glycan signals thereafter sorted according to the values of A and R, the most significant differing glycan signals between the two samples could be identified (Table 18). Among the most abundant protein-linked glycan signals in the data, the following three signals had emerged in II (new in Table 18): 1955, 2685, and 2905, corresponding to fucosylated complex-type N-glycans. The absolute differences of these signals were among the ten most large in the data, indicating that they were significant. The signals that experienced the highest relative increase in R were: 771 (R=2.4, corresponding to 3.4-fold increase), 1905 (R=2.2, corresponding to 3.2 fold increase), and 1485 (R=1.3, corresponding to 2.3 fold increase). The latter signal corresponded to complex-type N-glycans with terminal HexNAc. Significantly, its +2Hex counterpart 1809 was the most drastically reduced glycan signal in II with A=−8.9 and R=−0.4 (corresponding to 40% decrease in II), indicating a large change in terminal HexNAc expression. Moreover, the data easily shows that the glycan signals 1704, 1866, 1136, and 755 were not present in II.

Further, the obtained results, especially the identified major glycan signals indicative of either Sample II (high A and R) or Sample I (low A and R) were used to compile two alternative algorithms to produce glycan score with which lung cancer sample could be identified from normal lung sample based on the glycan signal values of the quantitative glycome data:

1. glycan score=I(1485)−I(1809),
wherein I(1485) is the relative abundance of glycan signal 1485 and I(1809) is the relative abundance of glycan signal 1809; and alternatively:
2. glycan score=I(1485)/I(1809)

These glycan score algorithms yield high numerical value when applied to lung cancer sample and low numerical value when applied to normal lung sample.

### Discussion

The present identification analysis produced selected glycan signal groups, from where indifferent glycan signals have been removed and that have reduced noise or background and less observation points, but have the resolving power of the initially obtained glycan profiles. Such selected signal groups and their patterns in different sample types can serve as a signature for the identification of for example 1) normal human glycosylation, 2) tissue-specific glycosylation, 3) disease states affecting tissue glycosylation, 4) malignant cancer, 5) malignancy in comparison to benign tumors, and grade of malignancy, or 6) glycan signals that have individual variation. Moreover, glycan signals can be identified that do not change between samples, including major glycans that can be considered as invariant or housekeeping glycans.

The present data analysis identified potential glycan marker signals for future identification of either the normal lung of the lung tumor glycome profiles. Further, glycan classes that are associated with e.g. disease state in humans can be identified. Specifically, the analysis revealed that within the total complex-type N-glycan structure class in the tissue glycomes, terminal HexNAc (N>H) were typical to small cell lung cancer.

The method also allows identification of major glycans or major changes within glycan structure classes. For example, the proportion of multifucosylated glycans within the total tissue glycome profile was increased in II (1.1%) compared to I (0.3%). The data analysis identified this change predominantly to the appearance of glycan signals 1955 and 2685 in II.

### Example 18

### Periodate Oxidation Analysis of N-Glycan Structures

### Experimental Procedures

Cancer cell derived N-glycans were obtained by N-glycosidase F digestion and purified as described in the preceding Examples. The glycan sample was dissolved in 10 μl of 8 mM sodium metaperiodate prepared in 0.1 M sodium acetate buffer, pH 5.5. The oxidation reaction was allowed to proceed at +4° C. in the dark for two days. The excess of periodate was then destroyed by adding 10 μl of 80 mM ethylene glycol and the mixture was allowed to stand for 6 hours. The reaction mixture was then neutralized by adding 10 μl of 0.1 M aqueous ammonia. The aldehyde groups which were generated in the reaction mixture were then reduced by adding 10 μl of 1.6M sodium borohydride and allowed to stand overnight at +4° C.

The carbohydrate material in the reaction mixture was isolated by solid phase extraction on a small column of graphitized carbon and subjected to MALDI-TOF mass spectrometry in 2,5-dihydroxybenzoic acid matrix.

### Results

Some of the major signals observed in the spectrum (FIG. 33) were assigned as follows:

The signal m/z 1831 represents an oxidized-reduced form of (Man)_{g}(GlcNAc)₂ species ([M+Na]⁺, m/z 1905), containing three terminal Man units. Because periodate oxidizes vicinal hydroxyl groups, the terminal units are oxidized so that they lose the C-3 as formaldehyde, each contributing to a loss of −28 Da in mass. In addition, 2-substituted Man units may get oxidized between C-3 and C-4, yielding two primary alcohol groups (leading to +2 Da mass change each). Typical mammalian (Man)₉(GlcNAc)₂ species carry four Manβ-1,2 residues, and here four 2-substituted Man units were oxidized this way and contribute to a mass increment of +8 Da. The reducing end of the glycan is also reduced, yielding +2 Da increment. No unoxidized (Man)₉(GlcNAc)₂ species can be observed in the spectrum.

The signal m/z 1831 is accompanied by a signal at m/z 1861. This signal is assigned as an oxidized-reduced structure where one of the terminal Man units is oxidized only between C₂-C₃ or C₃-C₄, but not liberating formaldehyde, thus yielding a structure 30 Da larger than m/z 1831 material.

The signal m/z 1667 represents an oxidized-reduced form of (Man)₈(GlcNAc)₂ species ([M+Na]⁺, m/z 1743), containing three terminal Man units. These terminal units are oxidized so, that they lose the C-3 as formaldehyde, each contributing to a loss of −28 Da. In addition, all three 2-substituted Man units got oxidized between C-3 and C-4, yielding two primary alcohol groups (+2 Da). The m/z 1697 signal is assigned as an oxidized-reduced structure where one of the terminal Man units is oxidized only between C2-C3 or C3-C4, but not liberating formaldehyde, thus yielding a structure 30 Da larger than m/z 1667 material.

Other major signals are derived similarly from three following structures:

m/z 1503 and 1533 from (Man)₇(GlcNAc)₂ species (intact m/z 1581);
m/z 1339 and 1369 from (Man)₆(GlcNAc)₂ species (intact m/z 1419);
m/z 1175 and 1205 from (Man)₅(GlcNAc)₂ species (intact m/z 1257);
m/z 1041 and 1071 from (Man)₄(GlcNAc)₂ species (intact m/z 1095).

A part of the mass spectrum shown in FIG. 33 is zoomed in FIG. 34, showing the mass range of m/z 800-950. A major signal m/z 835 originates from an oxidized-reduced form of m/z 917, a (Man)₂(Fuc)(GlcNAc)₂ species. Here, three formaldehyde units are lost, so the structure is preferably Manβ1-6Manα-4GlcNAcβ1-4(Fucα1-6)GlcNAc. The rationale is that both terminal Man and Fuc are oxidized to release formaldehyde, but also the penultimate 6-substituted Man is oxidized to release formaldehyde. As above, there is a signal +30 Da from m/z 835, i.e. m/z 865. In this species, one of the units mentioned is not completely oxidized to release formaldehyde, but only oxidized between C2-C3 or C3-C4, and reduced to alcohol groups.

The m/z 879 signal is derived from an oxidized-reduced form of m/z 933, a (Man)₃(GlcNAc)₂ species. Here, two terminal unsubstituted Man units are oxidized to release formaldehyde, other monosaccharide units are stable to periodate oxidation. Preferred structure corresponding to the original abundant signal is Manα3(Manα6)Manβ4GlcNAc4GlcNAc.

### Example 19

### Human Antibody Molecules Against type II N-Acetyllactosamine on Core 2 O-glycan

### Experimental Procedures

Affinity reagent for analyzing antibody molecules recognizing type II N-acetyllactosamine on Core 2 O-glycans was prepared by coupling Galβ4GlcNAcβ6(Galβ3)GalNAcα-O—(CH₂)₂-(p-amino)benzyl (IsoSep, Sweden) to N-hydroxysuccinimide activated Sepharose (Amersham Pharmacia, Sweden).

Serum samples were isolated from a person that had had malignant ovarian cancer and recovered from it, as well as control individuals.

Ig antibodies were analyzed by affinity isolating them from human sera, washing, eluting with acid, and detecting Ig subunits by protein detection after SDS-PAGE according to standard procedures.

### Results

Ovarian cancer patient samples showed substantial amounts of human Ig antibodies with affinity to the type II N-acetyllactosamine Core 2 O-glycan affinity material, and proteins corresponding to Ig subunits could be detected in SDS-PAGE.

### Example 20

### Analysis of Large Cancer Patient Sample Panel with Respect to Expression of Cancer-Associated Glycan Antigens

### Experimental Procedures

Protein-linked glycans were isolated from formalin-fixed and paraffin-embedded tissue sections and analyzed as describe in the preceding Examples. Following cancer types were analyzed: lung cancer, both small cell lung adenocarcinoma and non-small cell lung adenocarcinoma, and lung carcinoma liver metastases; breast cancer; ductale type breast adenocarcinoma and lymph node metastases thereof; lobulare type breast adenocarcinoma and lymph node metastases thereof; ovarian cystadenocarcinoma; colon cancer/carcinoma, carcinoma adenomatosum, and liver metastases thereof; kidney cancer/carcinoma, and kidney hypemephroma; gastric cancer/carcinoma, and lymph node metastases thereof, liver cancer/carcinoma; larynx cancer/carcinoma; pancreas cancer/carcinoma; melanoma and liver metastases thereof; gall bladder cancer/carcinoma, and liver metastases thereof; salivary gland cancer/carcinoma, and skin metastases thereof; and lymph node cancer/carcinoma (lymphoma).

### Results

Low-mannose type N-glycans were identified based on their indicative mass spectrometric glycan signals for [M+Na]⁺ ions of Hex₁₋₄HexNAc₂dHex₀₁, as described in to the present invention. In the present analyses the preferred indicative signals of the glycan structure group were m/z 771, 917, 933, 1079, and 1095 due to their abundance in mass spectrometric glycan profiles; 771, 933, and 1095 especially in case of non-fucosylated low-mannose type N-glycans; 917 and 1079 especially in case of detecting fucosylated low-mannose type N-glycans; 933, 1079, and 1095 especially in case of detecting low-mannose type N-glycans when N- and O-glycans were analyzed simultaneously; 1079 and 1095 as preferred sensitive indicators of the group; and/or 1079 as a preferred sensitive single indicator of the group.

Using these criteria, low-mannose type N-glycans were detected to be cancer-associated and expressed in malignant tumors in following cancer types: lung cancer, both small cell lung adenocarcinoma and non-small cell lung adenocarcinoma, and lung carcinoma liver metastases; breast cancer; ductale type breast adenocarcinoma and lymph node metastases thereof; lobulare type breast adenocarcinoma and lymph node metastases thereof; ovarian cystadenocarcinoma; colon cancer/carcinoma, carcinoma adenomatosum, and liver metastases thereof; kidney cancer/carcinoma, and kidney hypemephroma; gastric cancer/carcinoma, and lymph node metastases thereof, liver cancer/carcinoma; larynx cancer/carcinoma; pancreas cancer/carcinoma; melanoma and liver metastases thereof; gall bladder cancer/carcinoma, and liver metastases thereof; and lymph node cancer/carcinoma (lymphoma). Further the expression of the glycans was detected in abundant amounts in salivary gland cancer/carcinoma, and skin metastases thereof.

In all these cancer types, both fucosylated and non-fucosylated low-mannose type N-glycans were detected and expression levels were found to be higher in cancer in comparison to normal human tissue samples. In addition, in benign tumors of the ovary and colon, low-mannose type glycan signals were significantly lower than in the corresponding malignant tumors, indicating that low-mannose type glycans are specifically associated with malignancy in human cancers.

O-glycans were identified based on their major indicative mass spectrometric glycan signals 771, 917, 899, 1038, and 1329 corresponding to SA₀₋₂Hex₂HexNAc₂dHex₀₋₁ glycan compositions, as described in to the present invention. In the present analyses the preferred indicative signals of the glycan structure group were m/z 771, 917, and 899 due to their abundance in mass spectrometric glycan profiles; 771 especially in case of non-fucosylated neutral glycans; 917 especially in case of detecting fucosylated neutral glycans; and 899 especially in case of detecting sialylated glycans; 771 and 899 as preferred sensitive indicators of the group; and/or 771 as a preferred sensitive single indicator of the group.

Using these criteria, O-glycans were detected to be cancer-associated and expressed in malignant tumors in following cancer types: lung cancer/carcinoma, both small cell lung adenocarcinoma and non-small cell lung adenocarcinoma, and lung carcinoma liver metastases; breast cancer; ductale type breast adenocarcinoma and lymph node metastases thereof; lobulare type breast adenocarcinoma and lymph node metastases thereof; ovarian cystadenocarcinoma; colon cancer/carcinoma, and carcinoma adenomatosum; kidney cancer/carcinoma, and kidney hypemephroma; gastric cancer/carcinoma; larynx cancer/carcinoma; and pancreas cancer/carcinoma; melanoma and liver metastases thereof; gall bladder cancer/carcinoma, and liver metastases thereof. Further the expression of the glycans was detected in abundant amounts in salivary gland cancer/carcinoma, and skin metastases thereof.

In all these cancer types, neutral O-glycans were the most abundant cancer-associated structures, but both fucosylated and sialylated O-glycans were in all analyses detected to be expressed simultaneously; in most cases also their levels were found to be higher in cancer in comparison to normal human tissue samples. In addition, in benign tumors of the ovary and colon, O-glycan signals were significantly lower than in the corresponding malignant tumors, indicating that O-glycans are specifically associated with malignancy in human cancers.

Non-reducing terminal HexNAc glycan expression was detected along with one or both of the above mentioned glycan groups in the following analyzed cancer types: lung cancer, both small cell lung adenocarcinoma and non-small cell lung adenocarcinoma- and lung carcinoma liver metastases; breast cancer; ductale type breast adenocarcinoma and lymph node metastases thereof; lobulare type breast adenocarcinoma and lymph node metastases thereof; ovarian cystadenocarcinoma; colon cancer/carcinoma, carcinoma adenomatosum, and liver metastases thereof; kidney cancer/carcinoma, and kidney hypernephroma; gastric cancer/carcinoma, and lymph node metastases thereof, liver cancer/carcinoma; laryx cancer/carcinoma; pancreas cancer/carcinoma; melanoma and liver metastases thereof; gall bladder cancer/carcinoma, and liver metastases thereof; and lymph node cancer/carcinoma (lymphoma). Further the expression of the glycans was detected in abundant amounts in salivary gland cancer/carcinoma, and skin metastases thereof. The detection was based on their major indicative mass spectrometric glycan signals 1485 and/or 1850 corresponding to Hex₃HexNAc₄dHex₁ and Hex₄HexNAc₅dHex₁ glycan compositions, respectively, as described in the present invention.

### Example 21

### Neutral and Acidic Protein-Linked Tissue Glycan Profiles of Ductale and Lobulare Type Breast Carcinomas

Protein-linked glycans were isolated from formalin-fixed and paraffin-embedded tissue sections of ductale-type breast cancer and lymph node metastases derived therefrom, and analyzed as described in the preceding Examples.

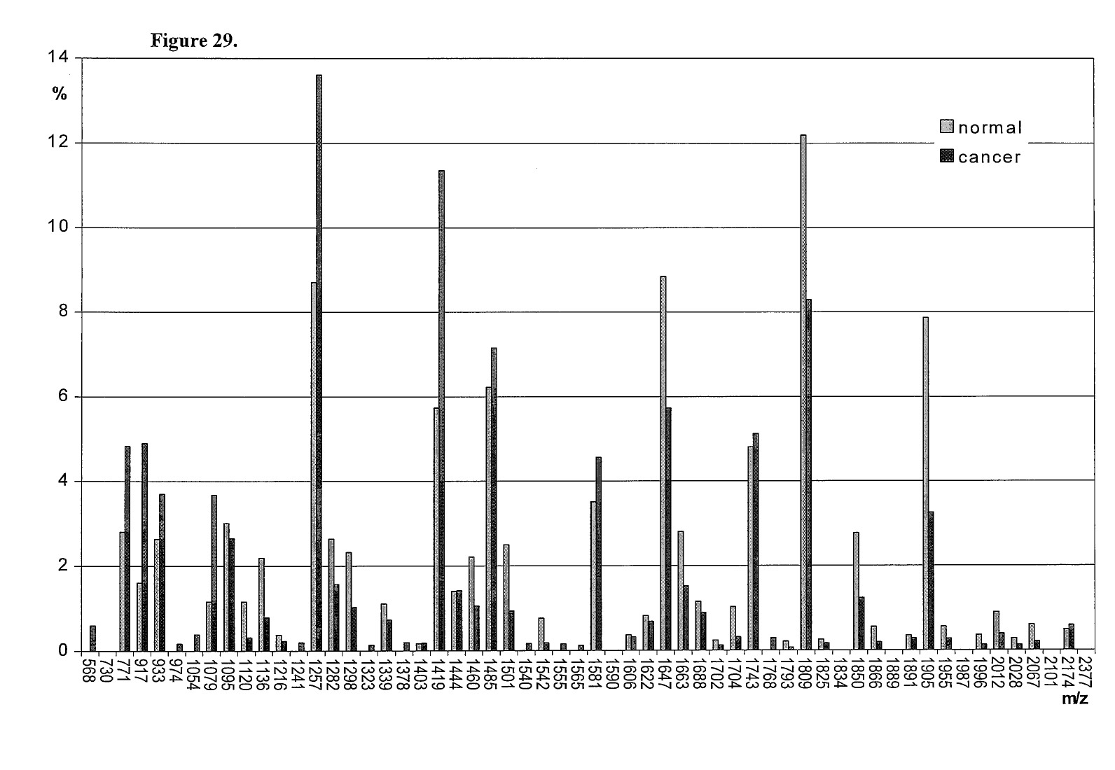

The results are described as protein-linked glycan profiles of the primary breast cancer tumors and normal brease tissues, as well as metastases and normal lymph node tissues from the same patients in FIGS. 29, 30, 31, and 32, respectively. The profiles indicate that major cancer- and metastasis-associated glycan signals and signal groups include low-mannose type glycans and O-glycas, based on the indicative signals for each glycan signal group, respectively.

Further, the acidic glycan profiles were quantitatively analyzed by composition classification into glycan structural features, as described in Table 20. Importantly, the classification analysis revealed that especially sulphation and/or phosphorylation and complex fucosylation of acidic glycans were associated with the present tumors as well as the corresponding lymph node metastases in comparison with either of the primary and the secondary normal tissues. Major glycan signals expressing these features were NeuAc₁Hex₂HexNAc₂SP₁ and NeuAc₁Hex₃HexNAc₃SP₂ wherein SP is either sulphate or phosphate ester, and NeuAc₁Hex₅HexNAc₄dHex₂ and NeuAc₁Hex₅HexNAc₄dHex₃, respectively.

### Example 22

### Metastasis-Associated Glycans in Malignancy and Site-Specific Metastasis to the Liver

### Experimental Procedures

A series of malignant primary tumors of the lung, colon, skin, and gall bladder, and corresponding liver metastases were analyzed in order to identify glycans associated with malignancy and metastasis formation, especially liver metastases. Protein-linked glycans were isolated from formalin-fixed and paraffin-embedded tissue sections or protein fractions of tumor tissues and analyzed by MALDI-TOF mass spectrometry as described in the preceding Examples.

By comparing the quantitative expression of indicative glycan signals of 1) liver metastases and normal liver tissue, and 2) malignant primary tumor and corresponding normal tissue, malignancy and metastasis-associated glycan signals and glycan structure groups were identified as described in the preceding Examples.

### Results and Discussion

Glycan groups that were associated with liver metastases were 1) low-mannose type glycans, 2) non-reducing terminal HexNAc glycans, especially non-reducing terminal GlcNAc glycans, and 3) neutral and acidic O-glycans, especially neutral and sialylated O-glycans. Preferential glycan signals associated with malignant liver metastases included 933, 1079, and 195 (15 (, 9, 8, 1 especially 771 and 899 (3); wherein the numbering (1-3) refers to the identified metastasis-associated glycan groups. The glycans were present in the primary tumor in elevated amounts in comparison to corresponding normal tissue, and significantly, further elevated in comparison to normal liver tissue i.e. enriched in the liver metastases. The results indicate that the present glycans identified in metastases are associated with metastasis formation, more specifically liver metastasis formation. Specifically, the present results indicate that low-mannose type, non-reducing terminal GlcNAc, and O-glycans are associated with malignant metastasis formation, more specifically liver metastasis formation; most specifically, low-mannose type and terminal GlcNAc glycans are associated with liver metastases.

### Example 23

### Metastasis-Associated Glycans in Malignancy and Site-Specific Metastasis to Lymph Nodes

### Experimental Procedures

A series of malignant primary tumors of the stomach and breast, and corresponding lymph node metastases were analyzed in order to identify glycans associated with malignancy and metastasis formation, especially lymph node metastases. Protein-linked glycans were isolated from formalin-fixed and paraffin-embedded tissue sections or protein fractions of tumor tissues and analyzed by MALDI-TOF mass spectrometry as described in the preceding Examples.

By comparing the quantitative expression of indicative glycan signals of 1) lymph node metastases and normal lymph node tissue, and 2) malignant primary tumor and corresponding normal tissue, malignancy and metastasis-associated glycan signals and glycan structure groups were identified as described in the preceding Examples.

### Results and Discussion

Glycan groups that were associated with lymph node metastases were 1) low-mannose type glycans and 2) neutral and acidic O-glycans, especially neutral and sialylated O-glycans. Preferential glycan signals associated with malignant liver metastases included 933, 1079, and 1095 (1); and 771, 917, 899, 1038, and 1329 (2), especially 771 and 899 (2); wherein the numbering (1-2) refers to the identified metastasis-associated glycan groups. The glycans were present in the primary tumor in elevated amounts in comparison to corresponding normal tissue, and significantly, further elevated in comparison to normal liver tissue i.e. enriched in the lymph node metastases. The results indicate that the present glycans identified in metastases are associated with metastasis formation, more specifically lymph node metastasis formation. Specifically, the present results indicate that low-mannose type and O-glycans are associated with malignant metastasis formation, more specifically lymph node metastasis formation; most specifically, low-mannose type and sialylated O-glycans are associated with lymph node metastases.

A sample array from different cancer patients with gastric cancer revealed an interesting phenomenon: lymph node metastases of primary gastric cancers transformed the lymph node glycan profiles with glycan signals originating from primary tissue specific glycosylation. For example, in lymph node metastases small O-glycan type signals with either blood group O, A, or B characteristics were detected. These signals in the primary tissue location included in both O, A, and B patients: 1063 (Hex2HexNAc2dHex2) i.e. increased amounts of dHex in glycans with 3 or less HexNAc residues and more clearly in glycans with 2 or less HexNAc residues; specifically in A patients: 1120 (Hex2HexNAc3dhex1) and 1266 (Hex2HexNAc3dHex2) i.e. increased amounts of HexNAc and dHex in glycans with 3 or less Hex residues and more clearly in glycans with 2 or less Hex residues; and specifically in B patients: 714 (Hex2HexNAc1dHex1) and/or 1225 (Hex3HexNAc2dHex2) i.e. increased amounts of Hex and dHex in glycans with 3 or less HexNAc residues and more clearly in glycans with 2 or less HexNAc residues. The present results demonstrated that the origin of the primary tumor is reflected in the glycan profile of the metastasis, and that the metastatic cancer cells carry with them abnormal glycan antigens to the site of the metastasis.

### Example 24

### In Vitro Lysis Assay to Detect Cytolytic Activity of the Antibodies Recognizing Terminal Mannose or Core 2 O-Glycan Structures According to the Present Invention

Model cancer cells comprising terminal mannose or Core 2 O-glycan structures on cell surface are harvested from a cancer patient or cultured to 80% confluent density and washed four times with HBSS (Hank's balanced salt solution). The viability of the cells is determined by trypan blue staining. Approximately 200 000 cells are incubated with antibodies binding the terminal mannose or Core 2 O-glycan structures according to the present invention on the cell surfaces. Rabbit complement is added to one set of cells to a final dilution of 1:5 and the other set of cells is adjusted to same volume with incubation media.

The cells are further incubated for 1 h at 37 degree of Celsius. Finally, protium iodide is added to final concentration of 20 micrograms per milliliter and the cells are analyzed for dye uptake. The cells are shown to be lysed by the terminal mannose or Core 2 O-glycan binding antibodies but not by non-cancer binding control antibodies. The terminal mannose or Core 2 O-glycan structures are available for antibody recognition and cytolysis on cell surface.

### Example 25

### Analysis of N-Glycan Composition Groups with Terminal HexNAc

Methods. To analyze the presence of terminal HexNAc containing N-glycans characterized by the formulae: n_{HexNAc}=n_{Hex}≧5 and n_{dHex}≧1 (group I), and to compare their occurrence to terminal HexNAc containing N-glycans characterized by the formulae: n_{HexNAc}=n_{Hex≧}5 and nd_{Hex}=0 (group II), protein-linked glycans were isolated, purified and analyzed by MALDI-TOF mass spectrometry as described in the preceding Examples. They were assigned monosaccharide compositions and their relative proportions within the obtained glycan profiles were determined by quantitative profile analysis as described above. The following glycan signals were used as indicators of the specific glycan groups (monoisotopic masses):

Ia, Hex₅HexNAc₅dHex₁: m/z for [M+Na]+ ion 2012.7
Ib, NeuAc₁Hex₅HexNAc₅dHex₁: m/z for [M−H]− ion 2279.8
Ic, NeuAc₂Hex₅HexNAc₅dHex₁: m/z for [M−H]− ion 2570.9

### IIa, NeuAc₁Hex₁HexNAc₅: m/z for [M−H]− ion 2133.8

Results. As an indicator of group I glycans, Ib was detected in various protein-linked glycan samples isolated from malignant tumor samples, including ductale and lobulare type breast carcinoma, and carcinomas of the pancreas, stomach, lung, kidney, colon, ovaries, and lymph nodes (lymphoma), as well as lymph node metastases from primary stomach carcinoma and lobulare and ductale type breast carcinomas. Specifically, Ib was overexpressed in the malignant tumors of ductale type breast carcinoma when compared to corresponding normal tissues. When the two breast cancer types were more specifically studied with regard to the relationship of group I and II glycan expression, it was found that they expressed 1a, 1b, and Ic, while IIa was not expressed in similar amounts in same tissue samples as group I glycans. More specifically, IIa was not detected in lobulare type breast cancer samples, where IIb was expressed and further overexpressed when compared to the corresponding normal tissue from same individual. Further, of these indicator signals only group I but not group II was expressed in lymph node metastases from these two breast cancer types

### Example 26

### Evaluation of Glycan Groups Associated with Metastases

A series of malignant primary tumors of the lung, colon, skin, and gall bladder, and corresponding liver metastases were analyzed in order to identify glycans associated with malignancy, metastasis formation, metastatic tumors, and metastatic cells, especially in liver metastases; a series of malignant primary tumors of the stomach and breast, and corresponding lymph node metastases were analyzed in order to identify glycans associated with malignancy, metastasis formation, metastatic tumors, and metastatic cells, especially in lymph node metastases. Protein-linked glycans were isolated from formalin-fixed and paraffin-embedded tissue sections or protein fractions of tumor tissues and analyzed by MALDI-TOF mass spectrometry as described in the preceding Examples. The analysis was performed by comparing quantitative expression of indicative glycan signals between samples as described in the preceding Examples.

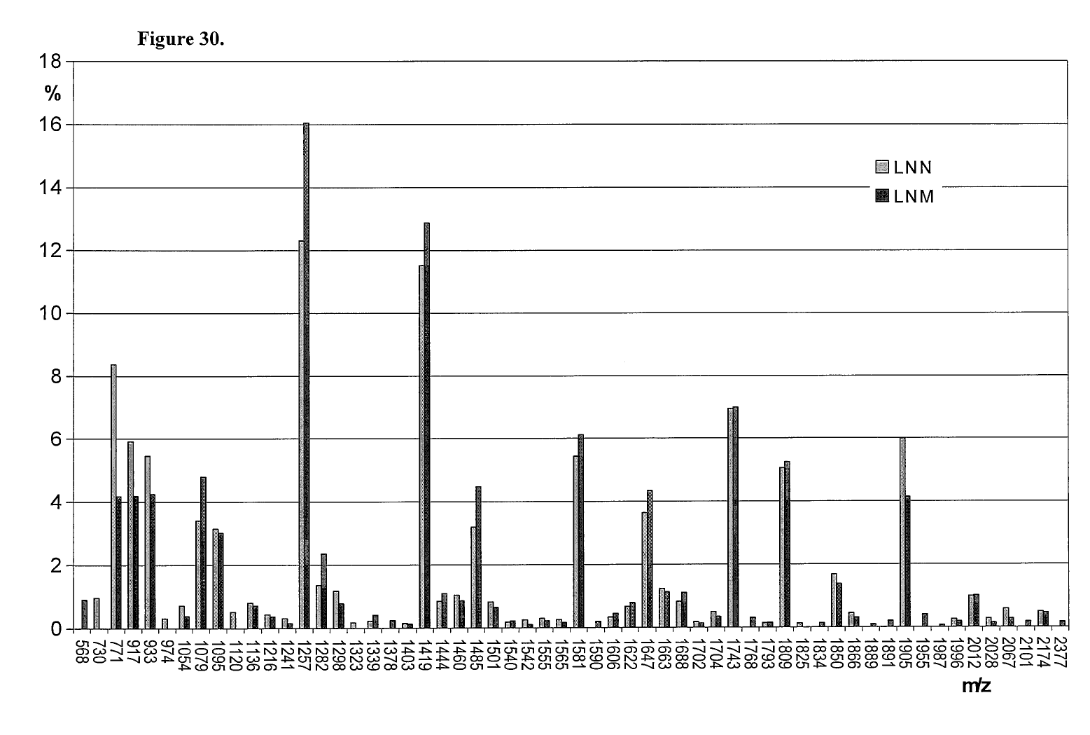

Structure groups that demonstrated quantitatively different in neutral protein-linked glycan expression profiles in metastases compared to either the primary tumor or site of metastasis were (exemplary glycan signals of each group in parenthesis; see FIGS. 29 and 30): high-mannose type N-glycans (1257); low-mannose type N-glycans, more specifically non-fucosylated (933) and fucosylated (1079) low-mannose type N-glycans were differentially expressed; hybrid-type or monoantennary N-glycans, more specifically non-fucosylated (1622) and fucosylated (1606) hybrid-typDe or monoantennary N-glycans were differentially expressed; complex-type N-glycans, more specifically non-fucosylated (1339), fucosylated (1485), and terminal HexNAc-type complex-type N-glycans (1485) were differentially expressed; neutral O-glycans according to the present invention (771, 917) were differentially expressed; and soluble glycans according to the present invention (1054) were differentially expressed. Specifically, these glycan groups contain terminal structures groups GalβGlcNAc, Gal₁GalNAc₁ HexNAcβincluding GlcNAcβ, and fucosylated variants thereof as described in the present invention, which are differentially expressed in specifically varying amounts. Glycan signals corresponding to these glycan groups were detected and grouped as listed in Table 14 and as described in the present invention. Such differently expressed glycan signals were also observed in the other metastasis types analyzed as listed above, including also liver metastases. Metastasis-associated neutral protein-linked glycan types of ductale type breast cancer were especially fucosylated low-mannose type N-glycans, Man₅₋₇ high-mannose type N-glycans, fucosylated hybrid-type and/or monoantennary N-glycans, and complex-type N-glycans with terminal HexNAc.

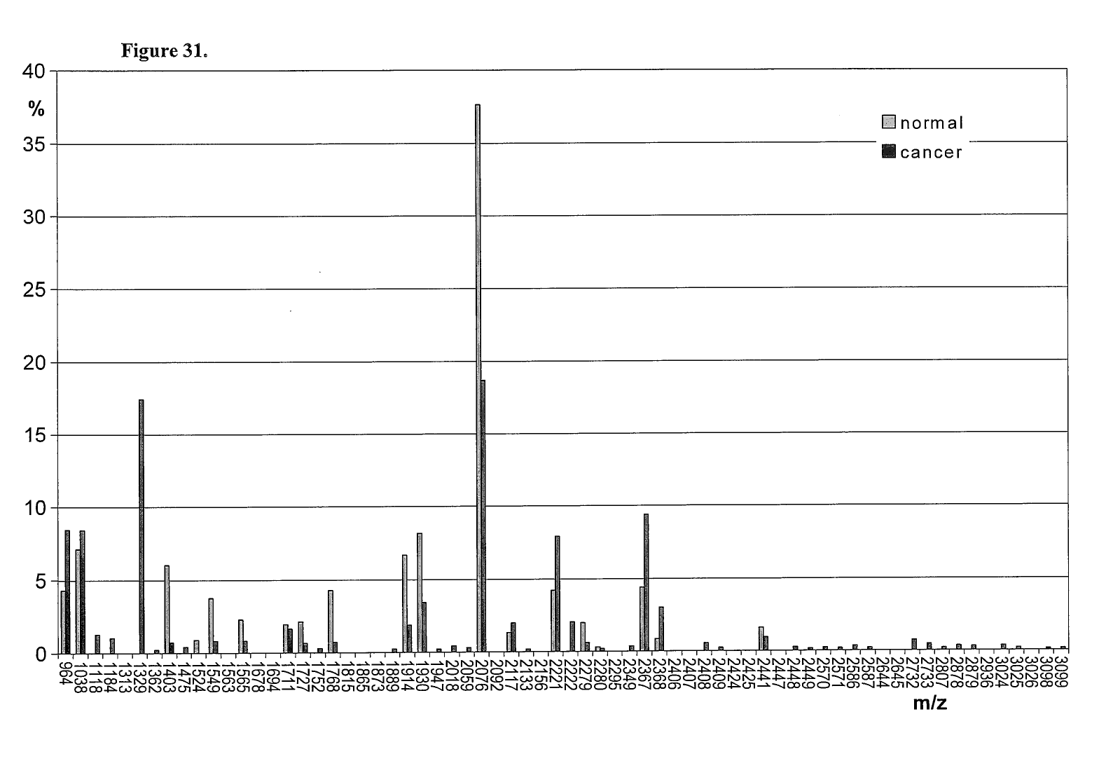

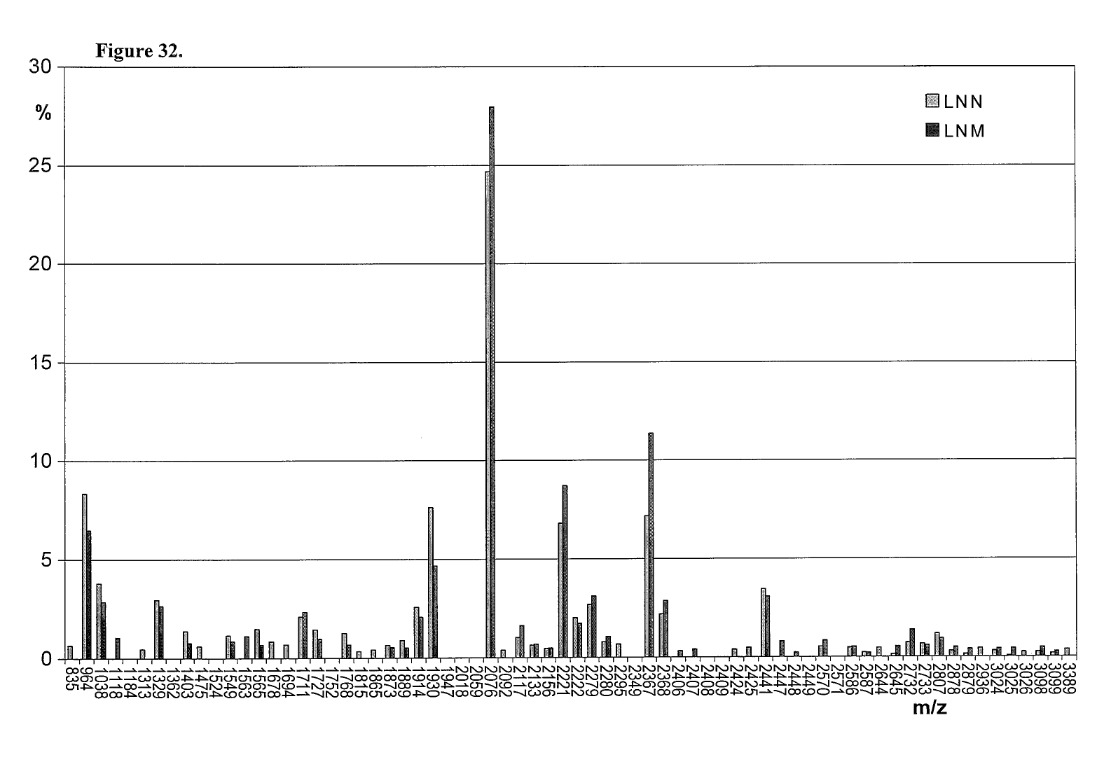

Structure groups that demonstrated quantitatively different in acidic protein-linked glycan expression profiles in metastases compared to either the primary tumor or site of metastasis were (exemplary glycan signals of each group in parenthesis; see FIGS. 31 and 32): hybrid-type or monoantennary N-glycans, more specifically non-fucosylated (1727) and fucosylated (1711) hybrid-type or monoantennary N-glycans were differently expressed; complex-type N-glycans, more specifically non-fucosylated (2221), fucosylated (2076), multifucosylated (2222), terminal HexNAc-type complex-type N-glycans (2279), and mono-(1930) and multisialylated (2367) N-glycans, were differently expressed; O-glycans according to the present invention including Core1 type (964), and larger O-glycans including Core2 type (1329), mono-(1038) and multisialylated (1329), fucosylated (1184), and sulphated and/or phosphorylated (1118) O-glycans were differently expressed; glycan signals corresponding to these glycan groups were detected and grouped as listed in Table 15 and as described in the present invention. Specifically, these glycan groups contain terminal structures groups GalβGlcNAc, GalβGalNAc, HexNAcβincluding GlcNAcβ, and sialylated and/or fucosylated and/or sulphated and/or phosphorylated variants thereof as described in the present invention, which are differentially expressed in specifically varying amounts. Such differently expressed glycan signals were also observed in the other metastasis types analyzed as listed above, including also liver metastases. Lymph node metastasis-associated acidic protein-linked glycan types of ductale type breast cancer were especially multisialylated, fucosylated and/or multifucosylated N-glycans, and O-glycans with larger core structure including Core2 structure, fucosylation, multisialylation, and/or sulphation and/or phosphorylation.

It is concluded that observation of these glycan groups is useful in characterizing metastases and metastatic cells. It was further concluded that observation of these glycans and glycan groups as listed in Tables 14 and 15 and as described in the present invention is useful in following metastases and metastatic cells. More specific glycan signals and glycan groups with regard to metastatic cell recognition are further specifically discussed in Examples 21-23 and listed in Table 20.

TABLE 1
Overexpression data of low-mannose type N-glycans in studied
cancer types.

| | m/z | | | | | |
|---|---|---|---|---|---|---|
| Cancer type | 917 | 933 | 1079 | 1095 | 1241 | 1403 |
| Non-small cell lung | + | − | + | − | + | + |
| adenocarcinoma | | | | | | |
| Ductale breast | + | + | + | + | + | + |
| adenocarcinoma | | | | | | |
| Lobulare breast | + | + | + | − | + | + |
| adenocarcinoma | | | | | | |
| Ovarian cystadenocarcinoma | + | + | + | − | + | + |
| Colon carcinoma | + | − | + | − | + | − |
| Kidney cancer | − | + | − | − | − | − |
| Gastric cancer | − | + | + | + | − | − |
| Liver cancer | − | + | − | − | − | − |
| Larynx cancer | + | + | + | − | − | − |
| Pancreas cancer | − | + | + | + | − | − |

| | |
|---|---|
| CODE: | |
| + | overexpressed in cancer |
| − | no overexpression detected |
| m/z 917 | Hex₂HexNAc₂dHex₁ |
| m/z 933 | Hex₃HexNAc₂ |
| m/z 1079 | Hex₃HexNAc₂dHex₁ |
| m/z 1095 | Hex₄HexNAc₂ |
| m/z 1241 | Hex₄HexNAc₂dHex₁ |
| m/z 1403 | Hex₅HexNAc₂dHex₁ |

TABLE 2
Statistical analysis of low-mannose type N-glycans in breast cancer.
DUCTALE BREAST ADENOCARCINOMA
9 sample pairs

| Approx. m/z | Composition | P | Test |
|---|---|---|---|
| 917 | Hex₂HexNAc₂dHex₁ | 0.0039 | Signed Rank |
| 933 | Hex₃HexNAc₂ | 0.0031 | Student's t |
| 1079 | Hex₃HexNAc₂dHex₁ | 0.0039 | Signed Rank |
| 1241 | Hex₄HexNAc₂dHex₁ | 0.0071 | Student's t |

TABLE 3
Overexpression data of neutral O-glycans in studied cancer types.

m/z

| Cancer type | 771 | 917 |
|---|---|---|
| Non-small cell lung adenocarcinoma | + | + |
| Ductale breast adenocarcinoma | + | + |
| Lobulare breast adenocarcinoma | + | + |
| Ovarian cystadenocarcinoma | + | + |
| Colon carcinoma | − | + |
| Kidney cancer | − | − |
| Gastric cancer | + | − |
| Liver cancer | − | − |
| Larynx cancer | − | + |
| Pancreas cancer | + | − |

CODE:
+ overexpressed in cancer
− no overexpression detected
m/z 771 Hex₂HexNAc₂
m/z 917 Hex₂HexNAc₂dHex₁

TABLE 4
Statistical analysis of neutral O-glycan overexpression
in lung cancer and in breast cancer.

| | | | |
|---|---|---|---|
| Approx. m/z | Composition | P | Test |

DUCTALE BREAST ADENOCARCINOMA
9 sample pairs

| | | | |
|---|---|---|---|
| 771 | Hex₂HexNAc₂ | 0.0039 | Sign |
| 917 | Hex₂HexNAc₂dHex₁ | 0.0078 | Signed Rank |

NON-SMALL CELL LUNG ADENOCARCINOMA
8 sample pairs

| | | | |
|---|---|---|---|
| 771 | Hex₂HexNAc₂ | <0.05 | Student's t |

TABLE 5
The indicative mass spectrometric signals of the glycans.
A) O-glycan fragments, positive ion mode (isotopic masses).

| | m/z | m/z | m/z | m/z | m/z |
|---|---|---|---|---|---|
| monosaccharide composition | [M + H]⁺ | [M + Na]⁺ | [M + K]⁺ | [M − H + 2Na]⁺ | [M − H + Na + K]⁺ |
| NeuNAc₁Hex₁HexNAc₁(deoxyamino)HexNAc₁ | 877.34 | 899.32 | 915.30 | 921.31 | 937.28 |
| NeuNAc₁Hex₁HexNAc₁(deoxyamino)HexNAc₁dHex₁ | 1023.40 | 1045.38 | 1061.36 | 1067.36 | 1083.34 |
| NeuNAc₁Hex₂HexNAc₂(deoxyamino)HexNAc₁ | | 1264.46 | | 1286.44 | |
| NeuNAc₁Hex₂HexNAc₂(deoxyamino)HexNAc₁dHex₁ | | 1410.51 | | 1432.50 | |
| NeuNAc₁Hex₂HexNAc₂(deoxyamino)HexNAc₁dHex₂ | | 1556.57 | | 1578.55 | |
| NeuNAc₁Hex₃HexNAc₃(deoxyamino)HexNAc₁ | | 1629.59 | | 1651.57 | |
| NeuNAc₁Hex₃HexNAc₃(deoxyamino)HexNAc₁dHex₁ | | 1775.65 | | 1797.63 | |

| | | |
|---|---|---|
| monosaccharide composition | m/z [M − H]⁻ | average |

B) O-glycan fragments, negative ion mode (isotopic and average masses).

| | | |
|---|---|---|
| euNAc₁Hex₁HexNAc₁(deoxyamino)HexNAc₁ | 875.33 | 875.80 |
| NeuNAc₁Hex₁HexNAc₁(deoxyamino)HexNAc₁dHex₁ | 1021.38 | 1021.94 |
| NeuNAc₁Hex₂HexNAc₂(deoxyamino)HexNAc₁ | 1240.46 | 1241.14 |
| NeuNAc₁Hex₂HexNAc₂(deoxyamino)HexNAc₁dHex₁ | 1386.52 | 1387.28 |
| NeuNAc₁Hex₂HexNAc₂(deoxyamino)HexNAc₁dHex₂ | 1532.57 | 1533.42 |
| NeuNAc₁Hex₃HexNAc₃(deoxyamino)HexNAc₁ | 1605.59 | 1606.47 |
| NeuNAc₁Hex₃HexNAc₃(deoxyamino)HexNAc₁dHex₁ | 1751.65 | 1752.61 |
| NeuNAc₁Hex₃HexNAc₃(deoxyamino)HexNAc₁dHex₁ | 1897.71 | 1898.75 |

C) Oligosaccharides, negative ion mode (isotopic and average masses).

| | | |
|---|---|---|
| NeuNAc₁Hex₂HexNAc₂ | 1038.36 | 1038.93 |
| NeuNAc₁Hex₂HexNAc₂dHex₁ | 1184.42 | 1185.07 |
| NeuNAc₂Hex₂HexNAc₂ | 1329.46 | 1330.18 |
| NeuNAc₁Hex₃HexNAc₃ | 1403.49 | 1404.26 |
| NeuNAc₂Hex₂HexNAc₂dHex₁ | 1475.52 | 1476.32 |
| NeuNAc₁Hex₃HexNAc₃dHex₁ | 1549.55 | 1550.40 |
| NeuNAc₂Hex₃HexNAc₃ | 1694.59 | 1695.52 |
| NeuNAc₂Hex₃HexNAc₃dHex₁ | 1840.65 | 1841.66 |

TABLE 6
Overexpression data of sialylated Core 2 type O-glycans
in studied cancer types.

| | m/z |
|---|---|
| Cancer type | 899 |
| Non-small cell lung adenocarcinoma | + |
| Ductale breast adenocarcinoma | + |
| Lobulare breast adenocarcinoma | + |
| Ovarian cystadenocarcinoma | + |
| Colon carcinoma | + |
| Kidney cancer | + |
| Gastric cancer | − |
| Liver cancer | − |
| Larynx cancer | − |
| Pancreas cancer | + |

CODE:
+ overexpressed in cancer
− no overexpression detected
m/z 771: Hex₂HexNAc₂
m/z 917: Hex₂HexNAc₂dHex₁

TABLE 7
Statistical analysis of sialylated Core 2 type O-glycan overexpression
in lung cancer and in two types of breast cancer.

| | | | |
|---|---|---|---|
| Approx. | | | |
| m/z | Composition | P | Test |

DUCTALE BREAST ADENOCARCINOMA
9 sample pairs

| | | | |
|---|---|---|---|
| 899 | NeuAc₁Hex₁HexNAc₁(deoxyamino)- | 0.0078 | Sign |
| | HexNAc₁ | | |

LOBULARE BREAST ADENOCARCINOMA
6 sample pairs

| | | | |
|---|---|---|---|
| 899 | NeuAc₁Hex₁HexNAc₁(deoxyamino)- | 0.0313 | Signed |
| | HexNAc₁ | | Rank |

NON-SMALL CELL LUNG ADENOCARCINOMA
8 sample pairs

| | | | |
|---|---|---|---|
| 899 | NeuAc₁Hex₁HexNAc₁(deoxyamino)- | <0.05 | Student's t |
| | HexNAc₁ | | |

TABLE 8
Proposed monosaccharide compositions for MALDI-TOF
mass spectrometric profiling of sialylated protein-linked
glycans isolated from A. healthy ovarian tissue, B. benign
ovarian cystadenoma, and C. malignant ovarian
cystadenocarcinoma in FIG. y1.

| No. | calc. m/z | exp. m/z | proposed composition |
|---|---|---|---|
| 1 | 1550.40 | 1550.36 | NeuAc1Hex3HexNAc3dHex1 |
| 2 | 1558.40 | 1558.37 | Hex4HexNAc4SO3 |
| 3 | 1566.40 | 1566.93 | NeuAc1Hex4HexNAc3 |
| 4 | | 1588.17 | unknown |
| 5 | | 1617.27 | unknown |
| 6 | | 1654.70 | unknown |
| 7 | 1712.54 | 1712.13 | NeuAc1Hex4HexNAc3dHex1 |
| 8 | | 1719.82 | unknown |
| 9 | 1728.54 | 1728.33 | NeuAc1Hex5HexNAc3 |
| 10 | 1753.60 | 1753.61 | NeuAc1Hex3HexNAc4dHex1 |
| 11 | 1769.60 | 1769.86 | NeuAc1Hex4HexNAc4 |
| 12 | 1785.59 | 1785.98 | NeuGc1Hex4HexNAc4 |
| 13 | 1816.61 | 1816.45 | NeuAc2Hex5HexNAc2 |
| 14 | 1857.66 | 1857.37 | NeuAc2Hex4HexNAc3 |
| 15 | 1874.68 | 1874.51 | NeuAc1Hex5HexNAc3dHex1 |
| 16 | 1882.69 | 1882.09 | Hex6HexNAc4SO3 |
| 17 | 1915.74 | 1915.68 | NeuAc1Hex4HexNAc4dHex1 |
| 18 | 1931.74 | 1931.73 | NeuAc1Hex5HexNAc4 |
| 19 | 1947.74 | 1947.80 | NeuGc1Hex5HexNAc4 |
| 20 | | 1978.32 | unknown |
| 21 | | 1988.68 | unknown |
| 22 | 2020.83 | 2020.05 | NeuAc1Hex5HexNAc3dHex2 |
| 23 | 2044.85 | 2044.17 | NeuAc2Hex3HexNAc4dHex1 |
| 24 | 2077.88 | 2077.81 | NeuAc1Hex5HexNAc4dHex1 |
| 25 | | 2110.12 | unknown |
| 26 | 2118.93 | 2118.81 | NeuAc1Hex4HexNAc5dHex1 |
| 27 | 2159.98 | 2159.72 | NeuAc1Hex3HexNAc6dHex1 |
| 28 | | 2198.95 | unknown |
| 29 | 2223.00 | 2223.03 | NeuAc2Hex5HexNAc4 |
| 30 | 2281.07 | 2281.08 | NeuAc1Hex5HexNAc5dHex1 |
| 31 | | 2308.09 | unknown |
| 32 | 2328.08 | 2326.60 | NeuAc2Hex3HexNAc6dHex1 |
| 33 | 2352.10 | 2350.72 | NeuAc3Hex45HexNAc4 |
| 34 | 2369.13 | 2369.21 | NeuAc2Hex5HexNAc4dHex1 |
| 35 | 2402.19 | 2401.80 | NeuAc1Hex4HexNAc6dHex1SO3 |
| 36 | 2410.18 | 2409.32 | NeuAc1Hex4HexNAc5dHex3 |
| 37 | 2443.21 | 2443.01 | NeuAc1Hex5HexNAc6dHex1 |
| 38 | 2451.24 | 2449.46 | NeuAc2Hex3HexNAc6dHex1 |
| 39 | | 2547.32 | unknown |
| 40 | 2572.32 | 2572.25 | NeuAc2Hex5HexNAc5dHex1 |
| 41 | 2588.32 | 2588.01 | NeuAc2Hex6HexNAc5 |
| 42 | 2734.46 | 2734.46 | NeuAc2Hex6HexNAc5dHex1 |
| 43 | 2775.52 | 2773.43 | NeuAc2Hex5HexNAc6dHex1 |
| 44 | 2808.54 | 2808.40 | NeuAc1Hex7HexNAc6dHex1 |
| 45 | 2816.57 | 2814.30 | NeuAc2Hex4HexNAc7dHex1 |
| 46 | 2879.58 | 2879.54 | NeuAc3Hex6HexNAc5 |
| 47 | 3025.72 | 3025.72 | NeuAc3Hex6HexNAc5dHex1 |
| 48 | 3107.82 | 3104.91 | NeuAc3Hex4HexNAc7dHex1 |
| 49 | 3244.91 | 3245.37 | NeuAc3Hex7HexNAc6 |
| 50 | 3391.05 | 3390.79 | NeuAc3Hex7HexNAc6dHex1 |
| 51 | 3536.17 | 3536.17 | NeuAc4Hex7HexNAc6 |
| 52 | 3682.31 | 3682.59 | NeuAc4Hex7HexNAc6dHex1 |

Experimental masses (exp. m/z) refer to spectrum B.;
SO3, sulfate/phosphate.

TABLE 9
Preferred neutral glycan compositions. Calculated
mass-to-charge ratios (calc. m/z) refer to the first
isotope signal of [M + Na]⁺ ion.

| Proposed composition | calc. m/z |
|---|---|
| HexHexNAc | 406.13 |
| Hex3 | 527.16 |
| HexHexNAcdHex | 552.19 |
| Hex2HexNAc | 568.19 |
| HexHexNAc2 | 609.21 |
| Hex4 | 689.21 |
| Hex2HexNAcdHex | 714.24 |
| Hex3HexNAc | 730.24 |
| HexHexNAc2dHex | 755.27 |
| Hex2HexNAc2 | 771.26 |
| HexHexNAc3 | 812.29 |
| Hex5 | 851.26 |
| Hex2HexNAcdHex2 | 860.30 |
| Hex4HexNAc | 892.29 |
| HexHexNAc2dHex2 | 901.33 |
| Hex2HexNAc2dHex | 917.32 |
| Hex3HexNAc2 | 933.32 |
| HexHexNAc3dHex | 958.35 |
| Hex2HexNAc3 | 974.34 |
| Hex2HexNAcdHex3 | 1006.36 |
| Hex6 | 1013.32 |
| HexHexNAc4 | 1015.37 |
| Hex3HexNAcdHex2 | 1022.35 |
| Hex5HexNAc | 1054.34 |
| Hex2HexNAc2dHex2 | 1063.38 |
| Hex3HexNAc2dHex | 1079.38 |
| Hex4HexNAc2 | 1095.37 |
| HexHexNAc3dHex2 | 1104.41 |
| Hex2HexNAc3dHex | 1120.40 |
| Hex3HexNAc3 | 1136.40 |
| Hex2HexNAcdHex4 | 1152.42 |
| HexHexNAc4dHex | 1161.43 |
| Hex7 | 1175.37 |
| Hex2HexNAc4 | 1177.42 |
| Hex2HexNAc2dHex3 | 1209.44 |
| Hex6HexNAc | 1216.40 |
| HexHexNAc5 | 1218.45 |
| Hex3HexNAc2dHex2 | 1225.43 |
| Hex4HexNAc2dHex | 1241.43 |
| Hex5HexNAc2 | 1257.42 |
| Hex2HexNAc3dHex2 | 1266.46 |
| Hex3HexNAc3dHex | 1282.45 |
| Hex4HexNAc3 | 1298.45 |
| HexHexNAc4dHex2 | 1307.49 |
| Hex2HexNAc4dHex | 1323.48 |
| Hex8 | 1337.42 |
| Hex3HexNAc4 | 1339.48 |
| Hex2HexNAc2dHex4 | 1355.50 |
| HexHexNAc5dHex | 1364.51 |
| Hex3HexNAc2dHex3 | 1371.49 |
| Hex7HexNAc | 1378.45 |
| Hex4HexNAc2dHex2 | 1387.49 |
| Hex2HexNAc5 | 1380.50 |
| Hex5NexNAc2dHex | 1403.48 |
| Hex2HexNAc3dHex3 | 1412.52 |
| Hex6HexNAc2 | 1419.48 |
| HexHexNAc6 | 1421.53 |
| Hex3HexNAc3dHex2 | 1428.51 |
| Hex4HexNAc3dHex | 1444.51 |
| HexHexNAc4dHex3 | 1453.54 |
| Hex5HexNAc3 | 1460.50 |
| Hex2HexNAc4dHex2 | 1469.54 |
| Hex3HexNAc4dHex | 1485.53 |
| Hex9 | 1499.48 |
| Hex4HexNAc4 | 1501.53 |
| HexHexNAc5dHex2 | 1510.57 |
| Hex3HexNAc2dHex4 | 1517.55 |
| Hex2HexNAc5dHex | 1526.56 |
| Hex4HexNAc2dHex3 | 1533.54 |
| Hex8HexNAc | 1540.50 |
| Hex3HexNAc5 | 1542.56 |
| Hex5HexNAc2dHex2 | 1549.54 |
| Hex6HexNAc2dHex | 1565.53 |
| Hex3HexNAc3dHex3 | 1574.57 |
| Hex7HexNAc2 | 1581.53 |
| Hex2HexNAc6 | 1583.58 |
| Hex4HexNAc3dHex2 | 1590.57 |
| Hex5HexNAc3dHex | 1606.56 |
| Hex2HexNAc4dHex3 | 1615.60 |
| Hex6HexNAc3 | 1622.56 |
| Hex3HexNAc4dHex2 | 1631.59 |
| Hex4HexNAc4dHex | 1647.59 |
| Hex10 | 1661.53 |
| Hex5HexNAc4 | 1663.58 |
| Hex2HexNAc5dHex2 | 1672.62 |
| Hex3HexNAc5dHex | 1688.61 |
| Hex5HexNAc2dHex3 | 1695.60 |
| Hex9HexNAc | 1702.56 |
| Hex4HexNAx5 | 1704.61 |
| Hex6HexNAc2dHex2 | 1711.59 |
| Hex3HexNAc3dHex4 | 1720.63 |
| Hex7HexNAc2dHex | 1727.59 |
| Hex2HexNAc6dHex | 1729.64 |
| Hex4HexNAc3dHex3 | 1736.62 |
| Hex8HexNAc2 | 1743.58 |
| Hex3HexNAc6 | 1745.64 |
| Hex5HexNAc3dHex2 | 1752.62 |
| Hex6HexNAc3dHex | 1768.61 |
| Hex3HexNAc4dHex3 | 1777.65 |
| Hex7HexNAc3 | 1784.61 |
| Hex4HexNAc4dHex2 | 1793.64 |
| Hex5HexNAc4dHex | 1809.64 |
| Hex2HexNAc5dHex3 | 1818.68 |
| Hex11 | 1823.58 |
| Hex6HexNAc4 | 1825.63 |
| Hex3HexNAc5dHex2 | 1834.67 |
| Hex4HexNAc5dHex | 1850.67 |
| Hex6HexNAc2dHex3 | 1857.65 |
| Hex10HexNAc | 1864.61 |
| Hex5HexNAc5 | 1866.66 |
| Hex7HexNAc2dHex2 | 1873.64 |
| Hex2HexNAc6dHex2 | 1875.70 |
| Hex4HexNAc3dHex4 | 1882.68 |
| Hex8HexNAc2dHex | 1889.64 |
| Hex3HexNAc6dHex | 1891.69 |
| Hex5HexNAc3dHex3 | 1898.68 |
| Hex9HexNAc2 | 1905.63 |
| Hex4HexNAc6 | 1907.69 |
| Hex6HexNAc3dHex2 | 1914.67 |
| Hex3HexNAc4dHex4 | 1923.71 |
| Hex7HexNAc3dHex | 1930.67 |
| Hex2HexNAc7dHex | 1932.72 |
| Hex4HexNAc4dHex3 | 1939.70 |
| Hex8HexNAc3 | 1946.66 |
| Hex5HexNAc4dHex2 | 1955.70 |
| Hex6HexNAc4dHex | 1971.69 |
| Hex3HexNAc5dHex3 | 1980.73 |
| Hex12 | 1985.63 |
| Hex7HexNAc4 | 1987.69 |
| Hex4HexNAc5dHex2 | 1996.72 |
| Hex5HexNAc5dHex | 2012.72 |
| Hex7HexNAc2dHex3 | 2019.70 |
| Hex2HexNAc6dHex3 | 2021.76 |
| Hex11HexNAc | 2026.66 |
| Hex6HexNAc5 | 2028.71 |
| Hex8HexNAc2dHex2 | 2035.70 |
| Hex3HexNAc6dHex2 | 2037.75 |
| Hex5HexNAc3dHex4 | 2044.73 |
| Hex4HexNAc6dHex | 2053.75 |
| Hex6HexNAc3dHex3 | 2060.73 |
| Hex10HexNAc2 | 2067.69 |
| Hex5HexNAc6 | 2069.74 |
| Hex7HexNAc3dHex2 | 2076.72 |
| Hex2HexNAc7dHex2 | 2078.78 |
| Hex4HexNAc4dHex4 | 2085.76 |
| Hex8HexNAc3dHex | 2092.72 |
| Hex3HexNAc7dHex | 2094.77 |
| Hex5HexNAc4dHex3 | 2101.76 |
| Hex9HexNAc3 | 2108.71 |
| Hex4HexNAc7 | 2110.77 |
| Hex6HexNAc4dHex2 | 2117.75 |
| Hex3HexNAc5dHex4 | 2126.79 |
| Hex7HexNAc4dHex | 2133.75 |
| Hex4HexNAc5dHex3 | 2142.78 |
| Hex13 | 2147.69 |
| Hex8HexNAc4 | 2149.74 |
| Hex5HexNAc5dHex2 | 2158.78 |
| Hex6HexNAc5dHex | 2174.77 |
| Hex8HexNAc2dHex3 | 2181.76 |
| Hex3HexNAc6dHex3 | 2183.81 |
| Hex12HexNac | 2188.71 |
| Hex7HexNAc5 | 2190.77 |
| Hex4HexNAc6dHex2 | 2199.80 |
| Hex5HexNAc6dHex | 2215.80 |
| Hex7HexNAc3dHex3 | 2222.78 |
| Hex2HexNAc7dHex3 | 2224.84 |
| Hex11HexNAc2 | 2229.74 |
| Hex6HexNAc6 | 2231.79 |
| Hex8HexNAc3dHex2 | 2238.78 |
| Hex3HexNAc7dHex2 | 2240.83 |
| Hex5HexNAc4dHex4 | 2247.81 |
| Hex4HexNAc7dHex | 2256.83 |
| Hex6HexNAc4dHex3 | 2263.81 |
| Hex5HexNAc7 | 2272.82 |
| Hex7HexNAc4dHex2 | 2279.80 |
| Hex4HexNAc5dHex4 | 2288.84 |
| Hex5HexNAc5dHex3 | 2304.84 |
| Hex14 | 2309.74 |
| Hex9HexNAc4 | 2311.79 |
| Hex6HexNAc5dHex2 | 2320.83 |
| Hex7HexNAc5dHex | 2336.82 |
| Hex4HexNAc6dHex3 | 2345.86 |
| Hex8HexNAc5 | 2352.82 |
| Hex5HexNAc6dHex2 | 2361.86 |
| Hex6HexNAc6dHex | 2377.85 |
| Hex8HexNAc3dHex3 | 2384.83 |
| Hex3HexNAc7dHex3 | 2386.89 |
| Hex12HexNac2 | 2391.79 |
| Hex7HexNAc6 | 2393.85 |
| Hex4HexNAc7dHex2 | 2402.88 |
| Hex6HexNAc4dHex4 | 2409.87 |
| Hex5HexNAc7dHex | 2418.88 |
| Hex7HexNAc4dHex3 | 2425.86 |
| Hex6HexNAc7 | 2434.87 |
| Hex5HexNAc5dHex4 | 2450.89 |
| Hex6HexNAc5dHex3 | 2466.89 |
| Hex15 | 2471.79 |
| Hex7HexNAc5dHex2 | 2482.88 |
| Hex8HexNAc5dHex | 2498.88 |
| Hex5HexNAc6dHex3 | 2507.91 |
| Hex6HexNAc6dHex2 | 2523.91 |
| Hex7HexNAc6dHex | 2539.90 |
| Hex4HexNAc7dHex3 | 2548.94 |
| Hex13HexNAc2 | 2553.85 |
| Hex8HexNAc6 | 2555.90 |
| Hex5HexNAc7dHex2 | 2564.94 |
| Hex6HexNAc7dHex | 2580.93 |
| Hex6HexNAc5dHex4 | 2612.95 |
| Hex7HexNAc5dHex3 | 2628.94 |
| Hex16 | 2633.85 |
| Hex8HexNAc5dHex2 | 2644.94 |
| Hex6HexNAc6dHex3 | 2669.97 |
| Hex7HexNAc6dHex2 | 2685.96 |
| Hex5HexNAc7dHex3 | 2710.99 |
| Hex14HexNAc2 | 2715.90 |
| Hex6HexNAc7dHex2 | 2726.99 |
| Hex7HexNAc7dHex | 2742.98 |
| Hex8HexNAc7 | 2758.98 |
| Hex7Hexnac5dHex4 | 2775.00 |
| Hex8HexNAc5dHex3 | 2790.99 |
| Hex17 | 2795.90 |
| Hex7HexNAc6dHex3 | 2832.02 |
| Hex16HexNAc | 2836.92 |
| Hex9HexNAc6dHex | 2864.01 |
| Hex6HexNAc7dHex3 | 2873.05 |
| Hex15HexNAc2 | 2877.95 |
| Hex8HexNAc7dHex | 2905.04 |
| Hex8Hexnac5dHex4 | 2937.05 |
| Hex18 | 2957.95 |
| Hex7HexNAc6dHex4 | 2978.08 |
| Hex17HexNAc | 2998.98 |
| Hex8HexNAc7dHex2 | 3051.09 |
| Hex9HexNAc8 | 3124.11 |
| Hex8HexNAc6dHex4 | 3140.13 |
| Hex8HexNAc7dHex3 | 3197.15 |
| Hex9HexNAc8dHex/ | 3270.17 |
| Hex7HexNAc6dHex6 | |
| Hex9HexNAc6dHex4 | 3302.18 |
| Hex8HexNAc7dHex4 | 3343.21 |
| Hex9HexNAc8dHex2 | 3416.23 |
| Hex10HexNAc6dHex4 | 3464.24 |
| Hex10HexNAc9 | 3489.24 |
| Hex9HexNAc8dHex3 | 3562.28 |
| Hex11HexNAc6dHex4 | 3626.29 |
| Hex10HexNAc9dHex | 3635.30 |
| Hex9HexNAc8dHex4 | 3708.34 |
| Hex10HexNAc9dHex2/ | 3781.36 |
| Hex8HexNAc7dHex7 | |
| Hex9HexNAc8dHex5/ | 3854.40 |
| Hex7HexNAc6dHex10 | |

TABLE 10
Preferred acidic glycan compositions. Calculated
mass-to-charge ratios (calc. m/z) refer to the first
isotope signal of [M − H]⁻ ion.

| Proposed composition | calc. m/z |
|---|---|
| NeuAcHexHexNAc | 673.23 |
| NeuAcHexHexNAcdHex | 819.29 |
| NeuAcHex2HexNAc | 835.28 |
| NeuAcHexHexNAc2 | 876.31 |
| NeuAc2HexHexNAc | 964.33 |
| NeuAcHexHexNAcdHex2 | 965.35 |
| NeuAcHex2HexNAcdHex | 981.34 |
| Hex3HexNAc2SP | 989.28 |
| NeuAcHex3HexNAc | 997.34 |
| NeuAcHexHexNAc2dHex | 1022.37 |
| NeuAcHex2HexNAc2 | 1038.36 |
| NeuAcHexHexNAc3 | 1079.39 |
| NeuAc2HexHexNAcdHex | 1110.38 |
| NeuAc2Hex2HexNAc | 1126.38 |
| NeuAcHex2HexNAcdHex2 | 1127.40 |
| NeuAcHex3HexNAcdHex | 1143.39 |
| Hex4HexNAc2SP | 1151.33 |
| NeuAcHex4HexNAc | 1159.39 |
| NeuAc2HexHexNAc2 | 1167.41 |
| NeuAcHexHexNAc2dHex2 | 1168.43 |
| NeuAcHex2HexNAc2dHex | 1184.42 |
| Hex3HexNAc3SP | 1192.36 |
| NeuAcHex3HexNAc2/ | 1200.42 |
| NeuGcHex2HexNAc2dHex | |
| NeuGcHex3HexNAc2 | 1216.41 |
| NeuAcHexHexNAc3dHex | 1225.45 |
| NeuAcHex2HexNAc3 | 1241.44 |
| NeuAc2Hex2HexNAcdHex | 1272.44 |
| NeuAcHexHexNAc4 | 1282.47 |
| NeuAc2Hex3HexNAc | 1288.43 |
| NeuAcHex4HexNAcdHex | 1305.45 |
| NeuAc2HexHexNAc2dHex | 1313.46 |
| NeuAcHex5HexNAc/ | 1321.44/ |
| NeuAcHex2HexNAc3SP | 1321.40 |
| NeuAc2Hex2HexNAc2/ | 1329.46 |
| NeuGcNeuAcHexHexNAc2dHex | |
| NeuAcHex2HexNAc2dHex2 | 1330.48 |
| Hex3HexNAc3dHexSP | 1338.41 |
| NeuAcHex3HexNAc2dHex | 1346.47 |
| Hex4HexNAc3SP | 1354.41 |
| NeuAcHex4HexNAc2 | 1362.47 |
| NeuAc2HexHexNAc3 | 1370.48 |
| NeuAcHex2HexNAc3dHex | 1387.50 |
| NeuAcHex3HexNAc3 | 1403.49 |
| NeuGcHex3HexNAc3 | 1419.49 |
| NeuAcHexHexNAc4dHex | 1428.53 |
| NeuAc2Hex3HexNAcdHex | 1434.49 |
| NeuAcHex2HexNAc4 | 1444.52 |
| NeuAcHex3HexNAc3Ac | 1445.51 |
| NeuAc2Hex4HexNAc | 1450.48 |
| Hex5HexNAc2dHexSP | 1459.44 |
| NeuAc2Hex2HexNAc2dHex | 1475.52 |
| NeuAcHex6HexNAc/ | 1483.49/ |
| NeuAcHex3HexNAc3SP | 1483.45 |
| NeuAc2Hex3HexNAc2 | 1491.51 |
| NeuAcHex3HexNAc2dHex2 | 1492.53 |
| Hex4HexNAc3dHexSP | 1500.47 |
| NeuAcHex4HexNAc2dHex | 1508.53 |
| NeuAc2HexHexNAc3dHex/ | 1516.54/ |
| Hex5HexNAc3SP | 1516.46 |
| NeuAcHex5HexNAc2 | 1524.52 |
| NeuAc2Hex2HexNAc3 | 1532.54 |
| NeuAcHex2HexNAc3dHex2 | 1533.56 |
| NeuAcHex3HexNAc3dHex | 1549.55 |
| NeuAc2Hex2HexNAc2dHexSP | 1555.47 |
| Hex4HexNAc4SP | 1557.49 |
| NeuAcHex3HexNAc3(SP)2 | 1563.41 |
| NeuAcHex4HexNAc3 | 1565.55 |
| NeuAc2HexHexNAc4 | 1573.56 |
| NeuGcHex4HexNAc3 | 1581.54 |
| NeuAcHex2HexNac4dHex | 1590.58 |
| NeuAc2Hex4HexNAcdHex | 1596.54 |
| NeuAcHex3HexNAc4 | 1606.57 |
| NeuAc2Hex2HexNAc2dHex2/ | 1621.57/ |
| Hex6HexNAc2dHexSP | 1621.49 |
| NeuAc2Hex3HexNAc2dHex | 1637.57 |
| NeuAcHex4HexNAc3SP | 1645.50 |
| NeuAcHex2HexNAc5 | 1647.60 |
| NeuAcHex4HexNAc2dHex2 | 1654.58 |
| Hex5HexNAc3dHexSP | 1662.52 |
| NeuAcHex5HexNAc2dHex | 1670.58 |
| NeuAc2Hex2HexNAc3dHex | 1678.60 |
| NeuAcHex2HexNAc3dHex3 | 1679.62 |
| NeuAcHex6HexNAc2 | 1686.57 |
| NeuAc2Hex3HexNAc3 | 1694.59 |
| Hex4HexNAc4dHexSP | 1703.55 |
| NeuAcHex3HexNAc3dHex(SP)2 | 1709.47 |
| NeuGcNeuAcHex3HexNAc3 | 1710.59 |
| NeuAcHex4HexNAc3dHex | 1711.61 |
| Hex5HexNAc4SP | 1719.54 |
| NeuAcHex4HexNAc3(SP)2 | 1725.46 |
| Hex4HexNAc3dHex2(SP)2/ | 1726.48/ |
| NeuGc2Hex3HexNAc3 | 1726.58 |
| NeuAcHex5HexNAc3/ | 1727.60 |
| NeuGcHex4HexNAc3dHex | |
| NeuAc2Hex2HexNAc4 | 1735.62 |
| NeuAcHex2HexNAc4dHex2 | 1736.64 |
| NeuGcHex5HexNAc3 | 1743.60 |
| NeuAcHex3HexNAc4dHex | 1752.63 |
| NeuAc2Hex2HexNAc3dHexSP | 1758.55 |
| NeuAcHex3HexNAc4(SP)2/ | 1766.49/ |
| NeuAcHex6HexNAc2SP | 1766.53 |
| Hex6HexNAc2dHex2SP/ | 1767.55/ |
| Hex3HexNAc4dHex2(SP)2/ | 1767.51 |
| NeuAc2Hex2HexNAc2dHex3 | |
| NeuAcHex4HexNAc4 | 1768.63 |
| NeuAc2Hex6HexNAc/ | 1774.59/ |
| NeuAc2Hex3HexNAc3SP | 1774.55 |
| Hex7HexNAc2dHexSP | 1783.55 |
| NeuGcHex4HexNac4 | 1784.62 |
| NeuAcHex4HexNAc3dHexSP | 1791.56 |
| NeuAcHex2HexNAc5dHex | 1793.66 |
| NeuAc2Hex4HexNAc2dHex/ | 1799.62 |
| Hex5HexNAc4(SP)2 | |
| NeuAcHex3HexNac5 | 1809.65 |
| NeuAc2Hex5HexNAc2/ | 1815.62 |
| NeuAc2Hex2HexNAc4SP | |
| NeuAcHex5HexNAc2dHex2/ | 1816.64 |
| NeuAcHex2HexNAc4dHex2SP | |
| Hex6NexNAc3dHexSP | 1824.57 |
| NeuGcHex3HexNAc5 | 1825.65 |
| NeuAcHex6HexNAc2dHex | 1832.63 |
| NeuAc2Hex3HexNAc3dHex | 1840.65 |
| NeuAcHex3HexNAc3dHex3 | 1841.67 |
| NeuAc2Hex4HexNAc3 | 1856.64 |
| NeuAcHex4HexNAc3dHex2 | 1857.66 |
| Hex5HexNAc4dHexSP | 1865.60 |
| NeuAcHex4HexNAc3dHex(SP)2 | 1871.52 |
| NeuAcHex5HexNAc3dHex/ | 1873.66 |
| NeuGcHex4HexNAc3dHex2 | |
| Hex6HexNAc4SP | 1881.65 |
| NeuAcHex5HexNAC3(SP)2 | 1887.51 |
| NeuAcHex6HexNAc3 | 1889.65 |
| NeuAcHex3HexNAc4dHex2 | 1898.69 |
| Hex4HexNAc5dHexSP | 1906.63 |
| NeuAcHex6HexNAc2dHexSP/ | 1912.59 |
| NeuAcHex3HexNAc4dHex(SP)2 | |
| NeuAcHex4HexNAc4dHex | 1914.68 |
| NeuAc2Hex3HexNAc3dHexSP | 1920.60 |
| Hex5HexNAc5SP | 1922.62 |
| NeuAcHex4HexNAc4(SP)2 | 1928.54 |
| NeuAcHex5HexNAc4 | 1930.68 |
| NeuGcHex5HexNAc4 | 1946.67 |
| NeuAcHex5HexNAc3dHexSP | 1953.62 |
| NeuAcHex3HexNAc5dHex | 1955.71 |
| NeuAc2Hex5HexNAc2dHex/ | 1961.67/ |
| Hex6HexNAc4(SP)2 | 1961.55 |
| NeuAcHex4HexNAc5 | 1971.71 |
| NeuAcHex5HexNAc4Ac | 1972.69 |
| NeuAcHex6HexNAc2dHex2/ | 1978.69/ |
| NeuAcHex3HexNAc4dHex2SP | 1978.65 |
| NeuAc2Hex4HexNAc3dHex/ | 2002.70/ |
| Hex8HexNAc3SP | 2002.62 |
| NeuAcHex4HexNAc3dHex3 | 2003.72 |
| NeuAcHex5HexNAc4SP | 2010.64 |
| Hex5HexNAc4dHex2SP | 2011.66 |
| NeuAc2Hex5HexNAc3/ | 2018.70 |
| NeuGcNeuAcHex4HexNAc3dHex | |
| NeuAcHex5HexNAc3dHex2 | 2019.72 |
| NeuGcHex5HexNAc4SP | 2026.63 |
| Hex6HexNAc4dHexSP | 2027.65 |
| NeuAcHex6HexNAc3dHex | 2035.71 |
| NeuAc2Hex3HexNAc4dHex/ | 2043.73/ |
| Hex7HexNAc4SP | 2043.65 |
| NeuAcHex7HexNAc3 | 2051.71 |
| Hex4HexNAc5dHex2SP | 2052.68 |
| NeuAc2Hex4HexNAc4 | 2059.72 |
| NeuAcHex4HexNAc4dHex2 | 2060.74 |
| Hex5HexNAc5dHexSP | 2068.68 |
| NeuAcHex4HexNAc4dHex(SP)2 | 2074.60 |
| NeuAcHex5HexNAc4dHex | 2076.74 |
| NeuAc2Hex4HexNAc3dHexSP | 2082.66 |
| NeuGc2Hex4HexNAc4 | 2091.71 |
| NeuAcHex6HexNAc4/ | 2092.73 |
| NeuGcHex5HexNAc4dHex | |
| NeuAc2Hex5HexNAc3SP/ | 2098.65 |
| NeuGcNeuAcHex4HexNAc3dHexSP | |
| NeuAcHex5HexNAc3dHex2SP/ | 2099.67 |
| NeuGcHex4HexNAc3dHex3SP | |
| NeuAc2Hex3HexNAc5 | 2100.75 |
| NeuAcHex3HexNAc5dHex2/ | 2101.77/ |
| NeuAc2Hex4HexNAc4Ac | 2101.73 |
| NeuAcHex6HexNAc3dHexSP | 2115.67 |
| NeuAcHex4HexNAc5dHex | 2117.76 |
| Hex7HexNAc3dHex2SP/ | 2132.68/ |
| NeuAc2Hex3HexNAc3dHex3 | 2132.76 |
| NeuAcHex5HexNAc5 | 2133.76 |
| Hex8HexNAc3dHexSP/ | 2148.68 |
| NeuAc2Hex4HexNAc3dHex2 | |
| NeuAcHex8Hexnac2dHex/ | 2156.74/ |
| NeuAcHex5HexNAc4dHexSP | 2156.69 |
| Hex5HexNAC4dHex3SP | 2157.71 |
| NeuAc2Hex5HexNAc3dHex | 2164.75 |
| NeuAcHex5HexNAc3dHex3 | 2165.77 |
| NeuAcHex9HexNAc2/ | 2172.73/ |
| NeuAcHex6HexNAc4SP/ | 2172.69 |
| NeuGcHex5HexNAc4dHexSP | |
| NeuAcHex4Hexnac6 | 2174.79 |
| NeuAc2Hex6HexNAc3/ | 2180.75 |
| NeuGc2Hex4HexNAc3dHex2 | |
| NeuAcHex6HexNAc3dHex2 | 2181.77 |
| NeuAc3Hex3HexNAc4/ | 2188.76/ |
| NeuGcHex6HexNAc4SP/ | 2188.68 |
| NeuAc2NeuGcHex2HexNAc4dHex | |
| NeuAc2Hex3HexNAc4dHex2/ | 2189.79/ |
| Hex7HexNAc4dHexSP | 2189.70 |
| NeuAcHex3HexNAc4dHex4 | 2190.81 |
| NeuGcNeuAcHex6HexNAc3/ | 2196.74 |
| NeuGc2Hex5HexNAc3dHex | |
| Hex4HexNAc5dHex3SP | 2198.74 |
| NeuAc2Hex4HexNAc4dHex | 2205.78 |
| NeuAcHex4HexNAc4dHex3 | 2206.80 |
| NeuAc2Hex4HexNAc4(SP)2 | 2219.64 |
| NeuAc2Hex5HexNAc4 | 2221.78 |
| NeuAcHex5HexNAc4dHex2 | 2222.80 |
| Hex6HexNAc5dHexSP | 2230.73 |
| NeuGcNeuAcHex5HexNAc4 | 2237.77 |
| NeuAcHex6HexNAc4dHex/ | 2238.79 |
| NeuGcHex5HexNAc4dHex2 | |
| NeuAc2Hex3HexNAc5dHex | 2246.81 |
| NeuAcHex3HexNAc5dHex3 | 2247.83 |
| NeuGc2Hex5Hexnac4 | 2253.76 |
| NeuAcHex7HexNAc4/ | 2254.79 |
| NeuGcHex6HexNAc4dHex | |
| NeuAc2Hex4HexNAc5 | 2262.80 |
| NeuAcHex4HexNAc5dHex2/ | 2263.82/ |
| NeuAc2Hex5HexNAc4Ac | 2263.79 |
| NeuAcHex5HexNAc5dHex | 2279.82 |
| NeuAc2Hex4HexNAc4dHexSP | 2285.74 |
| NeuAcHex4HexNAc4dHex3SP | 2286.76 |
| NeuAcHex8HexNAc3SP/ | 2293.72/ |
| NeuAc3Hex4HexNAc3dHex | 2293.80 |
| NeuAc2Hex4HexNAc3dHex3 | 2294.82 |
| NeuAcHex6HexNAc5 | 2295.81 |
| NeuAc2Hex5HexNAc4SP | 2301.73 |
| NeuAcHex5HexNAc4dHex2SP | 2302.75 |
| NeuAc2Hex5HexNAc4Ac2 | 2305.80 |
| NeuAc2Hex5HexNAc3dHex2/ | 2310.81 |
| NeuGcNeuAcHex4HexNAc3dHex3 | |
| NeuAcHex5HexNAc3dHex4/ | 2311.83 |
| NeuGcHex6HexNAc5 | |
| NeuAcHex6HexNAc4dHexSP | 2318.75 |
| Hex6HexNAc4dHex3SP/ | 2319.77 |
| NeuGcNeuAcHex3HexNAc6 | |
| NeuAcHex4HexNAc6dHex | 2320.84 |
| NeuAcHex5HexNAc5dHexAc | 2321.83 |
| NeuAc2Hex6HexNAc3dHex | 2326.81 |
| NeuAcHex6HexNAc3dHex3 | 2327.83 |
| NeuAcHex7HexNAc4SP/ | 2334.74/ |
| NeuGcHex6HexNAc4dHexSP/ | 2334.79 |
| NeuAcHex10HexNAc2 | |
| NeuAcHex5HexNAc6 | 2336.84 |
| NeuAc3Hex4HexNac4 | 2350.82 |
| NeuAc2Hex4HexNAc4dHex2/ | 2351.84/ |
| Hex8HexNAc4dHexSP | 2351.76 |
| NeuGcNeuAc2Hex4HexNAc4 | 2366.81 |
| NeuAc2Hex5HexNAc4dHex | 2367.83 |
| NeuAcHex5HexNAc4dHex3 | 2368.85 |
| NeuAcHex5HexNAc4dHex2(SP)2 | 2382.71 |
| NeuAc2Hex6HexNAc4/ | 2383.83 |
| NeuGcNeuAcHex5HexNAc4dHex | |
| NeuAcHex6HexNAc4dHex2/ | 2384.85 |
| NeuGcHex5HexNAc4dHex3 | |
| NeuAc3Hex5HexNAc3SP/ | 2389.75/ |
| NeuAc2Hex5HexNAc4Ac4 | 2389.82 |
| NeuAc2Hex5HexNAc3dHex2SP | 2390.77 |
| NeuAcHex5HexNAc3dHex4SP/ | 2391.79/ |
| NeuAc3Hex3HexNAc5 | 2391.84 |
| NeuAc2Hex3HexNAc5dHex2 | 2392.86 |
| NeuAcHex3HexNAc5dHex4 | 2393.89 |
| NeuGc2Hex5HexNAc4dHex | 2399.82 |
| Hex4HexNAc6dHex3SP | 2401.82 |
| NeuAc2Hex6HexNAc3dHexSP | 2406.76 |
| NeuAc2Hex4HexNAc5dHex | 2408.86 |
| NeuAcHex4HexNAc5dHex3/ | 2409.88/ |
| NeuAc2Hex5HexNAc4dHexAc | 2409.84 |
| NeuAc2Hex5HexNAc5 | 2424.85 |
| NeuAcHex5HexNAc5dHex2 | 2425.87 |
| NeuAcHex8HexNAc3dHexSP/ | 2439.77 |
| NeuAc3Hex4HexNAc3dHex2 | |
| NeuAcHex6HexNAc5dHex | 2441.87 |
| NeuAc2Hex8HexNAc2dHex/ | 2447.83/ |
| NeuAc2Hex5HexNAc4dHexSP | 2447.79 |
| NeuAcHex8HexNAc2dHex3/ | 2448.85/ |
| NeuAcHex5HexNAc4dHex3SP | 2448.81 |
| NeuAcHex3HexNAc6dHex3 | 2450.91 |
| NeuAc2Hex5HexNAc4dHexAc2 | 2451.85 |
| NeuAc2Hex5HexNAc3dHex3 | 2456.87 |
| NeuAcHex7HexNAc5 | 2457.86 |
| NeuAcHex5HexNAc5dHex2Ac | 2467.89 |
| NeuAc2Hex6HexNAc3dHex2 | 2472.86 |
| NeuAcHex6HexNAc3dHex4/ | 2473.88 |
| NeuGcHex7HexNAc5 | |
| NeuAcHex5HexNAc6dHex | 2482.90 |
| NeuAcHex6HexNAc5Ac | 2483.88 |
| NeuAc2Hex7HexNAc3dHex | 2488.86 |
| NeuAcHex7HexNAc3dHex3 | 2489.88 |
| NeuAcHex6HexNAc6/ | 2498.89 |
| NeuGcHex5hexNAc6dHex | |
| NeuAc3Hex5HexNAc4 | 2512.87 |
| NeuAc2Hex5HexNAc4dHex2 | 2513.89 |
| NeuAcHex5HexNAc4dHex4 | 2514.91 |
| NeuAcHex6HexNAc5dHexSP/ | 2521.83/ |
| NeuAcHex9HexNAc3dHex/ | 2521.87 |
| NeuAc3Hex2HexNAc5dHex2 | |
| Hex6HexNAc5dHex3SP | 2522.85 |
| NeuGcNeuAc2Hex5HexNAc4 | 2528.87 |
| NeuAc2Hex6HexNAc4dHex/ | 2529.89 |
| NeuGcNeuAcHex5HexNAc4dHex2 | |
| NeuAcHex6HexNAc4dHex3 | 2530.91 |
| NeuAc3Hex3HexNAc5dHex/ | 2537.90/ |
| NeuGcHex6HexNAc5dHexSP/ | 2537.82 |
| NeuAcHex7HexNAc5SP | |
| NeuAc2Hex3HexNAc5dHex3 | 2538.92 |
| NeuAcHex5HexNAc7/ | 2539.92 |
| NeuAcHex3HexNAc5dHex5 | |
| NeuGc2NeuAcHex5HexNAc4 | 2544.86 |
| NeuGc2Hex5Hexnac4dHex2/ | 2545.88 |
| NeuGcNeuAcHex6HexNAc4dHex | |
| NeuAc3Hex4HexNAc5 | 2553.90 |
| NeuAc2Hex4HexNAc5dHex2 | 2554.92 |
| NeuAcHex4HexNAc5dHex4 | 2555.94 |
| NeuGc3Hex5HexNAc4 | 2560.86 |
| NeuAc2Hex5HexNAc5dHex | 2570.91 |
| NeuAcHex5HexNAc5dHex3 | 2571.93 |
| NeuAc2Hex6HexNAc5 | 2586.91 |
| NeuAcHex6HexNAc5dHex2 | 2587.93 |
| Hex7HexNAc6dHexSP | 2595.86 |
| NeuGcNeuAcHex6HexNAc5 | 2602.90 |
| NeuAcHex7HexNAc5dHex/ | 2603.92/ |
| NeuGcHex6HexNAc5dHex2 | 603.92 |
| NeuGc2Hex6HexNac5 | 2618.90 |
| NeuAcHex8HexNAc5/ | 2619.92 |
| NeuGcHex7HexNAc5dHex | |
| NeuAc2Hex5HexNAc6 | 2627.93 |
| NeuAcHex5HexNAc6dHex2 | 2628.95 |
| NeuGcHex8HexNAc5/ | 2635.91/ |
| NeuAcHex4HexNAc5dHex4SP | 2635.89 |
| NeuAcHex6HexNAc6dHex | 2644.95 |
| NeuAc2Hex5HexNAc5dHexSP | 2650.87 |
| NeuAc2Hex5HexNAc4dHex3 | 2659.95 |
| NeuAcHex7HexNAc6 | 2660.94 |
| NeuGcNeuAc2Hex5HexNAc4dHex | 2674.92 |
| NeuAc3Hex6HexNAc4 | |
| NeuGcHex6HexNAc5dHexSP/ | 2683.88 |
| NeuAcHex7HexNAc5dHexSP | |
| NeuAcHex5HexNAc7dHex | 2685.98 |
| NeuAc2Hex7HexNAc4dHex | 2691.94 |
| NeuAcHex7HexNAc4dHex3 | 2692.96 |
| NeuAc2Hex4HexNAc5dHex2(SP)2 | 2714.83 |
| NeuAcHex4HexNAc5dHex4(SP)2/ | 2715.85/ |
| NeuAc3Hex5HexNAc5 | 2715.95 |
| NeuAc2Hex5HexNAc5dHex2 | 2716.97 |
| NeuAcHex5HexNAc5dHex4 | 2717.99 |
| NeuAc2Hex6HexNAc5dHex | 2732.97 |
| NeuAcHex6HexNAc5dHex3 | 2733.99 |
| NeuAcHex6HexNAc5dHex2(SP)2 | 2747.84 |
| NeuGcNeuAcHex6HexNAc5dHex | 2748.96 |
| NeuAc3Hex4HexNAc6 | 2756.98 |
| NeuAc2Hex4HexNAc6dHex2 | 2758.00 |
| NeuAcHex4HexNAc6dHex4 | 2759.02 |
| NeuAc3Hex6HexNAc3dHex2 | 2763.96 |
| NeuAc2Hex6HexNAc3dHex4/ | 2764.98/ |
| NeuGc2Hex6HexNAc5dHex/ | 2764.96 |
| NeuGcHex7HexNAc5 | |
| NeuAcHex8HexNAc5dHex | 2765.98 |
| NeuAc2Hex5HexNAc6dHex | 2773.99 |
| NeuAcHex5HexNAc6dHex3 | 2775.01 |
| NeuGc2Hex7HexNAc5 | 2780.95 |
| NeuGcHex8HexNAc5dHex/ | 2781.97 |
| NeuAcHex9HexNAc5 | |
| NeuAc2Hex6HexNAc6 | 2789.99 |
| NeuAcHex6HexNAc6dHex2 | 2791.01 |
| NeuAc4Hex5HexNAc4 | 2803.97 |
| NeuAc3Hex5HexNAc4dHex2/ | 2804.99/ |
| NeuAcHex6HexNAc6dHex(SP)2 | 2804.86 |
| Hex6HexNAc6dHex3SP2 | 2805.88 |
| NeuAc2Hex5HexNAc4dHex4 | 2806.01 |
| NeuAcHex7Hexnac6dHex | 2807.00 |
| NeuAc2Hex6HexNAc5dHexSP | 2812.92 |
| NeuAcHex6HexNAc5dHex3SP | 2813.94 |
| NeuGcNeuAc3Hex5HexNAc4 | 2819.96 |
| NeuAc3Hex6HexNAc4dHex/ | 2820.98 |
| NeuGcNeuAc2Hex5HexNAc4dHex2 | |
| NeuAc2Hex6HexNAc4dHex3 | 2822.00 |
| NeuAcHex8HexNAc6 | 2823.00 |
| NeuGc2NeuAc2Hex5HexNAc4 | 2835.96 |
| NeuGc2NeuAcHex5HexNAc4dHex2 | 2836.98 |
| NeuAc3Hex6HexNAc5 | 2878.00 |
| NeuAc2Hex8HexNAc5dHex2 | 2879.02 |
| NeuAcHex6HexNAc5dHex4 | 288.04 |
| NeuAcHex7HexNAc6dHexSP/ | 2886.96/ |
| NeuAcHex10HexNAc4dHex | 2887.00 |
| NeuGcNeuAc2Hex6HexNAc5 | 2894.00 |
| NeuAc2Hex7HexNAc5dHex/ | 2895.02 |
| NeuGcNeuAcHex6HexNAc5dHex2 | |
| NeuAc3Hex6HexNAc4dHexSP/ | 2900.94 |
| NeuGcNeuAc2Hex5HexNAc4dHex2SP | |
| NeuGc2NeuAcHex6HexNAc5 | 2909.99 |
| NeuGc2Hex6HexNAc5dHex2 | 2911.01 |
| NeuAc3Hex5HexNAc6 | 2919.03 |
| NeuAc2Hex5HexNAc6dHex2 | 2920.05 |
| NeuAcHex5HexNAc6dHex4 | 2921.07 |
| NeuGc3Hex6HexNAc5 | 2925.99 |
| NeuGcNeuAc2Hex5HexNAc6 | 2935.02 |
| NeuAc2Hex6HexNAc6dHex/ | 2936.04 |
| NeuGcNeuAcHex5HexNAc6dHex2 | |
| NeuAcHex6HexNAc6dHex3 | 2937.07 |
| NeuGc2NeuAcHex5HexNAc6/ | 2951.02/ |
| NeuAc3Hex5HexNAc4dHex3 | 2951.04 |
| NeuAc2Hex7HexNAc6 | 2952.04 |
| NeuAcHex7HexNAc6dHex2 | 2953.06 |
| NeuAc2Hex6HexNAc5dHex2SP | 2958.98 |
| NeuAcHex6HexNAc5dHex4SP | 2960.00 |
| NeuAc2Hex4HexNAc7dHex2 | 2961.08 |
| NeuAcHex4HexNAc7dHex4 | 2962.10 |
| NeuAcHex6HexNAc7dHex2 | 2994.09 |
| NeuAcHex7HexNAc7dHex | 3010.08 |
| NeuAc3Hex6HexNAc5dHex | 3024.06 |
| NeuAc2Hex6HexNAc5dHex3 | 3025.08 |
| NeuAcHex8HexNAc7 | 3026.08 |
| NeuAc3Hex5HexNAc6dHex | 3065.09 |
| NeuAc2Hex5HexNAc6dHex3 | 3066.11 |
| NeuAcHex7HexNAc8 | 3067.10 |
| NeuAc3Hex6HexNAc6 | 3081.08 |
| NeuAc2Hex6HexNAc6dHex2 | 3082.10 |
| NeuAc2Hex7HexNAc6dHex | 3098.10 |
| NeuAcHex7HexNAc6dHex3 | 3099.12 |
| NeuAc3Hex6HexNAc5dHexSP | 3104.02 |
| NeuAc2Hex6HexNAc5dHex3SP | 3105.04 |
| NeuAcHex8HexNAc7SP/ | 3106.03/ |
| NeuAc3Hex4HexNAc7dHex | 3106.11 |
| Hex8HexNAc7dHex2SP/ | 3107.05/ |
| NeuAc2Hex4HexNAc7dHex3 | 3107.13 |
| NeuAcHex7HexNAc7dHex2 | 3156.14 |
| NeuAc3Hex6HexNAc5dHex2 | 3170.12 |
| NeuAc2Hex6HexNAc5dHex4 | 3171.14 |
| NeuAcHex8HexNAc7dHex | 3172.13 |
| NeuAc2Hex7HexNAc6dHexSP | 3178.05 |
| NeuAc3Hex6HexNAc6dHex | 3227.14 |
| NeuAc2Hex6HexNAc6dHex3 | 3228.16 |
| NeuAcHex8HexNAc8 | 3229.16 |
| NeuAc3Hex7HexNAc6 | 3243.13 |
| NeuAc2Hex7HexNAc6dHex2 | 3244.16 |
| NeuAcHex7HexNAc6dHex4 | 3245.18 |
| NeuAc2Hex8HexNAc6dHex/ | 3260.15 |
| NeuGcNeuAcHex7HexNAc6dHex2 | |
| NeuAcHex8HexNAc6dHex3/ | 3261.17 |
| NeuGcHex7HexNAc6dHex4 | |
| NeuAc3Hex7HexNAc5dHexSP/ | 3266.07 |
| NeuGcNeuAc2Hex6HexNAc5dHex2SP | |
| NeuAc3Hex5HexNAc7dHex/ | 3268.17/ |
| NeuGcHex8HexNAc7dHexSP | 3268.09 |
| NeuAc2Hex5HexNAc7dHex3 | 3269.19 |
| NeuAcHex7HexNAc9 | 3270.18 |
| NeuGc2Hex7HexNAc6dHex2 | 3276.15 |
| NeuAc4Hex4HexNAc5dHex2(SP)2 | 3297.02 |
| NeuAc3Hex4HexNAc5dHex4(SP)2 | 3298.04 |
| NeuAc2Hex7HexNAc7dHex | 3301.18 |
| NeuAcHex7HexNAc7dHex3 | 3302.20 |
| NeuAc3Hex6HexNAc5dHex3 | 3316.18 |
| NeuAc2Hex8HexNAc7 | 3317.17 |
| NeuAcHex8HexNAc7dHex2 | 3318.19 |
| NeuAc3Hex7HexNAc6dHex | 3389.19 |
| NeuAc2Hex7HexNAc6dHex3 | 3390.21 |
| NeuAcHex7HexNAc6dHex5/ | 3391.23 |
| NeuAcHex9HexNAc8 | |
| NeuAc3Hex5HexNAc7dHex2 | 3414.22 |
| NeuAc2Hex5HexNAc7dHex4 | 3415.24 |
| NeuAcHex7HexNAc9dHex | 3416.24 |
| NeuAc3Hex6HexNAc7dHex | 3430.22 |
| NeuAc2Hex6HexNAc7dHex3 | 3431.24 |
| NeuAcHex8HexNAc9 | 3432.24 |
| NeuAc2Hex8Hexnac7dHex | 3463.23 |
| NeuAcHex8HexNAc7dHex3 | 3464.25 |
| NeuAc3Hex7HexNAc6dHexSP | 3469.15 |
| NeuAc2Hex7HexNAc6dHex3SP | 3470.17 |
| NeuAc3Hex5HexNAc8dHex | 3471.25 |
| NeuAc2Hex5HexNAc8dHex3 | 3472.27 |
| NeuAcHex7HexNAc10 | 3473.26 |
| NeuAc4Hex7HexNAc6 | 3534.23 |
| NeuAc3Hex7HexNAc6dHex2 | 3535.25 |
| NeuAc2Hex7HexNAc6dHex4 | 3536.27 |
| NeuAcHex9HexNAc8dHex | 3537.27 |
| NeuAc4Hex5HexNAc7dHex | 3559.26 |
| NeuAc3Hex5HexNAc7dHex3 | 3560.28 |
| NeuAc2Hex7HexNAc9 | 3561.28 |
| NeuAcHex7HexNAc9dHex2 | 3562.30 |
| NeuAc3Hex7HexNAc7dHex | 3592.27 |
| NeuAc2Hex7HexNAc7dHex3 | 3593.29 |
| NeuAcHex9HexNAc9 | 3594.29 |
| NeuAc3Hex8HexNAc7 | 3608.27 |
| NeuAc2Hex8HexNAc7dHex2 | 3609.29 |
| NeuAcHex8HexNAc7dHex4 | 3610.31 |
| NeuAc3Hex5HexNAc8dHex2 | 3617.30 |
| NeuAc2Hex5HexNAc8dHex4 | 3618.32 |
| NeuAcHex7HexNAc10dHex | 3619.32 |
| NeuAc3Hex6HexNAc8dHex | 3633.30 |
| NeuAc4Hex7HexNAc6dHex | 3680.29 |
| NeuAc3Hex7HexNAc6dHex3 | 3681.31 |
| NeuAc2Hex9HexNAc8 | 3682.30 |
| NeuAcHex9HexNAc8dHex2 | 3683.32 |
| NeuAc4Hex6HexNAc7dHex | 3721.31 |
| NeuAc3Hex6HexNAc7dHex3 | 3722.34 |
| NeuAc2Hex8HexNAc9 | 3723.33 |
| NeuAcHex8HexNAc9dHex2 | 3724.35 |
| NeuAc3Hex7HexNAc7dHex2 | 3738.33 |
| NeuAc2Hex7HexNAc7dHex4 | 3739.35 |
| NeuAcHex9HexNAc9dHex | 3740.35 |
| NeuAc3Hex8HexNAc7dHex | 3754.33 |
| NeuAc2Hex8HexNAc7dHex3 | 3755.35 |
| NeuAcHex10HexNAc9/ | 3756.34 |
| NeuAcHex8HexNAc7dHex5 | |
| NeuAc4Hex6HexNAc8 | 3778.34 |
| NeuAc3Hex6HexNAc8dHex2 | 3779.36 |
| NeuAc2Hex6HexNAc8dHex4 | 3780.38 |
| NeuAcHex8HexNAc10dHex | 3781.37 |
| NeuAc4Hex7HexNAc6dHex2 | 3826.35 |
| NeuAc3Hex7Hexnac6dHex4 | 3827.37 |
| NeuAc2Hex9HexNAc8dHex | 3828.36 |
| NeuAcHex9HexNAc8dHex3 | 3829.38 |
| NeuAc4Hex8HexNAc7 | 3899.36 |
| NeuAc3Hex8HexNAc7dHex2 | 3900.38 |
| NeuAc2Hex8HexNAc7dHex4 | 3901.40 |
| NeuAcHex10HexNAc9dHex | 3902.40 |
| NeuAc4Hex6HexNAc8dHex | 3924.39 |
| NeuAc3Hex6HexNAc8dHex3 | 3925.41 |
| NeuAc2Hex8HexNAc10 | 3926.41 |
| NeuAcHex8HexNAc10dHex2 | 3927.43 |
| NeuAc3Hex9HexNAc8 | 3973.40 |
| NeuAc2Hex9HexNAc8dHex2 | 3974.42 |
| NeuAcHex9HexNAc8dHex4 | 3975.44 |
| NeuAc4Hex8HexNAc7dHex | 4045.42 |
| NeuAc3Hex8HexNAc7dHex3 | 4046.44 |
| NeuAc2Hex10HexNAc9/ | 4047.44 |
| NeuAc2Hex8HexNAc7dHex5 | |
| NeuAcHex10HexNAc9dHex2 | 4048.46 |
| NeuAc3Hex9HexNAc8dHex | 4119.46 |
| NeuAc2Hex9HexNAc8dHex3 | 4120.48 |
| NeuAcHex11HexNAc10/ | 4121.47 |
| NeuAcHex9HexNAc8dHex5 | |
| NeuAc2Hex10HexNAc9dHex2 | 4339.55 |
| NeuAcHex10HexNAc9dHex4 | 4340.57 |
| NeuAc2Hex10HexNAc9dHex3 | 4485.61 |

TABLE 11
NMR analysis of large neutral N-glycan fraction from pancreatic
cancer sample.
Reference glycans A-C are as in FIG. 26.

| | | | ppm | | | |
|---|---|---|---|---|---|---|
| Residue | Linkage | Proton | A | B | C | Sample |
| D-GlcNAc | | H-1α | 5.188 | 5.191 | 5.187 | 5.187 |
| | | H-1β | 4.695 | 4.690 | 4.693 | 4.693 |
| | | NAc | 2.038 | 2.042 | 2.037 | 2.036 |
| | | | 2.036 | | | |
| β-D-GlcNAc | 4 | H-1 | 4.601 | 4.596 | 4.586 | 4.952 |
| | | | 4.592 | | | |
| | | NAc | 2.064 | 2.072 | 2.063 | 2.062 |
| | | | 2.063 | | | |
| β-D-Man | 4, 4 | H-1 | 4.780 | 4.775 | 4.771 | under HDO |
| | | H-2 | 4.250 | 4.238 | 4.234 | 4.232/4.253 |
| α-D-Man | 6, 4, 4 | H-1 | 4.870 | 4.869 | 4.870 | 4.870 |
| | | H-2 | 4.145 | 4.149 | 4.149 | 4.146 |
| α-D-Man | 6, 6, 4, 4 | H-1 | 4.907 | 5.153 | 5.151 | 4.907/5.148 |
| | | H-2 | 3.984 | 4.025 | 4.021 | 3.984 |
| α-D-Man | 2, 6, 6, 4, 4 | H-1 | — | 5.047 | 5.042 | 5.041 |
| | | H-2 | — | 4.074 | 4.069 | 4.067 |
| α-D-Man | 3, 6, 4, 4 | H-1 | 5.092 | 5.414 | 5.085 | 5.090/5.407 |
| | | H-2 | 4.065 | 4.108 | 4.069 | 4.067/4.108 |
| α-D-Man | 2, 3, 6, 4, 4 | H-1 | — | 5.047 | — | 5.041 |
| | | H-2 | — | 4.074 | — | 4.067 |
| α-D-Man | 3, 4, 4 | H-1 | 5.097 | 5.343 | 5.341 | 5.090/5.345 |
| | | H-2 | 4.076 | 4.108 | 4.099 | 4.067/4.108 |
| α-D-Man | 2, 3, 4, 4 | H-1 | — | 5.317 | 5.309 | 5.308 |
| | | H-2 | — | 4.108 | 4.099 | 4.108 |
| α-D-Man | 2, 2, 3, 4, 4 | H-1 | — | 5.047 | 5.042 | 5.041 |
| | | H-2 | — | 4.074 | 4.069 | 4.067 |

TABLE 12
NMR analysis of small N-glycan fraction from pancreatic cancer
sample. Reference glycans D-G are as in FIG. 27.

ppm

| Residue | Linkage | Proton | D | Sample |
|---|---|---|---|---|
| D-GlcNAc | | H-1α | 5.188 | 5.188 |
| | | H-1β | 4.696 | under HDO |
| | | NAc | 2.038 | 2.037 |
| β-D-GlcNAc | 4 | H-1α | 4.612 | 4.606 |
| | | H-1β | 4.603 | |
| | | NAc | 2.078 | 2.078 |
| β-D-Man | 4, 4 | H-1 | 4.780 | under HDO |
| | | H-2 | 4.254 | 4.250/4.231 |
| α-D-Man | 6, 4, 4 | H-1 | 4.915 | 4.915 |
| | | H-2 | 3.974 | 3.970 |
| α-D-Man | 3, 4, 4 | H-1 | 5.101 | 5.100/5.350 |
| | | H-2 | 4.067 | 4.067/4.103/4.082 |

| | | | ppm | | | |
|---|---|---|---|---|---|---|
| Residue | Linkage | Proton | E | F | G | Sample |
| β-D- | | H-1α | 5.213 | 5.212 | 5.212 | 5.210 |
| GlcNAc | | H-1β | 4.722 | 4.721 | 4.72 | under HDO |
| | | Nacα | 2.057 | 2.057 | 2.057 | 2.056 |
| | | Nacβ | 2.054 | 2.054 | 2.054 | |
| β-D-Man | 4 | H-1(α) | 4.791 | 4.781 | 4.780 | under HDO |
| | | H-1(β) | 4.783 | 4.774 | 4.773 | |
| | | H-2(α) | 4.265 | 4.246 | 4.244 | 4.250 |
| | | H-2(β) | 4.254 | 4.238 | 4.235 | 4.231 |
| α-D-Man | 6, 4 | H-1 | 4.917 | 4.918 | 4.917 | 4.915 |
| | | H-2 | 3.973 | 3.977 | 3.98 | 3.970 |
| α-D-Man | 3, 4 | H-1 | 5.104 | 5.352 | 5.345 | 5.100/5.350 |
| | | H-2 | 4.070 | 4.105 | 4.080 | 4.067/4.103/4.082 |
| α-D-Man | 2, 3, 4 | H-1 | — | 5.051 | 5.303 | 5.046/5.304 |
| | | H-2 | — | 4.069 | 4.105 | 4.067/4.101 |
| α-D-Man | 2, 2, 3, 4 | H-1 | — | — | 5.043 | 5.046 |
| | | H-2 | — | — | 4.064 | 4.067 |

TABLE 13
NMR analysis of acidic N-glycan fraction from pancreatic cancer sample. Reference
glycans A-E are as in FIG. 28.

| | | | ppm | | | | | |
|---|---|---|---|---|---|---|---|---|
| Residue | Linkage | Proton | A | B | C | D | E | Sample |
| D- | | H-1α | 5.189 | 5.188 | 5.188 | 5.189 | 5.181 | 5.188 |
| GlcNAc | | H-1β | 4.694 | n.a. | n.a. | 4.695 | n.a. | 4.692 |
| | | NAc | 2.038 | 2.038 | 2.038 | 2.038 | 2.039 | 2.036 |
| α-L-Fuc | 6 | H-1α | — | — | — | — | 4.892 | 4.894 |
| | | H-1β | — | — | — | — | 4.900 | |
| | | H-5α | — | — | — | — | 4.10 | |
| | | H-5b | — | — | — | — | n.a. | |
| | | CH3α | — | — | — | — | 1.211 | 1.209 |
| | | CH3β | — | — | — | — | 1.223 | 1.219 |
| β-D- | 4 | H-1α | 4.613 | 4.614 | 4.612 | 4.614 | 4.663 | |
| GlcNAc | | H-1β | 4.604 | 4.606 | 4.604 | 4.606 | n.a. | 4.604 |
| | | NAc(α/β) | 2.084 | 2.081 | 2.081 | 2.081 | 2.096 | 2.082/2.094 |
| | | | | | | | 2.093 | |
| β-D-Man | 4, 4 | H-1 | n.a. | n.a. | n.a. | n.a. | n.a. | n.a. |
| | | H-2 | 4.258 | 4.250 | 4.246 | 4.253 | 4.248 | 4.258 |
| α-D-Man | 6, 4, 4 | H-1 | 4.948 | 4.930 | 4.928 | 4.930 | 4.922 | 4.948 |
| | | H-2 | 4.117 | 4.112 | 4.11 | 4.112 | 4.11 | 4.115 |
| β-D- | 2, 6, 4, 4 | H-1 | 4.604 | 4.582 | 4.581 | 4.582 | 4.573 | 4.604 |
| GlcpNAc | | Nac | 2.066 | 2.047 | 2.047 | 2.047 | 2.043 | 2.066/2.047 |
| β-D-Gal | 4, 2, 6, 4, 4 | H-1 | 4.447 | 4.473 | 4.473 | 4.473 | 4.550 | 4.444/4.47 |
| | | H-3 | n.a. | n.a. | n.a. | n.a. | 4.119 | 4.115 |
| α-D- | 3, 4, 2, 6, 4, 4 | H-3a | — | — | — | — | 1.800 | 1.800 |
| Neup5Ac | | H-3e | — | — | — | — | 2.758 | 2.758 |
| | | NAc | — | — | — | — | 2.031 | 2.029 |
| α-D- | 6, 4, 2, 6, 4, 4 | H-3a | 1.719 | — | — | — | — | 1.722 |
| Neup5Ac | | H-3e | 2.673 | — | — | — | — | 2.668 |
| | | NAc | 2.029 | — | — | — | — | 2.029 |
| α-D-Man | 3, 4, 4 | H-1 | 5.133 | 5.123 | 5.118 | 5.135 | 5.116 | 5.132/5.11 |
| | | H-2 | 4.197 | 4.195 | 4.190 | 4.196 | 4.189 | 4.197 |
| β-D- | 2, 3, 4, 4 | H-1 | 4.604 | 4.606 | 4.573 | 4.606 | 4.573 | 4.604/4.570 |
| GlcpNAc | | NAc | 2.070 | 2.043 | 2.047 | 2.069 | 2.048 | 2.069/2.047 |
| β-D-Galp | 4, 2, 3, 4, 4 | H-1 | 4.443 | — | 4.545 | 4.445 | 4.544 | 4.444 |
| | | H-3 | n.a. | — | 4.113 | n.a. | 4.113 | 4.115 |
| α-D- | 6, 4, 2, 3, 4, 4 | H-3a | 1.719 | — | — | 1.719 | — | 1.722 |
| Neup5Ac | | H-3e | 2.667 | — | — | 2.668 | — | 2.668 |
| | | NAc | 2.029 | — | — | 2.030 | — | 2.029 |
| α-D- | 3, 4, 2, 3, 4, 4 | H-3a | — | 1.783 | 1.797 | — | 1.797 | 1.800 |
| Neup5Ac | | H-3e | — | 2.759 | 2.756 | — | 2.758 | 2.758 |
| | | NAc | — | 2.030 | 2.030 | — | 2.031 | 2.029 |

n.a., not assigned.

TABLE 14

| | | |
|---|---|---|
| Hex₅₋₉HexNAc₂ | | |
| (including high-mannose type | | |
| N-glycans) | Human | Human |

| Proposed composition | m/z | tissue | cell line |
|---|---|---|---|
| Hex5HexNAc2 | 1257 | + | + |
| Hex6HexNAc2 | 1419 | + | + |
| Hex7HexNAc2 | 1581 | + | + |
| Hex8HexNAc2 | 1743 | + | + |
| Hex9HexNAc2 | 1905 | + | + |

| | | |
|---|---|---|
| Hex₁₋₄HexNAc₂dHex₀₋₁ | | |
| (including low-mannose type | | |
| N-glycans) | Human | Human |

| Proposed composition | m/z | tissue | cell line |
|---|---|---|---|
| HexHexNAc2 | 609 | + | |
| HexHexNAc2dHex | 755 | + | |
| Hex2HexNAc2 | 771 | + | + |
| Hex2HexNAc2dHex | 917 | + | + |
| Hex3HexNAc2 | 933 | + | + |
| Hex3HexNAc2dHex | 1079 | + | + |
| Hex4HexNAc2 | 1095 | + | + |
| Hex4HexNAc2dHex | 1241 | + | + |

| | | |
|---|---|---|
| Hex₁₀₋₁₂HexNAc₂ | | |
| (including glucosylated high- | | |
| mannose type N-glycans) | Human | Human |

| Proposed composition | m/z | cells | cell line |
|---|---|---|---|
| Hex10HexNAc2 | 2067 | + | + |
| Hex11HexNAc2 | 2229 | + | |
| Hex12HexNAc2 | 2391 | + | |

| | | |
|---|---|---|
| Hex₅₋₉HexNAc₂dHex₁ | | |
| (including fucosylated high- | | |
| mannose type N-glycans) | Human | Human |

| Proposed composition | m/z | tissue | cell line |
|---|---|---|---|
| Hex5HexNAc2dHex | 1403 | + | + |
| Hex6HexNAc2dHex | 1565 | + | + |

| | | |
|---|---|---|
| HexNAc = 3 and Hex ≧ 2 | | |
| (including hybrid-type and | | |
| monoantennary N-glycans) | Human | Human |

| Proposed composition | m/z | cells | cell line |
|---|---|---|---|
| Hex2HexNAc3 | 974 | + | |
| Hex2HexNAc3dHex | 1120 | + | |
| Hex3HexNAc3 | 1136 | + | + |
| Hex2HexNAc3dHex2 | 1266 | + | |
| Hex3HexNAc3dHex | 1282 | + | + |
| Hex4HexNAc3 | 1298 | + | + |
| Hex3HexNAc3dHex2 | 1428 | + | |
| Hex4HexNAc3dHex | 1444 | + | + |
| Hex5HexNAc3 | 1460 | + | + |
| Hex4HexNAc3dHex2 | 1590 | + | + |
| Hex5HexNAc3dHex | 1606 | + | + |
| Hex6HexNAc3 | 1622 | + | + |
| Hex5HexNAc3dHex2 | 1752 | + | + |
| Hex6HexNAc3dHex | 1768 | + | + |
| Hex7HexNAc3 | 1784 | + | + |
| Hex7HexNAc3dHex | 1930 | + | + |
| Hex8HexNAc3 | 1946 | + | |

| | | |
|---|---|---|
| HexNAc ≧ 4 and Hex ≧ 3 | | |
| (including complex-type N- | | |
| glycans) | Human | Human |

| Proposed composition | m/z | cells | cell line |
|---|---|---|---|
| Hex3HexNAc4 | 1339 | + | + |
| Hex3HexNAc4dHex | 1485 | + | + |
| Hex4HexNAc4 | 1501 | + | + |
| Hex3HexNAc5 | 1542 | + | + |
| Hex4HexNAc4dHex | 1647 | + | + |
| Hex5HexNAc4 | 1663 | + | + |
| Hex3HexNAc5dHex | 1688 | + | + |
| Hex4HexNAx5 | 1704 | + | + |
| Hex4HexNAc4dHex2 | 1793 | + | + |
| Hex5HexNAc4dHex | 1809 | + | + |
| Hex6HexNAc4 | 1825 | + | + |
| Hex4HexNAc5dHex | 1850 | + | + |
| Hex5HexNAc5 | 1866 | + | |
| Hex3HexNAc6dHex | 1891 | + | + |
| Hex5HexNAc4dHex2 | 1955 | + | + |
| Hex6HexNAc4dHex | 1971 | + | + |
| Hex7HexNAc4 | 1987 | + | + |
| Hex4HexNAc5dHex2 | 1996 | + | + |
| Hex5HexNAc5dHex | 2012 | + | |
| Hex6HexNAc5 | 2028 | + | + |
| Hex5HexNAc4dHex3 | 2101 | + | + |
| Hex6HexNAc4dHex2 | 2117 | + | |
| Hex7HexNAc4dHex | 2133 | + | + |
| Hex4HexNAc5dHex3 | 2142 | + | |
| Hex8HexNAc4 | 2149 | + | + |
| Hex5HexNAc5dHex2 | 2158 | + | |
| Hex6HexNAc5dHex | 2174 | + | + |
| Hex7HexNAc5 | 2190 | + | + |
| Hex5HexNAc6dHex | 2215 | + | + |
| Hex6HexNAc6 | 2231 | + | |
| Hex5HexNAc4dHex4 | 2247 | + | + |
| Hex7HexNAc4dHex2 | 2279 | + | |
| Hex5HexNAc5dHex3 | 2304 | + | + |
| Hex6HexNAc5dHex2 | 2320 | + | + |
| Hex7HexNAc5dHex | 2336 | + | |
| Hex8HexNAc5 | 2352 | + | + |
| Hex7HexNAc6 | 2393 | + | + |
| Hex7HexNAc4dHex3 | 2425 | + | |
| Hex6HexNAc5dHex3 | 2466 | + | |
| Hex8HexNAc5dHex | 2498 | + | |
| Hex7HexNAc6dHex | 2539 | + | + |
| Hex6HexNAc5dHex4 | 2612 | + | + |
| Hex8HexNAc7 | 2758 | + | |
| Hex7Hexnac5dHex4 | 2775 | + | + |
| Hex8HexNAc5dHex4 | 2937 | + | + |
| Hex8HexNAc6dHex4 | 3140 | + | + |
| Hex9HexNAc6dHex4 | 3302 | + | + |
| Hex10HexNAc6dHex4 | 3464 | + | + |
| Hex11HexNAc6dHex4 | 3626 | + | + |

| | | |
|---|---|---|
| Hex₁₋₉HexNAc₁ | | |
| (including soluble glycans) | Human | Human |

| Proposed composition | m/z | cells | cell line |
|---|---|---|---|
| Hex2HexNAc | 568 | + | |
| Hex3HexNAc | 730 | + | + |
| Hex4HexNAc | 892 | + | |
| Hex5HexNAc | 1054 | + | + |
| Hex6HexNAc | 1216 | + | + |
| Hex7HexNAc | 1378 | + | + |
| Hex8HexNAc | 1540 | + | + |
| Hex9HexNAc | 1702 | + | |

| | | |
|---|---|---|
| HexNAc ≧ 3 and dHex ≧ 1 | | |
| (including fucosylated | | |
| hybrid/monoant. N-glycans) | Human | Human |

| Proposed composition | m/z | cells | cell line |
|---|---|---|---|
| Hex2HexNAc3dHex | 1120 | + | |
| Hex2HexNAc3dHex2 | 1266 | + | |
| Hex3HexNAc3dHex | 1282 | + | + |
| Hex3HexNAc3dHex2 | 1428 | + | |
| Hex4HexNAc3dHex | 1444 | + | + |
| Hex4HexNAc3dHex2 | 1590 | + | + |
| Hex5HexNAc3dHex | 1606 | + | + |
| Hex5HexNAc3dHex2 | 1752 | + | + |
| Hex6HexNAc3dHex | 1768 | + | + |
| Hex7HexNAc3dHex | 1930 | + | + |
| Hex3HexNAc4dHex | 1485 | + | + |
| Hex4HexNAc4dHex | 1647 | + | + |
| Hex3HexNAc5dHex | 1688 | + | + |
| Hex4HexNAc4dHex2 | 1793 | + | + |
| Hex5HexNAc4dHex | 1809 | + | + |
| Hex4HexNAc5dHex | 1850 | + | + |
| Hex3HexNAc6dHex | 1891 | + | + |
| Hex5HexNAc4dHex2 | 1955 | + | + |
| Hex6HexNAc4dHex | 1971 | + | + |
| Hex4HexNAc5dHex2 | 1996 | + | + |
| Hex5HexNAc5dHex | 2012 | + | |
| Hex5HexNAc4dHex3 | 2101 | + | + |
| Hex6HexNAc4dHex2 | 2117 | + | |
| Hex7HexNAc4dHex | 2133 | + | + |
| Hex4HexNAc5dHex3 | 2142 | + | |
| Hex5HexNAc5dHex2 | 2158 | + | |
| Hex6HexNAc5dHex | 2174 | + | + |
| Hex5HexNAc6dHex | 2215 | + | + |
| Hex5HexNAc4dHex4 | 2247 | + | + |
| Hex7HexNAc4dHex2 | 2279 | + | |
| Hex5HexNAc5dHex3 | 2304 | + | + |
| Hex6HexNAc5dHex2 | 2320 | + | + |
| Hex7HexNAc5dHex | 2336 | + | |
| Hex7HexNAc4dHex3 | 2425 | + | |
| Hex6HexNAc5dHex3 | 2466 | + | |
| Hex8HexNAc5dHex | 2498 | + | |
| Hex7HexNAc6dHex | 2539 | + | + |
| Hex6HexNAc5dHex4 | 2612 | + | + |
| Hex7Hexnac5dHex4 | 2775 | + | + |
| Hex8HexNAc5dHex4 | 2937 | + | + |
| Hex8HexNAc6dHex4 | 3140 | + | + |
| Hex9HexNAc6dHex4 | 3302 | + | + |
| Hex10HexNAc6dHex4 | 3464 | + | + |
| Hex11HexNAc6dHex4 | 3626 | + | + |

| | | |
|---|---|---|
| HexNAc = Hex ≧ 5 | | |
| (terminal HexNAc, N = H) | Human | Human |

| Proposed composition | m/z | cells | cell line |
|---|---|---|---|
| Hex5HexNAc5 | 1866 | + | |
| Hex5HexNAc5dHex | 2012 | + | |
| Hex5HexNAc5dHex2 | 2158 | + | |
| Hex6HexNAc6 | 2231 | + | |

| | | |
|---|---|---|
| HexNAc ≧ 3 and dHex ≧ 2 | | |
| (including multifucosylated | | |
| hybrid/monoant. N-glycans) | Human | Human |

| Proposed composition | m/z | cells | cell line |
|---|---|---|---|
| Hex2HexNAc3dHex2 | 1266 | + | |
| Hex3HexNAc3dHex2 | 1428 | + | |
| Hex4HexNAc3dHex2 | 1590 | + | + |
| Hex5HexNAc3dHex2 | 1752 | + | + |
| Hex4HexNAc4dHex2 | 1793 | + | + |
| Hex5HexNAc4dHex2 | 1955 | + | + |
| Hex4HexNAc5dHex2 | 1996 | + | + |
| Hex5HexNAc4dHex3 | 2101 | + | + |
| Hex6HexNAc4dHex2 | 2117 | + | |
| Hex4HexNAc5dHex3 | 2142 | + | |
| Hex5HexNAc5dHex2 | 2158 | + | |
| Hex5HexNAc4dHex4 | 2247 | + | + |
| Hex7HexNAc4dHex2 | 2279 | + | |
| Hex5HexNAc5dHex3 | 2304 | + | + |
| Hex6HexNAc5dHex2 | 2320 | + | + |
| Hex7HexNAc4dHex3 | 2425 | + | |
| Hex6HexNAc5dHex3 | 2466 | + | |
| Hex6HexNAc5dHex4 | 2612 | + | + |
| Hex7Hexnac5dHex4 | 2775 | + | + |
| Hex8HexNAc5dHex4 | 2937 | + | + |
| Hex8HexNAc6dHex4 | 3140 | + | + |
| Hex9HexNAc6dHex4 | 3302 | + | + |
| Hex10HexNAc6dHex4 | 3464 | + | + |
| Hex11HexNAc6dHex4 | 3626 | + | + |

| | | |
|---|---|---|
| HexNAc > Hex ≧ 2 | | |
| (terminal HexNAc, N > H) | Human | Human |

| Proposed composition | m/z | cells | cell line |
|---|---|---|---|
| Hex2HexNAc3 | 974 | + | |
| Hex2HexNAc3dHex | 1120 | + | |
| Hex2HexNAc3dHex2 | 1266 | + | |
| Hex3HexNAc4 | 1339 | + | + |
| Hex3HexNAc4dHex | 1485 | + | + |
| Hex3HexNAc5 | 1542 | + | + |
| Hex3HexNAc5dHex | 1688 | + | + |
| Hex4HexNAx5 | 1704 | + | + |
| Hex4HexNAc5dHex | 1850 | + | + |
| Hex3HexNAc6dHex | 1891 | + | + |
| Hex4HexNAc5dHex2 | 1996 | + | + |
| Hex4HexNAc5dHex3 | 2142 | + | |
| Hex5HexNAc6dHex | 2215 | + | + |

TABLE 15

| HexNAc = 3 and Hex ≧ 2 | | | |
|---|---|---|---|
| (including hybrid-type and | | | |
| monoantennary N-glycans) | | Human | Human |
| Proposed composition | m/z | cells | cell line |
| Hex3HexNAc3SP | 1192 | + | + |
| Hex3HexNAc3dHexSP | 1338 | + | + |
| Hex4HexNAc3SP | 1354 | + | + |
| NeuAcHex3HexNAc3 | 1403 | + | + |
| NeuGcHex3HexNAc3 | 1419 | + | |
| Hex4HexNAc3dHexSP | 1500 | + | + |
| Hex5HexNAc3SP | 1516 | + | + |
| NeuAcHex3HexNAc3dHex | 1549 | + | + |
| NeuAcHex3HexNAc3SP2 | 1563 | + | |
| NeuAcHex4HexNAc3 | 1565 | + | + |
| NeuGcHex4HexNAc3 | 1581 | + | + |
| Hex4HexNAc3dHex2SP | 1646 | + | |
| Hex5HexNAc3dHexSP | 1662 | + | + |
| Hex6HexNAc3SP and/or | 1678 | + | + |
| NeuAc2Hex2HexNAc3dHex | | | |
| NeuAc2Hex3HexNAc3 | 1694 | + | + |
| NeuAcHex3HexNAc3dHexSP2 | 1709 | + | |
| NeuAcHex4HexNAc3dHex | 1711 | + | + |
| NeuAcHex5HexNAc3 and/or | 1727 | + | + |
| NeuGcHex4HexNAc3dHex | | | |
| NeuGcHex5HexNAc3 | 1743 | + | + |
| NeuAcHex4HexNAc3dHexSP | 1791 | + | |
| Hex5HexNAc3dHex2SP | 1808 | + | |
| Hex6HexNAc3dHexSP | 1824 | + | + |
| NeuAc2Hex3HexNAc3dHex | 1840 | + | + |
| NeuAc2Hex4HexNAc3 | 1856 | + | |
| NeuAcHex4HexNAc3dHex2 | 1857 | + | |
| NeuAcHex5HexNAc3dHex | 1873 | + | + |
| and/or | | | |
| NeuGcHex4HexNAc3dHex2 | | | |
| NeuAcHex5HexNAc3SP2 | 1887 | + | |
| NeuAcHex6HexNAc3 | 1889 | + | + |
| Hex8HexNAc3SP and/or | 2002 | + | + |
| NeuAc2Hex4HexNAc3dHex | | | |
| NeuAcHex4HexNAc3dHex3 | 2003 | + | |
| NeuAc2Hex5HexNAc3 and/or | 2018 | + | + |
| NeuGcNeuAcHex4HexNAc3dHex | | | |
| NeuAcHex5HexNAc3dHex2 | 2019 | + | + |
| NeuGcNeuAcHex5HexNAc3 | 2034 | + | |
| and/or | | | |
| NeuGc2Hex4HexNAc3dHex | | | |
| NeuAcHex6HexNAc3dHex | 2035 | + | + |
| NeuGc2Hex5HexNAc3 | 2050 | + | |
| NeuAcHex7HexNAc3 | 2051 | + | + |
| NeuAc2Hex4HexNAc3dHexSP | 2082 | + | |
| and/or Hex8HexNAc3SP2 | | | |
| NeuAcHex6HexNAc3dHexSP | 2115 | + | |
| Hex8HexNAc3dHexSP and/or | 2148 | + | |
| NeuAc2Hex4HexNAc3dHex2 | | | |
| NeuAc2Hex5HexNAc3dHex | 2164 | + | + |
| and/or Hex6HexNAc5SP2 | | | |
| NeuAcHex5HexNAc3dHex3 | 2165 | + | + |
| NeuAcHex8HexNAc3SP | 2293 | + | |
| and/or | | | |
| NeuAc3Hex4HexNAc3dHex | | | |
| NeuAc2Hex5HexNAc3dHex2 | 2310 | + | |
| and/or | | | |
| NeuGcNeuAcHex4HexNAc3dHex3 | | | |
| NeuAc3Hex5HexNAc3SP | 2389 | + | |
| NeuAc2Hex5HexNAc3dHex2SP | 2390 | + | + |
| NeuAc2Hex6HexNAc3dHexSP | 2406 | + | |
| NeuAcHex8HexNAc3dHexSP | 2439 | + | |
| and/or | | | |
| NeuAc3Hex4HexNAc3dHex2 | | | |
| NeuAcHex9HexNAc3dHex | 2521 | + | |
| HexNAc ≧ 4 and Hex ≧ 3 | | | |
| (including complex-type N- | | | |
| glycans) | | Human | Human |
| Proposed composition | m/z | cells | cell line |
| Hex4HexNAc4SP | 1557 | + | + |
| NeuAcHex3HexNAc4 | 1606 | + | |
| Hex4HexNAc4SP2 | 1637 | + | |
| Hex4HexNAc4dHexSP | 1703 | + | + |
| Hex4HexNAc4SP3 and/or | 1717 | + | |
| Hex7HexNAc2SP2 | | | |
| Hex5HexNAc4SP | 1719 | + | + |
| NeuAcHex4HexNAc4 | 1768 | + | + |
| NeuGcHex4HexNac4 | 1784 | + | |
| Hex5HexNAc4SP2 and/or | 1799 | + | |
| Hex8HexNAc2SP | | | |
| NeuAcHex3HexNac5 | 1809 | + | |
| NeuGcHex3HexNAc5 | 1825 | + | + |
| Hex5HexNAc4dHexSP | 1865 | + | + |
| Hex6HexNAc4SP | 1881 | + | + |
| Hex4HexNAc5dHexSP | 1906 | + | |
| NeuAcHex4HexNAc4dHex | 1914 | + | + |
| NeuAcHex4HexNAc4SP2 | 1928 | + | |
| NeuAcHex5HexNAc4 | 1930 | + | + |
| NeuGcHex5HexNAc4 | 1946 | + | + |
| NeuAcHex4HexNAc5 | 1971 | + | |
| NeuAcHex5HexNAc4Ac | 1972 | + | |
| Hex5HexNAc5SP2 | 2002 | + | |
| NeuAcHex5HexNAc4SP | 2010 | + | + |
| Hex5HexNAc4dHex2SP | 2011 | + | |
| NeuGcHex5HexNAc4SP | 2026 | + | |
| Hex6HexNAc4dHexSP | 2027 | + | + |
| Hex7HexNAc4SP and/or | 2043 | + | |
| Hex4HexNAc6SP2 and/or | | | |
| NeuAc2Hex3HexNAc4dHex | | | |
| NeuAcHex4HexNAc5SP | 2051 | + | |
| Hex4HexNAc5dHex2SP | 2052 | + | |
| NeuAc2Hex4HexNAc4 | 2059 | + | |
| NeuAcHex4HexNAc4dHex2 | 2060 | + | + |
| NeuAcHex4HexNAc4dHexSP2 | 2074 | + | |
| NeuAcHex5HexNAc4dHex | 2076 | + | + |
| NeuAcHex6HexNAc4 and/or | 2092 | + | + |
| NeuGcHex5HexNAc4dHex | | | |
| NeuAcHex3HexNAc5dHex2 | 2101 | + | |
| and/or | | | |
| NeuAc2Hex4HexNAc4Ac | | | |
| NeuGcHex6HexNAc4 | 2108 | + | |
| NeuAcHex4HexNAc5dHex | 2117 | + | |
| Hex4HexNAc5dHex2SP2 | 2132 | + | |
| NeuAcHex5HexNAc5 | 2133 | + | + |
| NeuAc2Hex4HexNAc4SP | 2139 | + | |
| NeuAcHex5HexNAc4dHexSP | 2156 | + | + |
| Hex5HexNAc4dHex3SP | 2157 | + | |
| Hex6HexNAc5SP2 | 2164 | + | |
| NeuAcHex6HexNAc4SP | 2172 | + | + |
| and/or | | | |
| NeuGcHex5HexNAc4dHexSP | | | |
| Hex6HexNAc4dHex2SP | 2173 | + | |
| and/or | | | |
| Hex3HexNAc6dHex2SP2 | | | |
| NeuAcHex4HexNAc6 | 2174 | + | |
| NeuAc3Hex3HexNAc4 and/or | 2188 | + | |
| NeuGcHex6HexNAc4SP | | | |
| and/or | | | |
| NeuAc2NeuGcHex2HexNAc4dHex | | | |
| NeuAc2Hex3HexNAc4dHex2 | 2189 | + | + |
| and/or Hex7HexNAc4dHexSP | | | |
| and/or | | | |
| Hex4HexNAc6dHexSP2 | | | |
| NeuAcHex3HexNAc4dHex4 | 2190 | + | + |
| Hex4HexNAc5dHex3SP | 2198 | + | + |
| NeuAc2Hex4HexNAc4dHex | 2205 | + | |
| NeuAc2Hex4HexNAc4SP2 | 2219 | + | |
| NeuAc2Hex5HexNAc4 | 2221 | + | + |
| NeuAcHex5HexNAc4dHex2 | 2222 | + | + |
| Hex6HexNAc5dHexSP | 2230 | + | |
| NeuGcNeuAcHex5HexNAc4 | 2237 | + | |
| NeuAcHex6HexNAc4dHex | 2238 | + | + |
| and/or | | | |
| NeuGcHex5HexNAc4dHex2 | | | |
| NeuAc2Hex3HexNAc5dHex | 2246 | + | |
| and/or Hex7HexNAc5SP | | | |
| NeuGc2Hex5HexNAc4 | 2253 | + | |
| NeuAcHex7HexNAc4 and/or | 2254 | + | + |
| NeuGcHex6HexNAc4dHex | | | |
| NeuAc2Hex4HexNAc5 | 2262 | + | |
| NeuAcHex4HexNAc5dHex2 | 2263 | + | |
| and/or | | | |
| NeuAc2Hex5HexNAc4Ac | | | |
| Hex5HexNAc6dHexSP | 2271 | + | |
| NeuAcHex5HexNAc5dHex | 2279 | + | + |
| NeuAc2Hex4HexNAc4dHexSP | 2285 | + | |
| and/or Hex11HexNAc2SP | | | |
| NeuAcHex6HexNAc5 | 2295 | + | + |
| NeuAc2Hex5HexNAc4SP | 2301 | + | |
| NeuAcHex5HexNAc4dHex2SP | 2302 | + | |
| NeuAc2Hex5HexNAc4Ac2 | 2305 | + | |
| NeuAcHex6HexNAc4dHexSP | 2318 | + | + |
| Hex6HexNAc4dHex3SP | 2319 | + | |
| and/or | | | |
| NeuGcNeuAcHex3HexNAc6 | | | |
| NeuAcHex4HexNAc6dHex | 2320 | + | |
| NeuAcHex5HexNAc5dHexAc | 2321 | + | |
| Hex7HexNAc4dHex2SP | 2335 | + | |
| and/or | | | |
| Hex4HexNAc6dHex2SP2 | | | |
| NeuAcHex5HexNAc6 | 2336 | + | + |
| NeuAc3Hex4HexNac4 | 2350 | + | |
| NeuAc2Hex4HexNAc4dHexSP | 2365 | + | |
| NeuAc2Hex5HexNAc4dHex | 2367 | + | + |
| NeuAcHex5HexNAc4dHex3 | 2368 | + | + |
| NeuAc2Hex6HexNAc4 and/or | 2383 | + | + |
| NeuGcNeuAcHex5HexNAc4dHex | | | |
| NeuAcHex6HexNAc4dHex2 | 2384 | + | + |
| and/or | | | |
| NeuGcHex5HexNAc4dHex3 | | | |
| NeuAc2Hex3HexNAc5dHex2 | 2392 | + | + |
| and/or Hex7HexNAc5dHexSP | | | |
| NeuAcHex3HexNAc5dHex4 | 2393 | + | |
| NeuGc2Hex5HexNAc4dHex | 2399 | + | |
| NeuAcHex4HexNAc6dHexSP | 2400 | + | |
| and/or | | | |
| NeuGcHex6HexNAc4dHex2 | | | |
| and/or | | | |
| NeuAcHex7HexNAc4dHex | | | |
| Hex4HexNAc6dHex3SP | 2401 | + | |
| NeuAc2Hex4HexNAc5dHex | 2408 | + | |
| NeuAcHex4HexNAc5dHex3 | 2409 | + | |
| and/or | | | |
| NeuAc2Hex5HexNAc4dHexAc | | | |
| NeuAc2Hex5HexNAc5 | 2424 | + | |
| NeuAcHex5HexNAc5dHex2 | 2425 | + | + |
| NeuAcHex6HexNAc5dHex | 2441 | + | + |
| NeuAc2Hex5HexNAc4dHexSP | 2447 | + | + |
| NeuAcHex5HexNAc4dHex3SP | 2448 | + | |
| NeuAcHex7HexNAc5 and/or | 2457 | + | + |
| NeuGcHex6HexNAc5dHex | | | |
| NeuGcHex7HexNAc5 | 2473 | + | |
| NeuAcHex5HexNAc6dHex | 2482 | + | |
| NeuAcHex4HexNAc5dHex3SP | 2489 | + | |
| Hex6HexNAc7SP | 2490 | + | |
| NeuAc3Hex5HexNAc4 | 2512 | + | |
| NeuAc2Hex5HexNAc4dHex2 | 2513 | + | + |
| NeuAcHex5HexNAc4dHex4 | 2514 | + | |
| NeuAcHex6HexNAc5dHexSP | 2521 | + | |
| and/or | | | |
| NeuAc3Hex2HexNAc5dHex2 | | | |
| Hex6HexNAc5dHex3SP | 2522 | + | |
| NeuGcNeuAc2Hex5HexNAc4 | 2528 | + | |
| NeuAc2Hex6HexNAc4dHex | 2529 | + | |
| and/or | | | |
| NeuGcNeuAcHex5HexNAc4dHex2 | | | |
| NeuGc2NeuAcHex5HexNAc4 | 2544 | + | |
| NeuGc2Hex5HexNAc4dHex2 | 2545 | + | |
| and/or | | | |
| NeuGcNeuAcHex6HexNAc4dHex | | | |
| NeuGc3Hex5HexNAc4 | 2560 | + | |
| NeuGc2Hex6HexNAc4dHex | 2561 | + | |
| NeuAc2Hex5HexNAc5dHex | 2570 | + | + |
| NeuAcHex5HexNAc5dHex3 | 2571 | + | |
| NeuAc2Hex6HexNAc5 | 2586 | + | + |
| NeuAcHex6HexNAc5dHex2 | 2587 | + | + |
| Hex7HexNAc6dHexSP | 2595 | + | |
| NeuGcNeuAcHex6HexNAc5 | 2602 | + | |
| NeuAcHex7HexNAc5dHex | 2603 | + | + |
| and/or | | | |
| NeuGcHex6HexNAc5dHex2 | | | |
| NeuAcHex8HexNAc5 and/or | 2619 | + | |
| NeuGcHex7HexNAc5dHex | | | |
| NeuAc2Hex5HexNAc6 | 2627 | + | |
| NeuGcHex8HexNAc5 and/or | 2635 | + | |
| NeuAcHex4HexNAc5dHex4SP | | | |
| NeuAcHex6HexNAc6dHex | 2644 | + | + |
| NeuAc2Hex5HexNAc4dHex3 | 2659 | + | |
| NeuAcHex7HexNAc6 | 2660 | + | + |
| NeuGcNeuAc2Hex5HexNAc4dHex | 2674 | + | |
| and/or | | | |
| NeuAc3Hex6HexNAc4 | | | |
| NeuAc2Hex4HexNAc5dHex2SP2 | 2714 | + | |
| NeuAcHex4HexNAc5dHex4SP2 | 2715 | + | + |
| and/or | | | |
| NeuAc3Hex5HexNAc5 | | | |
| NeuAc2Hex5HexNAc5dHex2 | 2716 | + | |
| NeuAc2Hex6HexNAc5dHex | 2732 | + | + |
| NeuAcHex6HexNAc5dHex3 | 2733 | + | + |
| NeuGcNeuAcHex6HexNAc5dHex | 2748 | + | |
| NeuAcHex8HexNAc5dHex | 2765 | + | + |
| NeuGcHex8HexNAc5dHex | 2781 | + | |
| and/or NeuAcHex9HexNAc5 | | | |
| NeuAcHex6HexNAc6dHex2 | 2791 | + | |
| NeuAc3Hex5HexNAc4dHex2 | 2804 | + | |
| and/or | | | |
| NeuAcHex6HexNAc6dHexSP2 | | | |
| Hex6HexNAc6dHex3SP2 | 2805 | + | |
| NeuAcHex7HexNAc6dHex | 2807 | + | + |
| NeuAc2Hex6HexNAc5dHexSP | 2812 | + | |
| NeuAcHex6HexNAc5dHex3SP | 2813 | + | |
| NeuGcNeuAc3Hex5HexNAc4 | 2819 | + | |
| NeuAc3Hex6HexNAc4dHex | 2820 | + | |
| and/or | | | |
| NeuGcNeuAc2Hex5HexNAc4dHex2 | | | |
| NeuAc3Hex6HexNAc5 | 2878 | + | + |
| NeuAc2Hex6HexNAc5dHex2 | 2879 | + | + |
| NeuAcHex6HexNAc5dHex4 | 2880 | + | + |
| NeuGcNeuAc2Hex6HexNAc5 | 2894 | + | |
| NeuAc2Hex7HexNAc5dHex | 2895 | + | + |
| and/or | | | |
| NeuGcNeuAcHex6HexNAc5dHex2 | | | |
| NeuAc3Hex6HexNAc4dHexSP | 2900 | + | |
| and/or | | | |
| NeuGcNeuAc2Hex5HexNAc4dHex2SP | | | |
| NeuGc2Hex6HexNAc5dHex2 | 2911 | + | |
| NeuAc2Hex5HexNAc6dHex2 | 2920 | + | |
| NeuGc3Hex6HexNAc5 | 2925 | + | |
| NeuGcNeuAc2Hex5HexNAc6 | 2935 | + | |
| NeuAc2Hex6HexNAc6dHex | 2936 | + | |
| and/or | | | |
| NeuGcNeuAcHex5HexNAc6dHex2 | | | |
| NeuAcHex6HexNAc6dHex3 | 2937 | + | |
| NeuGc2NeuAcHex5HexNAc6 | 2951 | + | |
| and/or | | | |
| NeuAc3Hex5HexNAc4dHex3 | | | |
| NeuAc2Hex7HexNAc6 | 2952 | + | |
| NeuAcHex7HexNAc6dHex2 | 2953 | + | + |
| Hex8HexNAc7dHexSP | 2961 | + | |
| NeuAc2Hex4HexNAc7dHex2 | 2961 | + | |
| NeuAcHex7HexNAc7dHex | 3010 | + | |
| NeuAc3Hex6HexNAc5dHex | 3024 | + | + |
| NeuAc2Hex6HexNAc5dHex3 | 3025 | + | + |
| NeuAcHex8HexNAc7 | 3026 | + | |
| NeuGc3Hex6HexNAc5dHex | 3072 | + | |
| and/or | | | |
| NeuGc2NeuAcHex7HexNAc5 | | | |
| NeuAc3Hex6HexNAc6 | 3081 | + | + |
| NeuAc2Hex6HexNAc6dHex2 | 3082 | + | |
| NeuAc2Hex7HexNAc6dHex | 3098 | + | + |
| NeuAcHex7HexNAc6dHex3 | 3099 | + | + |
| NeuAc3Hex6HexNAc5dHexSP | 3104 | + | |
| NeuAc2Hex6HexNAc5dHex3SP | 3105 | + | |
| NeuAc3Hex6HexNAc5dHex2 | 3170 | + | |
| NeuAc2Hex6HexNAc5dHex4 | 3171 | + | |
| NeuAcHex8HexNAc7dHex | 3172 | + | |
| NeuAc3Hex6HexNAc6dHex | 3227 | + | |
| NeuAc2Hex6HexNAc6dHex3 | 3228 | + | |
| NeuAc3Hex7HexNAc6 | 3243 | + | |
| NeuAc2Hex7HexNAc6dHex2 | 3244 | + | + |
| NeuAcHex7HexNAc6dHex4 | 3245 | + | + |
| NeuAc2Hex7HexNAc7dHex | 3301 | + | |
| NeuAcHex7HexNAc7dHex3 | 3302 | + | |
| NeuAc2Hex8HexNAc7 | 3317 | + | |
| NeuAcHex8HexNAc7dHex2 | 3318 | + | |
| NeuAc3Hex7HexNAc6dHex | 3389 | + | + |
| NeuAc2Hex7HexNAc6dHex3 | 3390 | + | + |
| NeuAcHex7HexNAc6dHex5 | 3391 | + | |
| and/or NeuAcHex9HexNAc8 | | | |
| NeuAc2Hex8HexNAc7dHex | 3463 | + | |
| NeuAcHex8HexNAc7dHex3 | 3464 | + | |
| NeuAc2Hex7HexNAc6dHex4 | 3536 | + | |
| NeuAcHex9HexNAc8dHex | 3537 | + | |
| NeuAc3Hex8HexNAc7 | 3608 | + | |
| NeuAc2Hex8HexNac7dHex2 | 3609 | + | |
| NeuAcHex8HexNac7dHex4 | 3610 | + | |
| NeuAc4Hex7HexNAc6dHex | 3680 | + | |
| NeuAc3Hex7HexNAc6dHex3 | 3681 | + | |
| NeuAc2Hex9HexNAc8 | 3682 | + | |
| NeuAcHex9HexNAc8dHex2 | 3683 | + | |
| NeuAc3Hex8HexNAc7dHex | 3754 | + | |
| NeuAc2Hex8HexNAc7dHex3 | 3755 | + | |
| NeuAcHex10HexNAc9 and/or | 3756 | + | |
| NeuAcHex8HexNAc7dHex5 | | | |
| NeuAc4Hex6HexNAc8 | 3778 | + | |
| NeuAc3Hex7HexNAc6dHex4 | 3827 | + | |
| NeuAc2Hex9HexNAc8dHex | 3828 | + | |
| NeuAcHex9HexNAc8dHex3 | 3829 | + | |
| NeuAc2Hex8HexNAc7dHex4 | 3901 | + | |
| NeuAc2Hex9HexNAc8dHex2 | 3974 | + | |
| NeuAcHex9HexNAc8dHex4 | 3975 | + | |
| NeuAc4Hex8HexNAc7dHex | 4045 | + | |
| NeuAc3Hex8HexNAc7dHex3 | 4046 | + | |
| NeuAc2Hex10HexNAc9 | 4047 | + | |
| and/or | | | |
| NeuAc2Hex8HexNAc7dHex5 | | | |
| NeuAc3Hex9HexNAc8dHex | 4119 | + | |
| NeuAc2Hex9HexNAc8dHex3 | 4120 | + | |
| HexNAc ≧ 3 and dHex ≧ 1 | | | |
| (including fucosylated N- | | | |
| glycans) | | Human | Human |
| Proposed composition | m/z | cells | cell line |
| Hex3HexNAc3dHexSP | 1338 | + | + |
| Hex4HexNAc3dHexSP | 1500 | + | + |
| NeuAcHex3HexNAc3dHex | 1549 | + | + |
| Hex4HexNAc3dHex2SP | 1646 | + | |
| Hex5HexNAc3dHexSP | 1662 | + | + |
| Hex6HexNAc3SP and/or | 1678 | + | + |
| NeuAc2Hex2HexNAc3dHex | | | |
| NeuAcHex3HexNAc3dHexSP2 | 1709 | + | |
| NeuAcHex4HexNAc3dHex | 1711 | + | + |
| NeuAcHex5HexNAc3 and/or | 1727 | + | + |
| NeuGcHex4HexNAc3dHex | | | |
| NeuAcHex4HexNAc3dHexSP | 1791 | + | |
| Hex5HexNAc3dHex2SP | 1808 | + | |
| Hex6NexNAc3dHexSP | 1824 | + | + |
| NeuAc2Hex3HexNAc3dHex | 1840 | + | + |
| NeuAcHex4HexNAc3dHex2 | 1857 | + | |
| NeuAcHex5HexNAc3dHex | 1873 | + | + |
| and/or | | | |
| NeuGcHex4HexNAc3dHex2 | | | |
| Hex8HexNAc3SP and/or | 2002 | + | + |
| NeuAc2Hex4HexNAc3dHex | | | |
| NeuAcHex4HexNAc3dHex3 | 2003 | + | |
| NeuAc2Hex5HexNAc3 and/or | 2018 | + | + |
| NeuGcNeuAcHex4HexNAc3dHex | | | |
| NeuAcHex5HexNAc3dHex2 | 2019 | + | + |
| NeuGcNeuAcHex5HexNAc3 | 2034 | + | |
| and/or | | | |
| NeuGc2Hex4HexNAc3dHex | | | |
| NeuAcHex6HexNAc3dHex | 2035 | + | + |
| NeuAc2Hex4HexNAc3dHexSP | 2082 | + | |
| and/or Hex8HexNAc3SP2 | | | |
| NeuAcHex6HexNAc3dHexSP | 2115 | + | |
| Hex8HexNAc3dHexSP and/or | 2148 | + | |
| NeuAc2Hex4HexNAc3dHex2 | | | |
| NeuAc2Hex5HexNAc3dHex | 2164 | + | + |
| and/or Hex6HexNAc5SP2 | | | |
| NeuAcHex5HexNAc3dHex3 | 2165 | + | + |
| NeuAcHex8HexNAc3SP | 2293 | + | |
| and/or | | | |
| NeuAc3Hex4HexNAc3dHex | | | |
| NeuAc2Hex5HexNAc3dHex2 | 2310 | + | |
| and/or | | | |
| NeuGcNeuAcHex4HexNAc3dHex3 | | | |
| NeuAc2Hex5HexNAc3dHex2SP | 2390 | + | + |
| NeuAc2Hex6HexNAc3dHexSP | 2406 | + | |
| NeuAcHex8HexNAc3dHexSP | 2439 | + | |
| and/or | | | |
| NeuAc3Hex4HexNAc3dHex2 | | | |
| NeuAcHex9HexNAc3dHex | 2521 | + | |
| Hex4HexNAc4dHexSP | 1703 | + | + |
| Hex5HexNAc4dHexSP | 1865 | + | + |
| Hex4HexNAc5dHexSP | 1906 | + | |
| NeuAcHex4HexNAc4dHex | 1914 | + | + |
| Hex5HexNAc4dHex2SP | 2011 | + | |
| Hex6HexNAc4dHexSP | 2027 | + | + |
| Hex7HexNAc4SP and/or | 2043 | + | |
| Hex4HexNAc6SP2 and/or | | | |
| NeuAc2Hex3HexNAc4dHex | | | |
| Hex4HexNAc5dHex2SP | 2052 | + | |
| NeuAcHex4HexNAc4dHex2 | 2060 | + | + |
| NeuAcHex4HexNAc4dHexSP2 | 2074 | + | |
| NeuAcHex5HexNAc4dHex | 2076 | + | + |
| NeuAcHex6HexNAc4 and/or | 2092 | + | + |
| NeuGcHex5HexNAc4dHex | | | |
| NeuAcHex3HexNAc5dHex2 | 2101 | + | |
| and/or | | | |
| NeuAc2Hex4HexNAc4Ac | | | |
| NeuAcHex4HexNAc5dHex | 2117 | + | |
| Hex4HexNAc5dHex2SP2 | 2132 | + | |
| NeuAcHex5HexNAc4dHexSP | 2156 | + | + |
| Hex5HexNAc4dHex3SP | 2157 | + | |
| NeuAcHex6HexNAc4SP | 2172 | + | + |
| and/or | | | |
| NeuGcHex5HexNAc4dHexSP | | | |
| Hex6HexNAc4dHex2SP | 2173 | + | |
| and/or | | | |
| Hex3HexNAc6dHex2SP2 | | | |
| NeuAc3Hex3HexNAc4 and/or | 2188 | + | |
| NeuGcHex6HexNAc4SP | | | |
| and/or | | | |
| NeuAc2NeuGcHex2HexNAc4dHex | | | |
| NeuAc2Hex3HexNAc4dHex2 | 2189 | + | + |
| and/or Hex7HexNAc4dHexSP | | | |
| and/or | | | |
| Hex4HexNAc6dHexSP2 | | | |
| NeuAcHex3HexNAc4dHex4 | 2190 | + | + |
| Hex4HexNAc5dHex3SP | 2198 | + | + |
| NeuAc2Hex4HexNAc4dHex | 2205 | + | |
| NeuAcHex5HexNAc4dHex2 | 2222 | + | + |
| Hex6HexNAc5dHexSP | 2230 | + | |
| NeuAcHex6HexNAc4dHex | 2238 | + | + |
| and/or | | | |
| NeuGcHex5HexNAc4dHex2 | | | |
| NeuAc2Hex3HexNAc5dHex | 2246 | + | |
| and/or Hex7HexNAc5SP | | | |
| NeuAcHex7HexNAc4 and/or | 2254 | + | + |
| NeuGcHex6HexNAc4dHex | | | |
| NeuAcHex4HexNAc5dHex2 | 2263 | + | |
| and/or | | | |
| NeuAc2Hex5HexNAc4Ac | | | |
| Hex5HexNAc6dHexSP | 2271 | + | |
| NeuAcHex5HexNAc5dHex | 2279 | + | + |
| NeuAc2Hex4HexNAc4dHexSP | 2285 | + | |
| and/or Hex11HexNAc2SP | | | |
| NeuAcHex5HexNAc4dHex2SP | 2302 | + | |
| NeuAcHex6HexNAc4dHexSP | 2318 | + | + |
| Hex6HexNAc4dHex3SP | 2319 | + | |
| and/or | | | |
| NeuGcNeuAcHex3HexNAc6 | | | |
| NeuAcHex4HexNAc6dHex | 2320 | + | |
| NeuAcHex5HexNAc5dHexAc | 2321 | + | |
| Hex7HexNAc4dHex2SP | 2335 | + | |
| and/or | | | |
| Hex4HexNAc6dHex2SP2 | | | |
| NeuAc2Hex4HexNAc4dHexSP | 2365 | + | |
| NeuAc2Hex5HexNAc4dHex | 2367 | + | + |
| NeuAcHex5HexNAc4dHex3 | 2368 | + | + |
| NeuAc2Hex6HexNAc4 and/or | 2383 | + | + |
| NeuGcNeuAcHex5HexNAc4dHex | | | |
| NeuAcHex6HexNAc4dHex2 | 2384 | + | + |
| and/or | | | |
| NeuGcHex5HexNAc4dHex3 | | | |
| NeuAc2Hex3HexNAc5dHex2 | 2392 | + | + |
| and/or Hex7HexNAc5dHexSP | | | |
| NeuAcHex3HexNAc5dHex4 | 2393 | + | |
| NeuGc2Hex5HexNAc4dHex | 2399 | + | |
| NeuAcHex4HexNAc6dHexSP | 2400 | + | |
| and/or | | | |
| NeuGcHex6HexNAc4dHex2 | | | |
| and/or | | | |
| NeuAcHex7HexNAc4dHex | | | |
| Hex4HexNAc6dHex3SP | 2401 | + | |
| NeuAc2Hex4HexNAc5dHex | 2408 | + | |
| NeuAcHex4HexNAc5dHex3 | 2409 | + | |
| and/or | | | |
| NeuAc2Hex5HexNAc4dHexAc | | | |
| NeuAcHex5HexNAc5dHex2 | 2425 | + | + |
| NeuAcHex6HexNAc5dHex | 2441 | + | + |
| NeuAc2Hex5HexNAc4dHexSP | 2447 | + | + |
| NeuAcHex5HexNAc4dHex3SP | 2448 | + | |
| NeuAcHex7HexNAc5 and/or | 2457 | + | + |
| NeuGcHex6HexNAc5dHex | | | |
| NeuAcHex5HexNAc6dHex | 2482 | + | |
| NeuAcHex4HexNAc5dHex3SP | 2489 | + | |
| NeuAc2Hex5HexNAc4dHex2 | 2513 | + | + |
| NeuAcHex5HexNAc4dHex4 | 2514 | + | |
| NeuAcHex6HexNAc5dHexSP | 2521 | + | |
| and/or | | | |
| NeuAc3Hex2HexNAc5dHex2 | | | |
| Hex6HexNAc5dHex3SP | 2522 | + | |
| NeuAc2Hex6HexNAc4dHex | 2529 | + | |
| and/or | | | |
| NeuGcNeuAcHex5HexNAc4dHex2 | | | |
| NeuGc2Hex5HexNAc4dHex2 | 2545 | + | |
| and/or | | | |
| NeuGcNeuAcHex6HexNAc4dHex | | | |
| NeuGc2Hex6HexNAc4dHex | 2561 | + | |
| NeuAc2Hex5HexNAc5dHex | 2570 | + | + |
| NeuAcHex5HexNAc5dHex3 | 2571 | + | |
| NeuAcHex6HexNAc5dHex2 | 2587 | + | + |
| Hex7HexNAc6dHexSP | 2595 | + | |
| NeuAcHex7HexNAc5dHex | 2603 | + | + |
| and/or | | | |
| NeuGcHex6HexNAc5dHex2 | | | |
| NeuAcHex8HexNAc5 and/or | 2619 | + | |
| NeuGcHex7HexNAc5dHex | | | |
| NeuGcHex8HexNAc5 and/or | 2635 | + | |
| NeuAcHex4HexNAc5dHex4SP | | | |
| NeuAcHex6HexNAc6dHex | 2644 | + | + |
| NeuAc2Hex5HexNAc4dHex3 | 2659 | + | |
| NeuGcNeuAc2Hex5HexNAc4dHex | 2674 | + | |
| and/or | | | |
| NeuAc3Hex6HexNAc4 | | | |
| NeuAc2Hex4HexNAc5dHex2SP2 | 2714 | + | |
| NeuAcHex4HexNAc5dHex4SP2 | 2715 | + | + |
| and/or | | | |
| NeuAc3Hex5HexNAc5 | | | |
| NeuAc2Hex5HexNAc5dHex2 | 2716 | + | |
| NeuAc2Hex6HexNAc5dHex | 2732 | + | + |
| NeuAcHex6HexNAc5dHex3 | 2733 | + | + |
| NeuGcNeuAcHex6HexNAc5dHex | 2748 | + | |
| NeuAcHex8HexNAc5dHex | 2765 | + | + |
| NeuGcHex8HexNAc5dHex | 2781 | + | |
| and/or NeuAcHex9HexNAc5 | | | |
| NeuAcHex6HexNAc6dHex2 | 2791 | + | |
| NeuAc3Hex5HexNAc4dHex2 | 2804 | + | |
| and/or | | | |
| NeuAcHex6HexNAc6dHexSP2 | | | |
| Hex6HexNAc6dHex3SP2 | 2805 | + | |
| NeuAcHex7HexNAc6dHex | 2807 | + | + |
| NeuAc2Hex6HexNAc5dHexSP | 2812 | + | |
| NeuAcHex6HexNAc5dHex3SP | 2813 | + | |
| NeuAc3Hex6HexNAc4dHex | 2820 | + | |
| and/or | | | |
| NeuGcNeuAc2Hex5HexNAc4dHex2 | | | |
| NeuAc2Hex6HexNAc5dHex2 | 2879 | + | + |
| NeuAcHex6HexNAc5dHex4 | 2880 | + | + |
| NeuAc2Hex7HexNAc5dHex | 2895 | + | + |
| and/or | | | |
| NeuGcNeuAcHex6HexNAc5dHex2 | | | |
| NeuAc3Hex6HexNAc4dHexSP | 2900 | + | |
| and/or | | | |
| NeuGcNeuAc2Hex5HexNAc4dHex2SP | | | |
| NeuGc2Hex6HexNAc5dHex2 | 2911 | + | |
| NeuAc2Hex5HexNAc6dHex2 | 2920 | + | |
| NeuAc2Hex6HexNAc6dHex | 2936 | + | |
| and/or | | | |
| NeuGcNeuAcHex5HexNAc6dHex2 | | | |
| NeuAcHex6HexNAc6dHex3 | 2937 | + | |
| NeuGc2NeuAcHex5HexNAc6 | 2951 | + | |
| and/or | | | |
| NeuAc3Hex5HexNAc4dHex3 | | | |
| NeuAcHex7HexNAc6dHex2 | 2953 | + | + |
| Hex8HexNAc7dHexSP | 2961 | + | |
| NeuAc2Hex4HexNAc7dHex2 | 2961 | + | |
| NeuAcHex7HexNAc7dHex | 3010 | + | |
| NeuAc3Hex6HexNAc5dHex | 3024 | + | + |
| NeuAc2Hex6HexNAc5dHex3 | 3025 | + | + |
| NeuGc3Hex6HexNAc5dHex | 3072 | + | |
| and/or | | | |
| NeuGc2NeuAcHex7HexNAc5 | | | |
| NeuAc2Hex6HexNAc6dHex2 | 3082 | + | |
| NeuAc2Hex7HexNAc6dHex | 3098 | + | + |
| NeuAcHex7HexNAc6dHex3 | 3099 | + | + |
| NeuAc3Hex6HexNAc5dHexSP | 3104 | + | |
| NeuAc2Hex6HexNAc5dHex3SP | 3105 | + | |
| NeuAc3Hex6HexNAc5dHex2 | 3170 | + | |
| NeuAc2Hex6HexNAc5dHex4 | 3171 | + | |
| NeuAcHex8HexNAc7dHex | 3172 | + | |
| NeuAc3Hex6HexNAc6dHex | 3227 | + | |
| NeuAc2Hex6HexNAc6dHex3 | 3228 | + | |
| NeuAc2Hex7HexNAc6dHex2 | 3244 | + | + |
| NeuAcHex7HexNAc6dHex4 | 3245 | + | + |
| NeuAc2Hex7HexNAc7dHex | 3301 | + | |
| NeuAcHex7HexNAc7dHex3 | 3302 | + | |
| NeuAcHex8HexNAc7dHex2 | 3318 | + | |
| NeuAc3Hex7HexNAc6dHex | 3389 | + | + |
| NeuAc2Hex7HexNAc6dHex3 | 3390 | + | + |
| NeuAcHex7HexNAc6dHex5 | 3391 | + | |
| and/or NeuAcHex9HexNAc8 | | | |
| NeuAc2Hex8HexNAc7dHex | 3463 | + | |
| NeuAcHex8HexNAc7dHex3 | 3464 | + | |
| NeuAc2Hex7HexNAc6dHex4 | 3536 | + | |
| NeuAcHex9HexNAc8dHex | 3537 | + | |
| NeuAc2Hex8HexNac7dHex2 | 3609 | + | |
| NeuAcHex8HexNac7dHex4 | 3610 | + | |
| NeuAc4Hex7HexNAc6dHex | 3680 | + | |
| NeuAc3Hex7HexNAc6dHex3 | 3681 | + | |
| NeuAcHex9HexNAc8dHex2 | 3683 | + | |
| NeuAc3Hex8HexNAc7dHex | 3754 | + | |
| NeuAc2Hex8HexNAc7dHex3 | 3755 | + | |
| NeuAcHex10HexNAc9 and/or | 3756 | + | |
| NeuAcHex8HexNAc7dHex5 | | | |
| NeuAc3Hex7HexNAc6dHex4 | 3827 | + | |
| NeuAc2Hex9HexNAc8dHex | 3828 | + | |
| NeuAcHex9HexNAc8dHex3 | 3829 | + | |
| NeuAc2Hex8HexNAc7dHex4 | 3901 | + | |
| NeuAc2Hex9HexNAc8dHex2 | 3974 | + | |
| NeuAcHex9HexNAc8dHex4 | 3975 | + | |
| NeuAc4Hex8HexNAc7dHex | 4045 | + | |
| NeuAc3Hex8HexNAc7dHex3 | 4046 | + | |
| NeuAc2Hex10HexNAc9 | 4047 | + | |
| and/or | | | |
| NeuAc2Hex8HexNAc7dHex5 | | | |
| NeuAc3Hex9HexNAc8dHex | 4119 | + | |
| NeuAc2Hex9HexNAc8dHex3 | 4120 | + | |
| HexNAc ≧ 3 and dHex ≧ 2 | | | |
| (including multifucosylated N- | | | |
| glycans) | | Human | Human |
| Proposed composition | m/z | cells | cell line |
| Hex4HexNAc3dHex2SP | 1646 | + | |
| NeuAcHex3HexNAc3dHexSP2 | 1709 | + | |
| Hex5HexNAc3dHex2SP | 1808 | + | |
| NeuAcHex4HexNAc3dHex2 | 1857 | + | |
| NeuAcHex5HexNAc3dHex | 1873 | + | + |
| and/or | | | |
| NeuGcHex4HexNAc3dHex2 | | | |
| NeuAcHex4HexNAc3dHex3 | 2003 | + | |
| NeuAcHex5HexNAc3dHex2 | 2019 | + | + |
| Hex8HexNAc3dHexSP and/or | 2148 | + | |
| NeuAc2Hex4HexNAc3dHex2 | | | |
| NeuAcHex5HexNAc3dHex3 | 2165 | + | + |
| NeuAc2Hex5HexNAc3dHex2 | 2310 | + | |
| and/or | | | |
| NeuGcNeuAcHex4HexNAc3dHex3 | | | |
| NeuAc2Hex5HexNAc3dHex2SP | 2390 | + | + |
| NeuAcHex8HexNAc3dHexSP | 2439 | + | |
| and/or | | | |
| NeuAc3Hex4HexNAc3dHex2 | | | |
| Hex5HexNAc4dHex2SP | 2011 | + | |
| Hex4HexNAc5dHex2SP | 2052 | + | |
| NeuAcHex4HexNAc4dHex2 | 2060 | + | + |
| NeuAcHex3HexNAc5dHex2 | 2101 | + | |
| and/or | | | |
| NeuAc2Hex4HexNAc4Ac | | | |
| Hex4HexNAc5dHex2SP2 | 2132 | + | |
| Hex5HexNAc4dHex3SP | 2157 | + | |
| NeuAcHex6HexNAc4SP | 2172 | + | + |
| and/or | | | |
| NeuGcHex5HexNAc4dHexSP | | | |
| Hex6HexNAc4dHex2SP | 2173 | + | |
| and/or | | | |
| Hex3HexNAc6dHex2SP2 | | | |
| NeuAc2Hex3HexNAc4dHex2 | 2189 | + | + |
| and/or Hex7HexNAc4dHexSP | | | |
| and/or | | | |
| Hex4HexNAc6dHexSP2 | | | |
| NeuAcHex3HexNAc4dHex4 | 2190 | + | + |
| Hex4HexNAc5dHex3SP | 2198 | + | + |
| NeuAcHex5HexNAc4dHex2 | 2222 | + | + |
| NeuAcHex6HexNAc4dHex | 2238 | + | + |
| and/or | | | |
| NeuGcHex5HexNAc4dHex2 | | | |
| NeuAcHex4HexNAc5dHex2 | 2263 | + | |
| and/or | | | |
| NeuAc2Hex5HexNAc4Ac | | | |
| NeuAc2Hex4HexNAc4dHexSP | 2285 | + | |
| and/or Hex11HexNAc2SP | | | |
| NeuAcHex5HexNAc4dHex2SP | 2302 | + | |
| Hex6HexNAc4dHex3SP | 2319 | + | |
| and/or | | | |
| NeuGcNeuAcHex3HexNAc6 | | | |
| Hex7HexNAc4dHex2SP | 2335 | + | |
| and/or | | | |
| Hex4HexNAc6dHex2SP2 | | | |
| NeuAc2Hex4HexNAc4dHexSP | 2365 | + | |
| NeuAcHex5HexNAc4dHex3 | 2368 | + | + |
| NeuAcHex6HexNAc4dHex2 | 2384 | + | + |
| and/or | | | |
| NeuGcHex5HexNAc4dHex3 | | | |
| NeuAc2Hex3HexNAc5dHex2 | 2392 | + | + |
| and/or Hex7HexNAc5dHexSP | | | |
| NeuAcHex3HexNAc5dHex4 | 2393 | + | |
| NeuAcHex4HexNAc6dHexSP | 2400 | + | |
| and/or | | | |
| NeuGcHex6HexNAc4dHex2 | | | |
| and/or | | | |
| NeuAcHex7HexNAc4dHex | | | |
| Hex4HexNAc6dHex3SP | 2401 | + | |
| NeuAcHex4HexNAc5dHex3 | 2409 | + | |
| and/or | | | |
| NeuAc2Hex5HexNAc4dHexAc | | | |
| NeuAcHex5HexNAc5dHex2 | 2425 | + | + |
| NeuAcHex5HexNAc4dHex3SP | 2448 | + | |
| NeuAcHex4HexNAc5dHex3SP | 2489 | + | |
| NeuAc2Hex5HexNAc4dHex2 | 2513 | + | + |
| NeuAcHex5HexNAc4dHex4 | 2514 | + | |
| NeuAcHex6HexNAc5dHexSP | 2521 | + | |
| and/or | | | |
| NeuAc3Hex2HexNAc5dHex2 | | | |
| Hex6HexNAc5dHex3SP | 2522 | + | |
| NeuAc2Hex6HexNAc4dHex | 2529 | + | |
| and/or | | | |
| NeuGcNeuAcHex5HexNAc4dHex2 | | | |
| NeuGc2Hex5HexNAc4dHex2 | 2545 | + | |
| and/or | | | |
| NeuGcNeuAcHex6HexNAc4dHex | | | |
| NeuAcHex5HexNAc5dHex3 | 2571 | + | |
| NeuAcHex6HexNAc5dHex2 | 2587 | + | + |
| NeuAcHex7HexNAc5dHex | 2603 | + | + |
| and/or | | | |
| NeuGcHex6HexNAc5dHex2 | | | |
| NeuGcHex8HexNAc5 and/or | 2635 | + | |
| NeuAcHex4HexNAc5dHex4SP | | | |
| NeuAc2Hex5HexNAc4dHex3 | 2659 | + | |
| NeuAc2Hex4HexNAc5dHex2SP2 | 2714 | + | |
| NeuAcHex4HexNAc5dHex4SP2 | 2715 | + | + |
| and/or | | | |
| NeuAc3Hex5HexNAc5 | | | |
| NeuAc2Hex5HexNAc5dHex2 | 2716 | + | |
| NeuAcHex6HexNAc5dHex3 | 2733 | + | + |
| NeuAcHex6HexNAc6dHex2 | 2791 | + | |
| NeuAc3Hex5HexNAc4dHex2 | 2804 | + | |
| and/or | | | |
| NeuAcHex6HexNAc6dHexSP2 | | | |
| Hex6HexNAc6dHex3SP2 | 2805 | + | |
| NeuAcHex6HexNAc5dHex3SP | 2813 | + | |
| NeuAc3Hex6HexNAc4dHex | 2820 | + | |
| and/or | | | |
| NeuGcNeuAc2Hex5HexNAc4dHex2 | | | |
| NeuAc2Hex6HexNAc5dHex2 | 2879 | + | + |
| NeuAcHex6HexNAc5dHex4 | 2880 | + | + |
| NeuAc2Hex7HexNAc5dHex | 2895 | + | + |
| and/or | | | |
| NeuGcNeuAcHex6HexNAc5dHex2 | | | |
| NeuAc3Hex6HexNAc4dHexSP | 2900 | + | |
| and/or | | | |
| NeuGcNeuAc2Hex5HexNAc4dHex2SP | | | |
| NeuGc2Hex6HexNAc5dHex2 | 2911 | + | |
| NeuAc2Hex5HexNAc6dHex2 | 2920 | + | |
| NeuAc2Hex6HexNAc6dHex | 2936 | + | |
| and/or | | | |
| NeuGcNeuAcHex5HexNAc6dHex2 | | | |
| NeuAcHex6HexNAc6dHex3 | 2937 | + | |
| NeuGc2NeuAcHex5HexNAc6 | 2951 | + | |
| and/or | | | |
| NeuAc3Hex5HexNAc4dHex3 | | | |
| NeuAcHex7HexNAc6dHex2 | 2953 | + | + |
| NeuAc2Hex4HexNAc7dHex2 | 2961 | + | |
| NeuAc2Hex6HexNAc5dHex3 | 3025 | + | + |
| NeuAc2Hex6HexNAc6dHex2 | 3082 | + | |
| NeuAcHex7HexNAc6dHex3 | 3099 | + | + |
| NeuAc3Hex6HexNAc5dHexSP | 3104 | + | |
| NeuAc2Hex6HexNAc5dHex3SP | 3105 | + | |
| NeuAc3Hex6HexNAc5dHex2 | 3170 | + | |
| NeuAc2Hex6HexNAc5dHex4 | 3171 | + | |
| NeuAc2Hex6HexNAc6dHex3 | 3228 | + | |
| NeuAc2Hex7HexNAc6dHex2 | 3244 | + | + |
| NeuAcHex7HexNAc6dHex4 | 3245 | + | + |
| NeuAcHex7HexNAc7dHex3 | 3302 | + | |
| NeuAcHex8HexNAc7dHex2 | 3318 | + | |
| NeuAc2Hex7HexNAc6dHex3 | 3390 | + | + |
| NeuAcHex7HexNAc6dHex5 | 3391 | + | |
| and/or NeuAcHex9HexNAc8 | | | |
| NeuAcHex8HexNAc7dHex3 | 3464 | + | |
| NeuAc2Hex7HexNAc6dHex4 | 3536 | + | |
| NeuAc2Hex8HexNac7dHex2 | 3609 | + | |
| NeuAcHex8HexNac7dHex4 | 3610 | + | |
| NeuAc3Hex7HexNAc6dHex3 | 3681 | + | |
| NeuAcHex9HexNAc8dHex2 | 3683 | + | |
| NeuAc2Hex8HexNAc7dHex3 | 3755 | + | |
| NeuAcHex10HexNAc9 and/or | 3756 | + | |
| NeuAcHex8HexNAc7dHex5 | | | |
| NeuAc3Hex7HexNAc6dHex4 | 3827 | + | |
| NeuAcHex9HexNAc8dHex3 | 3829 | + | |
| NeuAc2Hex8HexNAc7dHex4 | 3901 | + | |
| NeuAc2Hex9HexNAc8dHex2 | 3974 | + | |
| NeuAcHex9HexNAc8dHex4 | 3975 | + | |
| NeuAc3Hex8HexNAc7dHex3 | 4046 | + | |
| NeuAc2Hex10HexNAc9 | 4047 | + | |
| and/or | | | |
| NeuAc2Hex8HexNAc7dHex5 | | | |
| NeuAc2Hex9HexNAc8dHex3 | 4120 | + | |
| HexNAc > Hex ≧ 3 | | | |
| (terminal HexNAc, N > H) | | Human | Human |
| Proposed composition | m/z | cells | cell line |
| Hex6HexNAc3SP and/or | 1678 | + | + |
| NeuAc2Hex2HexNAc3dHex | | | |
| NeuAcHex3HexNAc4 | 1606 | + | |
| NeuAcHex3HexNac5 | 1809 | + | |
| NeuGcHex3HexNAc5 | 1825 | + | + |
| Hex4HexNAc5dHexSP | 1906 | + | |
| NeuAcHex4HexNAc5 | 1971 | + | |
| Hex7HexNAc4SP and/or | 2043 | + | |
| Hex4HexNAc6SP2 and/or | | | |
| NeuAc2Hex3HexNAc4dHex | | | |
| NeuAcHex4HexNAc5SP | 2051 | + | |
| Hex4HexNAc5dHex2SP | 2052 | + | |
| NeuAcHex3HexNAc5dHex2 | 2101 | + | |
| and/or | | | |
| NeuAc2Hex4HexNAc4Ac | | | |
| NeuAcHex4HexNAc5dHex | 2117 | + | |
| Hex4HexNAc5dHex2SP2 | 2132 | + | |
| Hex6HexNAc4dHex2SP | 2173 | + | |
| and/or | | | |
| Hex3HexNAc6dHex2SP2 | | | |
| NeuAcHex4HexNAc6 | 2174 | + | |
| NeuAc3Hex3HexNAc4 and/or | 2188 | + | |
| NeuGcHex6HexNAc4SP | | | |
| and/or | | | |
| NeuAc2NeuGcHex2HexNAc4dHex | | | |
| NeuAc2Hex3HexNAc4dHex2 | 2189 | + | + |
| and/or Hex7HexNAc4dHexSP | | | |
| and/or | | | |
| Hex4HexNAc6dHexSP2 | | | |
| NeuAcHex3HexNAc4dHex4 | 2190 | + | + |
| Hex4HexNAc5dHex3SP | 2198 | + | + |
| NeuAc2Hex4HexNAc5 | 2262 | + | |
| NeuAcHex4HexNAc5dHex2 | 2263 | + | |
| and/or | | | |
| NeuAc2Hex5HexNAc4Ac | | | |
| Hex5HexNAc6dHexSP | 2271 | + | |
| NeuAcHex4HexNAc6dHex | 2320 | + | |
| Hex7HexNAc4dHex2SP | 2335 | + | |
| and/or | | | |
| Hex4HexNAc6dHex2SP2 | | | |
| NeuAcHex5HexNAc6 | 2336 | + | + |
| NeuAc2Hex3HexNAc5dHex2 | 2392 | + | + |
| and/or Hex7HexNAc5dHexSP | | | |
| NeuAcHex3HexNAc5dHex4 | 2393 | + | |
| NeuAcHex4HexNAc6dHexSP | 2400 | + | |
| and/or | | | |
| NeuGcHex6HexNAc4dHex2 | | | |
| and/or | | | |
| NeuAcHex7HexNAc4dHex | | | |
| Hex4HexNAc6dHex3SP | 2401 | + | |
| NeuAc2Hex4HexNAc5dHex | 2408 | + | |
| NeuAcHex4HexNAc5dHex3 | 2409 | + | |
| and/or | | | |
| NeuAc2Hex5HexNAc4dHexAc | | | |
| NeuAcHex5HexNAc6dHex | 2482 | + | |
| NeuAcHex4HexNAc5dHex3SP | 2489 | + | |
| Hex6HexNAc7SP | 2490 | + | |
| NeuGc3Hex5HexNAc4 | 2560 | + | |
| NeuAc2Hex5HexNAc6 | 2627 | + | |
| NeuAc2Hex4HexNAc5dHex2SP2 | 2714 | + | |
| NeuAcHex4HexNAc5dHex4SP2 | 2715 | + | + |
| and/or | | | |
| NeuAc3Hex5HexNAc5 | | | |
| NeuAc2Hex5HexNAc6dHex2 | 2920 | + | |
| NeuGcNeuAc2Hex5HexNAc6 | 2935 | + | |
| NeuAc2Hex4HexNAc7dHex2 | 2961 | + | |
| NeuAc4Hex6HexNAc8 | 3778 | + | |
| HexNAc = Hex ≧ 5 | | | |
| (terminal HexNAc, N = H) | | Human | Human |
| Proposed composition | m/z | cells | cell line |
| Hex5HexNAc5SP2 | 2002 | + | |
| NeuAcHex5HexNAc5 | 2133 | + | + |
| NeuAcHex5HexNAc5dHex | 2279 | + | + |
| NeuAcHex5HexNAc5dHexAc | 2321 | + | |
| NeuAc2Hex5HexNAc5 | 2424 | + | |
| NeuAcHex5HexNAc5dHex2 | 2425 | + | + |
| NeuAc2Hex5HexNAc5dHex | 2570 | + | + |
| NeuAcHex5HexNAc5dHex3 | 2571 | + | |
| NeuAcHex6HexNAc6dHex | 2644 | + | + |
| NeuAcHex4HexNAc5dHex4SP2 | 2715 | + | + |
| and/or | | | |
| NeuAc3Hex5HexNAc5 | | | |
| NeuAc2Hex5HexNAc5dHex2 | 2716 | + | |
| NeuAcHex6HexNAc6dHex2 | 2791 | + | |
| Hex6HexNAc6dHex3SP2 | 2805 | + | |
| NeuAc2Hex6HexNAc6dHex | 2936 | + | |
| and/or | | | |
| NeuGcNeuAcHex5HexNAc6dHex2 | | | |
| NeuAcHex6HexNAc6dHex3 | 2937 | + | |
| NeuAcHex7HexNAc7dHex | 3010 | + | |
| NeuAc3Hex6HexNAc6 | 3081 | + | + |
| NeuAc2Hex6HexNAc6dHex2 | 3082 | + | |
| NeuAc3Hex6HexNAc6dHex | 3227 | + | |
| NeuAc2Hex6HexNAc6dHex3 | 3228 | + | |
| NeuAc2Hex7HexNAc7dHex | 3301 | + | |
| NeuAcHex7HexNAc7dHex3 | 3302 | + | |
| SP ≧ 1 | | | |
| (including sulphated and/or | | | |
| phosphorylated glycans) | | Human | Human |
| Proposed composition | m/z | cells | cell line |
| Hex3HexNAc2SP | 989 | + | |
| Hex3HexNAc2dHexSP | 1135 | + | |
| Hex4HexNAc2SP | 1151 | + | |
| Hex3HexNAc3SP | 1192 | + | + |
| Hex5HexNAc2SP | 1313 | + | |
| Hex3HexNAc3dHexSP | 1338 | + | + |
| Hex4HexNAc3SP | 1354 | + | + |
| Hex5HexNAc2dHexSP | 1459 | + | + |
| Hex6HexNAc2SP | 1475 | + | |
| Hex4HexNAc3dHexSP | 1500 | + | + |
| Hex5HexNAc3SP | 1516 | + | |
| Hex6HexNAc2SP2 | 1555 | + | |
| Hex4HexNAc4SP | 1557 | + | + |
| NeuAcHex3HexNAc3SP2 | 1563 | + | |
| Hex6HexNAc2dHexSP | 1621 | + | + |
| Hex4HexNAc4SP2 and/or | 1637 | + | |
| Hex7HexNAc2SP | | | |
| Hex4HexNAc3dHex2SP | 1646 | + | |
| Hex5HexNAc3dHexSP | 1662 | + | + |
| Hex6HexNAc3SP | 1678 | + | |
| Hex4HexNAc4dHexSP | 1703 | + | + |
| NeuAcHex3HexNAc3dHexSP2 | 1709 | + | |
| Hex4HexNAc4SP3 and/or | 1717 | + | |
| Hex7HexNAc2SP2 | | | |
| Hex5HexNAc4SP | 1719 | + | + |
| Hex7HexNAc2dHexSP | 1783 | + | |
| NeuAcHex4HexNAc3dHexSP | 1791 | + | |
| Hex5HexNAc4SP2 and/or | 1799 | + | |
| Hex8HexNAc2SP | | | |
| Hex5HexNAc3dHex2SP | 1808 | + | |
| NeuAc2Hex5HexNAc2 and/or | 1815 | + | |
| NeuAc2Hex2HexNAc4SP | | | |
| Hex6NexNAc3dHexSP | 1824 | + | + |
| Hex5HexNAc4dHexSP | 1865 | + | + |
| Hex6HexNAc4SP | 1881 | + | + |
| Hex4HexNAc5dHexSP | 1906 | + | |
| NeuAcHex6HexNAc2dHexSP | 1912 | + | |
| and/or | | | |
| NeuAcHex3HexNAc4dHexSP2 | | | |
| NeuAcHex4HexNAc4SP2 | 1928 | + | |
| Hex8HexNAc3SP and/or | 2002 | + | + |
| Hex5HexNAc5SP2 and/or | | | |
| NeuAc2Hex4HexNAc3dHex | | | |
| NeuAcHex5HexNAc4SP | 2010 | + | + |
| Hex5HexNAc4dHex2SP | 2011 | + | |
| NeuGcHex5HexNAc4SP | 2026 | + | |
| Hex6HexNAc4dHexSP | 2027 | + | + |
| Hex7HexNAc4SP and/or | 2043 | + | |
| Hex4HexNAc6SP2 and/or | | | |
| NeuAc2Hex3HexNAc4dHex | | | |
| NeuAcHex7HexNAc3 and/or | 2051 | + | + |
| NeuAcHex4HexNAc5SP | | | |
| Hex4HexNAc5dHex2SP | 2052 | + | |
| NeuAcHex4HexNAc4dHexSP2 | 2074 | + | |
| NeuAc2Hex4HexNAc3dHexSP | 2082 | + | |
| and/or Hex8HexNAc3SP2 | | | |
| and/or Hex5HexNAc5SP3 | | | |
| NeuAcHex6HexNAc3dHexSP | 2115 | + | |
| Hex8HexNAc3dHexSP and/or | 2148 | + | |
| NeuAc2Hex4HexNAc3dHex2 | | | |
| NeuAcHex5HexNAc4dHexSP | 2156 | + | + |
| and/or | | | |
| NeuAcHex8HexNAc2dHex | | | |
| Hex5HexNAc4dHex3SP | 2157 | + | |
| NeuAc2Hex5HexNAc3dHex | 2164 | + | + |
| and/or Hex6HexNAc5SP2 | | | |
| NeuAcHex6HexNAc4SP | 2172 | + | + |
| and/or | | | |
| NeuGcHex5HexNAc4dHexSP | | | |
| and/or NeuAcHex9HexNAc2 | | | |
| Hex6HexNAc4dHex2SP | 2173 | + | |
| and/or | | | |
| Hex3HexNAc6dHex2SP2 | | | |
| NeuAc3Hex3HexNAc4 and/or | 2188 | + | |
| NeuGcHex6HexNAc4SP | | | |
| and/or | | | |
| NeuAc2NeuGcHex2HexNAc4dHex | | | |
| NeuAc2Hex3HexNAc4dHex2 | 2189 | + | + |
| and/or Hex7HexNAc4dHexSP | | | |
| and/or | | | |
| Hex4HexNAc6dHexSP2 | | | |
| Hex4HexNAc5dHex3SP | 2198 | + | + |
| NeuAc2Hex4HexNAc4SP2 | 2219 | + | |
| Hex6HexNAc5dHexSP | 2230 | + | |
| NeuAc2Hex3HexNAc5dHex | 2246 | + | |
| and/or Hex7HexNAc5SP | | | |
| NeuAc2Hex4HexNAc4dHexSP | 2285 | + | |
| and/or Hex11HexNAc2SP | | | |
| NeuAcHex8HexNAc3SP | 2293 | + | |
| and/or | | | |
| NeuAc3Hex4HexNAc3dHex | | | |
| NeuAc2Hex5HexNAc4SP | 2301 | + | |
| NeuAcHex5HexNAc4dHex2SP | 2302 | + | |
| NeuAcHex6HexNAc4dHexSP | 2318 | + | + |
| Hex6HexNAc4dHex3SP | 2319 | + | |
| Hex7HexNAc4dHex2SP | 2335 | + | |
| and/or | | | |
| Hex4HexNAc6dHex2SP2 | | | |
| NeuAc2Hex4HexNAc4dHexSP | 2365 | + | |
| NeuAc3Hex5HexNAc3SP | 2389 | + | |
| and/or | | | |
| NeuAc2Hex5HexNAc4Ac4 | | | |
| NeuAc2Hex5HexNAc3dHex2SP | 2390 | + | + |
| NeuAc2Hex3HexNAc5dHex2 | 2392 | + | + |
| and/or Hex7HexNAc5dHexSP | | | |
| NeuAcHex4HexNAc6dHexSP | 2400 | + | |
| and/or | | | |
| NeuGcHex6HexNAc4dHex2 | | | |
| and/or | | | |
| NeuAcHex7HexNAc4dHex | | | |
| NeuAc2Hex6HexNAc3dHexSP | 2406 | + | |
| NeuAcHex8HexNAc3dHexSP | 2439 | + | |
| and/or | | | |
| NeuAc3Hex4HexNAc3dHex2 | | | |
| NeuAc2Hex5HexNAc4dHexSP | 2447 | + | + |
| and/or | | | |
| NeuAc2Hex8HexNAc2dHex | | | |
| and/or Hex12HexNAc2SP | | | |
| NeuAcHex5HexNAc4dHex3SP | 2448 | + | |
| and/or | | | |
| NeuAcHex8HexNAc2dHex3 | | | |
| NeuAcHex7HexNAc3dHex3 | 2489 | + | |
| and/or | | | |
| NeuAcHex4HexNAc5dHex3SP | | | |
| Hex6HexNAc7SP | 2490 | + | |
| NeuAcHex6HexNAc5dHexSP | 2521 | + | |
| and/or | | | |
| NeuAcHex9HexNAc3dHex | | | |
| and/or | | | |
| NeuAc3Hex2HexNAc5dHex2 | | | |
| Hex6HexNAc5dHex3SP | 2522 | + | |
| Hex7HexNAc6dHexSP | 2595 | + | |
| NeuGcHex8HexNAc5 and/or | 2635 | + | |
| NeuAcHex4HexNAc5dHex4SP | | | |
| NeuAc2Hex5HexNAc5dHexSP | 2650 | + | |
| Hex7HexNAc7SP | 2652 | + | |
| Hex6HexNAc5dHex4SP | 2668 | + | |
| NeuGcHex6HexNAc5dHexSP | 2683 | + | |
| and/or | | | |
| NeuAcHex7HexNAc5dHexSP | | | |
| NeuAc2Hex4HexNAc5dHex2SP2 | 2714 | + | |
| NeuAcHex4HexNAc5dHex4SP2 | 2715 | + | |
| and/or | | | |
| NeuAc3Hex5HexNAc5 | | | |
| Hex6HexNAc6dHex3SP | 2725 | + | |
| Hex7HexNAc6dHex2SP | 2741 | + | |
| NeuAcHex6HexNAc5dHex2SP2 | 2747 | + | |
| NeuAc2Hex4HexNAc6dHex2 | 2757 | + | |
| and/or Hex8HexNAc6dHexSP | | | |
| Hex7HexNAc7dHexSP | 2798 | + | |
| NeuAc3Hex5HexNAc4dHex2 | 2804 | + | |
| and/or | | | |
| NeuAcHex6HexNAc6dHexSP2 | | | |
| Hex6HexNAc6dHex3SP2 | 2805 | + | |
| NeuAc2Hex6HexNAc5dHexSP | 2812 | + | |
| NeuAcHex6HexNAc5dHex3SP | 2813 | + | |
| Hex8HexNAc7SP | 2814 | + | |
| Hex6HexNAc6dHex4SP | 2871 | + | |
| NeuAcHex7HexNAc6dHexSP | 2887 | + | |
| and/or | | | |
| NeuAcHex10HexNAc4dHex | | | |
| Hex7HexNAc6dHex3SP | 2887 | + | |
| NeuAc3Hex6HexNAc4dHexSP | 2900 | + | |
| and/or | | | |
| NeuGcNeuAc2Hex5HexNAc4dHex2SP | | | |
| NeuAc3Hex4HexNAc6dHex | 2903 | + | |
| and/or | | | |
| NeuAcHex8HexNAc6SP | | | |
| Hex7HexNAc7dHex2SP | 2945 | + | |
| NeuAc2Hex6HexNAc5dHex2SP | 2958 | + | |
| NeuAcHex6HexNAc5dHex4SP | 2960 | + | |
| Hex8HexNAc7dHexSP | 2961 | + | |
| Hex8HexNAc8SP | 3018 | + | |
| Hex7HexNAc6dHex4SP | 3034 | + | |
| Hex7HexNAc7dHex3SP | 3091 | + | |
| NeuAc3Hex6HexNAc5dHexSP | 3104 | + | |
| NeuAc2Hex6HexNAc5dHex3SP | 3105 | + | |
| NeuAcHex8HexNAc7SP | 3106 | + | |
| and/or | | | |
| NeuAc3Hex4HexNAc7dHex | | | |
| Hex8HexNAc7dHex2SP | 3107 | + | |
| and/or | | | |
| NeuAc2Hex4HexNAc7dHex3 | | | |
| NeuAc2Hex7HexNAc6dHexSP | 3178 | + | |
| Hex7HexNAc7dHex4SP | 3237 | + | |
| NeuAc3Hex7HexNAc5dHexSP | 3266 | + | |
| and/or | | | |
| NeuGcNeuAc2Hex6HexNAc5dHex2SP | | | |
| NeuAc3Hex5HexNAc7dHex | 3268 | + | |
| and/or | | | |
| NeuGcHex8HexNAc7dHexSP | | | |
| NeuAc4Hex4HexNAc5dHex2SP2 | 3297 | + | |
| NeuAc3Hex4HexNAc5dHex4SP2 | 3298 | + | |
| Hex8HexNAc8dHex3SP | 3456 | + | |
| and/or | | | |
| NeuAc2Hex4HexNAc8dHex4 | | | |
| NeuAc3Hex7HexNAc6dHexSP | 3469 | + | |
| NeuAc2Hex7HexNAc6dHex3SP | 3470 | + | |

TABLE 16
Structural classification of neutral glycan fraction glycan signals isolated from
normal human lung tissue (1. column), human lung cancer tissue (2. column), normal
human serum (5. column), and a cultured human cell line (6. column). Acidic glycan
fraction glycans analyzed as neutral desialylated glycan signals together with the
corresponding neutral glycan fraction are similarly classified from the same human tissue
samples (3. and 4. column, total normal and total cancer).

| Structural features of | | % | | | | | |
|---|---|---|---|---|---|---|---|
| Neutral N-glycans | | normal | | | | | |
| structural feature | proposed composition | lung | lung cancer | total normal | total cancer | human serum | human cell line |
| Hex₅₋₉HexNAc₂ | high-mannose | 47.0 | 46.0 | 17.8 | 22.3 | 25.7 | 53.7 |
| Hex₁₋₄HexNAc₂dHex₀₋₁ | low-mannose | 28.0 | 19.5 | 15.5 | 24.4 | 0.7 | 8.5 |
| Hex₁₀₋₁₂HexNAc₂ | high-mannose/Glc | 0.0 | 0.0 | 0.0 | 0.0 | 0.0 | 1.9 |
| Hex₅₋₆HexNAc₂dHex₁ | low-mannose + Fuc | 0.7 | 0.0 | 0.3 | 0.2 | 0.0 | 1.0 |
| n_{HexNAc} = 3 ja n_{Hex} ≧ 2 | hybrid/monoantennary | 7.9 | 8.7 | 8.4 | 7.1 | 6.6 | 7.3 |
| n_{HexNAc} ≧ 4 ja n_{Hex} ≧ 2 | complex type | 15.8 | 24.4 | 57.8 | 46.0 | 66.2 | 9.3 |
| Hex₁₋₉HexNAc | soluble | 0.7 | 0.5 | 0.0 | 0.0 | 0.8 | 11.3 |
| other | — | 0.0 | 0.9 | 0.2 | 0.0 | 0.0 | 6.9 |
| n_{dHex} ≧ 1 | fucosylation | 19.4 | 33.6 | 42.8 | 34.6 | 50.5 | 13.9 |
| n_{dHex} ≧ 2 | α2/3/4-Fuc | 0.0 | 0.8 | 0.3 | 1.1 | 0.0 | 1.3 |
| n_{HexNAc} > n_{Hex} ≧ 2 | terminal HexNAc | 3.9 | 17.8 | 3.8 | 7.1 | 21.8 | 4.2 |
| n_{HexNAc} = n_{Hex} ≧ 3 | terminal HexNAc | 6.9 | 8.2 | 8.2 | 5.0 | 31.4 | 1.9 |

TABLE 17
N-glycan structural classification of lysosomal protein sample.

| Glycan feature | Proposed structure | Proportion, % |
|---|---|---|
| Neutral N-glycan structural features: | | |
| Hex₅₋₁₀HexNAc₂ | High-mannose type/Glc₁ | 46 |
| Hex₁₋₄HexNAc₂dHex₀₋₁ | Low-mannose type | 49 |
| n_{HexNAc} = 3 ja n_{Hex} ≧ 2 | Hybrid-type/Monoantennary | 2 |
| n_{HexNAc} ≧ 4 ja n_{Hex} ≧ 2 | Complex-type | 0.6 |
| Other | — | <3 |
| n_{dHex} ≧ 1 | Fucosylation | 29 |
| n_{dHex} ≧ 2 | α2/3/4-linked Fuc | 0.8 |
| n_{HexNAc} > n_{Hex} ≧ 2 | Terminal HexNAc (N > H) | 0.2 |
| n_{HexNAc} = n_{Hex} ≧ 5 | Terminal HexNAc (N = H) | — |
| Acidic N-glycan structural features: | | |
| n_{HexNAc} = 3 ja n_{Hex} ≧ 3 | Hybrid-type/Monoantennary | 46 |
| n_{HexNAc} ≧ 4 ja n_{Hex} ≧ 3 | Complex-type | 37 |
| muut | — | 17 |
| n_{dHex} ≧ 1 | Fucosylation | 80 |
| n_{dHex} ≧ 2 | α2/3/4-linked Fuc | 10 |
| n_{HexNAc} > n_{Hex} ≧ 2 | Terminal HexNAc (N > H) | 0.1 |
| n_{HexNAc} = n_{Hex} ≧ 5 | Terminal HexNAc (N = H) | 0.4 |
| +80 Da | Sulphate or phosphate ester | 17 |

TABLE 18
Identification of disease-specific glycosylation by quantitative glycome
analysis.

| | | | | | | | Abs. | | | Rel. |
|---|---|---|---|---|---|---|---|---|---|---|
| Composition | m/z | Class | I | II | m/z | Class | differ. | m/z | Class | differ. |
| Hex1HexNAc2 | 609 | NL | 0.00 | 0.00 | 771 | NL | 12.8 | 1955 | NCE | new |
| Hex2HexNAc1dHex1 | 714 | NOF | 0.00 | 0.00 | 1485 | NCFT | 3.5 | 2685 | NCE | new |
| Hex3HexNAc1 | 730 | NS | 0.00 | 0.00 | 1743 | NM | 2.1 | 2905 | NCF | new |
| Hex1HexNAc2dHex1 | 755 | NLF | 2.47 | 0.00 | 1905 | NM | 1.8 | 771 | NL | 2.4 |
| Hex2HexNAc2 | 771 | NL | 5.44 | 18.25 | 1419 | NM | 1.4 | 1905 | NM | 2.2 |
| Hex2HexNAc2dHex1 | 917 | NLF | 1.81 | 2.61 | 917 | NLF | 0.8 | 1485 | NCFT | 1.3 |
| Hex3HexNAc2 | 933 | NL | 2.47 | 1.12 | 1581 | NM | 0.5 | 2394 | NC | 1.3 |
| Hex2HexNAc3 | 974 | NH-T | 0.00 | 0.00 | 1955 | NCE | 0.4 | 1743 | NM | 1.2 |
| Hex2HexNAc2dHex2 | 1063 | NOE | 0.00 | 0.00 | 2685 | NCE | 0.4 | 917 | NLF | 0.4 |
| Hex3HexNAc2dHex1 | 1079 | NLF | 1.81 | 1.12 | 2905 | NCF | 0.4 | 1419 | NM | 0.4 |
| Hex4HexNAc2 | 1095 | NL | 1.48 | 1.30 | 2539 | NCF | 0.3 | 2539 | NCF | 0.4 |
| Hex2HexNAc3dHex1 | 1120 | NHFT | 0.00 | 0.00 | 2394 | NC | 0.2 | 1581 | NM | 0.2 |
| Hex3HexNAc3 | 1136 | NH | 0.82 | 0.00 | 2175 | NCF | 0.2 | 1282 | NHF | 0.1 |
| Hex2HexNAc2dHex3 | 1209 | NOE | 0.00 | 0.00 | 1622 | NH | 0.2 | 2012 | NCFB | 0.1 |
| Hex3HexNAc2dHex2 | 1225 | NOE | 0.00 | 0.00 | 1282 | NHF | 0.1 | 1622 | NH | 0.1 |
| Hex4HexNAc2dHex1 | 1241 | NLF | 0.00 | 0.00 | 2012 | NCFB | 0.1 | 1339 | NH-T | 0.1 |
| Hex5HexNAc2 | 1257 | NM | 8.90 | 7.64 | 1339 | NH-T | 0.0 | 2320 | NCE | 0.1 |
| Hex2HexNAc3dHex2 | 1266 | NHET | 0.00 | 0.00 | 2320 | NCE | 0.0 | 2175 | NCF | 0.0 |
| Hex3HexNAc3dHex1 | 1282 | NHF | 0.82 | 0.93 | 609 | NL | 0.0 | 609 | NL | 0.0 |
| Hex4HexNAc3 | 1298 | NH | 1.48 | 1.12 | 714 | NOF | 0.0 | 714 | NOF | 0.0 |
| Hex3HexNAc4 | 1339 | NH-T | 0.33 | 0.37 | 730 | NS | 0.0 | 730 | NS | 0.0 |
| Hex5HexNAc2dHex1 | 1403 | NMF | 0.33 | 0.19 | 974 | NH-T | 0.0 | 974 | NH-T | 0.0 |
| Hex6HexNAc2 | 1419 | NM | 3.95 | 5.40 | 1063 | NOE | 0.0 | 1063 | NOE | 0.0 |
| Hex3HexNAc3dHex2 | 1428 | NHE | 0.00 | 0.00 | 1120 | NHFT | 0.0 | 1120 | NHFT | 0.0 |
| Hex4HexNAc3dHex1 | 1444 | NHF | 1.65 | 1.30 | 1209 | NOE | 0.0 | 1209 | NOE | 0.0 |
| Hex5HexNAc3 | 1460 | NH | 2.47 | 2.42 | 1225 | NOE | 0.0 | 1225 | NOE | 0.0 |
| Hex3HexNAc4dHex1 | 1485 | NCFT | 2.64 | 6.15 | 1241 | NLF | 0.0 | 1241 | NLF | 0.0 |
| Hex4HexNAc4 | 1501 | NC | 1.32 | 0.93 | 1266 | NHET | 0.0 | 1266 | NHET | 0.0 |
| Hex3HexNAc5 | 1542 | NC-T | 0.00 | 0.00 | 1428 | NHE | 0.0 | 1428 | NHE | 0.0 |
| Hex7HexNAc2 | 1581 | NM | 2.31 | 2.79 | 1542 | NC-T | 0.0 | 1542 | NC-T | 0.0 |
| Hex6HexNAc3 | 1622 | NH | 1.15 | 1.30 | 1688 | NCFT | 0.0 | 1688 | NCFT | 0.0 |
| Hex4HexNAc4dHex1 | 1647 | NCF | 3.95 | 2.23 | 2028 | NC | 0.0 | 2028 | NC | 0.0 |
| Hex5HexNAc4 | 1663 | NC | 17.63 | 13.97 | 1460 | NH | −0.1 | 1460 | NH | 0.0 |
| Hex3HexNAc5dHex1 | 1688 | NCFT | 0.00 | 0.00 | 1850 | NCFT | −0.1 | 1095 | NL | −0.1 |
| Hex4HexNAc5 | 1704 | NC-T | 0.16 | 0.00 | 1403 | NMF | −0.1 | 1257 | NM | −0.1 |
| Hex8HexNAc2 | 1743 | NM | 1.81 | 3.91 | 1704 | NC-T | −0.2 | 1850 | NCFT | −0.2 |
| Hex5HexNAc4dHex1 | 1809 | NCF | 20.59 | 11.73 | 1095 | NL | −0.2 | 1663 | NC | −0.2 |
| Hex6HexNAc4 | 1825 | NC | 2.47 | 0.56 | 1444 | NHF | −0.3 | 1444 | NHF | −0.2 |
| Hex4HexNAc5dHex1 | 1850 | NCFT | 0.66 | 0.56 | 1298 | NH | −0.4 | 1298 | NH | −0.2 |
| Hex5HexNAc5 | 1866 | NC-B | 0.49 | 0.00 | 1501 | NC | −0.4 | 1501 | NC | −0.3 |
| Hex9HexNAc2 | 1905 | NM | 0.82 | 2.61 | 1866 | NC-B | −0.5 | 1079 | NLF | −0.4 |
| Hex5HexNAc4dHex2 | 1955 | NCE | 0.00 | 0.37 | 1079 | NLF | −0.7 | 1809 | NCF | −0.4 |
| Hex5HexNAc5dHex1 | 2012 | NCFB | 0.82 | 0.93 | 1136 | NH | −0.8 | 1647 | NCF | −0.4 |
| Hex6HexNAc5 | 2028 | NC | 1.32 | 1.30 | 1257 | NM | −1.3 | 1403 | NMF | −0.4 |
| Hex6HexNAc5dHex1 | 2175 | NCF | 4.12 | 4.28 | 933 | NL | −1.4 | 933 | NL | −0.5 |
| Hex6HexNAc5dHex2 | 2320 | NCE | 0.33 | 0.37 | 1647 | NCF | −1.7 | 1825 | NC | −0.8 |
| Hex7HexNAc6 | 2394 | NC | 0.16 | 0.37 | 1825 | NC | −1.9 | 1704 | NC-T | gone |
| Hex7HexNAc6dHex1 | 2539 | NCF | 0.82 | 1.12 | 755 | NLF | −2.5 | 1866 | NC-B | gone |
| Hex7HexNAc6dHex2 | 2685 | NCE | 0.00 | 0.37 | 1663 | NC | −3.7 | 1136 | NH | gone |
| Hex8HexNAc7dHex1 | 2905 | NCF | 0.00 | 0.37 | 1809 | NCF | −8.9 | 755 | NLF | gone |

TABLE 19
Detected tissue material N-linked and soluble glycome compositions.

| Glycan feature | Proposed structure | Proportion, % |
|---|---|---|
| Neutral N-glycan structural features: | | |
| Hex₅₋₁₀HexNAc₂ | High-mannose type/Glc₁ | 10-60 |
| Hex₁₋₄ | Low-mannose type | 0-50 |
| HexNAc₂dHex₀₋₁ | | |
| n_{HexNAc} = 3 ja n_{Hex} ≧ 2 | Hybrid-type/ | 5-20 |
| | Monoantennary | |
| n_{HexNAc} ≧ 4 ja n_{Hex} ≧ 2 | Complex-type | 5-75 |
| Hex₁₋₉HexNAc₁ | Soluble | 0-10 |
| n_{dHex} ≧ 1 | Fucosylation | 10-80 |
| n_{dHex} ≧ 2 | α2/3/4-linked Fuc | 0-40 |
| n_{HexNAc} > n_{Hex} ≧ 2 | Terminal HexNAc (N > H) | 1-30 |
| n_{HexNAc} = n_{Hex} ≧ 5 | Terminal HexNAc (N = H) | 1-40 |
| Acidic N-glycan structural features: | | all |
| n_{HexNAc} = 3 ja n_{Hex} ≧ 3 | Hybrid-type/ | 5-60 |
| | Monoantennary | |
| n_{HexNAc} ≧ 4 ja n_{Hex} ≧ 3 | Complex-type | 40-95 |
| n_{dHex} ≧ 1 | Fucosylation | 20-90 |
| n_{dHex} ≧ 2 | α2/3/4-linked Fuc | 0-50 |
| n_{HexNAc} > n_{Hex} ≧ 2 | Terminal HexNAc (N > H) | 0-40 |
| n_{HexNAc} = n_{Hex} ≧ 5 | Terminal HexNAc (N = H) | 0-40 |
| +80 Da | Sulphate or phosphate ester | 0-25 |

TABLE 20
Breast carcinoma acidic protein-linked glycan structural classification.

| Acidic N-glycan classification: | | | | | |
|---|---|---|---|---|---|
| Ductale type breast carcinoma, | | | | | |
| normal and tumor tissues | | | | | |
| Lymph node metastasis, | | | | | |
| normal and metastatic tissues | | Proportion of acidic glycans, % | | | |
| Composition | Classification | normal | cancer | LNN | LNM |
| n_{dHex} ≧ 1 | Fucosylated | 61 | 48 | 61 | 68 |
| n_{dHex} ≧ 2 | α2/3/4-Fuc | 1 | 8 | 6 | 7 |
| | (complex fucosylation) | | | | |
| +42 Da | Acetylated | 0.3 | 0.2 | 1 | 2 |
| +80 Da | Sulfated or | — | 2 | 1 | 4 |
| | phosphorylated | | | | |

TABLE 21
Lex/sLex antibodies.

| Code | Manufacturer specification | Manufacturer or literature ref. | Clone |
|---|---|---|---|
| | General Lex antibodies: | | |
| GF 305 | CBL144 (anti CD15) Le^{x} | Chemicon | 28 |
| GF 517 | ab34200 (CD15) | Abcam | TG-1 |
| GF 515 | 557895 anti-human CD15 | BD Pharmingen | W6D3 |
| GF 525 | ab17080-1 (CD15) | | MMA |
| | ab20138 | Abcam | 29 |
| | ab1252 | Abcam | BRA4F1 |
| | ab49758 | Abcam | BY87 |
| | ab51369 | Abcam | CLB- |
| | | | gran/2, |
| | | | B4 |
| | ab13453 | Abcam | DU- |
| | | | HL60-3 |
| | ab53997 | Abcam | LeuM1 |
| | ab6414 | Abcam | MC-1 |
| | ab665 | Abcam | MEM- |
| | | | 158 |
| | ab754 | Abcam | MY-1 |
| | ab15614 | Abcam | VIM-C6 |
| | Lewis x | Abcam | |
| | ab3358 | Abcam | P12 |
| | anti CD15 | Beckman Coulter | 80H5 |
| | anti CD15 | BioLegend | HI98 |
| | anti CD15 | Chemicon | ZC-18C |
| | anti CD15 | Chemicon | MCS-1 |
| | anti CD15 | Chemicon | DT07 & |
| | | | BC97 |
| | anti CD15 | Labvision | 15C02 |
| | anti CD15 | Labvision | SPM490 |
| | anti CD15 | Ancell | AHN1.1 |
| | anti CD15 | Quartett Immunodiagnostika, Berlin | Tu9 |
| | anti CD15 | Patricell | B-H8 |
| | anti CD15 | Patricell | HIM . . . |
| | anti CD15 | Santa Cruz | C3D-1 |
| | anti CD15 | Santa Cruz | 3G75 |
| | anti Lewis x | Santa Cruz | 4C9 |
| | anti CD15 | ScyTek Laboratories | FR4A5 |
| | antiCD15 | USBio | 5F17 |
| | antiCD15 | USBio | 8.S.288 |
| | anti CD15 | USBio | 0.N.80 |
| | Poly-LacNAc and/or glycolipid- | | |
| | specific Lex antibodies: | | |
| GF 518 | ab16285 (SSEA1) | Abcam | MC480 |
| | N-glycan specific Lex antibodies: | | |
| | Anti-Lex in neutral N-glycan | Lucka et al. Glycobiology 15: 87-100, | L5 |
| | | 2005 | |
| | Anti-Lex in neutral N-glycan | Lanctot et al. Current Opinion in | 3A8 |
| | | Chemical Biology 11, Issue 4, 2007, | |
| | | 373-380; Lanctot et al. 2006, Poster | |
| | | presentation in Glycobiology Society | |
| | | Meeting, Universal City, CA, poster | |
| | | 238 | |
| | Core 2 type O-glycan specific | | |
| | antibodies: | | |
| | Anti-Lex in Core 2 O-glycan | Sekine et al. Eur. J. Biochem. | SA024 |
| | | 268: 1129-1135, 2001 | |
| | Sulfo-Lex antibodies: | | |
| | antiCD15u = sulfoCD15 | USBio | 5F18 |
| | sLex antibodies: | | |
| GF 516 | 551344 anti-human CD15s | BD Pharmingen | CSLEX1 |
| GF 307 | MAB2096 (anti-sLewis X) | Chemicon | KM93 |
| | anti sLex | Seikagaku | 73-30 |
| | anti sLex | Meridianlifesciences | 258-12767 |
| | anti sLex | USBio | 2Q539 |
| | Core 2 O-glycan specific sLex | | |
| | antibodies: | | |
| GF 526 | MAB996 (anti-hP-selectin- | R&D systems | CHO131 |
| | glycoprotein ligand 1 ab) | | |

### REFERENCES

Arap, W., et al. (1998) Science 279(5349):377-80
Brockhausen, I. (1999) Biochim. Biophys. Acta 1473(1):67-95
Davies, et al. (1992) J. Chromatogr. 609(1-2):125-31
Domon, B. & Costello, C. E. (1988) Glycoconj. J. 5:397-409
Dube, D. H. & Bertozzi, C. R. (2005) Nat. Rev. Drug Discov. 4(6):477-88
Ernst, B., Hart, G. W., and Sinaÿ, P. (eds.) (2000) Carbohydrates in chemistry and biology, ISBN 3-527-29511-9, Weiley-VHC, Weinheim.
Fernandez, L. E., et al. (2003) Expert Rev. Vaccines 2(6):817-23
Harvey, D. J., et al. (1993) Rapid Commun. Mass Spectrom. 7(7):614-9
Holmberg, L. A. & Sandmeier, B. M. (2001) Expert Opin. Biol. Ther. 1(5):881-91
Huang, Y., et al. (2001) Anal. Chem. 73(24):6063-9
Kurokawa, T., et al. (2002) Eur. J. Biochem. 269:5459-73
Naven, T. J. & Harvey, D. J. (1996) Rapid Commun. Mass Spectrom. 10(11):1361-6
Nyman, T. A., et al. (1998) Eur. J. Biochem. 253(2):485-93
Papac, D. I., et al. (1996) Anal. Chem. 68(18):3215-23
Packer, N. H., et al. (1998) Glycoconj. J. 15(8):737-47
Plummer, T. H. Jr. & Tarentino, A. L. (1991) Glycobiology 1(3):257-63
Powell, A. K. & Harvey, D. J. (1996) Rapid Commun. Mass Spectrom. 10(9):1027-32
Saarinen, J., et al. (1999) Eur. J. Biochem. 259(3):829-40
Verostek et al. (2000) Anal. Biochem. 278(2): 111-22
Ylönen, A., et al. (2001) Glycobiology 11(7):523-3

1-97. (canceled)

98. A method of evaluating the malignancy of a patient sample comprising the step of detecting the presence or amount of cancer related glycan structures in the sample by determining the presence or amount of said glycan structure comprising any one of the structures from the following groups:
a) an O-glycan comprising N-acetyllactosamine Galβ4GlcNAc β6 or β3-linked to O-glycan core structure, optionally with the proviso that when the structures is core 2 sialyl-Lewis x the cancer is not primary colon cancer, or optionally,
when the structure is Galβ4GlcNAcβ6(Galβ3)GalNAcα or Galβ4GlcNAcβ6GalNAcα, then the cancer is not primary ovarian cancer;
b) a terminal mannose N-glycan
containing as non-reducing terminal monosaccharide residue or residues at least one Manα/β-residue(s) and optionally a non-reducing end branching Fucα6-residue and the core structure of the native glycan comprises Manβ4GlcNAcβ4GlcNAc,
c) glycan with nonreducing end terminal HexNAcβin said sample, selected from the group:
1) GlcNAcβor HexNAcβmonosaccharide is linked to another HexNAcβ monosaccharide forming a terminal HexNAcβHexNAcβ, optionally being further determined and it is neutral or sialylated di-N-acetyllactosediamine, (Neu5Acα)₀₋₁GalNAcβ4/3GlcNAcβ;
2) GlcNAcβ monosaccharide is linked to N-glycan core structure disaccharide epitope Manβ4GlcNAc, β2-linked to Manβ4, forming epitope GlcNAcβ2Manβ4;
3) GlcNAcβmonosaccharide is linked to N-glycan core structure disaccharide epitope Manβ4GlcNAc, 2-linked to GlcNAc of the epitope, forming epitope Manβ4(GlcNAc6)GlcNAc;
4) GlcNAcβmonosaccharide is linked to N-glycan core structure disaccharide epitope Manβ4GlcNAc, 4-linked to GlcNAc of the epitope, forming epitope GlcNAcβ4Manβ4GlcNAc, forming bisecting GlcNAc epitope;
5) N-glycan core marker structure is according to the Formula CGN:
wherein the Manβ4GlcNAc-epitope comprises the terminal GlcNAc substitutions, further optionally wherein the Manβ4GlcNAc-epitope comprises between 1-8% of the GlcNAc substitutions, or as defined in Tables, preferably 0-40% of the analyzed glycans.
wherein the detection is performed
i) by recognition of a terminal glycan epitope of the glycan by a specific binder reagent preferably by a monoclonal antibody, and/or
ii) by recognition of a characteristic monosaccharide composition corresponding to the glycan structures by chemical methods such as by mass spectrometry, and optionally further with specific binder molecule, and
iii) optionally by quantitative or relative quantitative mass spectrometric analysis, optionally further including glycome profile analysis according to the invention.

99. The method according to claim 98, wherein the O-glycan is sialylated or neutral, either:
a) neutral O-glycan oligosaccharide sequence having a structure of Formula:
Galβ4[(Fucα3)]ₙGlcNAcβX[(]ₘGalβ3[)]ₘGalNAc[αSer/Thr]ₚ
wherein p, n and m are either independently 0 or 1, [ ] indicates determinant either being present or absent depending on the value of m and n, ( ) indicates a branch in the structure.
X is 3, when m is 0; and X is 6 when m is 1; or
optionally when the structure is Galβ4GlcNAcβ6(Galβ3)GalNAcα or Galβ4GlcNAcβ6GalNAcα, then the cancer is not primary ovarian cancer;
b) sialylated O-glycan having a structure of Formula:
SAαX₁{Galβ4[(Fucα3)]ₙ₁GlcNAcβX₂}ₘGalβ4[(Fucα3)]ₙ₂GlcNAcβ6([SAαX₃]ₙ₃Galβ3) GalNAc[αSer/Thr]ₚ
wherein n1, n2, n3 and p are either independently 0 or 1 and m is 0, 1 or 2;
X₁, X₂, and X₃ are 3 or 6;
{ } and [ ] indicate determinant either being present or absent depending on the value of m, nil and n2; and
( ) indicates a branch in the structure,
optionally with the proviso that when n2 is 1 and m is 0 then the cancer is not primary colon cancer,
and X₂, and X₃ are preferably 3; X₁ is preferably 3, when n1 is 1; in preferred embodiment all X₁, X₂, and X₃ are 3;
preferably wherein the sialylated O-glycans have monosaccharide compositions SA₁₋₂HexₘHexNAcₘdHexₙ, wherein 2≦m≦4 and n<m, containing one or more terminal SAα3 residues;
and wherein the terminal O-glycan comprising GalβGlcNAc has a structure selected from the group consisting of: Galβ4(Fucα3)GlcNAcβ6(Galβ3)GalNAc,
Galβ4GlcNAcβ6(Galβ3)GalNAc, Galβ4(Fucα3)GlcNAcβ6GalNAc, Galβ4GlcNAcβ6GalNAc,
Galβ4(Fucα3)GlcNAcβ6(Galβ3)GalNAcαSer/Thr, Galβ4GlcNAcβ6(Galβ3)GalNAcαSer/Thr,
Galβ4(Fucα3)GlcNAcβ6GalNAcαSer/Thr, Galβ4GlcNAcβ6GalNAcαSer/Thr,
NeuNAcα3Galβ4(Fucα3)GlcNAcβ6(Galβ3)GalNAc,
NeuNAcα3/6Galβ4GlcNAcβ6(Galβ3)GalNAc, NeuNAcα3Galβ4(Fucα3)GlcNAcβ6GalNAc,
and NeuNAcα3/6Galβ4GlcNAcβ6GalNAc,
NeuNAcα3Galβ4(Fucα3)GlcNAcβ6(Galβ3)GalNAcαSer/Thr,
NeuNAcα3/6Galβ4GlcNAcβ6(Galβ3)GalNAcαSer/Thr,
NeuNAcα3Galβ4(Fucα3)GlcNAcβ6GalNAcαSer/Thr,
NeuNAcα3/6Galβ4GlcNAcβ6GalNAcαSer/Thr, or
other structure according to the formulae;
optionally with the proviso that when n4, n2 are 1 and m is 0, structure being NeuNAcα3Galβ4(Fucα3)GlcNAcβ6(Galβ3)GalNAcαSer/Thr, then the cancer is not primary colon cancer and optionally with the proviso that when m, n4, n3, n2 and p are 0, structure being Galβ4GlcNAcβ6(Galβ3)GalNAcα or Galβ4GlcNAcβ6GalNAcα, then the cancer is not primary ovarian cancer, or
c) wherein said N-glycan oligosaccharide sequence has a structure of Formula:
[Manα2]ₙ₁[Manα3]ₙ₂([Manα2]ₙ₃[Manα6)]ₙ₄)[Manα6]ₙ₅([Manα2]ₙ₆[Manα2]ₙ₇[Manα31]ₙ₈)Manβ4GlcNAc4[(Fucα6)]ₘGlcNAc[β-N-Asn]ₚ
wherein p, n1, n2, n3, n4, n5, n6, n7, n8, and m are independently either 0 or 1; with the proviso that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0; the sum of n1, n2, n3, n4, n5, n6, n7, and n8 is less than or equal to (m+3); and preferably n1, n3, n6, and n7 are 0 when m is 0; [ ] indicates determinant either being present or absent depending on the value of n1, n2, n3, n4, n5, n6, n7, n8, and m; and
( ) indicates a branch in the structure.

100. The method according to claim 98, wherein the glycan is recognized by a specific binding agent or binder molecule specific for a terminal non-reducing end structure of the glycan comprising at least two monosaccharide residues and glycosidic linkage between them, for analysis or diagnosis of cancer and/or for treatment of cancer; wherein the binder is selected from the group of: recombinant proteins, peptides, antibodies and lectins; substance binding to said oligosaccharide sequence is an aptamer, a peptide or a protein; an antibody, an enzyme, a lectin or a fragment thereof; aptamer, an enzyme, a human or humanized humanized antibody, a peptide, antibody fragment, or antibody derivative; and wherein the glycan is expressed in binder accessible form in a cancer or on a human cancer or the glycan is expressed in a cancer metastasis

101. The method according to claim 100, wherein the binder recognizes at least one terminal epitope either according to:
the Formula TE1
[SAαX₁]ₙ₄Galβ4[(Fucα3)]ₙ₂GlcNAc{β3Galβ[(Fucα3)]_{n1[}4GlcNAc]ₙ₅}_{m{}β6[GalNAc]ₙ₆}ₙ₇
or according to the Formula TE2
[SAαX₁]ₙ₄Galβ4[(Fucα3)]ₙ₂GlcNAc[β]ₙ₈,
or according to the formula TE3
[SAαX₁]ₙ₄Galβ4[(Fucα3)]ₙ₂GlcNAcβ6[GalNAc]ₙ₆,
or according to the formula TE4
[SAαX₁]ₙ₄Galβ4[(Fucα3)]ₙ₂GlcNAcβ6{([SAαX₃]ₙ₃Galβ3)}ₙ₉[GalNAc]ₙ₆,
or according to the formula TE5
[SAαX₁]ₙ₄Galβ4[(Fucα3)]ₙ₂GlcNAc{β3Galβ[(Fucα3)]_{n1[}4GlcNAc]ₙ₅}ₘ,
wherein n1, n2, n4, n5, n6 and n7 and p are either independently 0 or 1 and m is 0, 1 or 2 (repeating structure);
X₁, X₂, and X₃ are 3 or 6;
{ } and [ ] indicate determinant either being present or absent depending on the value of m, n1, n2, n3, n4, n5, n6, n7, n8, and n9; and
( ) indicates a branch in the structure;
or wherein the binder recognizes at least one terminal Manα6/Manα3/Manα/Manβepitope according to the Formula TEM1:
[Manα6]ₙ₁([Manα3]ₙ₂)Man[α]ₘ₂[β]_{n3[}4GlcNAcβ]ₙ₄[(Fucα6)]_{m[}4GlcNAc]ₙ₅[β-N-Asn]ₚ
or according to the Formula TEM2:
[Manα6]ₙ₁([Manα3]ₙ₂)Man,
or according to the Formula TEM3:
[Manα6]ₙ₁([Manα3]ₙ₂)Manα,
wherein the variables are as in Formula TEM1.
or according to the Formula TEM4:
[Manα6]ₙ₁([Manα3]ₙ₂)Manβ,
or according to the Formula TEM5:
[Manα6]ₙ₁([Manα3]ₙ₂)Manβ4G cNAcβ[(Fucα6)]_{m[}4GlcNAc]ₙ₅,
wherein the variables are as in Formula TEM1;
preferentially corresponding to a terminal epitope selected from the group consisting of:
GalβGlcNAc, Galβ4GlcNAcβ, Galβ4GlcNAcβ6, Galβ4GlcNAcβ6GalNAc,
Galβ4GlcNAcβ6(Galβ3)GalNAc, Galβ4GlcNAcβ6GalNAcα,
Galβ4GlcNAcβ6(Galβ3)GalNAcα, Galβ4(Fucα3)GlcNAc, Galβ4(Fucα3)GlcNAcβ,
Galβ4(Fucα3)GlcNAcβ6, Galβ4(Fucα3)GlcNAcβ6GalNAc, Galβ4(Fucα3)GlcNAcβ6(Galβ3)GalNAc, Galβ4(Fucα3)GlcNAcβ6GalNAcα,
Galβ4(Fucα3)GlcNAcβ6(Galβ3)GalNAcα, NeuNAcα3GalβGlcNAc,
NeuNAcα3Galβ4GlcNAcβ, NeuNAcαGalβ4GlcNAcβ6, NeuNAcα3Galβ4GlcNAcβ6GalNAc,
NeuNAcα3Galβ4GlcNAcβ6(Galβ3)GalNAc, NeuNAcα3Gal(34GlcNAcβ6GalNAcα,
NeuNAcα3Galβ4GlcNAcβ6(Galβ3)GalNAcα,
NeuNAcα3Galβ4(Fucα3)GlcNAc, NeuNAcα3Galβ4(FucαC3)GlcNAcβ,
NeuNAcα3Galβ4(Fucα3)GlcNAcβ6, NeuNAcα3Galβ4(Fucα3)GlcNAcβ6GalNAc,
NeuNAcα3Galβ4(Fucα3)GlcNAcβ6(Galβ3)GalNAc,
NeuNAcα3Galβ4(Fucα3)GlcNAcβ6GalNAcα,
NeuNAcα3Galβ4(Fucα3)GlcNAcβ6(Galβ3)GalNAcα,
Manβ, Manβ4GlcNAc, Manβ4GlcNAcβ, Manβ4GlcNAcβ4GlcNAc,
Manβ4GlcNAcβ4(Fucα6)GlcNAc, Manβ4GlcNAcβ4GlcNAcβ,
Manβ4GlcNAcβ4(Fucα6)₀ₒᵣ₁GlcNAcβ, Manβ4GlcNAcβ4GlcNAcβAsn,
Manβ4GlcNAcβ4(Fucα6)GlcNAcβAsn,
Manα3Man, Manα6Man, Manα3Manβ, Manα6Manβ, Manα3Manα, Manα6Manα,
Manα3 (Manα6)Man, Manα3 (Manα6)Manβ, and Manα3 (Manα6)Manα.

102. The method according to claim 98, wherein preferably the method involve mass spectrometric profiling of a cancer sample and determining monosaccharide compositions of both O-linked and N-linked glycans, preferably using internal standard molecule.

103. The method according to claim 98, wherein high relative expression rate of at least two oligosaccharide sequences, of which sequences at least two are selected from different groups a), b) or c), is indicative of malignant cancer; and when the method is used to evaluate the malignancy of ovarian tumor, a low relative expression rate of oligosaccharide structures with HexNAcβHexNAcβor di-N-acetyllactosediamine sequences is indicative of malignant ovarian tumor.

104. A method according to claim 98, wherein the detection comprises:
(a) contacting said patient sample with a substance binding to said oligosaccharide sequence, and determining the presence of a combination of said substance and said sample, or
(b1) releasing the oligosaccharide structures of said biological sample by enzymatic or chemical methods to form a fraction containing free oligosaccharide structures or conjugates from said sample,
(b2) determining the presence of said oligosaccharide sequences in said fraction, or
(b3) determining the relative amounts of said oligosaccharide sequences in said fraction compared to other oligosaccharide sequences present in said fraction;
wherein said biological sample is selected from the group of a blood, tissue or serum sample, a secreted glycoprotein, or other biological sample.

105. A glycan substance, optionally produced according to claim 104, comprising cancer associated oligosaccharide structures as defined in the Tables, Figures, Examples, Description, and claims of the invention; optionally comprising O-glycans of Cal 5-3 antigen for use as a standard as described in claim 98.

106. The antigenic or immunogenic substance according to claim 105 comprising one or several cancer glycan sequences, optionally in a chemically or biochemically synthesized polyvalent form, for uses selected from the group of: immunization in humans, producing polyclonal or monoclonal antibodies, purification of antibodies from serum, preferably from human serum, and detection and/or quantitation of antibodies.

107. The pharmaceutical composition according to claim 105 comprising one or several oligosaccharide sequences, or analogs or derivatives thereof for the treatment of cancer.

108. A binder substance, optionally produced according to claim 104, comprising binder against one or several of the oligosaccharide sequences as described in the invention, selected from the group of: antibody, monoclonal antibody, antibody fragment, antibody derivative, an aptamer, an enzyme, a humanized antibody, a peptide or other binder molecule; optionally comprising a standardized recombinant Ca15-3 antibody binding O-glycans as described in claim 98.

109. The substance according to claim 108, optionally comprising an antibody against one or several of the oligosaccharide sequences, for use as a diagnostic agent for the diagnosis of cancer or a cancer type or for the treatment of cancer, optionally comprising a pharmaceutically acceptable carrier and optionally an adjuvant, optionally when
administered to a human or animal patient in need of treatment in an amount sufficient to reduce the metastatic potential or growth of cancer cells or to eliminate a tumor or cancer, optionally wherein the antibody is human or humanized antibody; and optionally when said antibodies target a toxic agent or toxic agents to a tumor or cancer.

110. The immunogenic substance according to claim 107 for use as a cancer vaccine, optionally
comprising a pharmaceutically acceptable carrier and optionally an adjuvant; optionally according to Formula
[OS—(X)ₙ-L-Y]ₘ-Z (II),
wherein OS is an oligosaccharide sequence according to the invention, Y is a non-carbohydrate spacer or a non-glycosidically linked terminal conjugate, n is 0 or 1 and X is lactosyl-, galactosyl-, N-acetyllactosaminyl, mannosyl-, Man₂, Man₃-, Man₃GlcNAc, Man4GlcNAc, N-acetylglucosaminyl-, or N-acetylgalactosaminyl, preferably X is lactosyl-, galactosyl-, mannosyl-, or N-acetylgalactosaminyl and OS is β2-, or β4-, or β6 linked to the mannosyl residue, more preferably OS is β2-; most preferably OS is β3- or β6-linked to galactosyl residue or N-acetylgalactosaminyl residue or Gal-residue of lactose or N-acetyllactosamine for the treatment of cancer; optionally wherein the antigenic epitope structure is a recombinant terminal Man-glycan comprising protein produced in yeast of fungi,
and/or
a KLH-protein enriched with glycans comprising low-Man glycans, preferably a natural KLH-protein digested with a β-galactosidase enzyme.

111. The glycome composition according to claim 105 comprising cancer oligosaccharide structures further in complex with MALDI matrix, preferably for use in cancer analysis.

112. Method according to claim 98 for detecting cancer or cancer metastasis, preferentially gastrointestinal cancer, stomach cancer, ovarian cancer, breast cancer, or lymph node metastasis, based on detecting ABO blood group antigen expressing glycans, wherein the detected glycan structures are according to formula:
Fucα[Gal(NAc)_{z}α3]_{y}[(GalβGlcNAcβ)ₘGalβ3GalNAc], wherein m, x, y, and z depend on the
subject's ABO blood group according to the following rules:
in A blood group subjects, 1≦m≦2, 1≦x≦2. 0≦y≦1, and z=1; or
in B blood group subjects, 1≦m≦2, 1≦x≦2, 0≦y≦1, and z=0, or when m=0 then 1≦x≦2, 0≦y≦1, and z=0; and
additionally in all subjects with any ABO blood group, 1≦m≦2, 1≦x≦2, and y=z=0.

113. Method according to claim 112, wherein the detected glycan structures are selected according to the ABO blood type from the group of:
i) in all ABO blood group subjects non-reducing terminal Fucα2Galβand/or
Fucα2GalβGalNAc epitopes;
II) in A blood group subjects also non-reducing terminal Fucα2(GalNAcα3)Galβand/or Fucα2(GalNAcα3)Galβ3GalNAc epitopes; and
iii) in B blood group subjects also non-reducing terminal Fucα2(Galα3)Galβand/or
Fucα2(Galα3)GalαGalNAc epitopes;

114. The method according to the claim 112, wherein the oligosaccharide structures are selected from the group of:
i):
A1{Galβ4/3[(Fucα4/3)]ₙ₁GlcNAcβ3}ₘGalβ4[(Fucα3)]ₙ₂GlcNAcβ6([A2]ₙ₃Galβ3)ₙ₄GalNAc[αSe r/Thr]ₚ
ii) A1Galβ4[(Fucα3)]ₙ₂GlcNAcβ[6]ₙ₄[GalNAc]ₙ₃[αSer/Thr]ₚ,
preferably
[Gal(NAc)ₙα3]ᵣ(Fucα2)Galβ4GlcNAcβ6
[Gal(NAc)ₙα3]ᵣ(Fucα2)Galβ4(Fucα3)GlcNAcβ6GalNAc
[Gal(NAc)ₙα3]ᵣ(Fucα2)Galβ4(Fucα3)GlcNAcβ6GalNAc, and
[Gal(NAc)ₙα3]ᵣ(Fucα2)Galβ4GlcNAcβ6GalNAc
iii) A1{Galβ4/3[(Fucα4/3)]ₙ₁GlcNAcβ3}ₘ{Gal[β4[(Fucα3)]ₙ₂GlcNAcβ6]ₙ₄}ₙ₆
wherein n6 is 0 or 1,
either preferably type II N-acetyllactosamine epitopes
[Gal(NAc)ₙα3]ᵣ(Fucα2)Galβ4(Fucα3)GlcNAcβ3Gal
GalNAcα3 (Fucα2)Galβ4(Fucα3)GlcNAcβ3Gal
Galα3 (Fucα2)Galβ4(Fucα3)GlcNAcβ3Gal
Fucα2Galβ4(Fucα3)GlcNAcβ3Gal
[Gal(NAc)ₙα3]ᵣ(Fucα2)Galβ4GlcNAcβ3Gal
GalNAcα3 (Fucα2)Galβ4GlcNAcβ3Gal
Galβ3 (Fucα2)Galβ4GlcNAcβ3Gal
Fucα2Galβ4GlcNAcβ3Gal
or
Preferably type I N-acetyllactosamine epitopes
[Gal(NAc)ₙα3]ᵣ(Fucα2)Galβ3(Fucα4)GlcNAcβ3Gal
GalNAcα3 (Fucα2)Galβ3(Fucα4)GlcNAcβ3Gal
Galα3(Fucα2)Galβ3(Fucα4)GlcNAcβ3Gal
Fucα2Galβ3(Fucα4)GlcNAcβ3Gal (elongated Lewis b)
[Gal(NAc)ₙα3]ᵣ(Fucα2)Galβ3GlcNAcβ3Gal
GalNAcα3 (Fucα2)Galβ3GlcNAcβ3Gal
Galα3)(Fucα2)Galβ3GlcNAcβ3Gal
Fucα2Galβ3GlcNAcβ3Gal.
iv) core 1 O-glycans according to the Formula [Gal(NAc)ₙα3]ᵣ(Fucα2)Galβ3GalNAc[αSer/Thr]ₚ
wherein n, p, and r are 0 or 1 independently, preferably A,B and O (H)-antigens
GalNAcα3 (Fucα2)Galβ3GalNAc[αSer/Thr]ₚ
Galα3 (Fucα2)Galβ3GalNAc[αSer/Thr]ₚ
Fucα2Galβ3GalNAc[αSer/Thr]ₚ.
wherein A1 and A2 are independently [Gal(NAc)ₙα3]ᵣ(Fucα2)
n, n1, n2, n3, n4, p and r are either independently 0 or 1 and m is 0, 1 or 2;
{ } and [ ] indicate determinant either being present or absent depending on the value of m, n1 and n2; and
( ) indicates a branch in the structure, with the provision that preferably n is either 0 or 1 for both A1 and A2 or
non-reducing end terminal structure derived thereof.

115. The method according to claim 112, wherein carcinomas of the gastrointestinal tract and reproductive tissues, preferably stomach, colon, ovarian, cervical, and breast cancer, are recognized by high expression of especially type I and type I blood group or Lewis antigens, preferably A, B, H, and Lewis b-antigens, as described in claim 114; optionally these structures are detected by blood group/Lewis antibodies according to the invention.

116. The method according to claim 98 for recognizing LacdiNac structure by preferred antibody clone according to the invention, preferably selected from the group of: clone 259-2A1 (IgG3, against LacdiNAc); clones 273-3F2, 114-2H12-C, 100-2H5-A, and 99-2A5-B (IgM, against LacdiNAc); and clones 204-6A1, 290-2E6, and 294-2A1 (IgM, against α3Fuc-LacdiNAc; van Remoortere et al., 2000. Glycobiology 10, 601-609);
or recognizing Low-mannose glycan, wherein low-mannose type N-glycan specific antibody clone 100-4G11-A (mouse IgM, van Remoortere et al., 2003. Glycobiology 13, 217-225); clones L3 and L4 (Schmitz, B., et al., 1993. Glycobiology 3, 609-617); and Manα3Manβ4GlcNAc2 low-mannose type N-glycan specific antibody clone OMR5 and Manα6Manβ4GlcNAc2 low-mannose type N-glycan specific antibody clone OMR6;
or recognizing terminal GlcNAcβrecognition according to the invention including clone HGAC85, recognizing β-linked GlcNAc in oligo/polysaccharides and in O-GlcNAc (Nahm, M. H., et al., 1982. J. Immunol. 129, 1513-1518);
or recognizing Lex/sLex epitopes on different glycan backbones preferably by antibodies listed in Table 21, more preferably in the prioritized order of the Table.

117. The method according to claim 109 for detecting, imaging or treating cancer, tumors, and metastases by use of antibodies and other binding reagents recognizing LacdiNAc structures in human tumors, preferably in ovarian tumors and metastases thereof; preferably by antibodies.

118. A method of treatment of cancer by immunotherapy using molecules selected from the group of:
i) a binder substance according to claim 109, preferentially antibody for passive immunotherapy;
ii) immunogenic cancer vaccine substance according to claim 109 for active immunotherapy; or
iii) specific inhibitors of sialic acid biosynthesis, optionally reducing the metastatic potential and/or malignancy of the cancer cells.
